(19) United States
(12) Patent Application Publication
(10) Pub. No.: US 2024/0282425 A1
(43) Pub. Date: Aug. 22, 2024

(54) AUTHENTICATION METHODS AND SYSTEMS FOR DISPENSED PRESCRIPTIONS

(71) Applicant: Gordon Keith Low, Salt Lake City, UT (US)

(72) Inventor: Gordon Keith Low, Salt Lake City, UT (US)

(21) Appl. No.: 18/444,865

(22) Filed: Feb. 19, 2024

Related U.S. Application Data
(60) Provisional application No. 63/446,812, filed on Feb. 18, 2023.

(51) Int. Cl. G16H 20/13; G06K 7/14; G06K 19/06; G06K 19/07; G06Q 50/00; G16H 10/60; G16H 40/63

(52) U.S. Cl. CPC G16H 20/13; G06K 7/1417; G06K 19/06037; G06K 19/0723; G06Q 50/01; G16H 10/60; G16H 40/63

(57) ABSTRACT

The present disclosure contemplates a system and a medical device such as a smartphone or tablet that can operate as a secure medical device for tracking consumption of medications dispensed to patients by gathering and creating medical quality data about consumed medications that are accurate, precise, legally binding, and reliable. This medical quality data can be securely stored in a patient's digital medication diary.

### CROSS-REFERENCE TO RELATED APPLICATIONS

This application claims the benefit of U.S. Provisional Patent Application No. 63/446,812 filed on Feb. 18, 2023; which is herein incorporated by reference in entirety.

### FIELD OF INVENTION

The present invention relates to a computerized medical device, system, and method for helping patients to take their medications in the manner prescribed by their healthcare providers, to increase their persistence and adherence, and to a system and method to facilitate using smartphones and tablets to track actual dose consumption, and so forth.

### BACKGROUND OF THE INVENTION

The current systems available for dispensing, tracking, and consumption of prescription drugs are limited in scope with regards to improving compliance of patients to use their prescription drugs when they should. Once a patient leaves the pharmacy with their dispensed prescription drugs, there are no controls and limited feedback and information about whether and when dispensed drugs are consumed. As a result, prescription drugs are often not taken as prescribed, with some studies showing that only about half of prescribed drugs are taken properly. Some patients do not take their drugs as long as prescribed (persistency), and some do not take the right dose at the right time (adherence). As a result, too many patients experience negative medical events, some severe, including death. The cost to the healthcare system and the economy of these unnecessary severe medical events are estimated to exceed $250 billion annually.

Current smartphones and tablets are powerful in their ability to help individuals and groups to manage a variety of complex and interrelated activities and processes, including in healthcare. However, their ability to help manage prescriptions dispensed to patients is limited because, while there is intense tracking of drug from manufacturing through to the dispensing pharmacists, there is little tracking beyond that point. Once dispensed there is limited ability to keep track of individual doses of medication. Absent this ability to monitor and track individual doses and their consumption, it is difficult to rely on other information and measures which only approximate consumption of medication.

The present application seeks to provide solutions to some of the above problems, by enabling smartphones and tablets to become medical devices which can enable increasing patient persistence and adherence for prescription drugs, which in turn can improve health care outcomes and reduce healthcare system costs.

### SUMMARY OF THE INVENTION

The smartphone or tablet can become a secure medical device for medications dispensed to patients by gathering and creating medical quality data about consumed medications that are accurate, precise, legally binding, and reliable. This medical quality data can be securely stored in a patient's digital medication diary. This medical device for prescriptions dispensed to patients can use a patient's medication diary to help the patient to take the right drug at the right time through reminders and through the support of caregivers, family, friends, and medical providers. The medical device for prescriptions dispensed to patients can also provide personal or team games to encourage medication persistence and adherence. Financial incentives can also be initiated and tracked through the medical device for prescriptions dispensed to patients. The effect of patients' improved persistence (not stopping) and adherence (taking the right dose at the right time) can be improved medical outcomes for patients and lower healthcare system cost through fewer harmful and costly medical events. The medical device for prescriptions dispensed to patients can also be used for medical applications relative to the dispensed drug, including communicating with healthcare providers and prescription renewal. The medical device for prescriptions dispensed to patients can also enable positive collective activity including medication research, reduction of health care premiums, and social networking around common medications or conditions. The objective of this innovative method and device is to improve the health of patients and reduce overall healthcare costs.

### BRIEF DESCRIPTION OF DRAWINGS

FIGS. 1A-1C are block diagrams depicting embodiments of computers, databases and networked systems useful in connection with the methods and systems described herein;

FIG. 2 illustrates a flowchart of a method of using the medical device for prescriptions dispensed to patients;

FIG. 3 illustrates a screenshot of various patient attest statements;

FIGS. 4A-4B illustrate dispensed prescription labels;

FIGS. 5A-C illustrate various views of dose containers used to store and then later dispense medications;

FIGS. 6A.1-6C illustrate examples of authenticator indicia and positions where the authenticator indicia may be placed on a dose container;

FIGS. 6D.1-6D.2 illustrate yet another example of an authenticator indicia being placed on to the dose of medication itself;

FIGS. 6E.1-6E.2 illustrate yet another example of an authenticator indicia being placed on a multi-dose/vitamin/supplement dose container;

FIG. 7 illustrates a flowchart of a method of authenticating a dose of medicine;

FIGS. 8A-C are various diagrams illustrating steps and systems associated with method and system for authenticating a dose of medicine;

FIGS. 9A-C illustrates various screenshots of a medical device interactions with the patient as they interact with a dose of medicine and establish a secure communication between patient and doctor.

### DETAILED DESCRIPTION OF THE INVENTION

The detailed description set forth below in connection with the appended drawings is intended as a description of presently preferred embodiments of the invention and does not represent the only forms in which the present disclosure may be constructed and/or utilized. The description sets forth the functions and the sequence of steps for constructing and operating the invention in connection with the illustrated embodiments.

One of the objectives of the present invention is to provide assistance to help patients take their medications as prescribed by tracking consumption of each dose. This is of particular importance for those who are reliant on their medications for ongoing health, including those with chronic conditions, the elderly, and those with mental health conditions. Another objective of the present invention is to establish a secure communication channel with patients that can further be utilized to communicate with medical providers and support persons.

What is needed is a system and method that can create accurate and reliable information about each individual medication dose as it is consumed. This accurate medical quality information can then be relied upon by the patient, caregivers, family, friends, healthcare providers, and third parties for medical and other legitimate purposes. It can also be incorporated into a secure application which enables the smartphone to become a computerized medical device which can advise patients and third parties.

This can all begin with the creation of a precise and reliable online medications diary. The precision and reliability of the medication diary can enable the medical device to initiate activities which can increase persistence and adherence for medications dispensed to patients, including for generic or branded drugs, including for the drugs and the indication(s) for which each drug is approved listed in the 44^{th} edition (2024) of the FDA's Orange Book of Approved Drugs with Therapeutic Equivalence Evaluations which is incorporated herein in its entirety. A medications diary can be an accurate and authentic record of medications taken by a single patient, including a historical record of medications taken corresponding to the patient. The medications diary is stored on a memory accessible to the computerized medical device. Combining many medications diaries together can create a medications database. The medications diary and medications database can reside in the cloud, in a secure, HIPAA compliant database. It can only be accessed by the patient and those who have been given authorization by the patient.

The medications diary can include the data needed to track, measure, and manage dispensed medication use. It can include the patient's name and other identifying personal information, for example, date of birth, phone number, address, email, insurance information, and demographic information. It can include information about the patient's dispensed prescription, including prescriber identification, drug name, drug strength, dosage form, quantity prescribed, directions for use, date dispensed, quantity dispensed, dispensing pharmacy, dispensing pharmacy prescription number, and dispensing pharmacy phone number.

The medical device for prescriptions dispensed to patients can track that each dose of the dispensed medication was actually consumed at a specific date, time, and location. This can be done with a legally binding attestation by the patient. The legal attestation can be supported by evidence of the dose of medication and evidence of consumption.

As discussed further herein, there is a problem in the field of a lack of persistence and adherence to medicine dosing regimens. There is also a problem in the computerized field that it is easy to lie, manipulate, trick and otherwise fake or hack computerized systems due to a lack of verification and confirmation. This is a problem in all aspects of the computerized field as a computer system is only able to receive information and it is difficult to verify. The present invention solves this computerized problem in the medicine administration context by requiring evidence and/or attestation of taking the medicine, in addition to a simple confirmation which can be easily taken advantage of by a dishonest user.

The gathered evidence to confirm the dose of the medication may be done in any manner to confirm the taking of the medicine, a number of non-limiting examples follow. In one case, evidence of the dose of medication can be generated by using the medical device to take a picture of the medication consumed. Stronger evidence can be generated by having the patient enter into the device the pharmacy phone number and prescription number by scanning or manual input. For prescriptions which are dispensed in the manufacturer's packaging, even stronger evidence can be created by the patient using the camera of the medical device to scan the DSCSA GS1 datamatrix or manually the entering the dispensed package GTIN, serial number, expiration date, and lot number using the user input interface of the medical device. The strongest can be generated by a multi-factor dose authentication, where each dose is enclosed in an authenticable enclosure which requires an outside scan and possibly an inside scan. Multiple medications, vitamins and supplements can be sealed in the same enclosure, so long as they are to be consumed at the same time. The dose linkage to the legitimate prescription(s) for each patient can help confirm authenticity. Evidence of medication authenticity and dose consumption can be gathered through continuous video recording, which can start before outside authenticator scanning. In some embodiments, an artificial intelligence engine can review the recorded video to confirm that the medicine is taken. It can continue with breaking of the seal, removing the medication(s) from the enclosure, scanning the inside authenticator, and consuming of the medication(s). When multi-factor authentication is used with recording, attestation of the drug and consumption is also recorded at this point. The continuous recording can stop only after the patient shows that the medication(s) has been consumed. For example, with the continuous video of the smartphone medical device recording, the patient can swirl their tongue around their mouth and then open their mouth wide to show that the mouth is empty. Both front and back continuous video can be taken. If smartphone capability is limited, the two videos can be recorded interlaced at 15 fps each. The continuous video can include integrated audio using smartphone's microphone(s). In situations where there are questions about drug or consumption authenticity, all aspects of the recorded interaction can help to resolve uncertainties. Partial authentication of dose consumption can also be recorded well as non-consumption dose scans.

In a particular embodiment of creating a video record of medicine consumption, a user may use one or both of a front camera and a back camera of the computerized medical device, in this case a smartphone or tablet, using the camera(s) individually or simultaneously. The recording can start at the very beginning of the process, i.e. first taking the packaging of the medication, and record continuously through to a final attestation, and create a digital record of the evidence, the digital record being a data file saved on a memory accessible to the computerized medical device.

Inside and outside authenticators can provide evidence for two factor drug authentication. There can be a sealable enclosure for each dose or for multiple medications, vitamins, or supplements taken at the same time. Breaking of the seal can render a dose not authenticatable. Each authenticator can contain unique alpha-numeric characters. Authenticators can be scannable to internet addresses. Outside and inside authenticators can be linked to each other during packaging and recorded in the cloud or other accessible database.

Multi-factor authenticatable enclosures can be prepared by a pharmaceutical manufacturer. Authenticable doses can be linked to the sealed package DSCSA GS1 Data Matrix, which can typically be tracked through the supply chain through to the pharmacist. The pharmacist can link the DSCSA GS1 Data Matrix to the prescription number at time of dispensing, this linking can be recorded in the cloud or other accessible database.

Multi-factor authenticatable enclosures can also be prepared by the dispensing pharmacist. The pharmacist can place a single dose or multiple medications to be taken at the same time in an authenticatable enclosure which can have preprinted outside and inside authenticators. Outside and inside authenticators can be associated and recorded in a database when they were manufactured before they are supplied to the pharmacist. Outside authenticators can be linked to the dispensed prescription number by the pharmacist. Automation of the scanning and filling process can be used to improve efficiency.

The computerized medical device together with the digital medications diary created for patients and populations can improve our society by improving health care outcomes, improving economic results, and abating illegal drug trafficking activity. The medical device for prescriptions dispensed to patients can remind the patient when to take their medication. Specifically, the computerized medical device may be programmed to provide an output based on data in the medications diary of suggestions about taking medications, the suggestions based on prescription, medications history, and current date and time. Suggestions may include, but are not limited to audible reminders, reminders, suggestions, or tips presented on the display and/or as push notifications on the smartphone medical device, haptic reminders such as a vibration, automated messages to other devices such as a text message to a spouse, child, parent, caregiver, and the like, and information in these notifications to encourage habit forming behavior such as encouraging taking the medication along with a common event such as a meal, before bed, upon waking, after a shower, and so forth. These suggestions may be generated by a system server and sent via a network to the computerized medical device, or the medical device itself may be programmed to generate them based on the input information, and data gathered by one or more inputs received by the medical device. If patients are late in taking their medications, the medical device for prescriptions dispensed to patients can notify caregivers, family, or healthcare providers, who can contact the patient through the medical device to remind them to take the medication. Caregivers, family, or healthcare providers can also take other steps outside the medical device for prescriptions dispensed to patients to intervene as appropriate. When patients properly take their medications, caregivers, family, or healthcare providers can also provide positive reinforcement back to the patient through the medical device for prescriptions dispensed to patients. Through the medical device for prescriptions dispensed to patients, the patient can also be motivated through individual or team gamification. Gamification examples include, but are not limited to, individual or team games for patients to play on the computerized medical device which maintain or improve patience persistence and adherence. Through the medical device for prescriptions dispensed to patients, the patient can also receive rewards and/or financial incentives. In one embodiment, these incentives are presented to the patient via the user interface of the computerized medical device, and may be initiated via a prompt on a display of the computerized medical device and a receipt of an input to the user input interface, which can be relayed through third party networks by the computerized medical device. The result of these activities to increase persistency and adherence can provide patients with improved medical outcomes and lower overall medical costs. These reduced medical costs, typically for medical insurance companies, can in turn result in reduced premiums for individuals or group insurers, which are often employers.

The medical device for prescriptions dispensed to patients can enable remote monitoring and, where needed, control of patients taking medications. This can result in improved care given to those who are elderly or mentally compromised. It can also enable doctors to use automation to closely monitor the ongoing health of their patients on medications.

The medical device for prescriptions dispensed to patients can provide the most accurate medication consumption data to improve the quality of drug research. Researchers can know the exact drug, dose, date, and time of consumption by accessing the recorded data by the system. They can verify the authenticity of the drug and consumption of the medication from watching the multi-factor authentication process video which is recorded and saved as part of the patient record. The medical device for prescriptions dispensed to patients can enable a direct and immediate 2-way feedback channel between the patient and researchers. As such, the system is able use the gathered data, as well as inputs into the computerized medical device to enable a patient or population of patients the option to participate in medical research for conditions treated with prescription drugs.

The medical device for prescriptions dispensed to patients can enable the safer dispensing of controlled substances, like opioids. This can reduce the likelihood of addiction and provide a method of controlling the proper return or destruction of medications when the patient is finished their treatment. This can result in improved medical outcomes and a direct link to healthcare practitioners if issues arise. The medical device for prescriptions dispensed to patients can help those recovering from addiction to drugs, including opioids, to track their progress and communication directly with caregivers, family, friends, and healthcare providers.

The medical device for prescriptions dispensed to patients can abate controlled medications trafficking, especially opioids by tracking scans of all doses. It can also abate counterfeit medication production and distribution.

The medical device for prescriptions dispensed to patients can foster common medication and disease communities enabling patients and family to connect, share, and improve their experience with specific medications and disease conditions.

The data in each digital medication diary is owned by the patient. The patient may authorize others to have access to all or part of the data in their medication diary. The patient may revoke their authorization(s). Examples of authorizations to give access to others include caregivers, family, friends, healthcare providers, medical researchers or healthcare insurers.

Other objectives will also be noted to those of ordinary skill in the art as the various embodiments of systems and methods are described below. In some embodiments, the methods and systems described herein relate to dispensed medication tracking, increasing persistency, increasing adherence, and improving healthcare outcomes, and reducing healthcare costs. Before describing such methods and systems in detail, however, a description is provided of a computer and a network in which such methods and systems may be implemented.

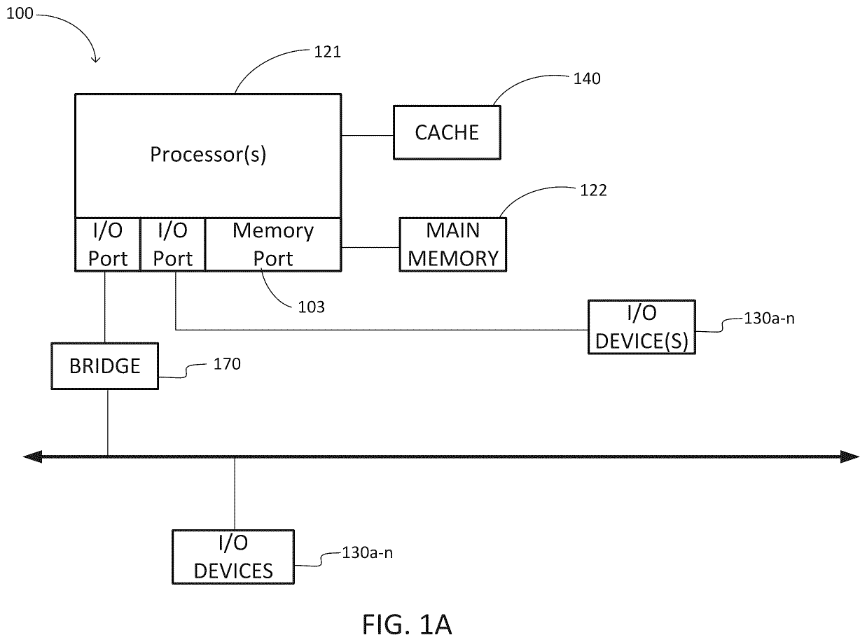

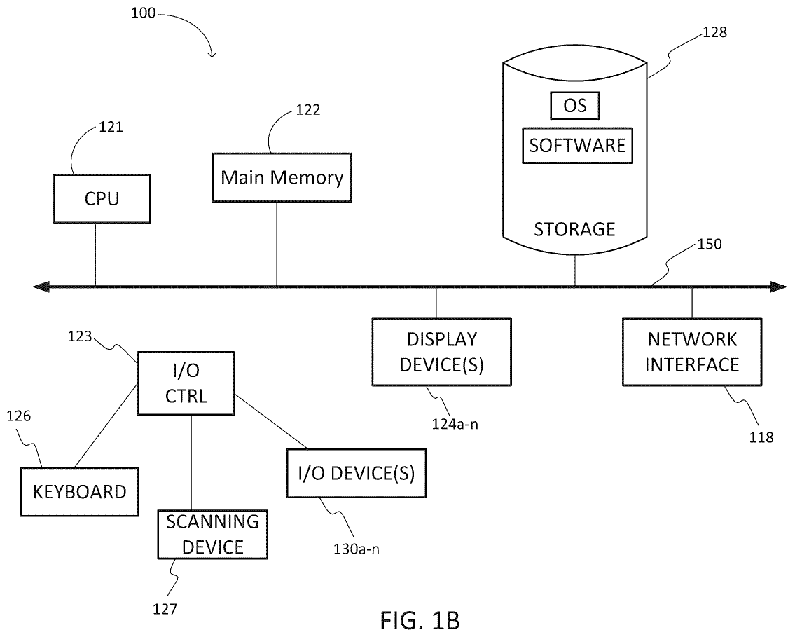

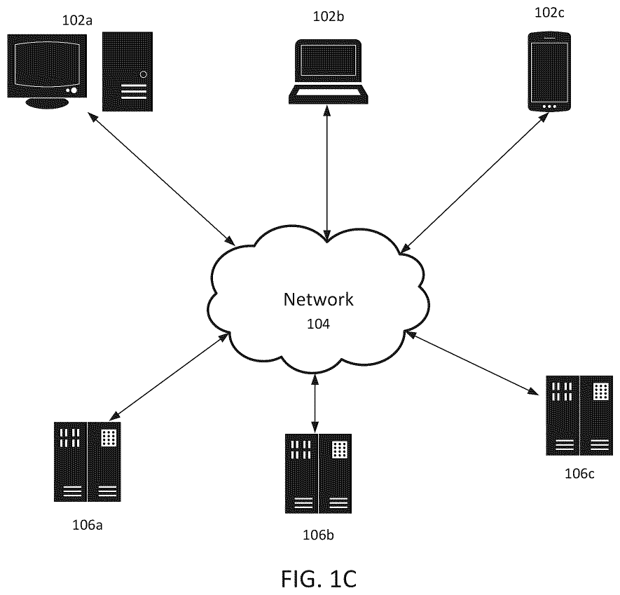

Referring to FIGS. 1A and 1B depict block diagrams of components of the computerized medical device, or smartphone/tablet configured as a computerized medical device contemplated in this disclosure and discussed throughout this disclosure. A computing device 100 used herein as the computerized medical device may utilize the user interface 102a-c and/or a computing device 106a-c, shown in FIG. 1C. As shown in FIGS. 1A and 1B, each computing device 100 includes a central processing unit 121, and a main memory unit 122. As shown in FIG. 1B, a computing device 100 may also include a storage device 128, a network interface 118, an I/O controller 123, display devices 124a-n, a keyboard 126 which may be in the form of a digital keyboard presented on a touch screen display, a scanning device 127, such as a camera(s) or 2D barcode scanner, and one or more other I/O devices 130a-n such as a mouse, trackpad, pen, RFID reader/writer and so forth. The storage device 128 may include, without limitation, an operating system and software. As shown in FIG. 1A, each computing device 100 may also include additional optional elements, such as a memory port 103, a bridge 170, one or more input/output devices 130a-n (generally referred to using reference numeral 130), and a cache memory 140 in communication with the central processing unit 121. These components communicate with the central processing unit 121 which comprises programming operable to control these components, and, when appropriate, save data to the memory unit 122 and/or send data through the network interface 118, and the like.

The central processing unit 121 is any logic circuitry that responds to and processes instructions fetched from the main memory unit 122. In many embodiments, the central processing unit 121 is provided with one or more microprocessor units such as: those manufactured by Intel® Corporation, Motorola® Corporation, International Business Machines®, Advanced Micro Devices®, Qualcomm®. The computing device 100 may be based on any of these processors, or any other processor capable of operating as described herein.

Main memory unit 122 may be one or more memory chips capable of storing data and allowing any storage location to be directly accessed by the microprocessor 121. The main memory 122 may be based on any available memory chips capable of operating as described herein. In the embodiment shown in FIG. 1B, the processor 121 communicates with main memory 122 via a system bus 150. FIG. 1A depicts an embodiment of a computing device 100 in which the processor communicates directly with main memory 122 via a memory port 103. FIG. 1A also depicts an embodiment in which the main processor 121 communicates directly with cache memory 140 via a secondary bus, sometimes referred to as a backside bus. In other embodiments, the main processor 121 communicates with cache memory 140 using the system bus 150.

In the embodiment shown in FIG. 1B, the processor 121 communicates with various 1/O devices 130 via a local system bus 150. Various buses may be used to connect the central processing unit 121 to any of the I/O devices 130, including a VESA VL bus, an ISA bus, an EISA bus, a MicroChannel Architecture (MCA) bus, a PCI bus, a PCI-X bus, a PCI-Express bus, or a NuBus. For embodiments in which the I/O device is a video display 124, the processor 121 may use an Advanced Graphics Port (AGP) to communicate with the display 124. FIG. 1A depicts an embodiment of a computer 100 in which the main processor 121 also communicates directly with an I/O device 130b via, for example, HYPERTRANSPORT, RAPIDIO, or INFINIBAND communications technology.

The computerized medical device, i.e. computing device 100 may comprise or be connected to one or more of a wide variety of I/O devices 130a-n, each of which may be of the same or different type and/or form. Input devices include touch screens, keyboards, mice, trackpads, trackballs, microphones, scanners, cameras, RFID readers/writers and drawing tablets. Output devices include video displays including touch screens, speakers, inkjet printers, laser printers, and dye-sublimation printers. The I/O devices may be controlled by an 1/O controller 123 as shown in FIG. 1B. Furthermore, an I/O device may also provide storage and/or an installation medium for the computing device 100. In some embodiments, for example, the computing device 100 may provide USB connections (not shown) to receive handheld USB storage devices.

Referring still to FIG. 1B, the computing device 100 may support any suitable installation device (Not Shown), such as a floppy disk drive for receiving floppy disks such as 3.5-inch disks, 5.25-inch disks or ZIP disks, a CD-ROM drive, a CD-R/RW drive, a DVD-ROM drive, tape drives of various formats, USB device, hard-drive or any other device suitable for installing software and programs. The computing device 100 may further comprise a storage device, such as one or more hard disk drives or redundant arrays of independent disks, for storing an operating system and other software.

Furthermore, the computing device 100 may include a network interface 118 to interface to the network 104 shown on FIG. 1C through a variety of connections including, but not limited to, standard telephone lines, LAN or WAN links (e.g., 802.11, T1, T3, 56 kb, X.25, SNA, DECNET), broadband connections (e.g., ISDN, Frame Relay, ATM, Gigabit Ethernet, Ethernet-over-SONET), wireless connections, or some combination of any or all of the above. Connections can be established using a variety of communication protocols (e.g., TCP/IP, IPX, SPX, NetBIOS, Ethernet, ARCNET, SONET, SDH, Fiber Distributed Data Interface (FDDI), RS232, EEE 802.11, IEEE 802.11a, IEEE 802.11b, IEEE 802.11g, IEEE 802. Ln. CDMA, GSM, WiMax, and direct asynchronous connections). In one embodiment, the computing device 100 communicates with other computing devices 100′ via any type and/or form of gateway or tunneling protocol such as Secure Socket Layer (SSL) or Transport Layer Security (TLS). The network interface 118 may comprise a built-in network adapter, network interface card, PCMCIA network card, card bus network adapter, wireless network adapter, USB network adapter, modem, or any other device suitable for interfacing the computing device 100 to any type of network capable of communication and performing the operations described herein.

In further embodiments, an I/O device 130 may be a bridge between the system bus 150 and an external communication bus, such as a USB bus, a FireWire bus, an Ethernet bus, a Gigabit Ethernet bus, a HIPPI bus, a Super HIPPI bus, a SerialPlus bus, a SCI/LAMP bus, a FibreChannel bus, or a Serial Attached small computer system interface bus, or any other type of bus currently available or to be created using the same architecture.

A computing device 100 of the kind depicted in FIGS. 1A and 1B typically operates under the control of operating systems, which control scheduling of tasks and access to system resources. The computing device 100 can be running any operating system such as any of the versions of the MICROSOFT WINDOWS operating systems, UNIX and LINUX operating systems, any version of the MAC OS, ANDROID operating system, IOS operating system, any real-time operating system, any open source operating system, any proprietary operating system, any operating systems for mobile computing devices, or any other operating system capable of running on the computing device and performing the operations described herein.

FIG. 1C illustrates an embodiment of a network environment. The network environment comprises one or more clients 102a-102c (also generally referred to as local machine(s) 102, client(s) 102, client node(s) 102, client machine(s) 102, client computer(s) 102, client device(s) 102, computing device(s) 102, endpoint(s) 102, or endpoint node(s) 102) in communication with one or more computing devices 106a-106c (also generally referred to as server(s) 106 or computing device(s) 106) via one or more networks 104.

The network 104 (also generally referred to as network(s) 104) can be a local-area network (LAN), such as a company Intranet, a metropolitan area network (MAN), or a wide area network (WAN), such as the Internet or the World Wide Web. In some embodiments, there are multiple networks 104 between the client 102 and the computing devices 106. In one of these embodiments, a network 104 may be a private network or a public network. As shown in FIG. 1C, a client 102a device on a network 104 may connect to other client devices 102b-c or to other computing devices 106a-c via one or more networks 104.

The network 104 may be any type and/or form of network and may include any of the following: a point-to-point network, a broadcast network, a wide area network, a local area network, a telecommunications network, a data communication network, a computer network, an ATM (Asynchronous Transfer Mode) network, a SONET (Synchronous Optical Network) network, an SDH (Synchronous Digital Hierarchy) network, a wireless network, and a wireline network. The network may comprise mobile telephone networks utilizing any protocol or protocols used to communicate among mobile devices, including AMPS, TDMA, CDMA, GSM, GPRS, or UMIS. In some embodiments, different types of data may be transmitted via different protocols. In other embodiments, the same types of data may be transmitted via different protocols.

A client 102 and a computing device 106 (referred to generally as computing devices 100) can be any workstation, desktop computer, laptop or notebook computer, server (including file servers, application servers, and media servers), portable computer, mobile telephone or other portable telecommunication device, media playing device, a gaming system, mobile computing device, smartphone, tablet computer, or any other type and/or form of computing, telecommunications or media device that is capable of communicating on any type and form of network and that has sufficient processor power and memory capacity to perform the operations described herein. In some embodiments, the computing device 100 may have different processors, operating systems, and input devices consistent with the device. Typical embodiments of the present invention, however, are carried out on smartphones or tablet computer devices configured and programmed to operate as the computerized medical device disclosed herein.

A client 102 i.e. a user may execute, operate or otherwise provide an application, which can be any type and/or form of software, program, or executable instructions, including, without limitation, any type and/or form of web browser, web-based client, client-server application, HTML, an ActiveX control, or a JAVA applet, PHP, and Javascript, or any other type and/or form of executable instructions capable of executing on client 102. Examples of browsers include INTERNET EXPLORER®, EDGE®, CHROME®, FIREFOX®, SAFARI® and other browsers known.

A computing device 106 may utilize a file server, application server, web server, proxy server, appliance, network appliance, gateway, application gateway, gateway server, virtualization server, deployment server, SSL VPN server, or firewall. In other embodiments, a computing device 106 is a blade server. In further embodiments, the computing device may be a virtualized server operating one processor of a multi-processor system. In some embodiments, the functionality described herein is provided as a virtual application using a virtualized processor.

In one embodiment, a computing device 106 provides functionality of a web server. In some embodiments, a web server 106 comprises an open-source web server.

As will be described below various tasks and steps can be processed on one or more computing devices 106, as well as clients 102. The processes and steps can be bifurcated across each, for example, retrieval of information from the medications diary can performed on 106, where request or query information can be received and analyzed by 106c. In some instances, clients 102a, b or c could also be assisting in real-time processing in the various steps or simply be the triggering device or interface to retrieve and/or initiate the processing of various steps as will be described below.

It should also be clear that the present embodiments can be based and operated in the cloud or cloud-based systems, of which several known cloud computing platforms that exist include Amazon Web Services® (AWS), Microsoft's Azure®, and IBM's Cloud®.

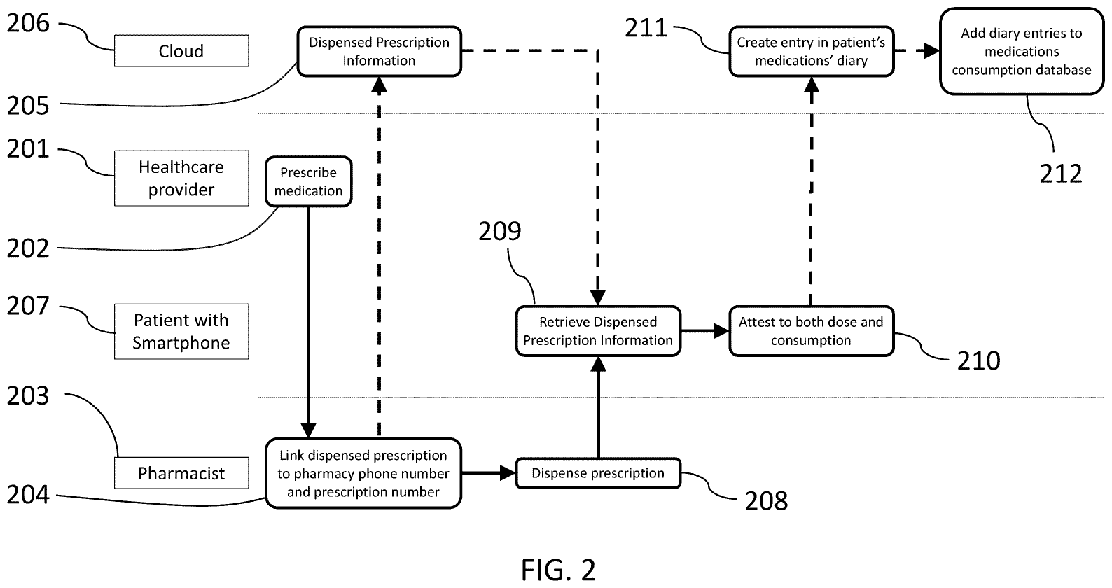

Referring to FIG. 2, the healthcare provider 201 can prescribe medication 202 for the patient. During the dispensing process by the pharmacist 203, the pharmacy phone number and the specific prescription number can be linked 204 to the dispensed prescription information and stored 205 in the cloud 206. After the patient 207 receives the dispensed medication 208, the patient 207 can use the computing device, in this embodiment, a smart phone, to retrieve the dispensed prescription information 209 by entering or scanning the pharmacy phone number and the unique prescription number. When the patient 207 takes their prescribed medication, the patient 207 can use their smartphone to attest 210 that this was the drug dispensed and that they are taking the drug at this date and time. An entry can be created 211 in the patient's medications diary, which can become part of the medications consumption database 212.

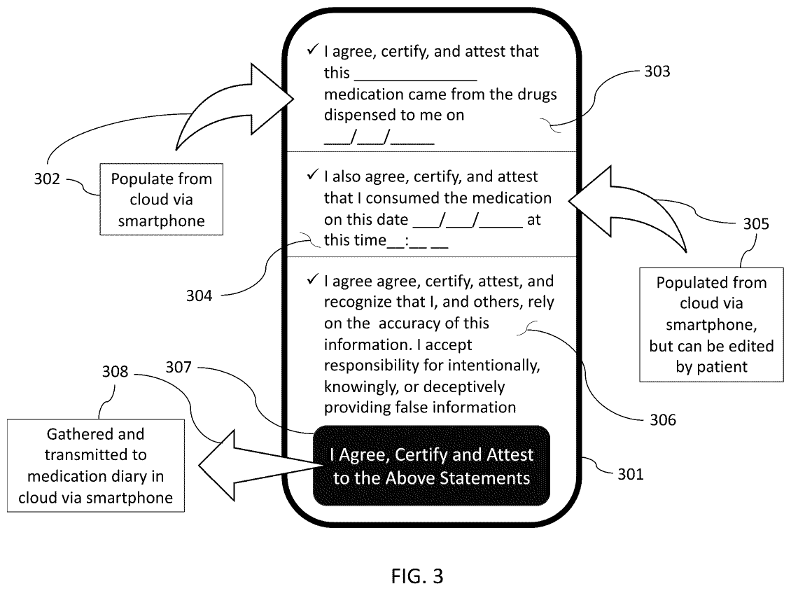

Referring to FIG. 3, the patient can use their smartphone 301 to retrieve prescription information and populate 302 an attestation statement 303 that the dispensed drug is consumed. Similarly, an attest statement 304 of date and time of consumption can be populated 305 from the smartphone operating system and edited, and edits can be noted in the record. The patient can also attest 306 to the integrity of the information provided. The patient can press the statement 307 to certify all onscreen attestations which also transmits 308 the information to the patient's medication diary in the cloud. In operation, the attestation is automatically generated by the programmed computerized medical device, which is operable to generate and display the attestation document and a prompt to provide a confirming input into the user interface to attest to the consumption of the medication. The prompt may, in either a single or multi-prompt process, provide a prompt to confirm the date and time that the patient consumed the medication. The attestation document may be saved to the patient's medication diary, which includes a digital record of medical prescription information and patient attestations of which medications are taken and when the medications are taken, the digital record being a data file saved on a memory accessible to the computerized medical device.

In a preferred embodiment, the attestation document is automatically generated in the system by the user interface and is signable and recordable all within the user interface of the system. This prevents a user from having to navigate away into, e.g. a browser signature program, third party system, or the like. This solves the problem in the computer arts of having to navigate away from a browser/user interface, which often leads to failure of the user to return to the user interface, which could lead a problem of reduced persistence and adherence to the medication administration regimen.

In some embodiments, the computerized medical device is operable to receive and record evidence that the patient has dispensed and taken the medication. The device may then append this gathered evidence data to the attestation, linking the two and saving this data to the patient's medications diary. In one embodiment, evidence of the patient's attestation that medication was dispensed to the patient can be received from the patient by the computerized medical device by operating the computerized medical device to take a photo of the drug or bottle using the camera; scanning, using the camera of the computerized medical device or entering, using the user input interface of the computerized medical device, one or more of: the pharmacy phone number and prescription number; the dispensed package DSCSA GS1 datamatrix, the dispensed package GTIN, serial number, expiration date, and lot number, a dose to confirm single factor authentication of the medication; and/or a dose to confirm multi-factor authentication of the medication.

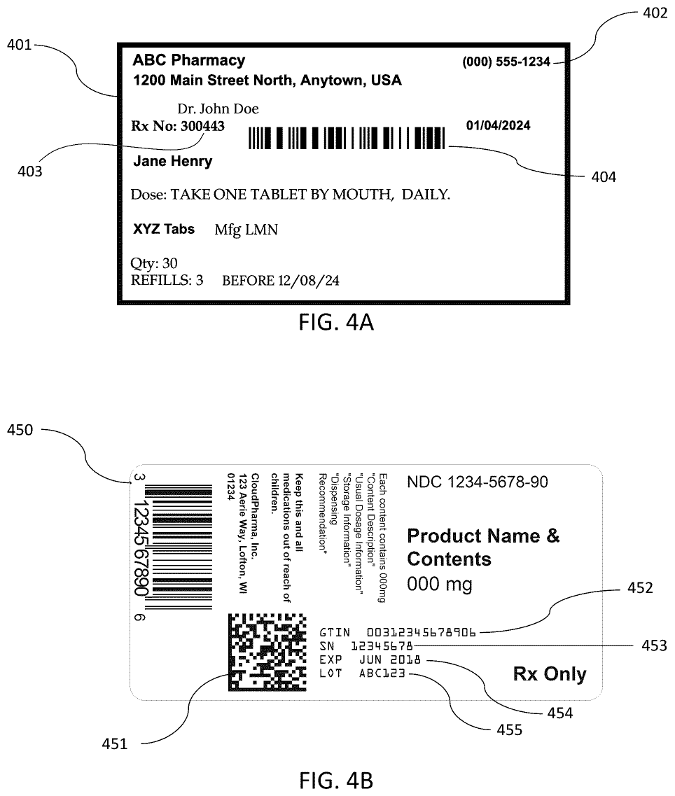

Referring to FIG. 4A, is a sample prescription label 401 which is typically attached to the dispensed medication package, bottle, or container. The patient can link to their prescription using their smartphone as the computerized medical device to enter the pharmacy phone number 402 and the prescription number 403. Alternatively, the patient can link to their prescription using their smartphone to enter the pharmacy phone number 402 and to scan, using a camera, the prescription number barcode 404. Often the barcodes 404 are part of internal pharmacy numbering and database system. The pharmacy phone number can be used to identify the type of pharmacy such as CVS®, RITEAID® and other sources to understand the type of pharmacy numbering system utilized for interpreting the barcode, so as to appropriately translate those numbers or to retrieve the specific prescription associated with that number.

Referring to FIG. 4B, the medication can be dispensed in the package that was sold to the pharmacy, in which case it can contain required DSCSA information 450. In this situation, the patient can link to their prescription using their smartphone to scan, using a camera, the unique GS1 DataMatrix 451. Alternatively, patient can link to their prescription using their smartphone to enter the GTIN number 452, the serial number 453, the expiration date 454, and the lot number 455. As discussed throughout, the input data into the computerized medical device may be stored in a memory and/or database, whether on the device or through a networked connection.

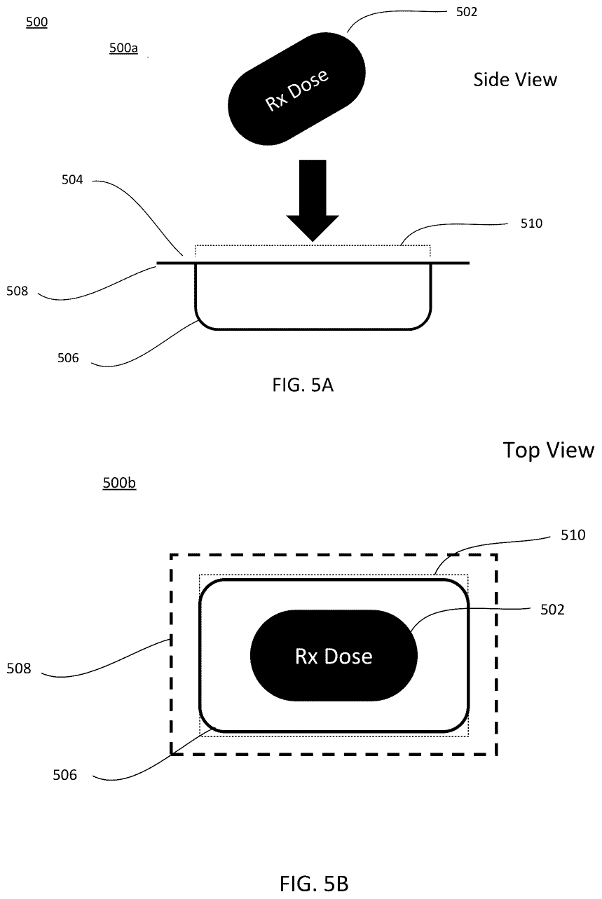

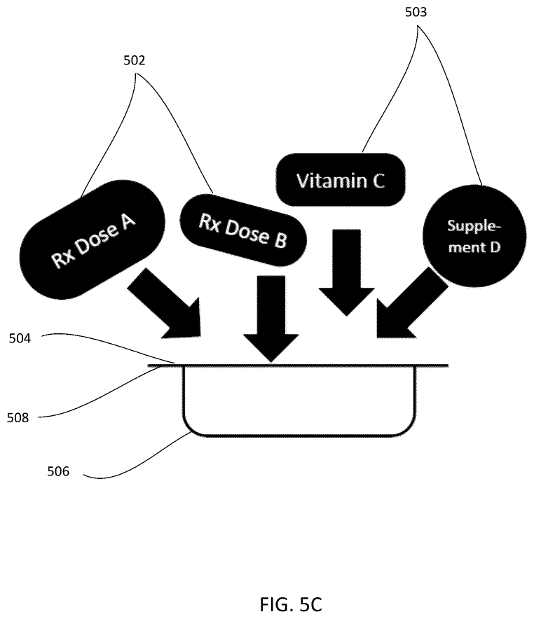

FIGS. 5A-B Illustrate various views of an authenticatable dose container 500 used to store and then later dispense medication. A top view of dose container in configuration 500a is shown where a dose of medication 502 can be placed therein. FIG. 5B illustrates a configuration 500b with the dose of medication 502 disposed inside a cavity portion 506 that is connected to support layer 508. A sealing layer 504, which may be comprised of multiple layers of material, can be disposed over the support layer 508. An observable surface can be formed in part by sealing layer 504 that is provided over the cavity 506, wherein the dose of medication 502 is to be stored for shipping and later dispensing. Some common dose containers include blister packs, which can provide one or more cavity portions aligned in a matrix having X number of rows and Y number of columns. Some blister packs have different configurations such as circular shapes and so forth that are formed of the plurality of cavities or blisters. Other dose containers that are known to the those skilled in the art include rolls, jars, bottles, vials, droppers, cartridges, syringes, and packets. The computerized medical device, in this and other embodiments discussed, is programmed to read and identify data on the authenticatable dose container 500 by, for example, image analysis as recorded by a camera photo or video, or other wireless communication such as RFID detection, and the like.

FIG. 5C illustrates another dose container like those of FIGS. 5A-C but configured to store doses of multiple medications 502 and doses of multiple vitamins or supplements 503 in the same cavity 506. It is common for patients to take a prescribed cocktail of medications, vitamins, and supplements as part of their health regiment. Increasingly there are dose containers that combine, where possible, multiple types of medications, vitamins, and supplements for convenience and to assist with adherence.

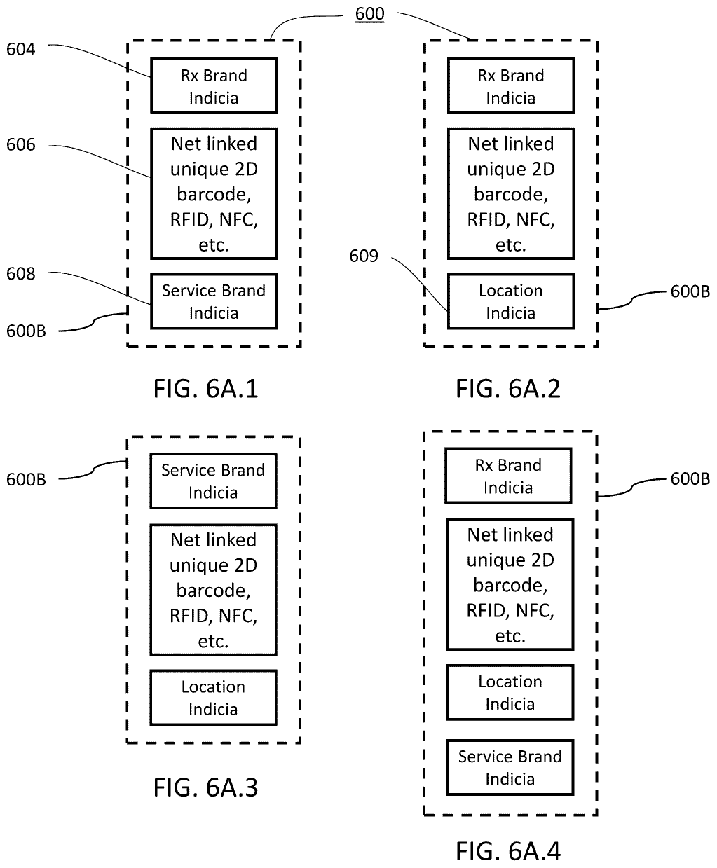

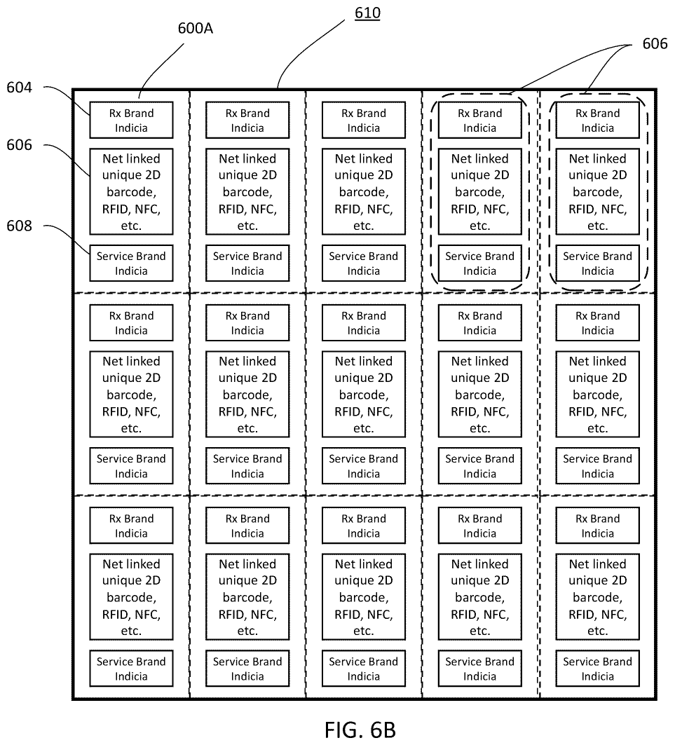

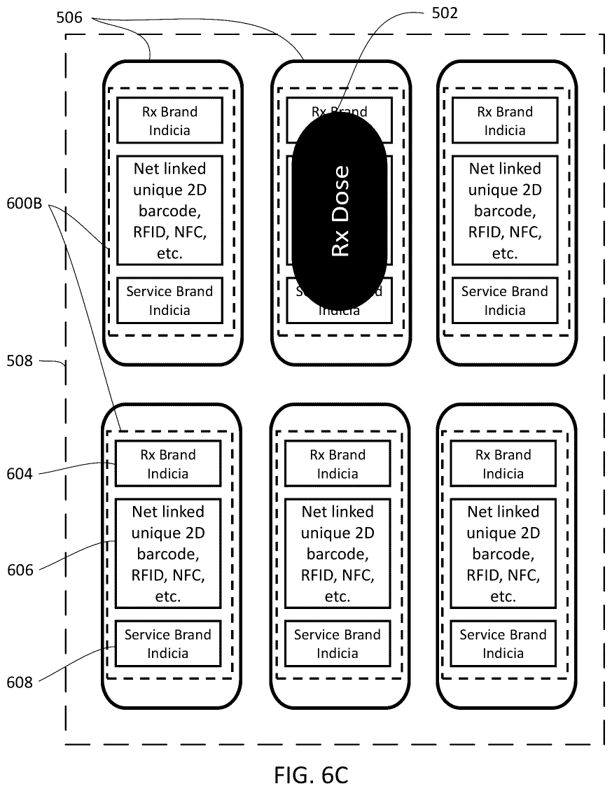

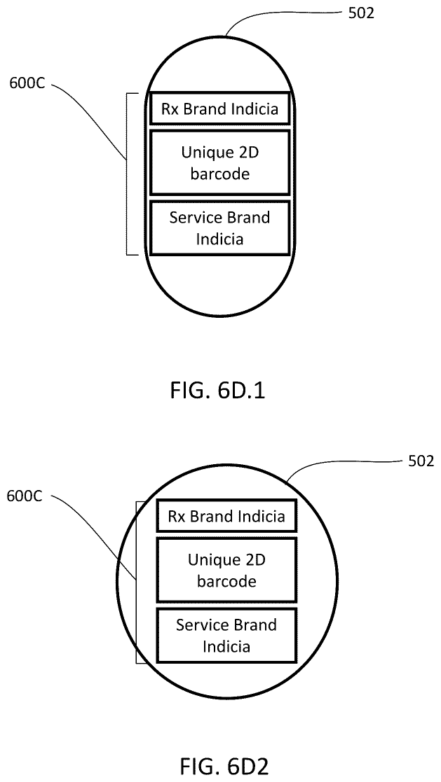

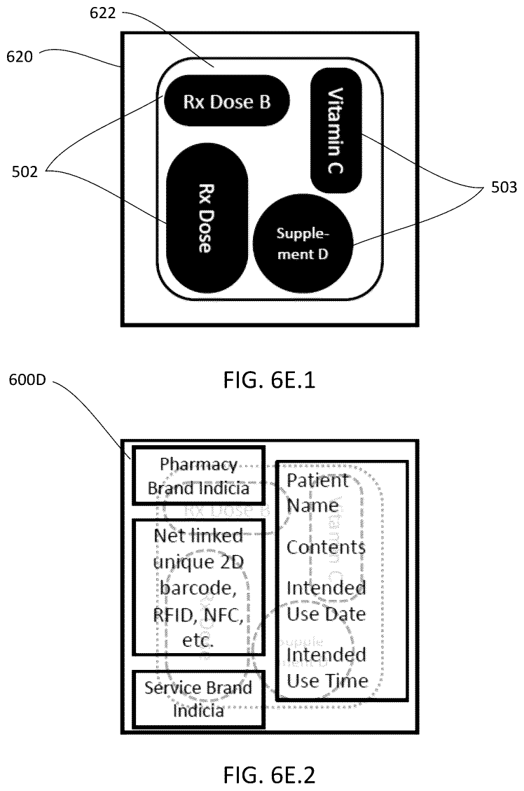

An authenticator indicia, such as the example shown in FIG. 6A.1 can be disposed on or within the dose container packaging to be used as part of the systems and methods herein for authenticating doses of medication and tracking doses of medication. The authenticator indicia 600 can be comprised of one or more sections, such as section 604 that is a brand indicia, which is indicative of the manufacturer of the dose of medication, section 606 as shown can be contain a unique identification number, linked to a website, that can be encoded into a 2D barcode or matrix, a 1-D barcode, electronically as an RFID tag, or other NFC, active or passive radio frequency tag, or electromagnetic spectrum emitting or responsive tag that is readable and recordable by the smartphone computerized medical device. Section 608 can comprise indicia associated with the brand of the service company providing the authentication systems or methods. Alternatively, a fourth indicia can be disposed thereon, such as a regional or location indicia 609. The regional indicia 609 can be indicative of an authorized distribution region, consumption region, or manufacturing region where the dose of medication is allowed to be distributed to, consumed in, or manufactured in. For example, the regional indicia can include a country name or a state name, that indicates the authorized distribution of that particular dose of medication in that country or state. Thus, if a user were to see the country Japan on the authenticator indicia on a dose container and they were in the United States, it might give them pause with regards to the ability to have, distribute or consume that particular dose of medication. It might also be indicative of the governing laws or standards by which that particular dose of medication was approved or authorized. FIGS. 6A.2-6A.4 illustrate other variations and combinations for exemplary purposes, but are not intended to be limiting in scope, as additional variations can be contemplated to those skilled in the art upon presenting these examples.

In a particular embodiment, the outside authenticator on a blister pack or sealed enclosure containing at least one dose of medication can be used for authentication that the medicine was taken. For example, the computerized medical device may be operable to associate the outside authenticator of each blister pack or sealed enclosure with each dose of medication in a database. Then, the device may associate, in a database, each outside authenticator with other outside authenticators within the same unit of sale, at manufacture, wholesale, or retail. The medical device may scan the outside authenticator, using the camera and then provide an authenticating communication system configured to receive scanned information from the outside authenticator. The medical device may be further programmed to determine whether the dose of medication is authentic based on available data, and to display medication authentication confirmation on the display of the computerized medical device.

In FIG. 6B a sheet 610 of authenticator indicia 600A can be disposed over a dose container, such as a blister pack, having a plurality of cavities 506 or blisters. The sheet 610 can formed as part of the sealing layer 504 discussed above or be a separate layer that is overlaid thereon. The authenticator indicia 600A can also be printed on, etched into, blazed into, or burned onto (using a laser or heat source) the sealing layer 504 or alternatively a portion of the sealing layer 504. In a preferred embodiment the authenticator indicia 610 are disposed over or within an observable surface that is directly or at least partially disposed over the dose of medication that is to be contained in the cavity or blister portion. These authenticator indicia shown in FIG. 6B may also be referred to as an outside authenticator 600A.

In another configuration, shown in FIG. 6C the authenticator indicia 600B can be disposed within the cavity 506, such as on the inside bottom surface of the cavity 506. As shown, when a dose of medication 502 is placed therein it can cover at least partially a portion of the authenticator from being viewed. Furthermore, when the sealing layer 504 is placed over top the cavity portion the ability to view the authenticator indicia 600B can be completely restricted. In other words, the sealing layer can be non-translucent or non-transparent layer to restrict viewing into the cavity. Alternatively, the outside authenticator 600A can block the view in addition to the dose of medication 502. The cavity or blister portion 506 can also have non-translucent, or non-transparent qualities as well.

In this configuration or location of the authenticator indicia, as shown in FIG. 6C, it can also be referred to as the inside authenticator 600B. The outside authenticator 600A and inside authenticator 600B each have their own unique identification number encoded therein. These unique identification numbers (one from 600A and one from 600B), as will be discussed in more detail below, can be associated with the single dose of medication 502 and used for authenticating the dose of medication.

FIGS. 6D.1-6D.2 illustrate yet another location or configuration of an authenticator indicia 600C, which is placing the Authenticator Indicia onto the dose of medication 502. The two figures shown use different shapes of doses of medication 502 to illustrate the Authenticator Indicia 600C can be adapted to various shapes and surfaces accordingly. Authenticator Indicia 600C can also be referred to as an on-dose authenticator. As disclosed so far, there can be at least a first, second and third versions of authenticators each having their own unique identification number and other indicia associated therewith. Again, additional authenticators in additional configurations and locations are possible, thus the examples are not meant to be exhaustive, but rather principles of what can be.

It should be noted that in one embodiment only a single outside authenticator is used for authenticating a dose of medication. In another embodiment only the inside authenticator is used for authenticating a dose of medication and in yet another embodiment both the outside and inside authenticators are used to authenticate the dose of medication. In yet another embodiment, the outside authenticator and the on-dose authenticator can be used. Alternatively, a first and second authenticator can be used or even a first, second and third authenticator can be used.

FIGS. 6E.1-6E.2 illustrate utilizing an authenticator indicia 600D with a dose container 620 having a cavity or pocket 622 configured to hold a plurality of medications 502 and/or vitamins/supplements 503. FIG. 6E.2 shows authenticator indicia 600D on the outside of the dose container 620, but similar to some of the embodiments previously described an inside authenticator could be disposed on the inside of the dose container 620 for example in the cavity or pocket 622 that is only viewable upon opening the dose container 620 (though not shown). Also, as described previously one or more medications 502 or vitamins/supplements 503 could have an on-pill authenticator indicia. As noted above the dose container 620 could be a blister pack, or a pill pack, or other type of dose container that has a cavity configured to hold multiple pills. In the version of a pill pack, each pill pack can be connected along and edge and formed into a roll or tape, which is then stored in another housing or container that dispenses each pack (containing a plurality of pills) one at time, that can be separated from the rest of the roll or tape. An authenticator Indicia can be placed on the container or housing where the tape or roll of pill packs is stored.

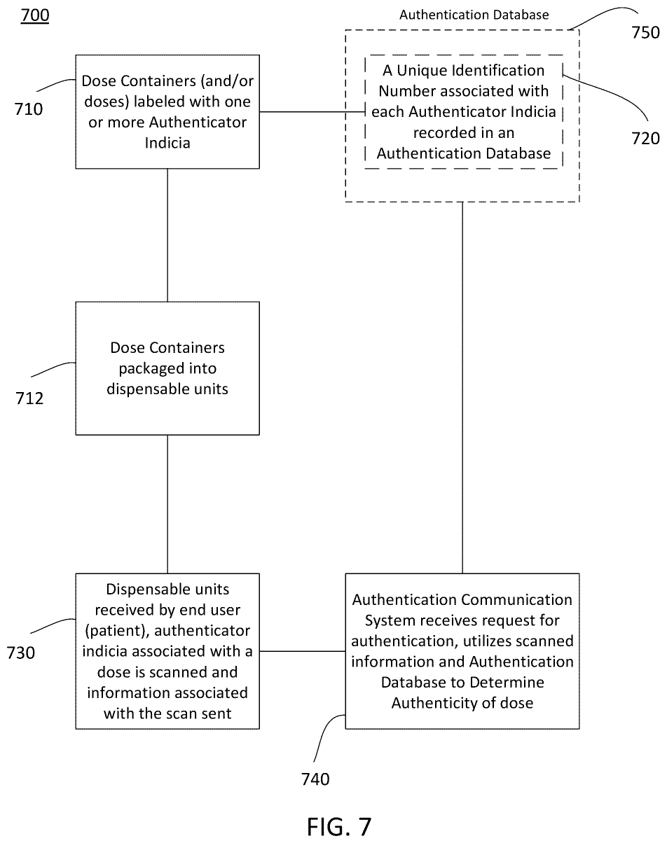

FIG. 7 illustrates a flowchart of a method 700 of preparing 710, packaging 712, dispensing 730, and initiating 740 the authentication process for doses of medicine. In step 710 dose containers are labeled or otherwise provided with authenticator indicia, such as 600, described above. A unique identification number that is associated with each authenticator indicia can be recorded in step 730 in an authentication database 750. The dose containers can then be packaged into dispensable units in step 712. Once the dispensable units are distributed, the end user, such as patient who was prescribed the medication by a medical professional, can scan the authenticator indicia using the smartphone computerized medical device and send scanned information via a networked connection to the authentication communication system in step 730. The scanned authenticator indicia and request for authentication can be received by the authentication communication system in step 740. The authentication communication system can then use the scanned authenticator indicia and information associated with the request, as well as the authentication database to determine the authenticity of the dose of medication. For purposes of this application, it should be understood that a medical professional can include medical doctors, nurses, pharmacists, nutritionists, dieticians, physician's assistants, dentists, optometrists, physical/occupational/addiction therapists, psychiatrists, and others that would be within this scope in view of these examples. It will be understood that these steps in this embodiment, and related embodiments, are carried out using the computerized system discussed above.

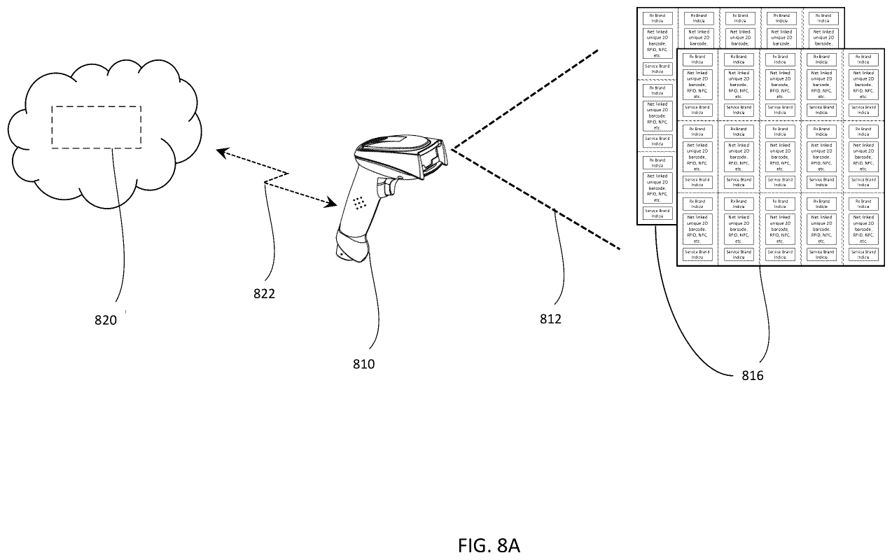

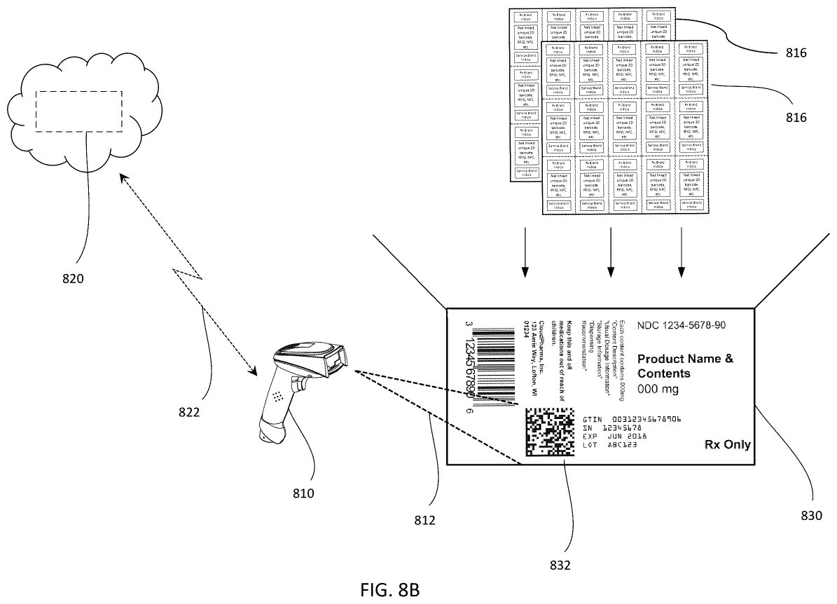

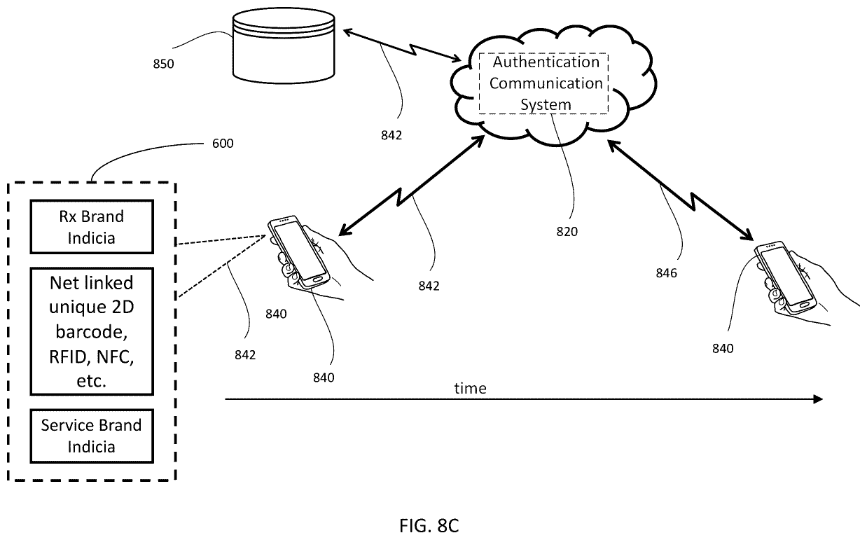

FIGS. 8A-C illustrate various structural and system components for implementing the method shown and described in FIG. 7 and above. FIG. 8A illustrates utilizing a scanning tool 810 to scan all of the external indicia of the authenticable doses 816. The scanned information can then be transmitted via a network 822 to the authenticator communication system 820, which can be hosted in the cloud. It should be noted that scanning tool 810 can be comprised of a variety of types including, 2D barcode scanning device, imaging camera on a smartphone, tablet or other computing device, laser scanner, printer scanner, or other device utilized to detect a visual authenticator indicator. In the embodiments where the authenticator indicia have a radio or electromagnetic component, an appropriate RFID, NFC, or other radio transceiving device can be used to account for the non-visual indicia components. As noted above, the information scanned from the authenticator indicia, such as the unique identification number can be transmitted by the scanning tool or a computing device associated with the scanning to the authenticator communication system 820 for authentication analysis and determination. Additionally, the scanning tool can transmit location-based information derived from GPS information, IP addresses, registries, cellular network or other location-based technologies along with the unique identification number. Time, date and user information can be transmitted. Information about product, lot and purchase order can also be transmitted.

FIG. 8B shows that DSCSA GS1 datamatrix 832 which manufactures can place on the unit of sale to pharmacy carton 830 can be linked to the external indicia of all of the authenticable doses 816 placed in that carton 830. This link can be transmitted to the authenticator communication system 820.

FIG. 8C illustrates an end user utilizing a smartphone 840 having a camera or other scanning tool to scan an authenticator indicia 600 associated with a dose of medication. The scanned information including the unique identification number and location information are transmitted 842 to the authentication communication system 820, which can then utilize the unique identification number along with stored information in the authentication database to determine the authenticity of the dose of medication. As previously noted, location information, time or date information and user information can also be utilized as part of authenticating determination process. Once an authentication determination has been realized system 820 can then transmit 846 authenticity information to a user portal accessible by smartphone 840. Additionally, and in some implementations of the system, system 820 can request access to a user profile, or the user profile associated with the end user that is prescribed the dose of medication, prior to transmitting authentication determination information.

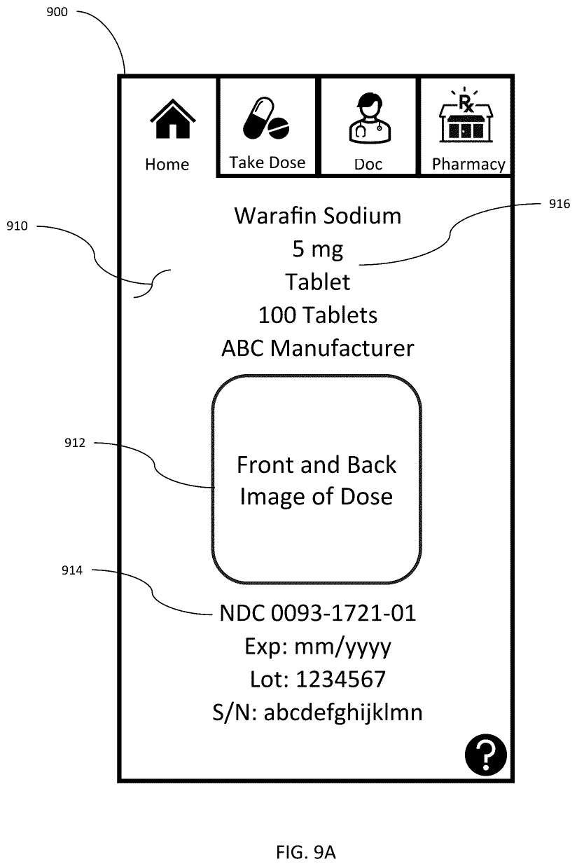

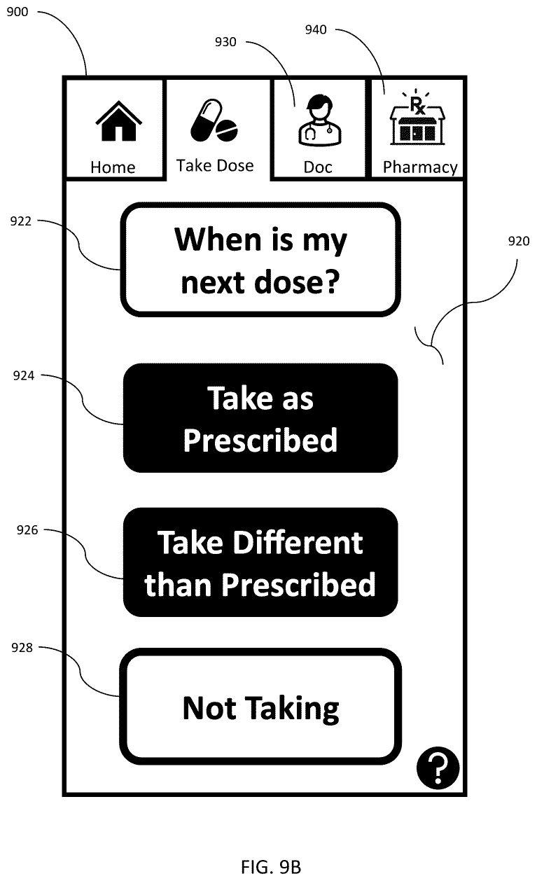

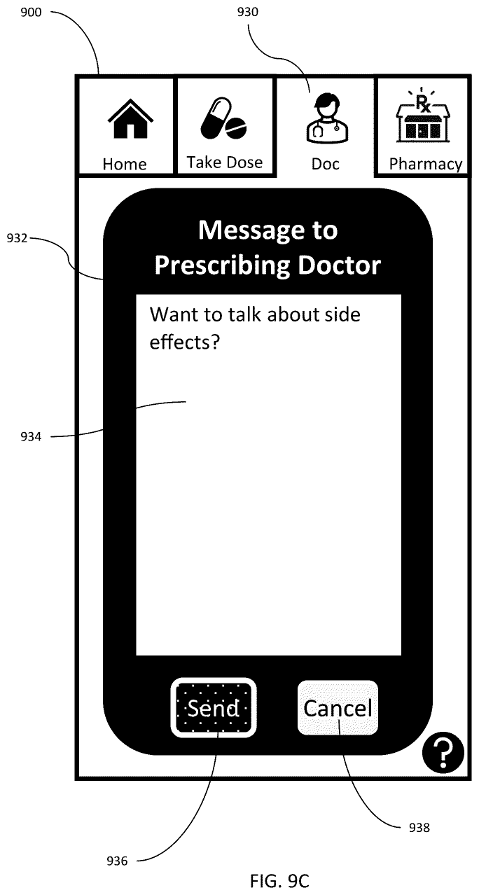

FIGS. 9A-C illustrate a user portal 900 for accessing and utilizing the computerized medical device for medications dispensed to patients. As shown in FIG. 9A, user portal 900 has can have a variety of interfaces, such as interface 910, which provides basic information about the medication. Another interface 920 shown in FIG. 9B can be utilized to initiate action relative to taking the next dose of medicine. Interface 920 can include transmitted information through the medical device regarding the appropriate time to take the next dose, history of previous doses taken, reminders and alerts related to taking the next dose, and tracking adherence or non-adherence to the prescribed medical script. Some of these features are shown in sections 922, 924, 926 and 928 by way of example and are not limiting. From this interface, other interfaces can be selected to initiate communication with a medical provider 930 or enabling and optionally initiating a prescription refill 940. This information can be stored in a database, such as a user profile database, that is accessible by the medical device and end user.

The user interface presented on the computerized medical device (in typical embodiments, a smartphone or tablet) is operable to contain all of its features within the continuous user interface of the system. Importantly, in many embodiments, a user need not navigate out of the user interface of the system to access other portions of the systems functionality. For example, camera activation and recording may be performed through the user interface, rather than having to use the phones standard camera recording operations. Similarly, data access of user information can be performed through the user interface, rather than having to access a third party system. Further, the attestation functionality may be generated and presented through the user interface, again without requiring the user navigate away into other systems, apps, or web pages. The other features disclosed herein similarly are integrated into the system's user interface. As is known in the art, there is a problem with computerized operations where users navigate away from a web page, app, or other user interface, never to return. This problem is particularly present in the present instance where the goal is to increase adherence and persistence in a medical regimen, where the slightest distraction can meaningfully reduce both adherence and persistence. Therefore, the present system solves this problem of both the computer arts and the medical treatment arts by providing a consistent user interface which provides the functionality in a single system, without requiring navigation away.

Interface 930 shown in FIG. 9C can be utilized to invite and communicate with third parties like medical providers or authorized support persons. Section 932 provides a messaging portal with a messaging section 934 and means to send 936 the message or cancel/delete 938 the message. These communications can be stored with the user profile information, stored locally on a device accessing the portal 900, or deleted instantly upon closing the portal.

As mentioned, the methods, systems and structure described assist with the creating a reliable record of consumption of doses of medication, as well as generating evidence of dispensed medication authenticity and its consumption.

It should be understood that the systems described above may provide multiple ones of any or each of those components and these components may be provided on either a standalone machine or, in some embodiments, on multiple machines in a distributed system. The phrases ‘in one embodiment,’ ‘in another embodiment,’ and the like, generally mean that the particular feature, structure, step, or characteristic following the phrase is included in at least one embodiment of the present disclosure and may be included in more than one embodiment of the present disclosure. Such phrases may, but do not necessarily, refer to the same embodiment.

The techniques described above may be implemented as a method, apparatus, or article of manufacture using programming and/or engineering techniques to produce software, firmware, hardware, or any combination thereof. The techniques described herein may be implemented in one or more computer programs executing on a programmable computer including a processor, a storage medium readable by the processor (including, for example, volatile and non-volatile memory and/or storage elements), at least one input device, and at least one output device. Program code may be applied to input entered using the input device to perform the functions described and to generate output. The output may be provided to one or more output devices. As indicated above, software (also referred to herein as components, modules, programs, program code, and applications) may include virtual machines and virtualized software.

Any of the functions disclosed herein may be implemented using means for performing those functions. Such means include, but are not limited to, any of the components disclosed herein.

Each computer program within the scope of the claims below may be implemented in any programming language, such as assembly language, machine language, a high-level procedural programming language, or an object-oriented programming language. The programming language may, for example, be LISP, PROLOG, PERL, C, C++, C#, JAVA, PYTHON, HTMLS or any compiled or interpreted programming language.

Each such computer program may be implemented in a computer program product tangibly embodied in a machine-readable storage device for execution by a computer processor. Method steps of the invention may be performed by a computer processor executing computer program instructions tangibly embodied on a non-transitory computer-readable medium to perform functions of the invention by operating on input and generating output. Cookies, operating systems, and other information may also be stored on non-transitory computer-readable medium. Suitable processors include, by way of example, both general and special purpose microprocessors. Generally, the processor receives instructions and data from a read-only memory and/or a random-access memory. Storage devices suitable for tangibly embodying computer program instructions include, for example, all forms of computer-readable devices, firmware, programmable logic, hardware (e.g., integrated circuit chip; electronic devices; a computer-readable non-volatile storage unit; non-volatile memory, such as semiconductor memory devices, including EPROM, EEPROM, and flash memory devices; magnetic disks such as internal hard disks and removable disks; magneto-optical disks; and CD-ROMs). Any of the foregoing may be supplemented by, or incorporated in, specially-designed ASICs (application-specific integrated circuits) or FPGAs (Field-Programmable Gate Arrays). A computer can generally also receive programs and data from a storage medium such as an internal disk (not shown) or a removable disk. These elements will also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium. A computer may also receive programs and data from a second computer providing access to the programs via a network transmission line, wireless transmission media, signals propagating through space, radio waves, infrared signals, etc.

While several variations of the present disclosure have been illustrated by way of example in preferred or particular embodiments, it is apparent that further embodiments could be developed within the spirit and scope of the present disclosure, or the inventive concept thereof. However, it is to be expressly understood that such modifications and adaptations are within the spirit and scope of the present disclosure, and are inclusive, but not limited to the following appended claims as set forth.

1.-20. (canceled)

21. A computerized medical device for treating a patient with a prescription dispensed to the patient, the computerized medical device operable to:
receive medication prescription information from a patient through a user input interface of the computerized medical device, the medication prescription information comprising patient user ID, drug name, and directions for use;
receive a notice of medication consumption, including the date and time that the patient consumed the medication (provided by the clock of the computerized medical device), in either of two ways,
via a prompt on a display of the computerized medical device and a receipt of an input to the user input interface, or
from the medication itself, its packaging, its container, or its label, by scanning, using the camera of the computerized medical device, of a unique internet addressable 2D barcode on the medication, its package, its container, or its label, by scanning or inputting the dispensed package DSCSA GS1 Data Matrix, GTIN, serial number, expiration date, lot number, UPC code, or any alphanumeric number or code, or by scanning, using image recognition, the medication, its package, its container, or it's label;
create a digital record of medication prescription information and patient notices of medication consumption, the digital record being a data file saved on a memory accessible to the computerized medical device;
retrieve a historical record of medication prescription information and patient notices of medication consumption corresponding to the patient from a memory accessible to the computerized medical device; and
provide an output from the computerized medical device comprising suggestions about a patient taking their medication, the suggestions based on medication prescription information, patient notices of medication consumption, and current date and time.

22. The medical device for treating a patient with a prescription dispensed to the patient of claim 21, wherein the computerized medical device operable to receive medication prescription information; the medication prescription information may be comprised of at least one of patient identification, prescriber identification, drug name, drug strength, dosage form, quantity prescribed, directions for use, date dispensed, quantity dispensed, dispensing pharmacy, dispensing pharmacy prescription number, or dispensing pharmacy phone number.

23. The medical device for treating a patient with a prescription dispensed to the patient of claim 21, wherein the computerized medical device operable to receive notices of medication consumption; the notices of medication consumption may include an attestation from the patient, that this is the medication which was dispensed for them, or an attestation as to the date and time that the patient consumed the medication, or the notices of medication consumption may include both attestations.

24. The medical device for treating a patient with a prescription dispensed to the patient of claim 21, wherein the computerized medical device is in communication with a network and operable to provide secure real-time or delayed communication with a server about medication prescription information and notices of medication consumption and, based on input to the computerized medical device by the patient.

25. The medical device for treating a patient with a prescription dispensed to the patient of claim 21, wherein the computerized medical device is in communication with a network and operable to request and receive medication prescription information for a patient from servers, by entering the unique pharmacy phone number and prescription number using the user input interface of the computerized medical device, or by scanning, using the camera of the computerized medical device.

26. The medical device for treating a patient with a prescription dispensed to the patient of claim 21, wherein the computerized medical device operable to enable a prescription refill process based on calculations from the approved medical device, that it's time for an automatic refill, based on input by the patient to the computerized medical device, or by scanning using the camera of the computerized medical device, through a networked communication with a pharmacy.

27. The medical device for treating a patient with a prescription dispensed to the patient of claim 21, wherein an evidence that that medication was dispensed to the patient can be received from the patient from the medication itself, its packaging, its container, or its label, by the computerized medical device, individually or in combination, by:
operating the computerized medical device to take a photo of the medication, its packaging, its container, or its label using the camera;
scanning, using the camera of the computerized medical device or entering, using the user input interface of the computerized medical device, the pharmacy phone number or prescription number, or both the pharmacy phone number and prescription number;
scanning, using the camera of the computerized medical device, of a unique internet addressable 2D barcode on the medication, its package, its container, or the label;
scanning, using the camera of the computerized medical device, of the dispensed package DSCSA GS1 Data Matrix or UPC code;
entering, using the user input interface of the computerized medical device, the dispensed package GTIN, serial number, expiration date, lot number, or UPC code, individually or in combination;
scanning, using the camera of the computerized medical device, using image recognition, of the medication, its container, its package, or the label;
scanning, using the camera of the computerized medical device, a dose to confirm single factor authentication of the medication; or
scanning, using the camera of the computerized medical device, a dose to confirm multi-factor authentication of the medication.

28. The medical device for treating a patient with a prescription dispensed to the patient of claim 27, wherein evidence that this medication was dispensed to this patient can be generated from a single-factor authenticatable dose comprising the steps of:
providing an outside or inside dose authenticator on or in a blister pack or sealed enclosure containing at least one dose of medication;
associating the dose authenticator of each blister pack or sealed enclosure with each dose of medication in a database;
associating in a database each dose authenticator with other dose authenticators within the same unit of sale, defined as within the same DSCSA serial number;
scanning the dose authenticator, using the camera of the computerized medical device;
providing an authenticating communication system configured to receive scanned information from the dose authenticator; and
determining whether the dose of medication is authentic based on available data.

29. The medical device for treating a patient with a prescription dispensed to the patient of claim 27, wherein evidence that this medication was dispensed to this patient can be generated by a multi-factor authenticatable dose comprising the steps of:
providing an outside authenticator on a blister pack or sealed enclosure containing at least one dose of medication;
providing an inside authenticator located inside the blister pack or sealed enclosure, wherein the inside authenticator is not completely viewable until the blister pack or sealed enclosure is opened, and wherein the inside authenticator may be on the medication itself via inscription, printing, molding, or attachment;
associating the outside and inside authenticators of each blister pack or sealed enclosure with each dose of medication in a database;
associating in a database a dose authenticator with other dose authenticators within the same unit of sale, defined as within the same DSCSA serial number;
scanning the outside and inside authenticators, using the camera of the computerized medical device;
providing an authenticating communication system configured to receive scanned information from the authenticators; and
determining whether the dose of medication is authentic based on available data.

30. The medical device for treating a patient with a prescription dispensed to the patient of claim 27, wherein other medications, vitamins, or nutritional supplements taken at the same time can also be placed in the blister pack or sealed enclosure.

31. The medical device for treating patients with prescriptions dispensed to patients of claim 27, wherein the device operable to create a digital record of evidence that the medication was dispensed to the patient, the digital record being a data file saved on a memory accessible to the computerized medical device.

32. The medical device for treating a patient with a prescription dispensed to the patient of claim 21, wherein evidence of notices of consumption can be received from the patient by enabling patients to create a video record of their consumption using one or both of a front camera and a back camera individually or simultaneously, which recording can start at the very beginning of the process and record continuously through to final completion, and create a digital record of the evidence, the digital record being a data file saved on a memory accessible to the computerized medical device.

33. The medical device for treating a patient with a prescription dispensed to the patient of claim 21, wherein increased persistence and adherence of a patient or population of patients can be enabled, resulting in improved healthcare outcomes or reduce healthcare system costs.

34. The medical device for treating a patient with a prescription dispensed to the patient of claim 21, the computerized medical device operable to provide automatic reminders via a prompt on a display of the computerized medical device for the patient to take the right dose at the right time, to warn the patient on a display of the computerized medical device that is the wrong medication, or to warn the patient on a display of the computerized medical device that this is not the time to take a medication.

35. The medical device for treating a patient with a prescription dispensed to the patient of claim 21, the computerized medical device operable to receive an input from the patient, via a prompt on a display of the computerized medical device and a receipt of an input to the user input interface wherein the patient is provided the option to automatically share drug consumption information with caregivers, family, friends, researchers, or healthcare providers via networks; thereby enabling caregivers, family, friends, researchers, or healthcare providers to respond as appropriate with positive reinforcement for taking the right drug at the right time, or encourage proper consumption for late or missed doses, which can be relayed back through networks and whose responses are output on the display of the computerized medical device.

36. The medical device for treating a patient with a prescription dispensed to the patient of claim 21, the computerized medical device operable to:
provide individual or team games for patients to play on the computerized medical device which maintain or improve patience persistence and adherence;
notify, track and deliver rewards, or financial incentives to patients for maintaining or improving their persistence and adherence; the delivery of rewards and financial incentives being initiated via a prompt on a display of the computerized medical device and a receipt of an input to the user input interface, which can be relayed through networks by the computerized medical device;
enable a patient or population of patients the option to participate in medical research for conditions treated with prescription drugs; and
create or link patients to a social network, which enables patients to communicate through the computerized medications medical device with other patients having similar conditions, including those with similar or different medication treatments.

37. A computerized medical device for treating a patients with a prescription dispensed to the patient, the computerized medical device operable to:
receive medication prescription information from a patient through a user input interface of the computerized medical device, the medication prescription information comprising patient user ID, drug name, and directions for use;
receive a notice of medication consumption, including the date and time that the patient consumed the medication (provided by the clock of the computerized medical device), via a prompt on a display of the computerized medical device and a receipt of an input to the user input interface—or by scanning, using the camera of the computerized medical device, of a unique internet addressable 2D barcode on the drug its package, or the label, by scanning the dispensed package DSCSA GS1 Data Matrix, or by scanning, using image recognition, of the container, package, label, or medication;
gather evidence that the medication consumed was authentic medication dispensed to the patient from the medication itself, its packaging or it's label;
create a digital record of medication prescription information, patient notices of medication consumption, and gathered evidence, the digital record being a data file saved on a memory accessible to the computerized medical device;
retrieve a historical record of medication prescription information, patient notices of medication consumption, and gathered evidence corresponding to the patient from a memory accessible to the computerized medical device;
provide an output from the computerized medical device comprising suggestions about a patient taking their medication, the suggestions based on medication prescription information, patient notices of medication consumption, gathered evidence, and current date and time;
wherein the documentation communication system is comprised of one or more processers associated with a set of computer executable instructions in memory configured to receive information and data associated with each dose, its prescription, its notices of consumption together with supporting evidence, if any, utilize the database comprising stored information about each dose, its prescription, dispensing, and its notices of consumption, together with supporting evidence, if any, generate and store user profile information associated with a plurality of end users in a user profile database, and create secure connections between end users and medical professionals or support persons.

38. The medical device for treating a patient with a prescription dispensed to the patient of claim 37, wherein an evidence that the medication was dispensed to the patient can be received from the patient by the computerized medical device, individually or in combination, by:
operating the computerized medical device to take a photo of the medication, its packaging, its container, or its label using the camera;
scanning, using the camera of the computerized medical device or entering, using the user input interface of the computerized medical device, the pharmacy phone number or prescription number, or both the pharmacy phone number and prescription number;
scanning, using the camera of the computerized medical device, of a unique internet addressable 2D barcode on the medication, its package, its container, or the label;
scanning, using the camera of the computerized medical device, of the dispensed package DSCSA GS1 Data Matrix or UPC code;
entering, using the user input interface of the computerized medical device, the dispensed package GTIN, serial number, expiration date, lot number, or UPC code, individually or in combination;
scanning, using the camera of the computerized medical device, using image recognition, of the medication, its container, its package, or the label;
scanning, using the camera of the computerized medical device, a dose to confirm single factor authentication of the medication; or
scanning, using the camera of the computerized medical device, a dose to confirm multi-factor authentication of the medication.

39. The medical device for treating a patient with a prescription dispensed to the patient of claim 37, wherein evidence of notices of consumption can be received from the patient by enabling patients to create a video record of their consumption using one or both of a front camera and a back camera individually or simultaneously, which recording can start at the very beginning of the process and record continuously through to final completion, and create a digital record of the evidence, the digital record being a data file saved on a memory accessible to the computerized medical device.

40. A method for treating a patient with medication(s) dispensed to the patient, who can use the medical device of claim 21, wherein increased persistence and adherence of the patient, or a population of patients, can be enabled, resulting in improved healthcare outcomes or reduced healthcare costs, comprising any one or more of the following drugs or indications:
for drugs—ABACAVIR SULFATE AND LAMIVUDINE, ABACAVIR SULFATE; ABACAVIR SULFATE, ABACAVIR SULFATE; ABACAVIR SULFATE, LAMIVUDINE AND ZIDOVUDINE; ABEMACICLIB; ABIRATERONE ACETATE, ABIRATERONE ACETATE; ACALABRUTINIB; ACALABRUTINIB MALEATE; ACAMPROSATE CALCIUM, ACAMPROSATE CALCIUM; ACARBOSE, ACARBOSE; ACEBUTOLOL HYDROCHLORIDE, ACEBUTOLOL HYDROCHLORIDE; ACETAMINOPHEN AND CODEINE PHOSPHATE, ACETAMINOPHEN; ACETAMINOPHEN AND IBUPROFEN, ACETAMINOPHEN (OTC); ACETAMINOPHEN, ACETAMINOPHEN; ACETAMINOPHEN, ACETAMINOPHEN (OTC); ACETAMINOPHEN, ASPIRIN AND CAFFEINE; ACETAZOLAMIDE SODIUM, ACETAZOLAMIDE SODIUM; ACETAZOLAMIDE, ACETAZOLAMIDE; ACETIC ACID 0.25% IN PLASTIC CONTAINER, ACETIC ACID; ACETIC ACID, ACETIC ACID, GLACIAL; ACETOHYDROXAMIC ACID; ACETYLCYSTEINE, ACETYLCYSTEINE; ACITRETIN, ACITRETIN; ACYCLOVIR SODIUM, ACYCLOVIR SODIUM; ACYCLOVIR, ACYCLOVIR; ADAGRASIB; ADAPALENE (OTC); ADAPALENE AND BENZOYL PEROXIDE, ADAPALENE; ADAPALENE, ADAPALENE; ADAPALENE, ADAPALENE (OTC); ADEFOVIR DIPIVOXIL, ADEFOVIR DIPIVOXIL; ADENOSINE, ADENOSINE; AFATINIB DIMALEATE; ALBENDAZOLE, ALBENDAZOLE; ALBUTEROL SULFATE AND IPRATROPIUM BROMIDE, ALBUTEROL SULFATE; ALBUTEROL SULFATE, ALBUTEROL SULFATE; ALCLOMETASONE DIPROPIONATE, ALCLOMETASONE DIPROPIONATE; ALECTINIB HYDROCHLORIDE; ALENDRONATE SODIUM, ALENDRONATE SODIUM; ALFENTANIL, ALFENTANIL HYDROCHLORIDE; ALFUZOSIN HYDROCHLORIDE, ALFUZOSIN HYDROCHLORIDE; ALISKIREN HEMIFUMARATE, ALISKIREN HEMIFUMARATE; ALITRETINOIN; ALLOPURINOL SODIUM, ALLOPURINOL SODIUM; ALLOPURINOL, ALLOPURINOL; ALMOTRIPTAN MALATE, ALMOTRIPTAN MALATE; ALOSETRON HYDROCHLORIDE, ALOSETRON HYDROCHLORIDE; ALPELISIB; ALPRAZOLAM, ALPRAZOLAM; ALPROSTADIL, ALPROSTADIL; ALVIMOPAN, ALVIMOPAN; AMANTADINE HYDROCHLORIDE, AMANTADINE HYDROCHLORIDE; AMBRISENTAN, AMBRISENTAN; AMCINONIDE, AMCINONIDE; AMIFAMPRIDINE PHOSPHATE; AMIKACIN SULFATE, AMIKACIN SULFATE; AMILORIDE HYDROCHLORIDE AND HYDROCHLOROTHIAZIDE, AMILORIDE HYDROCHLORIDE; AMILORIDE HYDROCHLORIDE, AMILORIDE HYDROCHLORIDE; AMINO ACIDS, AMINO ACIDS; AMINOACETIC ACID 1.5% IN PLASTIC CONTAINER, GLYCINE; AMINOCAPROIC ACID IN PLASTIC CONTAINER, AMINOCAPROIC ACID; AMINOCAPROIC ACID, AMINOCAPROIC ACID; AMINOPHYLLINE, AMINOPHYLLINE; AMIODARONE HYDROCHLORIDE, AMIODARONE HYDROCHLORIDE; AMITRIPTYLINE HYDROCHLORIDE, AMITRIPTYLINE HYDROCHLORIDE; AMLODIPINE AND OLMESARTAN MEDOXOMIL, AMLODIPINE BESYLATE; AMLODIPINE BESYLATE AND ATORVASTATIN CALCIUM, AMLODIPINE BESYLATE; AMLODIPINE BESYLATE AND BENAZEPRIL HYDROCHLORIDE, AMLODIPINE BESYLATE; AMLODIPINE BESYLATE AND VALSARTAN, AMLODIPINE BESYLATE; AMLODIPINE BESYLATE, AMLODIPINE BESYLATE; AMLODIPINE BESYLATE, VALSARTAN AND HYDROCHLOROTHIAZIDE; AMMONIA N 13, AMMONIA N-13; AMMONIUM CHLORIDE IN PLASTIC CONTAINER, AMMONIUM CHLORIDE; AMMONIUM LACTATE, AMMONIUM LACTATE; AMOXAPINE, AMOXAPINE; AMOXICILLIN AND CLAVULANATE POTASSIUM, AMOXICILLIN; AMOXICILLIN PEDIATRIC, AMOXICILLIN; AMOXICILLIN, AMOXICILLIN; AMPHETAMINE SULFATE, AMPHETAMINE SULFATE; AMPHOTERICIN B, AMPHOTERICIN B; AMPICILLIN AND SULBACTAM, AMPICILLIN SODIUM; AMPICILLIN SODIUM, AMPICILLIN SODIUM; AMPICILLIN TRIHYDRATE, AMPICILLIN/AMPICILLIN TRIHYDRATE; AN-SULFUR COLLOID, TECHNETIUM TC-99M SULFUR COLLOID KIT; ANAGRELIDE HYDROCHLORIDE, ANAGRELIDE HYDROCHLORIDE; ANASTROZOLE, ANASTROZOLE; ANIDULAFUNGIN; APALUTAMIDE; APIXABAN, APIXABAN; APOMORPHINE HYDROCHLORIDE, APOMORPHINE HYDROCHLORIDE; APRACLONIDINE HYDROCHLORIDE, APRACLONIDINE HYDROCHLORIDE; APREMILAST, APREMILAST; APREPITANT, APREPITANT; ARFORMOTEROL TARTRATE, ARFORMOTEROL TARTRATE; ARGATROBAN, ARGATROBAN; ARIPIPRAZOLE LAUROXIL; ARIPIPRAZOLE, ARIPIPRAZOLE; ARMODAFINIL, ARMODAFINIL; ARSENIC TRIOXIDE, ARSENIC TRIOXIDE; ARTEMETHER; ARTESUNATE, ARTESUNATE; ASCIMINIB HYDROCHLORIDE; ASCORBIC ACID; ASENAPINE; ASENAPINE MALEATE, ASENAPINE MALEATE; ASPIRIN AND DIPYRIDAMOLE, ASPIRIN; ATAZANAVIR SULFATE, ATAZANAVIR SULFATE; ATENOLOL AND CHLORTHALIDONE, ATENOLOL; ATENOLOL, ATENOLOL; ATOMOXETINE HYDROCHLORIDE, ATOMOXETINE HYDROCHLORIDE; ATORVASTATIN CALCIUM, ATORVASTATIN CALCIUM; ATOVAQUONE AND PROGUANIL HYDROCHLORIDE, ATOVAQUONE; ATOVAQUONE, ATOVAQUONE; ATRACURIUM BESYLATE PRESERVATIVE FREE, ATRACURIUM BESYLATE; ATRACURIUM BESYLATE, ATRACURIUM BESYLATE; ATROPINE SULFATE, ATROPINE SULFATE; AURANOFIN; AVACOPAN; AVAPRITINIB; AVATROMBOPAG MALEATE; AXITINIB; AZACITIDINE, AZACITIDINE; AZATHIOPRINE SODIUM, AZATHIOPRINE SODIUM; AZATHIOPRINE, AZATHIOPRINE; AZELAIC ACID, AZELAIC ACID; AZELASTINE HYDROCHLORIDE AND FLUTICASONE PROPIONATE, AZELASTINE HYDROCHLORIDE; AZELASTINE HYDROCHLORIDE, AZELASTINE HYDROCHLORIDE; AZILSARTAN KAMEDOXOMIL; AZITHROMYCIN, AZITHROMYCIN; AZTREONAM, AZTREONAM; BACITRACIN ZINC AND POLYMYXIN B SULFATE, BACITRACIN ZINC; BACITRACIN-NEOMYCIN-POLYMYXIN W/HYDROCORTISONE ACETATE, BACITRACIN ZINC; BACITRACIN, BACITRACIN; BACLOFEN, BACLOFEN; BACTERIOSTATIC SODIUM CHLORIDE 0.9% IN PLASTIC CONTAINER, SODIUM CHLORIDE; BACTERIOSTATIC WATER FOR INJECTION IN PLASTIC CONTAINER, STERILE WATER FOR INJECTION; BALANCED SALT, CALCIUM CHLORIDE; BALOXAVIR MARBOXIL; BALSALAZIDE DISODIUM, BALSALAZIDE DISODIUM; BARICITINIB; BAZEDOXIFENE ACETATE; BECLOMETHASONE DIPROPIONATE; BEDAQUILINE FUMARATE; BELUMOSUDIL MESYLATE; BELZUTIFAN; BENAZEPRIL HYDROCHLORIDE AND HYDROCHLOROTHIAZIDE, BENAZEPRIL HYDROCHLORIDE; BENAZEPRIL HYDROCHLORIDE, BENAZEPRIL HYDROCHLORIDE; BENDAMUSTINE HYDROCHLORIDE, BENDAMUSTINE HYDROCHLORIDE; BENZNIDAZOLE, BENZNIDAZOLE; BENZONATATE, BENZONATATE; BENZOYL PEROXIDE; BENZPHETAMINE HYDROCHLORIDE, BENZPHETAMINE HYDROCHLORIDE; BENZTROPINE MESYLATE, BENZTROPINE MESYLATE; BEPOTASTINE BESILATE, BEPOTASTINE BESILATE; BEROTRALSTAT HYDROCHLORIDE; BESIFLOXACIN HYDROCHLORIDE; BETAINE, BETAINE; BETAMETHASONE ACETATE AND BETAMETHASONE SODIUM PHOSPHATE, BETAMETHASONE ACETATE; BETAMETHASONE DIPROPIONATE, BETAMETHASONE DIPROPIONATE; BETAMETHASONE VALERATE, BETAMETHASONE VALERATE; BETAXOLOL HYDROCHLORIDE, BETAXOLOL HYDROCHLORIDE; BETHANECHOL CHLORIDE, BETHANECHOL CHLORIDE; BEXAROTENE, BEXAROTENE; BICALUTAMIDE, BICALUTAMIDE; BICTEGRAVIR SODIUM; BIMATOPROST, BIMATOPROST; BINIMETINIB; BISMUTH SUBCITRATE POTASSIUM; BISMUTH SUBCITRATE POTASSIUM, METRONIDAZOLE AND TETRACYCLINE HYDROCHLORIDE, BISMUTH; BISMUTH SUBSALICYLATE, METRONIDAZOLE AND TETRACYCLINE HYDROCHLORIDE, BISMUTH SUBSALICYLATE; BISOPROLOL FUMARATE AND HYDROCHLOROTHIAZIDE, BISOPROLOL FUMARATE; BISOPROLOL FUMARATE, BISOPROLOL FUMARATE; BIVALIRUDIN, BIVALIRUDIN; BLEOMYCIN SULFATE, BLEOMYCIN SULFATE; BORTEZOMIB, BORTEZOMIB; BOSENTAN, BOSENTAN; BOSUTINIB MONOHYDRATE; Brand, ABACAVIR SULFATE; Brand, ABALOPARATIDE; Brand, ABIRATERONE ACETATE; Brand, ABROCITINIB; Brand, ACETAMINOPHEN; Brand, ACETAMINOPHEN (OTC); Brand, ACETIC ACID, GLACIAL; Brand, ACETYLCHOLINE CHLORIDE; Brand, ACETYLCYSTEINE; Brand, ACLIDINIUM BROMIDE; Brand, ACYCLOVIR; Brand, ADAPALENE; Brand, AFAMELANOTIDE; Brand, AIR POLYMER-TYPE A; Brand, ALBUMIN HUMAN; Brand, ALBUTEROL SULFATE; Brand, ALCAFTADINE (OTC); Brand, ALCOHOL; Brand, ALCOHOL (OTC); Brand, ALENDRONATE SODIUM; Brand, ALFENTANIL HYDROCHLORIDE; Brand, ALFUZOSIN HYDROCHLORIDE; Brand, ALISKIREN HEMIFUMARATE; Brand, ALLOPURINOL; Brand, ALOGLIPTIN BENZOATE; Brand, ALOSETRON HYDROCHLORIDE; Brand, ALPHA-TOCOPHEROL ACETATE; Brand, ALPRAZOLAM; Brand, ALPROSTADIL; Brand, AMANTADINE HYDROCHLORIDE; Brand, AMBRISENTAN; Brand, AMIFOSTINE; Brand, AMILORIDE HYDROCHLORIDE; Brand, AMINO ACIDS; Brand, AMINOCAPROIC ACID; Brand, AMINOLEVULINIC ACID HYDROCHLORIDE; Brand, AMINOSALICYLIC ACID; Brand, AMIODARONE HYDROCHLORIDE; Brand, AMISULPRIDE; Brand, AMLODIPINE BENZOATE; Brand, AMLODIPINE BESYLATE; Brand, AMOXICILLIN; Brand, AMPHETAMINE; Brand, AMPHETAMINE ASPARTATE; Brand, AMPHETAMINE SULFATE; Brand, AMPHOTERICIN B; Brand, AMPICILLIN SODIUM; Brand, ANAGRELIDE HYDROCHLORIDE; Brand, ANASTROZOLE; Brand, ANGIOTENSIN II ACETATE; Brand, APOMORPHINE HYDROCHLORIDE; Brand, APREPITANT; Brand, ARFORMOTEROL TARTRATE; Brand, ARGATROBAN; Brand, ARGININE HYDROCHLORIDE; Brand, ARMODAFINIL; Brand, ARSENIC TRIOXIDE; Brand, ARTICAINE HYDROCHLORIDE; Brand, ASCORBIC ACID; Brand, ASENAPINE MALEATE; Brand, ASPIRIN; Brand, ASPIRIN (OTC); Brand, ATENOLOL; Brand, ATOGEPANT; Brand, ATOMOXETINE HYDROCHLORIDE; Brand, ATORVASTATIN CALCIUM; Brand, ATOVAQUONE; Brand, ATROPINE; Brand, ATROPINE SULFATE; Brand, AVACINCAPTAD PEGOL SODIUM; Brand, AVANAFIL; Brand, AVIBACTAM SODIUM; Brand, AVOBENZONE (OTC); Brand, AZACITIDINE; Brand, AZATHIOPRINE; Brand, AZELASTINE HYDROCHLORIDE; Brand, AZELASTINE HYDROCHLORIDE (OTC); Brand, AZITHROMYCIN; Brand, AZTREONAM; Brand, BACITRACIN ZINC; Brand, BACLOFEN; Brand, BALSALAZIDE DISODIUM; Brand, BARIUM SULFATE; Brand, BECLOMETHASONE DIPROPIONATE; Brand, BELINOSTAT; Brand, BEMPEDOIC ACID; Brand, BENAZEPRIL HYDROCHLORIDE; Brand, BENDAMUSTINE HYDROCHLORIDE; Brand, BENOXINATE HYDROCHLORIDE; Brand, BENTOQUATAM (OTC); Brand, BENZONATATE; Brand, BENZOYL PEROXIDE; Brand, BEPOTASTINE BESILATE; Brand, BETAMETHASONE ACETATE; Brand, BETAMETHASONE DIPROPIONATE; Brand, BETAMETHASONE VALERATE; Brand, BETAXOLOL HYDROCHLORIDE; Brand, BETHANECHOL CHLORIDE; Brand, BEXAGLIFLOZIN; Brand, BEXAROTENE; Brand, BICALUTAMIDE; Brand, BIMATOPROST; Brand, BIRCH TRITERPENES; Brand, BISOPROLOL FUMARATE; Brand, BIVALIRUDIN; Brand, BORTEZOMIB; Brand, BREMELANOTIDE ACETATE; Brand, BREXANOLONE; Brand, BRILLIANT BLUE G; Brand, BRIMONIDINE TARTRATE; Brand, BRIMONIDINE TARTRATE (OTC); Brand, BRINCIDOFOVIR; Brand, BRINZOLAMIDE; Brand, BROMFENAC SODIUM; Brand, BROMOCRIPTINE MESYLATE; Brand, BROMPHENIRAMINE MALEATE; Brand, BUDESONIDE; Brand, BUDESONIDE (OTC); Brand, BUMETANIDE; Brand, BUPIVACAINE; Brand, BUPIVACAINE HYDROCHLORIDE; Brand, BUPRENORPHINE; Brand, BUPRENORPHINE HYDROCHLORIDE; Brand, BUPROPION HYDROCHLORIDE; Brand, BUSULFAN; Brand, BUTENAFINE HYDROCHLORIDE (OTC); Brand, BUTOCONAZOLE NITRATE; Brand, CABAZITAXEL; Brand, CABOTEGRAVIR; Brand, CABOTEGRAVIR SODIUM; Brand, CAFFEINE CITRATE; Brand, CALCIFEDIOL; Brand, CALCIPOTRIENE; Brand, CALCITONIN SALMON; Brand, CALCITRIOL; Brand, CALCIUM ACETATE; Brand, CALCIUM CARBONATE (OTC); Brand, CALCIUM CHLORIDE; Brand, CANDESARTAN CILEXETIL; Brand, CANGRELOR; Brand, CANTHARIDIN; Brand, CAPECITABINE; Brand, CAPIVASERTIB; Brand, CAPSAICIN; Brand, CARBACHOL; Brand, CARBAMAZEPINE; Brand, CARBIDOPA; Brand, CARBINOXAMINE MALEATE; Brand, CARBOPROST TROMETHAMINE; Brand, CARFILZOMIB; Brand, CARGLUMIC ACID; Brand, CARISOPRODOL; Brand, CARMUSTINE; Brand, CARVEDILOL; Brand, CARVEDILOL PHOSPHATE; Brand, CASIMERSEN; Brand, CASPOFUNGIN ACETATE; Brand, CEFIXIME; Brand, CEFTAZIDIME; Brand, CELECOXIB; Brand, CETIRIZINE HYDROCHLORIDE; Brand, CETIRIZINE HYDROCHLORIDE (OTC); Brand, CETRORELIX ACETATE; Brand, CEVIMELINE HYDROCHLORIDE; Brand, CHLORDIAZEPOXIDE HYDROCHLORIDE; Brand, CHLORHEXIDINE GLUCONATE; Brand, CHLORHEXIDINE GLUCONATE (OTC); Brand, CHLOROPROCAINE HYDROCHLORIDE; Brand, CHLOROTHIAZIDE; Brand, CHLORPHENIRAMINE MALEATE; Brand, CHLORPHENIRAMINE MALEATE (OTC); Brand, CHLORTHALIDONE; Brand, CHOLESTYRAMINE; Brand, CHOLINE FENOFIBRATE; Brand, CICLESONIDE; Brand, CICLOPIROX; Brand, CILASTATIN SODIUM; Brand, CIMETIDINE (OTC); Brand, CINACALCET HYDROCHLORIDE; Brand, CIPROFLOXACIN; Brand, CIPROFLOXACIN HYDROCHLORIDE; Brand, CISATRACURIUM BESYLATE; Brand, CITALOPRAM HYDROBROMIDE; Brand, CITRIC ACID; Brand, CLADRIBINE; Brand, CLASCOTERONE; Brand, CLEVIDIPINE; Brand, CLINDAMYCIN PHOSPHATE; Brand, CLOBAZAM; Brand, CLOBETASOL PROPIONATE; Brand, CLOCORTOLONE PIVALATE; Brand, CLOFARABINE; Brand, CLOMIPRAMINE HYDROCHLORIDE; Brand, CLONAZEPAM; Brand, CLONIDINE; Brand, CLONIDINE HYDROCHLORIDE; Brand, CLOPIDOGREL BISULFATE; Brand, CLORAZEPATE DIPOTASSIUM; Brand, CLOTRIMAZOLE (OTC); Brand, CLOZAPINE; Brand, COCAINE HYDROCHLORIDE; Brand, CODEINE PHOSPHATE; Brand, COLCHICINE; Brand, COLESEVELAM HYDROCHLORIDE; Brand, COLESTIPOL HYDROCHLORIDE; Brand, CONIVAPTAN HYDROCHLORIDE; Brand, COPPER; Brand, COPPER CU-64 DOTATATE; Brand, COSYNTROPIN; Brand, CRISABOROLE; Brand, CROMOLYN SODIUM; Brand, CROTAMITON; Brand, CUPRIC SULFATE; Brand, CYANOCOBALAMIN; Brand, CYCLOBENZAPRINE HYDROCHLORIDE; Brand, CYCLOPENTOLATE HYDROCHLORIDE; Brand, CYCLOPHOSPHAMIDE; Brand, CYCLOSERINE; Brand, CYCLOSPORINE; Brand, CYSTEINE HYDROCHLORIDE; Brand, CYTARABINE; Brand, DACTINOMYCIN; Brand, DALFAMPRIDINE; Brand, DALFOPRISTIN; Brand, DANTROLENE SODIUM; Brand, DAPAGLIFLOZIN; Brand, DAPRODUSTAT; Brand, DAPSONE; Brand, DARIDOREXANT HYDROCHLORIDE; Brand, DASATINIB; Brand, DASIGLUCAGON HYDROCHLORIDE; Brand, DAUNORUBICIN HYDROCHLORIDE; Brand, DEFERASIROX; Brand, DEFEROXAMINE MESYLATE; Brand, DEFIBROTIDE SODIUM; Brand, DEOXYCHOLIC ACID; Brand, DESFLURANE; Brand, DESIPRAMINE HYDROCHLORIDE; Brand, DESLORATADINE; Brand, DESMOPRESSIN ACETATE; Brand, DESOGESTREL; Brand, DESONIDE; Brand, DESOXIMETASONE; Brand, DESVENLAFAXINE SUCCINATE; Brand, DEUCRAVACITINIB; Brand, DEUTETRABENAZINE; Brand, DEXAMETHASONE; Brand, DEXCHLORPHENIRAMINE MALEATE; Brand, DEXLANSOPRAZOLE; Brand, DEXMEDETOMIDINE HYDROCHLORIDE; Brand, DEXMETHYLPHENIDATE HYDROCHLORIDE; Brand, DEXTROAMPHETAMINE; Brand, DEXTROAMPHETAMINE SULFATE; Brand, DEXTROMETHORPHAN HYDROBROMIDE (OTC); Brand, DEXTROMETHORPHAN POLISTIREX (OTC); Brand, DEXTROSE; Brand, DIATRIZOATE MEGLUMINE; Brand, DIAZEPAM; Brand, DIAZOXIDE; Brand, DICLOFENAC EPOLAMINE; Brand, DICLOFENAC POTASSIUM; Brand, DICLOFENAC SODIUM; Brand, DICLOFENAC SODIUM (OTC); Brand, DICYCLOMINE HYDROCHLORIDE; Brand, DIFELIKEFALIN ACETATE; Brand, DIFLUPREDNATE; Brand, DIHYDROERGOTAMINE MESYLATE; Brand, DILTIAZEM HYDROCHLORIDE; Brand, DIMERCAPROL; Brand, DIMETHYL FUMARATE; Brand, DIMETHYL SULFOXIDE; Brand, DINOPROSTONE; Brand, DIPHENHYDRAMINE CITRATE (OTC); Brand, DIPHENHYDRAMINE HYDROCHLORIDE (OTC); Brand, DIPYRIDAMOLE; Brand, DIROXIMEL FUMARATE; Brand, DISOPYRAMIDE PHOSPHATE; Brand, DIVALPROEX SODIUM; Brand, DOCETAXEL; Brand, DOCOSANOL (OTC); Brand, DOFETILIDE; Brand, DONEPEZIL HYDROCHLORIDE; Brand, DORZOLAMIDE HYDROCHLORIDE; Brand, DOXAPRAM HYDROCHLORIDE; Brand, DOXEPIN HYDROCHLORIDE; Brand, DOXERCALCIFEROL; Brand, DOXORUBICIN HYDROCHLORIDE; Brand, DOXYCYCLINE; Brand, DOXYCYCLINE HYCLATE; Brand, DOXYLAMINE SUCCINATE; Brand, DOXYLAMINE SUCCINATE (OTC); Brand, DRONABINOL; Brand, DROSPIRENONE; Brand, DROXIDOPA; Brand, DULOXETINE HYDROCHLORIDE; Brand, DURLOBACTAM SODIUM; Brand, DUTASTERIDE; Brand, DYCLONINE HYDROCHLORIDE; Brand, ECHOTHIOPHATE IODIDE; Brand, ECONAZOLE NITRATE; Brand, EDARAVONE; Brand, EDOXABAN TOSYLATE; Brand, EFAVIRENZ; Brand, EFLORNITHINE HYDROCHLORIDE; Brand, ELAGOLIX SODIUM, ESTRADIOL, NORETHINDRONE ACETATE; Brand, ELBASVIR; Brand, ELETRIPTAN HYDROBROMIDE; Brand, ELTROMBOPAG CHOLINE; Brand, EMPAGLIFLOZIN; Brand, EMTRICITABINE; Brand, ENALAPRIL MALEATE; Brand, ENOXAPARIN SODIUM; Brand, ENTACAPONE; Brand, EPHEDRINE HYDROCHLORIDE; Brand, EPHEDRINE SULFATE; Brand, EPINEPHRINE; Brand, EPINEPHRINE (OTC); Brand, EPINEPHRINE BITARTRATE; Brand, EPIRUBICIN HYDROCHLORIDE; Brand, EPLERENONE; Brand, EPLONTERSEN SODIUM; Brand, EPOPROSTENOL SODIUM; Brand, ERAVACYCLINE DIHYDROCHLORIDE; Brand, ERGOCALCIFEROL; Brand, ERIBULIN MESYLATE; Brand, ERTAPENEM SODIUM; Brand, ERTUGLIFLOZIN; Brand, ERYTHROMYCIN; Brand, ERYTHROMYCIN ETHYLSUCCINATE; Brand, ERYTHROMYCIN LACTOBIONATE; Brand, ESCITALOPRAM OXALATE; Brand, ESKETAMINE HYDROCHLORIDE; Brand, ESMOLOL HYDROCHLORIDE; Brand, ESOMEPRAZOLE MAGNESIUM; Brand, ESOMEPRAZOLE MAGNESIUM (OTC); Brand, ESOMEPRAZOLE SODIUM; Brand, ESTRADIOL; Brand, ESTRADIOL ACETATE; Brand, ESTRADIOL VALERATE; Brand, ESTROPIPATE; Brand, ESZOPICLONE; Brand, ETELCALCETIDE; Brand, ETEPLIRSEN; Brand, ETHACRYNATE SODIUM; Brand, ETHACRYNIC ACID; Brand, ETHAMBUTOL HYDROCHLORIDE; Brand, ETHINYL ESTRADIOL; Brand, ETHIODIZED OIL; Brand, ETHOSUXIMIDE; Brand, ETOMIDATE; Brand, ETONOGESTREL; Brand, ETOPOSIDE PHOSPHATE; Brand, ETRASIMOD ARGININE; Brand, EVEROLIMUS; Brand, EXEMESTANE; Brand, EXENATIDE SYNTHETIC; Brand, EZETIMIBE; Brand, FAMOTIDINE; Brand, FAMOTIDINE (OTC); Brand, FEBUXOSTAT; Brand, FELBAMATE; Brand, FENOFIBRATE; Brand, FENOLDOPAM MESYLATE; Brand, FENOPROFEN CALCIUM; Brand, FENTANYL CITRATE; Brand, FERRIC CARBOXYMALTOSE; Brand, FERRIC DERISOMALTOSE; Brand, FERRIC HEXACYANOFERRATE(II); Brand, FERRIC MALTOL; Brand, FERRIC OXYHYDROXIDE; Brand, FERUMOXYTOL; Brand, FESOTERODINE FUMARATE; Brand, FEXOFENADINE HYDROCHLORIDE; Brand, FEXOFENADINE HYDROCHLORIDE (OTC); Brand, FEZOLINETANT; Brand, FINASTERIDE; Brand, FINGOLIMOD HYDROCHLORIDE; Brand, FINGOLIMOD LAURYL SULFATE; Brand, FLIBANSERIN; Brand, FLORBETABEN F-18; Brand, FLORBETAPIR F-18; Brand, FLORTAUCIPIR F-18; Brand, FLOTUFOLASTAT F-18 GALLIUM; Brand, FLUCICLOVINE F-18; Brand, FLUCONAZOLE; Brand, FLUCYTOSINE; Brand, FLUOCINOLONE ACETONIDE; Brand, FLUOCINONIDE; Brand, FLUORESCEIN SODIUM; Brand, FLUOROESTRADIOL F-18; Brand, FLUOROMETHOLONE; Brand, FLUOROMETHOLONE ACETATE; Brand, FLUOROURACIL; Brand, FLUOXETINE HYDROCHLORIDE; Brand, FLURANDRENOLIDE; Brand, FLUTEMETAMOL F-18; Brand, FLUTICASONE FUROATE (OTC); Brand, FLUTICASONE PROPIONATE; Brand, FLUTICASONE PROPIONATE (OTC); Brand, FLUVASTATIN SODIUM; Brand, FLUVOXAMINE MALEATE; Brand, FONDAPARINUX SODIUM; Brand, FORMOTEROL FUMARATE; Brand, FOSAPREPITANT DIMEGLUMINE; Brand, FOSCARNET SODIUM; Brand, FOSDENOPTERIN HYDROBROMIDE; Brand, FOSFOMYCIN TROMETHAMINE; Brand, FOSNETUPITANT CHLORIDE HYDROCHLORIDE; Brand, FOSPHENYTOIN SODIUM; Brand, FROVATRIPTAN SUCCINATE; Brand, FRUQUINTINIB; Brand, FULVESTRANT; Brand, FUROSEMIDE; Brand, GABAPENTIN; Brand, GADOBENATE DIMEGLUMINE; Brand, GADOBUTROL; Brand, GADODIAMIDE; Brand, GADOPICLENOL; Brand, GADOTERATE MEGLUMINE; Brand, GADOTERIDOL; Brand, GADOXETATE DISODIUM; Brand, GALLIUM DOTATATE GA-68; Brand, GALLIUM GA-68 GOZETOTIDE; Brand, GANCICLOVIR; Brand, GANIRELIX ACETATE; Brand, GATIFLOXACIN; Brand, GEFITINIB; Brand, GEMFIBROZIL; Brand, GEPIRONE HYDROCHLORIDE; Brand, GIVOSIRAN SODIUM; Brand, GLIMEPIRIDE; Brand, GLIPIZIDE; Brand, GLUCAGON; Brand, GLYBURIDE; Brand, GLYCOPYRROLATE; Brand, GLYCOPYRRONIUM TOSYLATE; Brand, GOLODIRSEN; Brand, GOSERELIN ACETATE; Brand, GRAMICIDIN; Brand, GRANISETRON; Brand, GRISEOFULVIN, MICROCRYSTALLINE; Brand, GRISEOFULVIN, ULTRAMICROCRYSTALLINE; Brand, GRISEOFULVIN, ULTRAMICROSIZE; Brand, GUAIFENESIN (OTC); Brand, GUANFACINE HYDROCHLORIDE; Brand, HALCINONIDE; Brand, HALOBETASOL PROPIONATE; Brand, HALOPERIDOL DECANOATE; Brand, HEPARIN SODIUM; Brand, HEXACHLOROPHENE; Brand, HEXAMINOLEVULINATE HYDROCHLORIDE; Brand, HISTRELIN ACETATE; Brand, HOMATROPINE METHYLBROMIDE; Brand, HYDRALAZINE HYDROCHLORIDE; Brand, HYDROCHLOROTHIAZIDE; Brand, HYDROCODONE BITARTRATE; Brand, HYDROCORTISONE; Brand, HYDROCORTISONE ACETATE; Brand, HYDROCORTISONE BUTYRATE; Brand, HYDROCORTISONE PROBUTATE; Brand, HYDROMORPHONE HYDROCHLORIDE; Brand, HYDROXOCOBALAMIN; Brand, HYDROXYCHLOROQUINE SULFATE; Brand, HYDROXYUREA; Brand, HYDROXYZINE PAMOATE; Brand, IBREXAFUNGERP CITRATE; Brand, IBUPROFEN; Brand, IBUPROFEN (OTC); Brand, IBUPROFEN LYSINE; Brand, IBUPROFEN SODIUM (OTC); Brand, IBUTILIDE FUMARATE; Brand, ICATIBANT ACETATE; Brand, ICODEXTRIN; Brand, IDARUBICIN HYDROCHLORIDE; Brand, IFOSFAMIDE; Brand, ILOPROST; Brand, IMATINIB MESYLATE; Brand, IMIPRAMINE HYDROCHLORIDE; Brand, IMIQUIMOD; Brand, INAMRINONE LACTATE; Brand, INCLISIRAN SODIUM; Brand, INDIGOTINDISULFONATE SODIUM; Brand, INDIUM IN-111 PENTETREOTIDE KIT; Brand, INDOCYANINE GREEN; Brand, INDOMETHACIN; Brand, IOBENGUANE I-131; Brand, IOBENGUANE SULFATE I-123; Brand, IODINE POVACRYLEX (OTC); Brand, IODIXANOL; Brand, IOFLUPANE I-123; Brand, IOHEXOL; Brand, IOPAMIDOL; Brand, IOPROMIDE; Brand, IOTHALAMATE MEGLUMINE; Brand, IOTHALAMATE SODIUM I-125; Brand, IOVERSOL; Brand, IPTACOPAN HYDROCHLORIDE; Brand, IRBESARTAN; Brand, IRINOTECAN HYDROCHLORIDE; Brand, ISOFLURANE; Brand, ISOPROPYL ALCOHOL (OTC); Brand, ISOSORBIDE DINITRATE; Brand, ISOSORBIDE MONONITRATE; Brand, ISOTRETINOIN; Brand, ITRACONAZOLE; Brand, IVACAFTOR; Brand, IVERMECTIN; Brand, IVERMECTIN (OTC); Brand, IXABEPILONE; Brand, KETAMINE HYDROCHLORIDE; Brand, KETOCONAZOLE; Brand, KETOCONAZOLE (OTC); Brand, KETOROLAC TROMETHAMINE; Brand, KETOTIFEN FUMARATE (OTC); Brand, LACOSAMIDE; Brand, LAMIVUDINE; Brand, LAMOTRIGINE; Brand, LANSOPRAZOLE; Brand, LANTHANUM CARBONATE; Brand, LAPATINIB DITOSYLATE; Brand, LASMIDITAN SUCCINATE; Brand, LATANOPROST; Brand, LATANOPROSTENE BUNOD; Brand, LEDIPASVIR; Brand, LEFLUNOMIDE; Brand, LEMBOREXANT; Brand, LETROZOLE; Brand, LEUPROLIDE ACETATE; Brand, LEUPROLIDE MESYLATE; Brand, LEVALBUTEROL HYDROCHLORIDE; Brand, LEVALBUTEROL TARTRATE; Brand, LEVETIRACETAM; Brand, LEVOBUNOLOL HYDROCHLORIDE; Brand, LEVOCARNITINE; Brand, LEVOCETIRIZINE DIHYDROCHLORIDE (OTC); Brand, LEVOLEUCOVORIN; Brand, LEVOLEUCOVORIN CALCIUM; Brand, LEVONORGESTREL; Brand, LEVONORGESTREL (OTC); Brand, LEVOTHYROXINE SODIUM; Brand, LEVOTHYROXINE SODIUM **; Brand, LIDOCAINE; Brand, LIDOCAINE HYDROCHLORIDE; Brand, LIFITEGRAST; Brand, LINCOMYCIN HYDROCHLORIDE; Brand, LINEZOLID; Brand, LIRAGLUTIDE RECOMBINANT; Brand, LISDEXAMFETAMINE DIMESYLATE; Brand, LISINOPRIL; Brand, LITHIUM CARBONATE; Brand, LOPERAMIDE HYDROCHLORIDE (OTC); Brand, LOPINAVIR; Brand, LORATADINE (OTC); Brand, LORAZEPAM; Brand, LOSARTAN POTASSIUM; Brand, LOTEPREDNOL ETABONATE; Brand, LOTILANER; Brand, LOXAPINE; Brand, LUBIPROSTONE; Brand, LULICONAZOLE; Brand, LUMASIRAN SODIUM; Brand, LURASIDONE HYDROCHLORIDE; Brand, LURBINECTEDIN; Brand, LUTETIUM LU 177 DOTATATE; Brand, LUTETIUM LU-177 VIPIVOTIDE TETRAXETAN; Brand, MAGNESIUM CHLORIDE; Brand, MAGNESIUM SULFATE; Brand, MANNITOL; Brand, MECLIZINE HYDROCHLORIDE; Brand, MEDROXYPROGESTERONE ACETATE; Brand, MEFENAMIC ACID; Brand, MEGESTROL ACETATE; Brand, MELOXICAM; Brand, MELPHALAN HYDROCHLORIDE; Brand, MEMANTINE HYDROCHLORIDE; Brand, MENTHOL (OTC); Brand, MEPERIDINE HYDROCHLORIDE; Brand, MEPIVACAINE HYDROCHLORIDE; Brand, MERCAPTOPURINE; Brand, MEROPENEM; Brand, MESALAMINE; Brand, METFORMIN HYDROCHLORIDE; Brand, METHACHOLINE CHLORIDE; Brand, METHAMPHETAMINE HYDROCHLORIDE; Brand, METHENAMINE HIPPURATE; Brand, METHOCARBAMOL; Brand, METHOHEXITAL SODIUM; Brand, METHOTREXATE; Brand, METHOTREXATE SODIUM; Brand, METHOXSALEN; Brand, METHSUXIMIDE; Brand, METHYLENE BLUE; Brand, METHYLERGONOVINE MALEATE; Brand, METHYLPHENIDATE; Brand, METHYLPHENIDATE HYDROCHLORIDE; Brand, METOCLOPRAMIDE HYDROCHLORIDE; Brand, METOPROLOL SUCCINATE; Brand, METOPROLOL TARTRATE; Brand, METRONIDAZOLE; Brand, METYROSINE; Brand, MICAFUNGIN SODIUM; Brand, MICONAZOLE; Brand, MICONAZOLE NITRATE; Brand, MICONAZOLE NITRATE (OTC); Brand, MIDAZOLAM HYDROCHLORIDE; Brand, MIDODRINE HYDROCHLORIDE; Brand, MIFEPRISTONE; Brand, MIGLITOL; Brand, MIGLUSTAT; Brand, MILTEFOSINE; Brand, MINOCYCLINE HYDROCHLORIDE; Brand, MINOXIDIL (OTC); Brand, MIRABEGRON; Brand, MIRTAZAPINE; Brand, MISOPROSTOL; Brand, MITOMYCIN; Brand, MODAFINIL; Brand, MOMELOTINIB DIHYDROCHLORIDE; Brand, MOMETASONE FUROATE; Brand, MOMETASONE FUROATE (OTC); Brand, MONOMETHYL FUMARATE; Brand, MONTELUKAST SODIUM; Brand, MORPHINE SULFATE; Brand, MOTIXAFORTIDE ACETATE; Brand, MOXIFLOXACIN HYDROCHLORIDE; Brand, MUPIROCIN; Brand, NABILONE; Brand, NADOLOL; Brand, NAFCILLIN SODIUM; Brand, NAFTIFINE HYDROCHLORIDE; Brand, NALOXONE HYDROCHLORIDE (OTC); Brand, NAPHAZOLINE HYDROCHLORIDE (OTC); Brand, NAPROXEN; Brand, NAPROXEN SODIUM; Brand, NAPROXEN SODIUM (OTC); Brand, NEBIVOLOL HYDROCHLORIDE; Brand, NEDOSIRAN SODIUM; Brand, NELARABINE; Brand, NEOMYCIN SULFATE; Brand, NEOSTIGMINE METHYLSULFATE; Brand, NEPAFENAC; Brand, NEVIRAPINE; Brand, NIACIN; Brand, NICARDIPINE HYDROCHLORIDE; Brand, NICOTINE; Brand, NICOTINE (OTC); Brand, NICOTINE POLACRILEX (OTC); Brand, NIFEDIPINE; Brand, NIFURTIMOX; Brand, NILUTAMIDE; Brand, NIMODIPINE; Brand, NIRMATRELVIR; Brand, NIROGACESTAT HYDROBROMIDE; Brand, NISOLDIPINE; Brand, NITAZOXANIDE; Brand, NITISINONE; Brand, NITRIC OXIDE; Brand, NITROFURANTOIN; Brand, NITROGLYCERIN; Brand, NIZATIDINE (OTC); Brand, NOREPINEPHRINE BITARTRATE; Brand, NORETHINDRONE; Brand, NORTRIPTYLINE HYDROCHLORIDE; Brand, NUSINERSEN SODIUM; Brand, NYSTATIN; Brand, OCTREOTIDE ACETATE; Brand, OFLOXACIN; Brand, OLANZAPINE; Brand, OLANZAPINE PAMOATE; Brand, OLICERIDINE; Brand, OLMESARTAN MEDOXOMIL; Brand, OLODATEROL HYDROCHLORIDE; Brand, OLOPATADINE HYDROCHLORIDE (OTC); Brand, OMEGA-3-ACID ETHYL ESTERS; Brand, OMEPRAZOLE; Brand, OMEPRAZOLE (OTC); Brand, OMEPRAZOLE MAGNESIUM; Brand, OMEPRAZOLE MAGNESIUM (OTC); Brand, ORITAVANCIN DIPHOSPHATE; Brand, ORLISTAT; Brand, ORLISTAT (OTC); Brand, OSELTAMIVIR PHOSPHATE; Brand, OSPEMIFENE; Brand, OXACILLIN SODIUM; Brand, OXALIPLATIN; Brand, OXAPROZIN; Brand, OXCARBAZEPINE; Brand, OXICONAZOLE NITRATE; Brand, OXYBUTYNIN; Brand, OXYBUTYNIN (OTC); Brand, OXYCODONE; Brand, OXYCODONE HYDROCHLORIDE; Brand, OXYMETAZOLINE HYDROCHLORIDE; Brand, OXYMETAZOLINE HYDROCHLORIDE (OTC); Brand, OXYTOCIN; Brand, OZANIMOD HYDROCHLORIDE; Brand, OZENOXACIN; Brand, PACLITAXEL; Brand, PAFOLACIANINE SODIUM; Brand, PALOVAROTENE; Brand, PANTOPRAZOLE SODIUM; Brand, PARICALCITOL; Brand, PAROXETINE HYDROCHLORIDE; Brand, PAROXETINE MESYLATE; Brand, PASIREOTIDE PAMOATE; Brand, PATISIRAN SODIUM; Brand, PEGCETACOPLAN; Brand, PEMETREXED; Brand, PEMETREXED DISODIUM; Brand, PENCICLOVIR; Brand, PENICILLAMINE; Brand, PENTAMIDINE ISETHIONATE; Brand, PENTOSTATIN; Brand, PERAMIVIR; Brand, PERFLUOROHEXYLOCTANE; Brand, PERFLUTREN; Brand, PERMETHRIN; Brand, PERMETHRIN (OTC); Brand, PHENDIMETRAZINE TARTRATE; Brand, PHENELZINE SULFATE; Brand, PHENOBARBITAL SODIUM; Brand, PHENOXYBENZAMINE HYDROCHLORIDE; Brand, PHENTERMINE HYDROCHLORIDE; Brand, PHENTOLAMINE MESYLATE; Brand, PHENYLEPHRINE HYDROCHLORIDE; Brand, PHENYTOIN SODIUM; Brand, PIFLUFOLASTAT F-18; Brand, PILOCARPINE HYDROCHLORIDE; Brand, PIMECROLIMUS; Brand, PIOGLITAZONE HYDROCHLORIDE; Brand, PIPERACILLIN SODIUM; Brand, PIRFENIDONE; Brand, PIROXICAM; Brand, PITAVASTATIN MAGNESIUM; Brand, PLAZOMICIN SULFATE; Brand, PLECANATIDE; Brand, PLERIXAFOR; Brand, POLIDOCANOL; Brand, POLYETHYLENE GLYCOL 3350; Brand, POLYETHYLENE GLYCOL 3350 (OTC); Brand, PONESIMOD; Brand, PORFIMER SODIUM; Brand, POSACONAZOLE; Brand, POTASSIUM CHLORIDE; Brand, POTASSIUM CITRATE; Brand, POTASSIUM IODIDE (OTC); Brand, POVIDONE-IODINE; Brand, PRALATREXATE; Brand, PRALIDOXIME CHLORIDE; Brand, PRAMIPEXOLE DIHYDROCHLORIDE; Brand, PRASTERONE; Brand, PRASUGREL HYDROCHLORIDE; Brand, PRAZIQUANTEL; Brand, PRAZOSIN HYDROCHLORIDE; Brand, PREDNISOLONE; Brand, PREDNISOLONE ACETATE; Brand, PREDNISOLONE SODIUM PHOSPHATE; Brand, PREDNISONE; Brand, PREGABALIN; Brand, PRIMIDONE; Brand, PROBENECID; Brand, PROCHLORPERAZINE MALEATE; Brand, PROGESTERONE; Brand, PROPARACAINE HYDROCHLORIDE; Brand, PROPOFOL; Brand, PROPRANOLOL HYDROCHLORIDE; Brand, PRUCALOPRIDE SUCCINATE; Brand, PSEUDOEPHEDRINE HYDROCHLORIDE (OTC); Brand, PURIFIED WATER (OTC); Brand, PYRIDOSTIGMINE BROMIDE; Brand, PYRIMETHAMINE; Brand, QUAZEPAM; Brand, QUETIAPINE FUMARATE; Brand, QUININE SULFATE; Brand, RABEPRAZOLE SODIUM; Brand, RADIUM RA-223 DICHLORIDE; Brand, RALOXIFENE HYDROCHLORIDE; Brand, RAMELTEON; Brand, RAMIPRIL; Brand, RANOLAZINE; Brand, RASAGILINE MESYLATE; Brand, REGADENOSON; Brand, REMDESIVIR; Brand, REMIFENTANIL HYDROCHLORIDE; Brand, REMIMAZOLAM BESYLATE; Brand, REPOTRECTINIB; Brand, RETAPAMULIN; Brand, REZAFUNGIN ACETATE; Brand, RIBAVIRIN; Brand, RIBOFLAVIN 5′-PHOSPHATE; Brand, RIFABUTIN; Brand, RIFAMPIN; Brand, RIFAMYCIN SODIUM; Brand, RILPIVIRINE HYDROCHLORIDE; Brand, RILUZOLE; Brand, RIMEGEPANT SULFATE; Brand, RISEDRONATE SODIUM; Brand, RISPERIDONE; Brand, RITLECITINIB TOSYLATE; Brand, RIVASTIGMINE; Brand, RIZATRIPTAN BENZOATE; Brand, ROFLUMILAST; Brand, ROMIDEPSIN; Brand, ROPIVACAINE HYDROCHLORIDE; Brand, RUBIDIUM CHLORIDE RB-82; Brand, RUFINAMIDE; Brand, SALMETEROL XINAFOATE; Brand, SAPROPTERIN DIHYDROCHLORIDE; Brand, SARECYCLINE HYDROCHLORIDE; Brand, SAXAGLIPTIN HYDROCHLORIDE; Brand, SCOPOLAMINE; Brand, SECRETIN SYNTHETIC HUMAN; Brand, SEMAGLUTIDE; Brand, SERTACONAZOLE NITRATE; Brand, SERTRALINE HYDROCHLORIDE; Brand, SETMELANOTIDE ACETATE; Brand, SEVELAMER CARBONATE; Brand, SEVELAMER HYDROCHLORIDE; Brand, SEVOFLURANE; Brand, SILDENAFIL CITRATE; Brand, SILODOSIN; Brand, SILVER SULFADIAZINE; Brand, SIMVASTATIN; Brand, SINCALIDE; Brand, SINECATECHINS; Brand, SIPONIMOD; Brand, SIROLIMUS; Brand, SITAGLIPTIN; Brand, SODIUM BENZOATE; Brand, SODIUM IODIDE I-131; Brand, SODIUM NITRITE; Brand, SODIUM NITROPRUSSIDE; Brand, SODIUM OXYBATE; Brand, SODIUM PHENYLBUTYRATE; Brand, SODIUM POLYSTYRENE SULFONATE; Brand, SODIUM TETRADECYL SULFATE; Brand, SODIUM THIOSULFATE; Brand, SOFOSBUVIR; Brand, SOLIFENACIN SUCCINATE; Brand, SORAFENIB TOSYLATE; Brand, SOTAGLIFLOZIN; Brand, SOTALOL HYDROCHLORIDE; Brand, SOYBEAN OIL; Brand, SPINOSAD; Brand, SPIRONOLACTONE; Brand, STREPTOZOCIN; Brand, STRONTIUM CHLORIDE SR-89; Brand, SUCCINYLCHOLINE CHLORIDE; Brand, SUFENTANIL CITRATE; Brand, SUGAMMADEX SODIUM; Brand, SULCONAZOLE NITRATE; Brand, SULFACETAMIDE SODIUM; Brand, SULFAMETHOXAZOLE; Brand, SULFASALAZINE; Brand, SULFUR HEXAFLUORIDE LIPID-TYPE A MICROSPHERES; Brand, SUMATRIPTAN; Brand, SUMATRIPTAN SUCCINATE; Brand, SUNITINIB MALATE; Brand, TACROLIMUS; Brand, TADALAFIL; Brand, TAFENOQUINE SUCCINATE; Brand, TAFLUPROST; Brand, TALC; Brand, TAMSULOSIN HYDROCHLORIDE; Brand, TAPENTADOL HYDROCHLORIDE; Brand, TAPINAROF; Brand, TASIMELTEON; Brand, TAVABOROLE; Brand, TAZAROTENE; Brand, TECHNETIUM TC-99M BICISATE KIT; Brand, TECHNETIUM TC-99M EXAMETAZIME KIT; Brand, TECHNETIUM TC-99M LABELED CARBON; Brand, TECHNETIUM TC-99M MEBROFENIN KIT; Brand, TECHNETIUM TC-99M MEDRONATE; Brand, TECHNETIUM TC-99M MEDRONATE KIT; Brand, TECHNETIUM TC-99M MERTIATIDE KIT; Brand, TECHNETIUM TC-99M OXIDRONATE KIT; Brand, TECHNETIUM TC-99M PENTETATE KIT; Brand, TECHNETIUM TC-99M PYROPHOSPHATE KIT; Brand, TECHNETIUM TC-99M RED BLOOD CELL KIT; Brand, TECHNETIUM TC-99M SESTAMIBI KIT; Brand, TECHNETIUM TC-99M SODIUM PERTECHNETATE GENERATOR; Brand, TECHNETIUM TC-99M SUCCIMER; Brand, TECHNETIUM TC-99M TETROFOSMIN KIT; Brand, TECHNETIUM TC-99M TILMANOCEPT; Brand, TECOVIRIMAT; Brand, TELAVANCIN HYDROCHLORIDE; Brand, TELMISARTAN; Brand, TEMAZEPAM; Brand, TEMOZOLOMIDE; Brand, TEMSIROLIMUS; Brand, TENAPANOR HYDROCHLORIDE; Brand, TERBINAFINE (OTC); Brand, TERBINAFINE HYDROCHLORIDE (OTC); Brand, TERBUTALINE SULFATE; Brand, TERIFLUNOMIDE; Brand, TERIPARATIDE; Brand, TERLIPRESSIN ACETATE; Brand, TESTOSTERONE; Brand, TESTOSTERONE ENANTHATE; Brand, TESTOSTERONE UNDECANOATE; Brand, TETRABENAZINE; Brand, TETRAHYDROZOLINE HYDROCHLORIDE; Brand, THEOPHYLLINE; Brand, THIOTEPA; Brand, TIAGABINE HYDROCHLORIDE; Brand, TIGECYCLINE; Brand, TIMOLOL; Brand, TIMOLOL MALEATE; Brand, TINIDAZOLE; Brand, TIOCONAZOLE (OTC); Brand, TIOPRONIN; Brand, TIOTROPIUM BROMIDE; Brand, TIROFIBAN HYDROCHLORIDE; Brand, TIRZEPATIDE; Brand, TIZANIDINE HYDROCHLORIDE; Brand, TOBRAMYCIN; Brand, TOFACITINIB CITRATE; Brand, TOFERSEN; Brand, TOLCAPONE; Brand, TOLTERODINE TARTRATE; Brand, TOLVAPTAN; Brand, TOPIRAMATE; Brand, TOPOTECAN HYDROCHLORIDE; Brand, TOREMIFENE CITRATE; Brand, TORSEMIDE; Brand, TRABECTEDIN; Brand, TRAMADOL HYDROCHLORIDE; Brand, TRANEXAMIC ACID; Brand, TRANYLCYPROMINE SULFATE; Brand, TRAVOPROST; Brand, TREPROSTINIL; Brand, TRETINOIN; Brand, TRIAMCINOLONE ACETONIDE; Brand, TRIAMCINOLONE ACETONIDE (OTC); Brand, TRIAMTERENE; Brand, TRIAZOLAM; Brand, TRICLABENDAZOLE; Brand, TRIENTINE HYDROCHLORIDE; Brand, TRIFAROTENE; Brand, TRIFLURIDINE; Brand, TRILACICLIB DIHYDROCHLORIDE; Brand, TRIMETHOBENZAMIDE HYDROCHLORIDE; Brand, TROMETHAMINE; Brand, TROPICAMIDE; Brand, TRYPAN BLUE; Brand, ULIPRISTAL ACETATE; Brand, UREA, C-14; Brand, URIDINE TRIACETATE; Brand, URSODIOL; Brand, VALACYCLOVIR HYDROCHLORIDE; Brand, VALGANCICLOVIR HYDROCHLORIDE; Brand, VALRUBICIN; Brand, VALSARTAN; Brand, VAMOROLONE; Brand, VANCOMYCIN HYDROCHLORIDE; Brand, VARENICLINE TARTRATE; Brand, VASOPRESSIN; Brand, VENLAFAXINE HYDROCHLORIDE; Brand, VERAPAMIL HYDROCHLORIDE; Brand, VERTEPORFIN; Brand, VIGABATRIN; Brand, VILAZODONE HYDROCHLORIDE; Brand, VILOXAZINE HYDROCHLORIDE; Brand, VILTOLARSEN; Brand, VITAMIN A PALMITATE; Brand, VONOPRAZAN FUMARATE; Brand, VORICONAZOLE; Brand, VOSORITIDE; Brand, VUTRISIRAN SODIUM; Brand, XENON XE-129 HYPERPOLARIZED; Brand, ZAFIRLUKAST; Brand, ZALEPLON; Brand, ZAVEGEPANT HYDROCHLORIDE; Brand, ZICONOTIDE ACETATE; Brand, ZIDOVUDINE; Brand, ZILUCOPLAN SODIUM; Brand, ZINC ACETATE; Brand, ZIPRASIDONE HYDROCHLORIDE; Brand, ZIPRASIDONE MESYLATE; Brand, ZOLEDRONIC ACID; Brand, ZOLMITRIPTAN; Brand, ZOLPIDEM TARTRATE; Brand, ZONISAMIDE; Brand, ZURANOLONE; BREXPIPRAZOLE, BREXPIPRAZOLE; BRIGATINIB; BRIMONIDINE TARTRATE AND TIMOLOL MALEATE, BRIMONIDINE TARTRATE; BRIMONIDINE TARTRATE, BRIMONIDINE TARTRATE; BRINZOLAMIDE, BRINZOLAMIDE; BRIVARACETAM; BROMFENAC SODIUM, BROMFENAC SODIUM; BROMOCRIPTINE MESYLATE, BROMOCRIPTINE MESYLATE; BROMPHENIRAMINE MALEATE, PSEUDOEPHEDRINE HYDROCHLORIDE AND DEXTROMETHORPHAN HYDROBROMIDE; BUDESONIDE, BUDESONIDE; BUDESONIDE, BUDESONIDE (OTC); BUMETANIDE, BUMETANIDE; BUPIVACAINE HYDROCHLORIDE AND EPINEPHRINE, BUPIVACAINE HYDROCHLORIDE; BUPIVACAINE HYDROCHLORIDE PRESERVATIVE FREE, BUPIVACAINE HYDROCHLORIDE; BUPIVACAINE HYDROCHLORIDE, BUPIVACAINE HYDROCHLORIDE; BUPRENORPHINE HYDROCHLORIDE AND NALOXONE HYDROCHLORIDE, BUPRENORPHINE HYDROCHLORIDE; BUPRENORPHINE HYDROCHLORIDE, BUPRENORPHINE HYDROCHLORIDE; BUPRENORPHINE, BUPRENORPHINE; BUPROPION HYDROBROMIDE; BUPROPION HYDROCHLORIDE, BUPROPION HYDROCHLORIDE; BUSPIRONE HYDROCHLORIDE, BUSPIRONE HYDROCHLORIDE; BUSULFAN, BUSULFAN; BUTALBITAL AND ACETAMINOPHEN, ACETAMINOPHEN; BUTALBITAL, ACETAMINOPHEN AND CAFFEINE, ACETAMINOPHEN; BUTALBITAL, ACETAMINOPHEN, CAFFEINE AND CODEINE PHOSPHATE, ACETAMINOPHEN; BUTALBITAL, ASPIRIN AND CAFFEINE, ASPIRIN; BUTALBITAL, ASPIRIN, CAFFEINE, AND CODEINE PHOSPHATE, ASPIRIN; BUTENAFINE HYDROCHLORIDE, BUTENAFINE HYDROCHLORIDE (OTC); BUTORPHANOL TARTRATE PRESERVATIVE FREE, BUTORPHANOL TARTRATE; BUTORPHANOL TARTRATE, BUTORPHANOL TARTRATE; CABAZITAXEL, CABAZITAXEL; CABERGOLINE, CABERGOLINE; CABOTEGRAVIR; CABOZANTINIB S-MALATE; CAFFEINE; CAFFEINE CITRATE, CAFFEINE CITRATE; CALCIPOTRIENE AND BETAMETHASONE DIPROPIONATE, BETAMETHASONE DIPROPIONATE; CALCIPOTRIENE AND BETHAMETHASONE DIPROPIONATE, BETAMETHASONE DIPROPIONATE; CALCIPOTRIENE, CALCIPOTRIENE; CALCITONIN-SALMON, CALCITONIN SALMON; CALCITRIOL, CALCITRIOL; CALCIUM ACETATE, CALCIUM ACETATE; CALCIUM CHLORIDE 10% IN PLASTIC CONTAINER, CALCIUM CHLORIDE; CALCIUM CHLORIDE 10%, CALCIUM CHLORIDE; CALCIUM GLUCONATE IN SODIUM CHLORIDE, CALCIUM GLUCONATE; CALCIUM GLUCONATE, CALCIUM GLUCONATE; CALCIUM OXYBATE; CANAGLIFLOZIN; CANDESARTAN CILEXETIL AND HYDROCHLOROTHIAZIDE, CANDESARTAN CILEXETIL; CANDESARTAN CILEXETIL, CANDESARTAN CILEXETIL; CANNABIDIOL; CAPECITABINE, CAPECITABINE; CAPMATINIB HYDROCHLORIDE; CAPTOPRIL AND HYDROCHLOROTHIAZIDE, CAPTOPRIL; CAPTOPRIL, CAPTOPRIL; CARBAMAZEPINE, CARBAMAZEPINE; CARBIDOPA AND LEVODOPA, CARBIDOPA; CARBIDOPA, CARBIDOPA; CARBIDOPA, LEVODOPA AND ENTACAPONE, CARBIDOPA; CARBINOXAMINE MALEATE, CARBINOXAMINE MALEATE; CARBOPLATIN, CARBOPLATIN; CARBOPROST TROMETHAMINE, CARBOPROST TROMETHAMINE; CARFILZOMIB, CARFILZOMIB; CARGLUMIC ACID, CARGLUMIC ACID; CARIPRAZINE HYDROCHLORIDE; CARISOPRODOL, CARISOPRODOL; CARMUSTINE, CARMUSTINE; CARTEOLOL HYDROCHLORIDE, CARTEOLOL HYDROCHLORIDE; CARVEDILOL PHOSPHATE, CARVEDILOL PHOSPHATE; CARVEDILOL, CARVEDILOL; CASPOFUNGIN ACETATE, CASPOFUNGIN ACETATE; CEDAZURIDINE; CEFACLOR, CEFACLOR; CEFADROXIL, CEFADROXIL/CEFADROXIL HEMIHYDRATE; CEFAZOLIN AND DEXTROSE, CEFAZOLIN SODIUM; CEFAZOLIN IN PLASTIC CONTAINER, CEFAZOLIN SODIUM; CEFAZOLIN SODIUM, CEFAZOLIN SODIUM; CEFDINIR, CEFDINIR; CEFEPIME AND DEXTROSE IN DUPLEX CONTAINER, CEFEPIME HYDROCHLORIDE; CEFEPIME HYDROCHLORIDE IN PLASTIC CONTAINER, CEFEPIME HYDROCHLORIDE; CEFEPIME HYDROCHLORIDE, CEFEPIME HYDROCHLORIDE; CEFEPIME IN PLASTIC CONTAINER, CEFEPIME HYDROCHLORIDE; CEFIDEROCOL SULFATE TOSYLATE; CEFIXIME, CEFIXIME; CEFOTETAN DISODIUM; CEFOXITIN AND DEXTROSE IN DUPLEX CONTAINER, CEFOXITIN SODIUM; CEFOXITIN IN PLASTIC CONTAINER, CEFOXITIN SODIUM; CEFOXITIN, CEFOXITIN SODIUM; CEFPODOXIME PROXETIL, CEFPODOXIME PROXETIL; CEFPROZIL, CEFPROZIL; CEFTAROLINE FOSAMIL; CEFTAZIDIME, CEFTAZIDIME; CEFTOLOZANE SULFATE; CEFTRIAXONE AND DEXTROSE IN DUPLEX CONTAINER, CEFTRIAXONE SODIUM; CEFTRIAXONE IN PLASTIC CONTAINER, CEFTRIAXONE SODIUM; CEFTRIAXONE SODIUM, CEFTRIAXONE SODIUM; CEFTRIAXONE, CEFTRIAXONE SODIUM; CEFUROXIME AXETIL, CEFUROXIME AXETIL; CEFUROXIME SODIUM, CEFUROXIME SODIUM; CELECOXIB, CELECOXIB; CENOBAMATE; CEPHALEXIN, CEPHALEXIN; CERITINIB; CETIRIZINE HYDROCHLORIDE ALLERGY, CETIRIZINE HYDROCHLORIDE (OTC); CETIRIZINE HYDROCHLORIDE AND PSEUDOEPHEDRINE HYDROCHLORIDE, CETIRIZINE HYDROCHLORIDE; CETIRIZINE HYDROCHLORIDE HIVES RELIEF, CETIRIZINE HYDROCHLORIDE (OTC); CETIRIZINE HYDROCHLORIDE HIVES, CETIRIZINE HYDROCHLORIDE (OTC); CETIRIZINE HYDROCHLORIDE, CETIRIZINE HYDROCHLORIDE; CETIRIZINE HYDROCHLORIDE, CETIRIZINE HYDROCHLORIDE (OTC); CETRORELIX ACETATE, CETRORELIX ACETATE; CEVIMELINE HYDROCHLORIDE, CEVIMELINE HYDROCHLORIDE; CHENODIOL, CHENODIOL; CHILDREN'S CETIRIZINE HYDROCHLORIDE ALLERGY, CETIRIZINE HYDROCHLORIDE; CHILDREN'S CETIRIZINE HYDROCHLORIDE HIVES RELIEF, CETIRIZINE HYDROCHLORIDE (OTC); CHILDREN'S FEXOFENADINE HYDROCHLORIDE ALLERGY, FEXOFENADINE HYDROCHLORIDE; CHILDREN'S FEXOFENADINE HYDROCHLORIDE HIVES, FEXOFENADINE HYDROCHLORIDE; CHILDREN'S IBUPROFEN, IBUPROFEN (OTC); CHLORAMBUCIL; CHLORAMPHENICOL SODIUM SUCCINATE, CHLORAMPHENICOL SODIUM SUCCINATE; CHLORDIAZEPOXIDE AND AMITRIPTYLINE HYDROCHLORIDE, AMITRIPTYLINE HYDROCHLORIDE; CHLORDIAZEPOXIDE HYDROCHLORIDE AND CLIDINIUM BROMIDE, CHLORDIAZEPOXIDE HYDROCHLORIDE; CHLORDIAZEPOXIDE HYDROCHLORIDE, CHLORDIAZEPOXIDE HYDROCHLORIDE; CHLORHEXIDINE GLUCONATE, CHLORHEXIDINE GLUCONATE; CHLORHEXIDINE GLUCONATE, CHLORHEXIDINE GLUCONATE (OTC); CHLOROPROCAINE HYDROCHLORIDE, CHLOROPROCAINE HYDROCHLORIDE; CHLOROQUINE PHOSPHATE, CHLOROQUINE PHOSPHATE; CHLOROTHIAZIDE SODIUM, CHLOROTHIAZIDE SODIUM; CHLORPHENIRAMINE MALEATE, CHLORPHENIRAMINE MALEATE (OTC); CHLORPROMAZINE HYDROCHLORIDE, CHLORPROMAZINE HYDROCHLORIDE; CHLORTHALIDONE, CHLORTHALIDONE; CHLORZOXAZONE, CHLORZOXAZONE; CHOLESTYRAMINE LIGHT, CHOLESTYRAMINE; CHOLESTYRAMINE, CHOLESTYRAMINE; CHOLIC ACID; CHOLINE C-11, CHOLINE C-11; CHROMIC CHLORIDE IN PLASTIC CONTAINER, CHROMIC CHLORIDE; CICLOPIROX, CICLOPIROX; CIDOFOVIR, CIDOFOVIR; CILASTATIN SODIUM; CILOSTAZOL, CILOSTAZOL; CIMETIDINE HYDROCHLORIDE, CIMETIDINE HYDROCHLORIDE; CIMETIDINE, CIMETIDINE; CIMETIDINE, CIMETIDINE (OTC); CINACALCET HYDROCHLORIDE, CINACALCET HYDROCHLORIDE; CIPROFLOXACIN AND DEXAMETHASONE, CIPROFLOXACIN; CIPROFLOXACIN EXTENDED RELEASE, CIPROFLOXACIN; CIPROFLOXACIN HYDROCHLORIDE, CIPROFLOXACIN HYDROCHLORIDE; CIPROFLOXACIN IN DEXTROSE 5% IN PLASTIC CONTAINER, CIPROFLOXACIN; CIPROFLOXACIN, CIPROFLOXACIN; CISATRACURIUM BESYLATE PRESERVATIVE FREE, CISATRACURIUM BESYLATE; CISATRACURIUM BESYLATE, CISATRACURIUM BESYLATE; CISPLATIN, CISPLATIN; CITALOPRAM HYDROBROMIDE, CITALOPRAM HYDROBROMIDE; CITRIC ACID; CLADRIBINE, CLADRIBINE; CLARITHROMYCIN, CLARITHROMYCIN; CLEMASTINE FUMARATE, CLEMASTINE FUMARATE; CLINDAMYCIN HYDROCHLORIDE, CLINDAMYCIN HYDROCHLORIDE; CLINDAMYCIN PALMITATE HYDROCHLORIDE, CLINDAMYCIN PALMITATE HYDROCHLORIDE; CLINDAMYCIN PHOSPHATE AND BENZOYL PEROXIDE, BENZOYL PEROXIDE; CLINDAMYCIN PHOSPHATE AND TRETINOIN, CLINDAMYCIN PHOSPHATE; CLINDAMYCIN PHOSPHATE IN 0.9% SODIUM CHLORIDE, CLINDAMYCIN PHOSPHATE; CLINDAMYCIN PHOSPHATE IN 5% DEXTROSE IN PLASTIC CONTAINER, CLINDAMYCIN PHOSPHATE; CLINDAMYCIN PHOSPHATE IN DEXTROSE 5%, CLINDAMYCIN PHOSPHATE; CLINDAMYCIN PHOSPHATE, CLINDAMYCIN PHOSPHATE; CLOBAZAM, CLOBAZAM; CLOBETASOL PROPIONATE (EMOLLIENT), CLOBETASOL PROPIONATE; CLOBETASOL PROPIONATE, CLOBETASOL PROPIONATE; CLOCORTOLONE PIVALATE, CLOCORTOLONE PIVALATE; CLOFARABINE, CLOFARABINE; CLOMIPHENE CITRATE, CLOMIPHENE CITRATE; CLOMIPRAMINE HYDROCHLORIDE, CLOMIPRAMINE HYDROCHLORIDE; CLONAZEPAM, CLONAZEPAM; CLONIDINE HYDROCHLORIDE, CLONIDINE HYDROCHLORIDE; CLONIDINE, CLONIDINE; CLOPIDOGREL BISULFATE, CLOPIDOGREL BISULFATE; CLORAZEPATE DIPOTASSIUM, CLORAZEPATE DIPOTASSIUM; CLOTRIMAZOLE AND BETAMETHASONE DIPROPIONATE, BETAMETHASONE DIPROPIONATE; CLOTRIMAZOLE, CLOTRIMAZOLE; CLOTRIMAZOLE, CLOTRIMAZOLE (OTC); CLOZAPINE, CLOZAPINE; COBICISTAT; COBIMETINIB FUMARATE; CODEINE SULFATE, CODEINE SULFATE; COLCHICINE, COLCHICINE; COLESEVELAM HYDROCHLORIDE, COLESEVELAM HYDROCHLORIDE; COLESTIPOL HYDROCHLORIDE, COLESTIPOL HYDROCHLORIDE; COLISTIMETHATE SODIUM, COLISTIMETHATE SODIUM; COLISTIN SULFATE; CORTICOTROPIN; COSYNTROPIN, COSYNTROPIN; CRIZOTINIB; CROFELEMER; CROMOLYN SODIUM, CROMOLYN SODIUM; CROMOLYN SODIUM, CROMOLYN SODIUM (OTC); CROTAMITON; CUPRIC CHLORIDE IN PLASTIC CONTAINER, CUPRIC CHLORIDE; CUPRIC CHLORIDE, CUPRIC CHLORIDE; CUPRIC SULFATE, CUPRIC SULFATE; CYANOCOBALAMIN, CYANOCOBALAMIN; CYCLOBENZAPRINE HYDROCHLORIDE, CYCLOBENZAPRINE HYDROCHLORIDE; CYCLOPHOSPHAMIDE, CYCLOPHOSPHAMIDE; CYCLOSPORINE, CYCLOSPORINE; CYPROHEPTADINE HYDROCHLORIDE, CYPROHEPTADINE HYDROCHLORIDE; CYSTEAMINE BITARTRATE; CYSTEAMINE HYDROCHLORIDE; CYTARABINE, CYTARABINE; DABIGATRAN ETEXILATE MESYLATE, DABIGATRAN ETEXILATE MESYLATE; DABRAFENIB MESYLATE; DACARBAZINE, DACARBAZINE; DACOMITINIB; DACTINOMYCIN, DACTINOMYCIN; DALBAVANCIN HYDROCHLORIDE; DALFAMPRIDINE, DALFAMPRIDINE; DALTEPARIN SODIUM; DANAZOL, DANAZOL; DANTROLENE SODIUM, DANTROLENE SODIUM; DAPSONE, DAPSONE; DAPTOMYCIN IN 0.9% SODIUM CHLORIDE, DAPTOMYCIN; DAPTOMYCIN, DAPTOMYCIN; DARIFENACIN HYDROBROMIDE, DARIFENACIN HYDROBROMIDE; DARIFENACIN, DARIFENACIN HYDROBROMIDE; DAROLUTAMIDE; DARUNAVIR, DARUNAVIR; DASATINIB; DAUNORUBICIN HYDROCHLORIDE, DAUNORUBICIN HYDROCHLORIDE; DECITABINE, DECITABINE; DEFERASIROX, DEFERASIROX; DEFERIPRONE, DEFERIPRONE; DEFEROXAMINE MESYLATE, DEFEROXAMINE MESYLATE; DEFLAZACORT; DEGARELIX ACETATE; DELAFLOXACIN MEGLUMINE; DEMECLOCYCLINE HYDROCHLORIDE, DEMECLOCYCLINE HYDROCHLORIDE; DEOXYCHOLIC ACID, DEOXYCHOLIC ACID; DESFLURANE, DESFLURANE; DESIPRAMINE HYDROCHLORIDE, DESIPRAMINE HYDROCHLORIDE; DESLORATADINE AND PSEUDOEPHEDRINE SULFATE 24 HOUR, DESLORATADINE; DESLORATADINE, DESLORATADINE; DESMOPRESSIN ACETATE (NEEDS NO REFRIGERATION), DESMOPRESSIN ACETATE; DESMOPRESSIN ACETATE, DESMOPRESSIN ACETATE; DESOGESTREL; DESOGESTREL AND ETHINYL ESTRADIOL, DESOGESTREL; DESONIDE, DESONIDE; DESOXIMETASONE, DESOXIMETASONE; DESVENLAFAXINE SUCCINATE, DESVENLAFAXINE SUCCINATE; DESVENLAFAXINE, DESVENLAFAXINE; DEUTETRABENAZINE; DEXAMETHASONE INTENSOL, DEXAMETHASONE; DEXAMETHASONE SODIUM PHOSPHATE PRESERVATIVE FREE, DEXAMETHASONE SODIUM PHOSPHATE; DEXAMETHASONE SODIUM PHOSPHATE, DEXAMETHASONE SODIUM PHOSPHATE; DEXAMETHASONE, DEXAMETHASONE; DEXCHLORPHENIRAMINE MALEATE, DEXCHLORPHENIRAMINE MALEATE; DEXLANSOPRAZOLE, DEXLANSOPRAZOLE; DEXMEDETOMIDINE HYDROCHLORIDE, DEXMEDETOMIDINE HYDROCHLORIDE; DEXMETHYLPHENIDATE HYDROCHLORIDE, DEXMETHYLPHENIDATE HYDROCHLORIDE; DEXRAZOXANE HYDROCHLORIDE, DEXRAZOXANE HYDROCHLORIDE; DEXTROAMP SACCHARATE, AMP ASPARTATE, DEXTROAMP SULFATE AND AMP SULFATE, AMPHETAMINE; DEXTROAMPHETAMINE SULFATE, DEXTROAMPHETAMINE SULFATE; DEXTROMETHORPHAN HYDROBROMIDE; DEXTROMETHORPHAN HYDROBROMIDE AND QUINIDINE SULFATE, DEXTROMETHORPHAN HYDROBROMIDE; DEXTROMETHORPHAN POLISTIREX, DEXTROMETHORPHAN POLISTIREX (OTC); DEXTROSE; DEXTROSE 10% AND SODIUM CHLORIDE 0.11% IN PLASTIC CONTAINER, DEXTROSE; DEXTROSE 10% AND SODIUM CHLORIDE 0.2% IN PLASTIC CONTAINER, DEXTROSE; DEXTROSE 10% AND SODIUM CHLORIDE 0.33% IN PLASTIC CONTAINER, DEXTROSE; DEXTROSE 10% AND SODIUM CHLORIDE 0.45% IN PLASTIC CONTAINER, DEXTROSE; DEXTROSE 10% AND SODIUM CHLORIDE 0.9% IN PLASTIC CONTAINER, DEXTROSE; DEXTROSE 10% IN PLASTIC CONTAINER, DEXTROSE; DEXTROSE 2.5% AND SODIUM CHLORIDE 0.11% IN PLASTIC CONTAINER, DEXTROSE; DEXTROSE 2.5% AND SODIUM CHLORIDE 0.2% IN PLASTIC CONTAINER, DEXTROSE; DEXTROSE 2.5% AND SODIUM CHLORIDE 0.33% IN PLASTIC CONTAINER, DEXTROSE; DEXTROSE 2.5% AND SODIUM CHLORIDE 0.45% IN PLASTIC CONTAINER, DEXTROSE; DEXTROSE 2.5% AND SODIUM CHLORIDE 0.9% IN PLASTIC CONTAINER, DEXTROSE; DEXTROSE 2.5% IN HALF-STRENGTH LACTATED RINGER'S IN PLASTIC CONTAINER, CALCIUM CHLORIDE; DEXTROSE 20% IN PLASTIC CONTAINER, DEXTROSE; DEXTROSE 25%, DEXTROSE; DEXTROSE 3.3% AND SODIUM CHLORIDE 0.3% IN PLASTIC CONTAINER, DEXTROSE; DEXTROSE 30% IN PLASTIC CONTAINER, DEXTROSE; DEXTROSE 40% IN PLASTIC CONTAINER, DEXTROSE; DEXTROSE 5% AND ELECTROLYTE NO. 48 IN PLASTIC CONTAINER, DEXTROSE; DEXTROSE 5% AND LACTATED RINGER'S IN PLASTIC CONTAINER, CALCIUM CHLORIDE; DEXTROSE 5% AND POTASSIUM CHLORIDE 0.075% IN PLASTIC CONTAINER, DEXTROSE; DEXTROSE 5% AND POTASSIUM CHLORIDE 0.075%, DEXTROSE; DEXTROSE 5% AND POTASSIUM CHLORIDE 0.15% IN PLASTIC CONTAINER, DEXTROSE; DEXTROSE 5% AND POTASSIUM CHLORIDE 0.15%, DEXTROSE; DEXTROSE 5% AND POTASSIUM CHLORIDE 0.224% IN PLASTIC CONTAINER, DEXTROSE; DEXTROSE 5% AND POTASSIUM CHLORIDE 0.3% IN PLASTIC CONTAINER, DEXTROSE; DEXTROSE 5% AND SODIUM CHLORIDE 0.11% IN PLASTIC CONTAINER, DEXTROSE; DEXTROSE 5% AND SODIUM CHLORIDE 0.2% IN PLASTIC CONTAINER, DEXTROSE; DEXTROSE 5% AND SODIUM CHLORIDE 0.225% IN PLASTIC CONTAINER, DEXTROSE; DEXTROSE 5% AND SODIUM CHLORIDE 0.225%, DEXTROSE; DEXTROSE 5% AND SODIUM CHLORIDE 0.3% IN PLASTIC CONTAINER, DEXTROSE; DEXTROSE 5% AND SODIUM CHLORIDE 0.33% IN PLASTIC CONTAINER, DEXTROSE; DEXTROSE 5% AND SODIUM CHLORIDE 0.45% IN PLASTIC CONTAINER, DEXTROSE; DEXTROSE 5% AND SODIUM CHLORIDE 0.45%, DEXTROSE; DEXTROSE 5% AND SODIUM CHLORIDE 0.9% IN PLASTIC CONTAINER, DEXTROSE; DEXTROSE 5% AND SODIUM CHLORIDE 0.9%, DEXTROSE; DEXTROSE 5% IN LACTATED RINGER'S IN PLASTIC CONTAINER, CALCIUM CHLORIDE; DEXTROSE 5% IN PLASTIC CONTAINER, DEXTROSE; DEXTROSE 5% IN SODIUM CHLORIDE 0.2% IN PLASTIC CONTAINER, DEXTROSE; DEXTROSE 5% IN SODIUM CHLORIDE 0.33% IN PLASTIC CONTAINER, DEXTROSE; DEXTROSE 5% IN SODIUM CHLORIDE 0.45% IN PLASTIC CONTAINER, DEXTROSE; DEXTROSE 5% IN SODIUM CHLORIDE 0.9% IN PLASTIC CONTAINER, DEXTROSE; DEXTROSE 5%, SODIUM CHLORIDE 0.2% AND POTASSIUM CHLORIDE 10 MEQ, DEXTROSE; DEXTROSE 5%, SODIUM CHLORIDE 0.2% AND POTASSIUM CHLORIDE 15 MEQ (K), DEXTROSE; DEXTROSE 5%, SODIUM CHLORIDE 0.2% AND POTASSIUM CHLORIDE 20 MEQ (K), DEXTROSE; DEXTROSE 5%, SODIUM CHLORIDE 0.2% AND POTASSIUM CHLORIDE 20 MEQ, DEXTROSE; DEXTROSE 5%, SODIUM CHLORIDE 0.2% AND POTASSIUM CHLORIDE 30 MEQ, DEXTROSE; DEXTROSE 5%, SODIUM CHLORIDE 0.2% AND POTASSIUM CHLORIDE 40 MEQ, DEXTROSE; DEXTROSE 5%, SODIUM CHLORIDE 0.2% AND POTASSIUM CHLORIDE 5 MEQ (K), DEXTROSE; DEXTROSE 5%, SODIUM CHLORIDE 0.2% AND POTASSIUM CHLORIDE 5 MEQ, DEXTROSE; DEXTROSE 5%, SODIUM CHLORIDE 0.33% AND POTASSIUM CHLORIDE 10 MEQ IN PLASTIC CONTAINER; DEXTROSE 5%, SODIUM CHLORIDE 0.33% AND POTASSIUM CHLORIDE 15 MEQ IN PLASTIC CONTAINER; DEXTROSE 5%, SODIUM CHLORIDE 0.33% AND POTASSIUM CHLORIDE 20 MEQ IN PLASTIC CONTAINER; DEXTROSE 5%, SODIUM CHLORIDE 0.33% AND POTASSIUM CHLORIDE 30 MEQ IN PLASTIC CONTAINER; DEXTROSE 5%, SODIUM CHLORIDE 0.33% AND POTASSIUM CHLORIDE 40 MEQ IN PLASTIC CONTAINER; DEXTROSE 5%, SODIUM CHLORIDE 0.33% AND POTASSIUM CHLORIDE 5 MEQ IN PLASTIC CONTAINER; DEXTROSE 5%, SODIUM CHLORIDE 0.45% AND POTASSIUM CHLORIDE 20 MEQ (K) IN PLASTIC CONTAINER; DEXTROSE 50% IN PLASTIC CONTAINER, DEXTROSE; DEXTROSE 50%, DEXTROSE; DEXTROSE 70% IN PLASTIC CONTAINER, DEXTROSE; DIAZEPAM INTENSOL, DIAZEPAM; DIAZEPAM, DIAZEPAM; DIAZOXIDE, DIAZOXIDE; DICHLORPHENAMIDE, DICHLORPHENAMIDE; DICLOFENAC POTASSIUM, DICLOFENAC POTASSIUM; DICLOFENAC SODIUM AND MISOPROSTOL, DICLOFENAC SODIUM; DICLOFENAC SODIUM, DICLOFENAC SODIUM; DICLOFENAC SODIUM, DICLOFENAC SODIUM (OTC); DICLOXACILLIN SODIUM, DICLOXACILLIN SODIUM; DICYCLOMINE HYDROCHLORIDE (PRESERVATIVE FREE), DICYCLOMINE HYDROCHLORIDE; DICYCLOMINE HYDROCHLORIDE, DICYCLOMINE HYDROCHLORIDE; DIENOGEST; DIETHYLPROPION HYDROCHLORIDE, DIETHYLPROPION HYDROCHLORIDE; DIFLORASONE DIACETATE, DIFLORASONE DIACETATE; DIFLUNISAL, DIFLUNISAL; DIFLUPREDNATE, DIFLUPREDNATE; DIGOXIN, DIGOXIN; DIHYDROERGOTAMINE MESYLATE, DIHYDROERGOTAMINE MESYLATE; DILTIAZEM HYDROCHLORIDE, DILTIAZEM HYDROCHLORIDE; DIMENHYDRINATE, DIMENHYDRINATE; DIMETHYL FUMARATE, DIMETHYL FUMARATE; DIPENTUM, OLSALAZINE SODIUM; DIPHENHYDRAMINE HYDROCHLORIDE PRESERVATIVE FREE, DIPHENHYDRAMINE HYDROCHLORIDE; DIPHENHYDRAMINE HYDROCHLORIDE, DIPHENHYDRAMINE HYDROCHLORIDE; DIPHENOXYLATE HYDROCHLORIDE AND ATROPINE SULFATE, ATROPINE SULFATE; DIPYRIDAMOLE, DIPYRIDAMOLE; DISOPYRAMIDE PHOSPHATE, DISOPYRAMIDE PHOSPHATE; DISULFIRAM, DISULFIRAM; DIVALPROEX SODIUM, DIVALPROEX SODIUM; DOBUTAMINE HYDROCHLORIDE IN DEXTROSE 5% IN PLASTIC CONTAINER, DOBUTAMINE HYDROCHLORIDE; DOBUTAMINE HYDROCHLORIDE, DOBUTAMINE HYDROCHLORIDE; DOCETAXEL, DOCETAXEL; DOCOSANOL, DOCOSANOL (OTC); DOFETILDE, DOFETILIDE; DOFETILIDE, DOFETILIDE; DOLASETRON MESYLATE; DOLUTEGRAVIR SODIUM; DONEPEZIL HYDROCHLORIDE, DONEPEZIL HYDROCHLORIDE; DOPAMINE HYDROCHLORIDE IN DEXTROSE 5% IN PLASTIC CONTAINER, DOPAMINE HYDROCHLORIDE; DOPAMINE HYDROCHLORIDE, DOPAMINE HYDROCHLORIDE; DORAVIRINE; DORZOLAMIDE HYDROCHLORIDE AND TIMOLOL MALEATE, DORZOLAMIDE HYDROCHLORIDE; DORZOLAMIDE HYDROCHLORIDE, DORZOLAMIDE HYDROCHLORIDE; DOXAPRAM HYDROCHLORIDE, DOXAPRAM HYDROCHLORIDE; DOXAZOSIN MESYLATE, DOXAZOSIN MESYLATE; DOXEPIN HYDROCHLORIDE, DOXEPIN HYDROCHLORIDE; DOXERCALCIFEROL, DOXERCALCIFEROL; DOXORUBICIN HYDROCHLORIDE (LIPOSOMAL), DOXORUBICIN HYDROCHLORIDE; DOXORUBICIN HYDROCHLORIDE, DOXORUBICIN HYDROCHLORIDE; DOXYCYCLINE CALCIUM; DOXYCYCLINE HYCLATE, DOXYCYCLINE HYCLATE; DOXYCYCLINE, DOXYCYCLINE; DOXYCYCLINE, DOXYCYCLINE HYCLATE; DOXYLAMINE SUCCINATE AND PYRIDOXINE HYDROCHLORIDE, DOXYLAMINE SUCCINATE; DOXYLAMINE SUCCINATE, DOXYLAMINE SUCCINATE (OTC); DRONABINOL, DRONABINOL; DRONEDARONE HYDROCHLORIDE; DROPERIDOL, DROPERIDOL; DROSPIRENONE; DROSPIRENONE AND ETHINYL ESTRADIOL, DROSPIRENONE; DROSPIRENONE, ETHINYL ESTRADIOL AND LEVOMEFOLATE CALCIUM, DROSPIRENONE; DROXIDOPA, DROXIDOPA; DULOXETINE HYDROCHLORIDE, DULOXETINE HYDROCHLORIDE; DUTASTERIDE AND TAMSULOSIN HYDROCHLORIDE, DUTASTERIDE; DUTASTERIDE, DUTASTERIDE; DUVELISIB; ECONAZOLE NITRATE, ECONAZOLE NITRATE; EDARAVONE; EDETATE CALCIUM DISODIUM, EDETATE CALCIUM DISODIUM; EFAVIRENZ, EFAVIRENZ; EFAVIRENZ, EMTRICITABINE, AND TENOFOVIR DISOPROXIL FUMARATE, EFAVIRENZ; EFAVIRENZ, LAMIVUDINE AND TENOFOVIR DISOPROXIL FUMARATE, EFAVIRENZ; EFAVIRENZ, EMTRICITABINE AND TENOFOVIR DISOPROXIL FUMARATE, EFAVIRENZ; EFINACONAZOLE, EFINACONAZOLE; ELACESTRANT DIHYDROCHLORIDE; ELAGOLIX SODIUM; ELETRIPTAN HYDROBROMIDE, ELETRIPTAN HYDROBROMIDE; ELEXACAFTOR, IVACAFTOR, TEZACAFTOR; ELIGLUSTAT TARTRATE, ELIGLUSTAT TARTRATE; ELTROMBOPAG OLAMINE; ELUXADOLINE; EMPAGLIFLOZIN; EMTRICITABINE AND TENOFOVIR DISOPROXIL FUMARATE, EMTRICITABINE; EMTRICITABINE, EMTRICITABINE; ENALAPRIL MALEATE AND HYDROCHLOROTHIAZIDE, ENALAPRIL MALEATE; ENALAPRIL MALEATE, ENALAPRIL MALEATE; ENALAPRILAT, ENALAPRILAT; ENASIDENIB MESYLATE; ENCORAFENIB; ENFUVIRTIDE; ENOXAPARIN SODIUM (PRESERVATIVE FREE), ENOXAPARIN SODIUM; ENOXAPARIN SODIUM, ENOXAPARIN SODIUM; ENTACAPONE, ENTACAPONE; ENTECAVIR, ENTECAVIR; ENTRECTINIB; ENZALUTAMIDE; EPHEDRINE SULFATE, EPHEDRINE SULFATE; EPINASTINE HYDROCHLORIDE, EPINASTINE HYDROCHLORIDE; EPINEPHRINE (AUTOINJECTOR), EPINEPHRINE; EPINEPHRINE, EPINEPHRINE; EPIRUBICIN HYDROCHLORIDE, EPIRUBICIN HYDROCHLORIDE; EPLERENONE, EPLERENONE; EPOPROSTENOL SODIUM, EPOPROSTENOL SODIUM; EPTIFIBATIDE, EPTIFIBATIDE; ERDAFITINIB; ERGOCALCIFEROL, ERGOCALCIFEROL; ERGOLOID MESYLATES, ERGOLOID MESYLATES; ERGOTAMINE TARTRATE; ERGOTAMINE TARTRATE AND CAFFEINE, CAFFEINE; ERLOTINIB HYDROCHLORIDE, ERLOTINIB HYDROCHLORIDE; ERTAPENEM SODIUM, ERTAPENEM SODIUM; ERYTHROMYCIN AND BENZOYL PEROXIDE, BENZOYL PEROXIDE; ERYTHROMYCIN ETHYLSUCCINATE, ERYTHROMYCIN ETHYLSUCCINATE; ERYTHROMYCIN LACTOBIONATE, ERYTHROMYCIN LACTOBIONATE; ERYTHROMYCIN STEARATE; ERYTHROMYCIN, ERYTHROMYCIN; ESCITALOPRAM OXALATE, ESCITALOPRAM OXALATE; ESLICARBAZEPINE ACETATE, ESLICARBAZEPINE ACETATE; ESMOLOL HYDROCHLORIDE DOUBLE STRENGTH IN PLASTIC CONTAINER, ESMOLOL HYDROCHLORIDE; ESMOLOL HYDROCHLORIDE IN PLASTIC CONTAINER, ESMOLOL HYDROCHLORIDE; ESMOLOL HYDROCHLORIDE, ESMOLOL HYDROCHLORIDE; ESOMEPRAZOLE MAGNESIUM, ESOMEPRAZOLE MAGNESIUM; ESOMEPRAZOLE MAGNESIUM, ESOMEPRAZOLE MAGNESIUM (OTC); ESOMEPRAZOLE SODIUM, ESOMEPRAZOLE SODIUM; ESTAZOLAM, ESTAZOLAM; ESTRADIOL AND NORETHINDRONE ACETATE, ESTRADIOL; ESTRADIOL AND NORGESTIMATE, ESTRADIOL; ESTRADIOL CYPIONATE; ESTRADIOL VALERATE, ESTRADIOL VALERATE; ESTRADIOL, ESTRADIOL; ESTRAMUSTINE PHOSPHATE SODIUM; ESTROGENS, CONJUGATED; ESTROGENS, ESTERIFIED; ESZOPICLONE, ESZOPICLONE; ETHACRYNATE SODIUM, ETHACRYNATE SODIUM; ETHACRYNIC ACID, ETHACRYNIC ACID; ETHAMBUTOL HYDROCHLORIDE, ETHAMBUTOL HYDROCHLORIDE; ETHAMOLIN, ETHANOLAMINE OLEATE; ETHINYL ESTRADIOL; ETHINYL ESTRADIOL AND NORELGESTROMIN, ETHINYL ESTRADIOL; ETHINYL ESTRADIOL, ETONOGESTREL, ETHINYL ESTRADIOL; ETHIONAMIDE; ETHOSUXIMIDE, ETHOSUXIMIDE; ETHYNODIOL DIACETATE AND ETHINYL ESTRADIOL, ETHINYL ESTRADIOL; ETODOLAC, ETODOLAC; ETOMIDATE, ETOMIDATE; ETOPOSIDE, ETOPOSIDE; ETRAVIRINE, ETRAVIRINE; EVEROLIMUS, EVEROLIMUS; EXEMESTANE, EXEMESTANE; EXTENDED PHENYTOIN SODIUM, PHENYTOIN SODIUM; EZETIMIBE AND SIMVASTATIN, EZETIMIBE; EZETIMIBE, EZETIMIBE; FAMCICLOVIR, FAMCICLOVIR; FAMOTIDINE PRESERVATIVE FREE IN PLASTIC CONTAINER, FAMOTIDINE; FAMOTIDINE PRESERVATIVE FREE, FAMOTIDINE; FAMOTIDINE, CALCIUM CARBONATE, AND MAGNESIUM HYDROXIDE, CALCIUM CARBONATE (OTC); FAMOTIDINE, FAMOTIDINE; FAMOTIDINE, FAMOTIDINE (OTC); FEBUXOSTAT, FEBUXOSTAT; FEDRATINIB HYDROCHLORIDE; FELBAMATE, FELBAMATE; FELODIPINE, FELODIPINE; FENFLURAMINE HYDROCHLORIDE; FENOFIBRATE (MICRONIZED), FENOFIBRATE; FENOFIBRATE, FENOFIBRATE; FENOFIBRIC ACID, CHOLINE FENOFIBRATE; FENOLDOPAM MESYLATE, FENOLDOPAM MESYLATE; FENOPROFEN CALCIUM, FENOPROFEN CALCIUM; FENTANYL CITRATE PRESERVATIVE FREE, FENTANYL CITRATE; FENTANYL CITRATE, FENTANYL CITRATE; FENTANYL-100, FENTANYL; FENTANYL-12, FENTANYL; FENTANYL-25, FENTANYL; FENTANYL-37, FENTANYL; FENTANYL-50, FENTANYL; FENTANYL-62, FENTANYL; FENTANYL-75, FENTANYL; FENTANYL-87, FENTANYL; FERRIC CITRATE; FERRIC OXYHYDROXIDE; FERUMOXYTOL, FERUMOXYTOL; FESOTERODINE FUMARATE, FESOTERODINE FUMARATE; FEXINIDAZOLE, FEXINIDAZOLE; FEXOFENADINE HYDROCHLORIDE ALLERGY, FEXOFENADINE HYDROCHLORIDE (OTC); FEXOFENADINE HYDROCHLORIDE AND PSEUDOEPHEDRINE HYDROCHLORIDE, FEXOFENADINE HYDROCHLORIDE; FEXOFENADINE HYDROCHLORIDE HIVES, FEXOFENADINE HYDROCHLORIDE (OTC); FEXOFENADINE HYDROCHLORIDE, FEXOFENADINE HYDROCHLORIDE; FEXOFENADINE HYDROCHLORIDE, FEXOFENADINE HYDROCHLORIDE (OTC); FIDAXOMICIN; FINASTERIDE, FINASTERIDE; FINERENONE; FINGOLIMOD HYDROCHLORIDE, FINGOLIMOD HYDROCHLORIDE; FISH OIL; FISH OIL TRIGLYCERIDES; FLAVORED FENOFIBRIC ACID, COLESTIPOL HYDROCHLORIDE; FLAVOXATE HYDROCHLORIDE, FLAVOXATE HYDROCHLORIDE; FLECAINIDE ACETATE, FLECAINIDE ACETATE; FLOXURIDINE, FLOXURIDINE; FLUCINOLONE ACETONIDE, FLUOCINOLONE ACETONIDE; FLUCONAZOLE IN SODIUM CHLORIDE 0.9% IN PLASTIC CONTAINER, FLUCONAZOLE; FLUCONAZOLE IN SODIUM CHLORIDE 0.9%, FLUCONAZOLE; FLUCONAZOLE, FLUCONAZOLE; FLUCYTOSINE, FLUCYTOSINE; FLUDARABINE PHOSPHATE, FLUDARABINE PHOSPHATE; FLUDEOXYGLUCOSE F18, FLUDEOXYGLUCOSE F-18; FLUDROCORTISONE ACETATE, FLUDROCORTISONE ACETATE; FLUMAZENIL, FLUMAZENIL; FLUNISOLIDE, FLUNISOLIDE; FLUOCINOLONE ACETONIDE, FLUOCINOLONE ACETONIDE; FLUOCINONIDE ACETONIDE, FLUOCINOLONE ACETONIDE; FLUOCINONIDE EMULSIFIED BASE, FLUOCINONIDE; FLUOCINONIDE, FLUOCINONIDE; FLUORESCEIN SODIUM AND BENOXINATE HYDROCHLORIDE, BENOXINATE HYDROCHLORIDE; FLUORESCEIN SODIUM, FLUORESCEIN SODIUM; FLUORODOPA F18, FLUORODOPA F-18; FLUOROMETHOLONE, FLUOROMETHOLONE; FLUOROURACIL, FLUOROURACIL; FLUOXETINE HYDROCHLORIDE, FLUOXETINE HYDROCHLORIDE; FLUPHENAZINE DECANOATE, FLUPHENAZINE DECANOATE; FLUPHENAZINE HYDROCHLORIDE, FLUPHENAZINE HYDROCHLORIDE; FLURANDRENOLIDE, FLURANDRENOLIDE; FLURAZEPAM HYDROCHLORIDE, FLURAZEPAM HYDROCHLORIDE; FLURBIPROFEN SODIUM, FLURBIPROFEN SODIUM; FLURBIPROFEN, FLURBIPROFEN; FLUTAMIDE, FLUTAMIDE; FLUTICASONE FUROATE; FLUTICASONE PROPIONATE AND SALMETEROL XINAFOATE, FLUTICASONE PROPIONATE; FLUTICASONE PROPIONATE, FLUTICASONE PROPIONATE; FLUTICASONE PROPIONATE, FLUTICASONE PROPIONATE (OTC); FLUVASTATIN SODIUM, FLUVASTATIN SODIUM; FLUVOXAMINE MALEATE, FLUVOXAMINE MALEATE; FOLIC ACID, FOLIC ACID; FOMEPIZOLE, FOMEPIZOLE; FONDAPARINUX SODIUM, FONDAPARINUX SODIUM; FORMOTEROL FUMARATE, FORMOTEROL FUMARATE; FOSAMPRENAVIR CALCIUM, FOSAMPRENAVIR CALCIUM; FOSAPREPITANT DIMEGLUMINE, FOSAPREPITANT DIMEGLUMINE; FOSCARNET SODIUM, FOSCARNET SODIUM; FOSFOMYCIN TROMETHAMINE, FOSFOMYCIN TROMETHAMINE; FOSINOPRIL SODIUM AND HYDROCHLOROTHIAZIDE, FOSINOPRIL SODIUM; FOSINOPRIL SODIUM, FOSINOPRIL SODIUM; FOSPHENYTOIN SODIUM, FOSPHENYTOIN SODIUM; FOSTAMATINIB DISODIUM; FOSTEMSAVIR TROMETHAMINE; FROVATRIPTAN SUCCINATE, FROVATRIPTAN SUCCINATE; FULVESTRANT, FULVESTRANT; FUROSEMIDE, FUROSEMIDE; FUTIBATINIB; GABAPENTIN ENACARBIL; GABAPENTIN, GABAPENTIN; GADOBUTROL, GADOBUTROL; GADOTERATE MEGLUMINE, GADOTERATE MEGLUMINE; GALANTAMINE HYDROBROMIDE, GALANTAMINE HYDROBROMIDE; GALLIUM CITRATE GA 67, GALLIUM CITRATE GA-67; GALLIUM GA 68 EDOTREOTIDE, GALLIUM GA-68 EDOTREOTIDE; GALLIUM GA 68 GOZETOTIDE, GALLIUM GA-68 GOZETOTIDE; GANAXOLONE; GANCICLOVIR; GANCICLOVIR SODIUM, GANCICLOVIR SODIUM; GANIRELIX ACETATE, GANIRELIX ACETATE; GATIFLOXACIN, GATIFLOXACIN; GEFITINIB, GEFITINIB; GEMCITABINE HYDROCHLORIDE, GEMCITABINE HYDROCHLORIDE; GEMFIBROZIL, GEMFIBROZIL; GENTAMICIN SULFATE IN SODIUM CHLORIDE 0.9% IN PLASTIC CONTAINER, GENTAMICIN SULFATE; GENTAMICIN SULFATE, GENTAMICIN SULFATE; GILTERITINIB FUMARATE; GLASDEGIB MALEATE; GLATIRAMER ACETATE, GLATIRAMER ACETATE; GLECAPREVIR; GLIMEPIRIDE, GLIMEPIRIDE; GLIPIZIDE AND METFORMIN HYDROCHLORIDE, GLIPIZIDE; GLIPIZIDE, GLIPIZIDE; GLUCAGON HYDROCHLORIDE; GLUCAGON, GLUCAGON; GLUCAGON, GLUCAGON HYDROCHLORIDE; GLYBURIDE (MICRONIZED), GLYBURIDE; GLYBURIDE AND METFORMIN HYDROCHLORIDE, GLYBURIDE; GLYBURIDE, GLYBURIDE; GLYCEROL PHENYLBUTYRATE; GLYCINE 1.5% IN PLASTIC CONTAINER, GLYCINE; GLYCOPYRROLATE, GLYCOPYRROLATE; GRANISETRON; GRANISETRON HYDROCHLORIDE PRESERVATIVE FREE, GRANISETRON HYDROCHLORIDE; GRANISETRON HYDROCHLORIDE, GRANISETRON HYDROCHLORIDE; GRISEOFULVIN, GRISEOFULVIN, MICROSIZE; GRISEOFULVIN, GRISEOFULVIN, MICROSIZE; GRISEOFULVIN, ULTRAMICROSIZE, GRISEOFULVIN, ULTRAMICROSIZE; GRISEOFULVIN, ULTRAMICROSIZE, GRISEOFULVIN, ULTRAMICROSIZE; GUAIFENESIN AND DEXTROMETHORPHAN HYDROBROMIDE, DEXTROMETHORPHAN HYDROBROMIDE (OTC); GUAIFENESIN AND PSEUDOEPHEDRINE HYDROCHLORIDE, GUAIFENESIN; GUAIFENESIN, GUAIFENESIN (OTC); GUANFACINE HYDROCHLORIDE, GUANFACINE HYDROCHLORIDE; HALCINONIDE, HALCINONIDE; HALOBETASOL PROPIONATE, HALOBETASOL PROPIONATE; HALOPERIDOL DECANOATE, HALOPERIDOL DECANOATE; HALOPERIDOL, HALOPERIDOL; HALOPERIDOL, HALOPERIDOL LACTATE; HEPARIN SODIUM 1,000 UNITS AND SODIUM CHLORIDE 0.9% IN PLASTIC CONTAINER, HEPARIN SODIUM; HEPARIN SODIUM 1,000 UNITS IN SODIUM CHLORIDE 0.9% IN PLASTIC CONTAINER, HEPARIN SODIUM; HEPARIN SODIUM 10,000 UNITS IN DEXTROSE 5% IN PLASTIC CONTAINER, HEPARIN SODIUM; HEPARIN SODIUM 12,000 UNITS IN DEXTROSE 5% IN PLASTIC CONTAINER, HEPARIN SODIUM; HEPARIN SODIUM 12,000 UNITS IN SODIUM CHLORIDE 0.45% IN PLASTIC CONTAINER, HEPARIN SODIUM; HEPARIN SODIUM 2,000 UNITS AND SODIUM CHLORIDE 0.9% IN PLASTIC CONTAINER, HEPARIN SODIUM; HEPARIN SODIUM 2,000 UNITS IN SODIUM CHLORIDE 0.9% IN PLASTIC CONTAINER, HEPARIN SODIUM; HEPARIN SODIUM 20,000 UNITS IN DEXTROSE 5% IN PLASTIC CONTAINER, HEPARIN SODIUM; HEPARIN SODIUM 25,000 UNITS IN DEXTROSE 5% IN PLASTIC CONTAINER, HEPARIN SODIUM; HEPARIN SODIUM 25,000 UNITS IN SODIUM CHLORIDE 0.45% IN PLASTIC CONTAINER, HEPARIN SODIUM; HEPARIN SODIUM IN PLASTIC CONTAINER, HEPARIN SODIUM; HEPARIN SODIUM PRESERVATIVE FREE, HEPARIN SODIUM; HEPARIN SODIUM, HEPARIN SODIUM; HEPSERA, ADEFOVIR DIPIVOXIL; HOMATROPINE METHYLBROMIDE AND HYDROCODONE BITARTRATE, HOMATROPINE METHYLBROMIDE; HYDRALAZINE HYDROCHLORIDE, HYDRALAZINE HYDROCHLORIDE; HYDROCHLOROTHIAZIDE, HYDROCHLOROTHIAZIDE; HYDROCODONE BITARTRATE AND ACETAMINOPHEN, ACETAMINOPHEN; HYDROCODONE BITARTRATE AND ASPIRIN, ASPIRIN; HYDROCODONE BITARTRATE AND HOMATROPINE METHYLBROMIDE, HOMATROPINE METHYLBROMIDE; HYDROCODONE BITARTRATE AND IBUPROFEN, HYDROCODONE BITARTRATE; HYDROCODONE BITARTRATE, CHLORPHENIRAMINE MALEATE AND PSEUDOEPHEDRINE HYDROCHLORIDE; HYDROCODONE BITARTRATE, HYDROCODONE BITARTRATE; HYDROCODONE POLISTIREX AND CHLORPHENIRAMINE POLISTIREX, CHLORPHENIRAMINE POLISTIREX; HYDROCODONE, HYDROCODONE BITARTRATE; HYDROCORTISONE; HYDROCORTISONE ACETATE; HYDROCORTISONE AND ACETIC ACID, ACETIC ACID, GLACIAL; HYDROCORTISONE BUTYRATE, HYDROCORTISONE BUTYRATE; HYDROCORTISONE IN ABSORBASE, HYDROCORTISONE; HYDROCORTISONE SODIUM SUCCINATE; HYDROCORTISONE VALERATE, HYDROCORTISONE VALERATE; HYDROCORTISONE, HYDROCORTISONE; HYDROMORPHONE HYDROCHLORIDE, HYDROMORPHONE HYDROCHLORIDE; HYDROXOCOBALAMIN, HYDROXOCOBALAMIN; HYDROXYCHLOROQUINE SULFATE, HYDROXYCHLOROQUINE SULFATE; HYDROXYPROPYL CELLULOSE; HYDROXYUREA, HYDROXYUREA; HYDROXYZINE HYDROCHLORIDE, HYDROXYZINE HYDROCHLORIDE; HYDROXYZINE PAMOATE, HYDROXYZINE PAMOATE; IBANDRONATE SODIUM, IBANDRONATE SODIUM; IBRUTINIB; IBUPROFEN AND DIPHENHYDRAMINE CITRATE, DIPHENHYDRAMINE CITRATE (OTC); IBUPROFEN AND DIPHENHYDRAMINE HYDROCHLORIDE, DIPHENHYDRAMINE HYDROCHLORIDE; IBUPROFEN AND FAMOTIDINE, FAMOTIDINE; IBUPROFEN AND PHENYLEPHRINE HYDROCHLORIDE, IBUPROFEN; IBUPROFEN AND PSEUDOEPHEDRINE HYDROCHLORIDE, IBUPROFEN; IBUPROFEN LYSINE, IBUPROFEN LYSINE; IBUPROFEN, IBUPROFEN; IBUPROFEN, IBUPROFEN (OTC); IBUTILIDE FUMARATE, IBUTILIDE FUMARATE; ICATIBANT ACETATE, ICATIBANT ACETATE; ICOSAPENT ETHYL, ICOSAPENT ETHYL; IDARUBICIN HYDROCHLORIDE, IDARUBICIN HYDROCHLORIDE; IDELALISIB; IFOSFAMIDE, IFOSFAMIDE; ILOPERIDONE; IMATINIB MESYLATE, IMATINIB MESYLATE; IMIPENEM AND CILASTATIN, CILASTATIN SODIUM; IMIPRAMINE HYDROCHLORIDE, IMIPRAMINE HYDROCHLORIDE; IMIPRAMINE PAMOATE, IMIPRAMINE PAMOATE; IMIQUIMOD, IMIQUIMOD; INDAPAMIDE, INDAPAMIDE; INDIUM IN 111 CHLORIDE, INDIUM IN-111 CHLORIDE; INDIUM IN 111 OXYQUINOLINE, INDIUM IN-111 OXYQUINOLINE; INDOCYANINE GREEN, INDOCYANINE GREEN; INDOMETHACIN SODIUM, INDOMETHACIN SODIUM; INDOMETHACIN, INDOMETHACIN; INOTERSEN SODIUM; IODIXANOL, IODIXANOL; IOFLUPANE I-123, IOFLUPANE I-123; IOPAMIDOL, IOPAMIDOL; IPRATROPIUM BROMIDE, IPRATROPIUM BROMIDE; IRBESARTAN AND HYDROCHLOROTHIAZIDE, HYDROCHLOROTHIAZIDE; IRBESARTAN, IRBESARTAN; IRINOTECAN HYDROCHLORIDE, IRINOTECAN HYDROCHLORIDE; ISAVUCONAZONIUM SULFATE; ISOCARBOXAZID; ISOFLURANE, ISOFLURANE; ISONIAZID, ISONIAZID; ISOPROTERENOL HYDROCHLORIDE, ISOPROTERENOL HYDROCHLORIDE; ISOSORBIDE DINITRATE AND HYDRALAZINE HYDROCHLORIDE, HYDRALAZINE HYDROCHLORIDE; ISOSORBIDE DINITRATE, ISOSORBIDE DINITRATE; ISOSORBIDE MONONITRATE, ISOSORBIDE MONONITRATE; ISOSULFAN BLUE, ISOSULFAN BLUE; ISOTRETINOIN, ISOTRETINOIN; ISRADIPINE, ISRADIPINE; ISTRADEFYLLINE; ITRACONAZOLE, ITRACONAZOLE; IVABRADINE; IVABRADINE HYDROCHLORIDE, IVABRADINE HYDROCHLORIDE; IVACAFTOR; IVERMECTIN, IVERMECTIN; IVERMECTIN, IVERMECTIN (OTC); IVOSIDENIB; IXAZOMIB CITRATE; KETAMINE HYDROCHLORIDE, KETAMINE HYDROCHLORIDE; KETOCONAZOLE, KETOCONAZOLE; KETOPROFEN, KETOPROFEN; KETOROLAC TROMETHAMINE AND PHENYLEPHRINE HYDROCHLORIDE, KETOROLAC TROMETHAMINE; KETOROLAC TROMETHAMINE, KETOROLAC TROMETHAMINE; KETOTIFEN FUMARATE, KETOTIFEN FUMARATE (OTC); L-GLUTAMINE; LABETALOL HYDROCHLORIDE IN DEXTROSE, LABETALOL HYDROCHLORIDE; LABETALOL HYDROCHLORIDE IN SODIUM CHLORIDE, LABETALOL HYDROCHLORIDE; LABETALOL HYDROCHLORIDE, LABETALOL HYDROCHLORIDE; LACOSAMIDE, LACOSAMIDE; LACTATED RINGER'S AND DEXTROSE 5% IN PLASTIC CONTAINER, CALCIUM CHLORIDE; LACTATED RINGER'S IN PLASTIC CONTAINER, CALCIUM CHLORIDE; LACTULOSE, LACTULOSE; LAMIVUDINE AND ZIDOVUDINE, LAMIVUDINE; LAMIVUDINE, LAMIVUDINE; LAMOTRIGINE, LAMOTRIGINE; LANREOTIDE ACETATE, LANREOTIDE ACETATE; LANSOPRAZOLE, AMOXICILLIN AND CLARITHROMYCIN (COPACKAGED), AMOXICILLIN; LANSOPRAZOLE, LANSOPRAZOLE; LANSOPRAZOLE, LANSOPRAZOLE (OTC); LANTHANUM CARBONATE, LANTHANUM CARBONATE; LAPATINIB DITOSYLATE, LAPATINIB DITOSYLATE; LAROTRECTINIB SULFATE; LATANOPROST, LATANOPROST; LEFAMULIN ACETATE; LEFLUNOMIDE, LEFLUNOMIDE; LENACAPAVIR SODIUM; LENALIDOMIDE, LENALIDOMIDE; LENIOLISIB PHOSPHATE; LENVATINIB MESYLATE; LETERMOVIR; LETROZOLE, LETROZOLE; LEUCOVORIN CALCIUM PRESERVATIVE FREE, LEUCOVORIN CALCIUM; LEUCOVORIN CALCIUM, LEUCOVORIN CALCIUM; LEUPROLIDE ACETATE FOR DEPOT SUSPENSION, LEUPROLIDE ACETATE; LEUPROLIDE ACETATE, LEUPROLIDE ACETATE; LEVALBUTEROL HYDROCHLORIDE, LEVALBUTEROL HYDROCHLORIDE; LEVETIRACETAM IN SODIUM CHLORIDE, LEVETIRACETAM; LEVETIRACETAM, LEVETIRACETAM; LEVOBUNOLOL HYDROCHLORIDE, LEVOBUNOLOL HYDROCHLORIDE; LEVOCARNITINE SF, LEVOCARNITINE; LEVOCARNITINE, LEVOCARNITINE; LEVOCETIRIZINE DIHYDROCHLORIDE, LEVOCETIRIZINE DIHYDROCHLORIDE; LEVOCETIRIZINE DIHYDROCHLORIDE, LEVOCETIRIZINE DIHYDROCHLORIDE (OTC); LEVOCETIRIZINE HYDROCHLORIDE, LEVOCETIRIZINE DIHYDROCHLORIDE; LEVODOPA; LEVOFLOXACIN IN DEXTROSE 5% IN PLASTIC CONTAINER, LEVOFLOXACIN; LEVOFLOXACIN, LEVOFLOXACIN; LEVOKETOCONAZOLE; LEVOLEUCOVORIN CALCIUM, LEVOLEUCOVORIN CALCIUM; LEVOMILNACIPRAN HYDROCHLORIDE, LEVOMILNACIPRAN HYDROCHLORIDE; LEVONORGESTREL AND ETHINYL ESTRADIOL AND ETHINYL ESTRADIOL, ETHINYL ESTRADIOL; LEVONORGESTREL AND ETHINYL ESTRADIOL AND FERROUS FUMARATE, ETHINYL ESTRADIOL; LEVONORGESTREL AND ETHINYL ESTRADIOL, ETHINYL ESTRADIOL; LEVONORGESTREL, LEVONORGESTREL (OTC); LEVORPHANOL TARTRATE, LEVORPHANOL TARTRATE; LEVOTHYROXINE SODIUM **; LEVOTHYROXINE SODIUM, LEVOTHYROXINE SODIUM; LEVOTHYROXINE SODIUM, LEVOTHYROXINE SODIUM **; LIDOCAINE; LIDOCAINE AND PRILOCAINE, LIDOCAINE; LIDOCAINE HYDROCHLORIDE 0.2% AND DEXTROSE 5% IN PLASTIC CONTAINER, LIDOCAINE HYDROCHLORIDE; LIDOCAINE HYDROCHLORIDE 0.4% AND DEXTROSE 5% IN PLASTIC CONTAINER, LIDOCAINE HYDROCHLORIDE; LIDOCAINE HYDROCHLORIDE 0.8% AND DEXTROSE 5% IN PLASTIC CONTAINER, LIDOCAINE HYDROCHLORIDE; LIDOCAINE HYDROCHLORIDE 5% AND DEXTROSE 7.5%, LIDOCAINE HYDROCHLORIDE; LIDOCAINE HYDROCHLORIDE AND EPINEPHRINE, EPINEPHRINE; LIDOCAINE HYDROCHLORIDE IN PLASTIC CONTAINER, LIDOCAINE HYDROCHLORIDE; LIDOCAINE HYDROCHLORIDE PRESERVATIVE FREE IN PLASTIC CONTAINER, LIDOCAINE HYDROCHLORIDE; LIDOCAINE HYDROCHLORIDE PRESERVATIVE FREE, LIDOCAINE HYDROCHLORIDE; LIDOCAINE HYDROCHLORIDE VISCOUS, LIDOCAINE HYDROCHLORIDE; LIDOCAINE HYDROCHLORIDE, LIDOCAINE HYDROCHLORIDE; LIDOCAINE VISCOUS, LIDOCAINE HYDROCHLORIDE; LIDOCAINE, LIDOCAINE; LIFITEGRAST, LIFITEGRAST; LINACLOTIDE; LINAGLIPTIN AND METFORMIN HYDROCHLORIDE, LINAGLIPTIN; LINAGLIPTIN, LINAGLIPTIN; LINCOMYCIN HYDROCHLORIDE, LINCOMYCIN HYDROCHLORIDE; LINEZOLID IN SODIUM CHLORIDE 0.9% IN PLASTIC CONTAINER, LINEZOLID; LINEZOLID, LINEZOLID; LIOTHYRONINE SODIUM, LIOTHYRONINE SODIUM; LIRAGLUTIDE RECOMBINANT; LISDEXAMFETAMINE DIMESYLATE, LISDEXAMFETAMINE DIMESYLATE; LISINOPRIL AND HYDROCHLOROTHIAZIDE, HYDROCHLOROTHIAZIDE; LISINOPRIL, LISINOPRIL; LITHIUM CARBONATE, LITHIUM CARBONATE; LITHIUM CITRATE, LITHIUM CITRATE; LODOXAMIDE TROMETHAMINE; LOFEXIDINE HYDROCHLORIDE; LOMITAPIDE MESYLATE; LOMUSTINE; LONAFARNIB; LOPERAMIDE HYDROCHLORIDE AND SIMETHICONE, LOPERAMIDE HYDROCHLORIDE; LOPERAMIDE HYDROCHLORIDE, LOPERAMIDE HYDROCHLORIDE; LOPERAMIDE HYDROCHLORIDE, LOPERAMIDE HYDROCHLORIDE (OTC); LOPINAVIR AND RITONAVIR, LOPINAVIR; LORATADINE AND PSEUDOEPHEDRINE SULFATE, LORATADINE (OTC); LORATADINE REDIDOSE, LORATADINE (OTC); LORATADINE, LORATADINE (OTC); LORAZEPAM INTENSOL, LORAZEPAM; LORAZEPAM, LORAZEPAM; LORLATINIB; LOSARTAN POTASSIUM AND HYDROCHLOROTHIAZIDE, HYDROCHLOROTHIAZIDE; LOSARTAN POTASSIUM, LOSARTAN POTASSIUM; LOTEPREDNOL ETABONATE, LOTEPREDNOL ETABONATE; LOVASTATIN, LOVASTATIN; LOXAPINE SUCCINATE, LOXAPINE SUCCINATE; LUBIPROSTONE, LUBIPROSTONE; LUMATEPERONE TOSYLATE; LURASIDONE HYDROCHLORIDE, LURASIDONE HYDROCHLORIDE; LUSUTROMBOPAG; MACITENTAN, MACITENTAN; MAFENIDE ACETATE; MAGNESIUM CHLORIDE; MAGNESIUM SULFATE IN DEXTROSE 5% IN PLASTIC CONTAINER, MAGNESIUM SULFATE; MAGNESIUM SULFATE IN PLASTIC CONTAINER, MAGNESIUM SULFATE; MAGNESIUM SULFATE, MAGNESIUM SULFATE; MALATHION, MALATHION; MANGANESE CHLORIDE IN PLASTIC CONTAINER, MANGANESE CHLORIDE; MANNITOL; MANNITOL 10% IN PLASTIC CONTAINER, MANNITOL; MANNITOL 15% IN PLASTIC CONTAINER, MANNITOL; MANNITOL 20% IN PLASTIC CONTAINER, MANNITOL; MANNITOL 25%, MANNITOL; MANNITOL 5% IN PLASTIC CONTAINER, MANNITOL; MARALIXIBAT CHLORIDE; MARAVIROC, MARAVIROC; MARIBAVIR; MAVACAMTEN; MEBENDAZOLE; MECAMYLAMINE HYDROCHLORIDE, MECAMYLAMINE HYDROCHLORIDE; MECHLORETHAMINE HYDROCHLORIDE; MECLIZINE HYDROCHLORIDE, MECLIZINE HYDROCHLORIDE; MECLOFENAMATE SODIUM, MECLOFENAMATE SODIUM; MEDROXYPROGESTERONE ACETATE, MEDROXYPROGESTERONE ACETATE; MEFENAMIC ACID, MEFENAMIC ACID; MEFLOQUINE HYDROCHLORIDE, MEFLOQUINE HYDROCHLORIDE; MEGESTROL ACETATE, MEGESTROL ACETATE; MELOXICAM, MELOXICAM; MELPHALAN HYDROCHLORIDE, MELPHALAN HYDROCHLORIDE; MELPHALAN, MELPHALAN; MEMANTINE HYDROCHLORIDE AND DONEPEZIL HYDROCHLORIDE, DONEPEZIL HYDROCHLORIDE; MEMANTINE HYDROCHLORIDE, MEMANTINE HYDROCHLORIDE; MEPERIDINE HYDROCHLORIDE PRESERVATIVE FREE, MEPERIDINE HYDROCHLORIDE; MEPERIDINE HYDROCHLORIDE, MEPERIDINE HYDROCHLORIDE; MEPROBAMATE, MEPROBAMATE; MERCAPTOPURINE, MERCAPTOPURINE; MEROPENEM AND SODIUM CHLORIDE IN DUPLEX CONTAINER, MEROPENEM; MEROPENEM, MEROPENEM; MESALAMINE, MESALAMINE; MESNA, MESNA; METAPROTERENOL SULFATE, METAPROTERENOL SULFATE; METARAMINOL BITARTRATE, METARAMINOL BITARTRATE; METAXALONE, METAXALONE; METFORMIN HYDROCHLORIDE, METFORMIN HYDROCHLORIDE; METFORMIN HYDROCHLORIDE, SAXAGLIPTIN HYDROCHLORIDE, METFORMIN HYDROCHLORIDE; METHADONE HYDROCHLORIDE INTENSOL, METHADONE HYDROCHLORIDE; METHADONE HYDROCHLORIDE, METHADONE HYDROCHLORIDE; METHAMPHETAMINE HYDROCHLORIDE, METHAMPHETAMINE HYDROCHLORIDE; METHAZOLAMIDE, METHAZOLAMIDE; METHENAMINE HIPPURATE, METHENAMINE HIPPURATE; METHIMAZOLE, METHIMAZOLE; METHOCARBAMOL, METHOCARBAMOL; METHOTREXATE PRESERVATIVE FREE, METHOTREXATE SODIUM; METHOTREXATE SODIUM PRESERVATIVE FREE, METHOTREXATE SODIUM; METHOTREXATE SODIUM, METHOTREXATE SODIUM; METHOXSALEN, METHOXSALEN; METHSCOPOLAMINE BROMIDE, METHSCOPOLAMINE BROMIDE; METHSUXIMIDE, METHSUXIMIDE; METHYLENE BLUE, METHYLENE BLUE; METHYLERGONOVINE MALEATE, METHYLERGONOVINE MALEATE; METHYLNALTREXONE BROMIDE; METHYLPHENIDATE HYDROCHLORIDE, METHYLPHENIDATE HYDROCHLORIDE; METHYLPHENIDATE, METHYLPHENIDATE; METHYLPREDNISOLONE ACETATE, METHYLPREDNISOLONE ACETATE; METHYLPREDNISOLONE SODIUM SUCCINATE, METHYLPREDNISOLONE SODIUM SUCCINATE; METHYLPREDNISOLONE, METHYLPREDNISOLONE; METHYLTESTOSTERONE, METHYLTESTOSTERONE; METOCLOPRAMIDE HYDROCHLORIDE, METOCLOPRAMIDE HYDROCHLORIDE; METOCLOPRAMIDE, METOCLOPRAMIDE HYDROCHLORIDE; METOLAZONE, METOLAZONE; METOPROLOL SUCCINATE, METOPROLOL SUCCINATE; METOPROLOL TARTRATE AND HYDROCHLOROTHIAZIDE, HYDROCHLOROTHIAZIDE; METOPROLOL TARTRATE, METOPROLOL TARTRATE; METRONIDAZOLE IN PLASTIC CONTAINER, METRONIDAZOLE; METRONIDAZOLE, METRONIDAZOLE; METYRAPONE; METYROSINE, METYROSINE; MEXILETINE HYDROCHLORIDE, MEXILETINE HYDROCHLORIDE; MICAFUNGIN IN SODIUM CHLORIDE 0.9%, MICAFUNGIN SODIUM; MICAFUNGIN SODIUM, MICAFUNGIN SODIUM; MICAFUNGIN, MICAFUNGIN SODIUM; MICONAZOLE 3 COMBINATION PACK, MICONAZOLE NITRATE (OTC); MICONAZOLE 3, MICONAZOLE NITRATE (OTC); MICONAZOLE 7, MICONAZOLE NITRATE (OTC); MICONAZOLE NITRATE COMBINATION PACK, MICONAZOLE NITRATE (OTC); MICONAZOLE NITRATE, MICONAZOLE NITRATE; MICONAZOLE NITRATE, MICONAZOLE NITRATE (OTC); MIDAZOLAM; MIDAZOLAM HYDROCHLORIDE PRESERVATIVE FREE, MIDAZOLAM HYDROCHLORIDE; MIDAZOLAM HYDROCHLORIDE, MIDAZOLAM HYDROCHLORIDE; MIDAZOLAM IN 0.8% SODIUM CHLORIDE, MIDAZOLAM; MIDAZOLAM IN 0.9% SODIUM CHLORIDE, MIDAZOLAM; MIDODRINE HYDROCHLORIDE, MIDODRINE HYDROCHLORIDE; MIDOSTAURIN; MIDOZALAM HYDROCHLORIDE, MIDAZOLAM HYDROCHLORIDE; MIFEPRISTONE, MIFEPRISTONE; MIGALASTAT HYDROCHLORIDE; MIGLITOL, MIGLITOL; MIGLUSTAT, MIGLUSTAT; MILNACIPRAN HYDROCHLORIDE; MILRINONE LACTATE IN DEXTROSE 5% IN PLASTIC CONTAINER, MILRINONE LACTATE; MILRINONE LACTATE IN DEXTROSE 5%, MILRINONE LACTATE; MILRINONE LACTATE IN PLASTIC CONTAINER, MILRINONE LACTATE; MILRINONE LACTATE, MILRINONE LACTATE; MINOCYCLINE HYDROCHLORIDE, MINOCYCLINE HYDROCHLORIDE; MINOXIDIL (FOR MEN), MINOXIDIL (OTC); MINOXIDIL (FOR WOMEN), MINOXIDIL (OTC); MINOXIDIL EXTRA STRENGTH (FOR MEN), MINOXIDIL (OTC); MINOXIDIL, MINOXIDIL; MINOXIDIL, MINOXIDIL (OTC); MIRABEGRON, MIRABEGRON; MIRTAZAPINE, MIRTAZAPINE; MISOPROSTOL, MISOPROSTOL; MITAPIVAT SULFATE; MITOMYCIN, MITOMYCIN; MITOTANE; MITOXANTRONE HYDROCHLORIDE, MITOXANTRONE HYDROCHLORIDE; MIVACURIUM CHLORIDE, MIVACURIUM CHLORIDE; MOBOCERTINIB SUCCINATE; MODAFINIL, MODAFINIL; MOEXIPRIL HYDROCHLORIDE AND HYDROCHLOROTHIAZIDE, HYDROCHLOROTHIAZIDE; MOEXIPRIL HYDROCHLORIDE, MOEXIPRIL HYDROCHLORIDE; MOLINDONE HYDROCHLORIDE, MOLINDONE HYDROCHLORIDE; MOMETASONE FUROATE, MOMETASONE FUROATE; MONOBASIC SODIUM PHOSPHATE AND DIBASIC SODIUM PHOSPHATE, SODIUM PHOSPHATE, DIBASIC; MONTELUKAST SODIUM, MONTELUKAST SODIUM; MORPHINE SULFATE, MORPHINE SULFATE; MOXIDECTIN, MOXIDECTIN; MOXIFLOXACIN HYDROCHLORIDE IN SODIUM CHLORIDE 0.8% IN PLASTIC CONTAINER, MOXIFLOXACIN; MOXIFLOXACIN HYDROCHLORIDE, MOXIFLOXACIN HYDROCHLORIDE; MPI INDIUM DTPA IN 111, INDIUM IN-111 PENTETATE DISODIUM; MULTIPLE ELECTROLYTES INJECTION TYPE 1 USP PH 5.5, MAGNESIUM CHLORIDE; MULTIPLE ELECTROLYTES INJECTION TYPE 1 USP PH 7.4, MAGNESIUM CHLORIDE; MUPIROCIN, MUPIROCIN; MUPIROCIN, MUPIROCIN CALCIUM; MYCOPHENOLATE MOFETIL HYDROCHLORIDE, MYCOPHENOLATE MOFETIL HYDROCHLORIDE; MYCOPHENOLATE MOFETIL, MYCOPHENOLATE MOFETIL; MYCOPHENOLIC SODIUM, MYCOPHENOLIC SODIUM; NABUMETONE, NABUMETONE; NADOLOL, NADOLOL; NAFARELIN ACETATE; NAFCILLIN SODIUM, NAFCILLIN SODIUM; NAFTIFINE HYDROCHLORIDE, NAFTIFINE HYDROCHLORIDE; NALBUPHINE HYDROCHLORIDE, NALBUPHINE HYDROCHLORIDE; NALDEMEDINE TOSYLATE; NALMEFENE HYDROCHLORIDE, NALMEFENE HYDROCHLORIDE; NALOXEGOL OXALATE; NALOXONE HYDROCHLORIDE (OTC); NALOXONE HYDROCHLORIDE AND PENTAZOCINE HYDROCHLORIDE, NALOXONE HYDROCHLORIDE; NALOXONE HYDROCHLORIDE, NALOXONE HYDROCHLORIDE; NALOXONE HYDROCHLORIDE, NALOXONE HYDROCHLORIDE (OTC); NALOXONE, NALOXONE HYDROCHLORIDE; NALTREXONE HYDROCHLORIDE, NALTREXONE HYDROCHLORIDE; NALTREXONE, NALTREXONE; NAPHAZOLINE HYDROCHLORIDE AND PHENIRAMINE MALEATE, NAPHAZOLINE HYDROCHLORIDE; NAPROXEN AND ESOMEPRAZOLE MAGNESIUM, ESOMEPRAZOLE MAGNESIUM; NAPROXEN SODIUM AND DIPHENHYDRAMINE HYDROCHLORIDE, DIPHENHYDRAMINE HYDROCHLORIDE; NAPROXEN SODIUM AND PSEUDOEPHEDRINE HYDROCHLORIDE, NAPROXEN SODIUM; NAPROXEN SODIUM, NAPROXEN SODIUM; NAPROXEN SODIUM, NAPROXEN SODIUM (OTC); NAPROXEN, NAPROXEN; NARATRIPTAN, NARATRIPTAN HYDROCHLORIDE; NATAMYCIN; NATEGLINIDE, NATEGLINIDE; NEBIVOLOL HYDROCHLORIDE, NEBIVOLOL HYDROCHLORIDE; NEFAZODONE HYDROCHLORIDE, NEFAZODONE HYDROCHLORIDE; NELARABINE, NELARABINE; NELFINAVIR MESYLATE; NEMBUTAL SODIUM, PENTOBARBITAL SODIUM; NEOMYCIN AND POLYMYXIN B SULFATE, NEOMYCIN SULFATE; NEOMYCIN AND POLYMYXIN B SULFATES AND BACITRACIN ZINC, BACITRACIN ZINC; NEOMYCIN AND POLYMYXIN B SULFATES AND DEXAMETHASONE, DEXAMETHASONE; NEOMYCIN AND POLYMYXIN B SULFATES AND GRAMICIDIN, GRAMICIDIN; NEOMYCIN AND POLYMYXIN B SULFATES AND HYDROCORTISONE, HYDROCORTISONE; NEOMYCIN AND POLYMYXIN B SULFATES, BACITRACIN ZINC AND HYDROCORTISONE, BACITRACIN ZINC; NEOMYCIN SULFATE, NEOMYCIN SULFATE; NEOSTIGMINE METHYLSULFATE, NEOSTIGMINE METHYLSULFATE; NEOSTIGMINE METHYLSULFATE, NEOSTIGMINE METHYLSULFATE; NERATINIB MALEATE; NETARSUDIL MESYLATE; NETUPITANT; NEVIRAPINE, NEVIRAPINE; NIACIN, NIACIN; NICARDIPINE HYDROCHLORIDE IN 0.9% SODIUM CHLORIDE, NICARDIPINE HYDROCHLORIDE; NICARDIPINE HYDROCHLORIDE, NICARDIPINE HYDROCHLORIDE; NICOTINE POLACRILEX, NICOTINE POLACRILEX (OTC); NICOTINE, NICOTINE (OTC); NIFEDIPINE, NIFEDIPINE; NILOTINIB HYDROCHLORIDE, NILOTINIB HYDROCHLORIDE; NILUTAMIDE, NILUTAMIDE; NIMODIPINE, NIMODIPINE; NINTEDANIB ESYLATE; NIRAPARIB TOSYLATE; NISOLDIPINE, NISOLDIPINE; NITAZOXANIDE, NITAZOXANIDE; NITISINONE, NITISINONE; NITROFURANTOIN (MONOHYDRATE/MACROCRYSTALS), NITROFURANTOIN; NITROFURANTOIN, NITROFURANTOIN; NITROFURANTOIN, NITROFURANTOIN, MACROCRYSTALLINE; NITROGLYCERIN IN DEXTROSE 5%, NITROGLYCERIN; NITROGLYCERIN, NITROGLYCERIN; NIZATIDINE, NIZATIDINE; NOREPINEPHRINE BITARTRATE IN 0.9% SODIUM CHLORIDE, NOREPINEPHRINE BITARTRATE; NOREPINEPHRINE BITARTRATE IN 5% DEXTROSE, NOREPINEPHRINE BITARTRATE; NOREPINEPHRINE BITARTRATE, NOREPINEPHRINE BITARTRATE; NORETHINDRONE; NORETHINDRONE ACETATE AND ETHINYL ESTRADIOL AND FERROUS FUMARATE, ETHINYL ESTRADIOL; NORETHINDRONE ACETATE AND ETHINYL ESTRADIOL, ETHINYL ESTRADIOL; NORETHINDRONE ACETATE, NORETHINDRONE ACETATE; NORETHINDRONE AND ETHINYL ESTRADIOL (10/11), ETHINYL ESTRADIOL; NORETHINDRONE AND ETHINYL ESTRADIOL AND FERROUS FUMARATE, ETHINYL ESTRADIOL; NORETHINDRONE AND ETHINYL ESTRADIOL, ETHINYL ESTRADIOL; NORETHINDRONE, NORETHINDRONE; NORGESTIMATE AND ETHINYL ESTRADIOL, ETHINYL ESTRADIOL; NORTRIPTYLINE HYDROCHLORIDE, NORTRIPTYLINE HYDROCHLORIDE; NYSTATIN AND TRIAMCINOLONE ACETONIDE, NYSTATIN; NYSTATIN, NYSTATIN; OBETICHOLIC ACID; OCTREOTIDE ACETATE (PRESERVATIVE FREE), OCTREOTIDE ACETATE; OCTREOTIDE ACETATE, OCTREOTIDE ACETATE; ODEVIXIBAT; OFLOXACIN, OFLOXACIN; OLANZAPINE AND FLUOXETINE HYDROCHLORIDE, FLUOXETINE HYDROCHLORIDE; OLANZAPINE, OLANZAPINE; OLAPARIB; OLIVE OIL; OLMESARTAN MEDOXOMIL AND HYDROCHLOROTHIAZIDE, HYDROCHLOROTHIAZIDE; OLMESARTAN MEDOXOMIL, AMLODIPINE AND HYDROCHLOROTHIAZIDE; OLMESARTAN MEDOXOMIL, OLMESARTAN MEDOXOMIL; OLODATEROL HYDROCHLORIDE; OLOPATADINE HYDROCHLORIDE, OLOPATADINE HYDROCHLORIDE; OLOPATADINE HYDROCHLORIDE, OLOPATADINE HYDROCHLORIDE (OTC); OLUTASIDENIB; OMACETAXINE MEPESUCCINATE; OMADACYCLINE TOSYLATE; OMAVELOXOLONE; OMEGA-3-ACID ETHYL ESTERS, OMEGA-3-ACID ETHYL ESTERS; OMEPRAZOLE AND SODIUM BICARBONATE, OMEPRAZOLE; OMEPRAZOLE AND SODIUM BICARBONATE, OMEPRAZOLE (OTC); OMEPRAZOLE MAGNESIUM, OMEPRAZOLE MAGNESIUM (OTC); OMEPRAZOLE, OMEPRAZOLE; OMEPRAZOLE, OMEPRAZOLE (OTC); ONDANSETRON HYDROCHLORIDE PRESERVATIVE FREE, ONDANSETRON HYDROCHLORIDE; ONDANSETRON HYDROCHLORIDE, ONDANSETRON HYDROCHLORIDE; ONDANSETRON, ONDANSETRON; OPICAPONE; ORPHENADRINE CITRATE, ORPHENADRINE CITRATE; OSELTAMIVIR PHOSPHATE, OSELTAMIVIR PHOSPHATE; OSILODROSTAT PHOSPHATE; OSIMERTINIB MESYLATE; OTESECONAZOLE; OXACILLIN SODIUM, OXACILLIN SODIUM; OXALIPLATIN, OXALIPLATIN; OXAPROZIN, OXAPROZIN; OXAZEPAM, OXAZEPAM; OXCARBAZEPINE, OXCARBAZEPINE; OXICONAZOLE NITRATE, OXICONAZOLE NITRATE; OXYBUTYNIN CHLORIDE, OXYBUTYNIN CHLORIDE; OXYCODONE AND ACETAMINOPHEN, ACETAMINOPHEN; OXYCODONE AND ASPIRIN, ASPIRIN; OXYCODONE HYDROCHLORIDE, OXYCODONE HYDROCHLORIDE; OXYMORPHONE HYDROCHLORIDE, OXYMORPHONE HYDROCHLORIDE; OXYTOCIN, OXYTOCIN; PACLITAXEL, PACLITAXEL; PACRITINIB CITRATE; PALBOCICLIB, PALBOCICLIB; PALIPERIDONE PALMITATE; PALIPERIDONE, PALIPERIDONE; PALONOSETRON HYDROCHLORIDE, PALONOSETRON HYDROCHLORIDE; PAMIDRONATE DISODIUM, PAMIDRONATE DISODIUM; PANCURONIUM BROMIDE, PANCURONIUM BROMIDE; PANTOPRAZOLE SODIUM, PANTOPRAZOLE SODIUM; PARICALCITOL, PARICALCITOL; PAROMOMYCIN SULFATE, PAROMOMYCIN SULFATE; PAROXETINE HYDROCHLORIDE, PAROXETINE HYDROCHLORIDE; PAROXETINE MESYLATE, PAROXETINE MESYLATE; PAROXETINE, PAROXETINE HYDROCHLORIDE; PASIREOTIDE DIASPARTATE; PATIROMER SORBITEX CALCIUM; PAZOPANIB HYDROCHLORIDE, PAZOPANIB HYDROCHLORIDE; PEG 3350 AND ELECTROLYTES, POLYETHYLENE GLYCOL 3350; PEG-3350, POTASSIUM CHLORIDE, SODIUM BICARBONATE, SODIUM CHLORIDE, POLYETHYLENE GLYCOL; PEG-3350, SODIUM SULFATE, SODIUM CHLORIDE, POTASSIUM CHLORIDE, SODIUM ASCORBATE AND; PEMETREXED DISODIUM, PEMETREXED DISODIUM; PEMETREXED DITROMETHAMINE, PEMETREXED DITROMETHAMINE; PEMETREXED, PEMETREXED; PEMETREXED, PEMETREXED DISODIUM; PEMIGATINIB; PENCICLOVIR, PENCICLOVIR; PENICILLAMINE, PENICILLAMINE; PENICILLIN G BENZATHINE; PENICILLIN G POTASSIUM IN PLASTIC CONTAINER, PENICILLIN G POTASSIUM; PENICILLIN G POTASSIUM, PENICILLIN G POTASSIUM; PENICILLIN G PROCAINE, PENICILLIN G PROCAINE; PENICILLIN G SODIUM, PENICILLIN G SODIUM; PENICILLIN V POTASSIUM, PENICILLIN V POTASSIUM; PENICILLIN-VK, PENICILLIN V POTASSIUM; PENTAMIDINE ISETHIONATE, PENTAMIDINE ISETHIONATE; PENTOBARBITAL SODIUM, PENTOBARBITAL SODIUM; PENTOSAN POLYSULFATE SODIUM; PENTOSTATIN, PENTOSTATIN; PENTOXIFYLLINE, PENTOXIFYLLINE; PERAMPANEL; PERINDOPRIL ERBUMINE, PERINDOPRIL ERBUMINE; PERMETHRIN, PERMETHRIN; PERMETHRIN, PERMETHRIN (OTC); PERPHENAZINE AND AMITRIPTYLINE HYDROCHLORIDE, AMITRIPTYLINE HYDROCHLORIDE; PERPHENAZINE, PERPHENAZINE; PEXIDARTINIB HYDROCHLORIDE; PHENDIMETRAZINE TARTRATE, PHENDIMETRAZINE TARTRATE; PHENELZINE SULFATE, PHENELZINE SULFATE; PHENOXYBENZAMINE HYDROCHLORIDE, PHENOXYBENZAMINE HYDROCHLORIDE; PHENTERMINE HYDROCHLORIDE, PHENTERMINE HYDROCHLORIDE; PHENTOLAMINE MESYLATE, PHENTOLAMINE MESYLATE; PHENYLEPHRINE HYDROCHLORIDE AND PROMETHAZINE HYDROCHLORIDE, PHENYLEPHRINE HYDROCHLORIDE; PHENYLEPHRINE HYDROCHLORIDE, PHENYLEPHRINE HYDROCHLORIDE; PHENYTOIN SODIUM, PHENYTOIN SODIUM; PHENYTOIN, PHENYTOIN; PHYTONADIONE, PHYTONADIONE; PILOCARPINE HYDROCHLORIDE, PILOCARPINE HYDROCHLORIDE; PIMAVANSERIN TARTRATE; PIMECROLIMUS, PIMECROLIMUS; PIMOZIDE, PIMOZIDE; PINDOLOL, PINDOLOL; PIOGLITAZONE HYDROCHLORIDE AND GLIMEPIRIDE, GLIMEPIRIDE; PIOGLITAZONE HYDROCHLORIDE AND METFORMIN HYDROCHLORIDE, METFORMIN HYDROCHLORIDE; PIOGLITAZONE HYDROCHLORIDE, PIOGLITAZONE HYDROCHLORIDE; PIPERACILLIN AND TAZOBACTAM, PIPERACILLIN SODIUM; PIPERACILLIN, PIPERACILLIN SODIUM; PIRFENIDONE, PIRFENIDONE; PIROXICAM, PIROXICAM; PIRTOBRUTINIB; PITAVASTATIN CALCIUM, PITAVASTATIN CALCIUM; PITOLISANT HYDROCHLORIDE; PLERIXAFOR, PLERIXAFOR; PODOFILOX, PODOFILOX; POLYETHYLENE GLYCOL 3350 AND ELECTROLYTES, POLYETHYLENE GLYCOL 3350; POLYETHYLENE GLYCOL 3350, POLYETHYLENE GLYCOL 3350 (OTC); POLYMYXIN B SULFATE, POLYMYXIN B SULFATE; POMALIDOMIDE; PONATINIB HYDROCHLORIDE; POSACONAZOLE, POSACONAZOLE; POTASSIUM ACETATE, POTASSIUM ACETATE; POTASSIUM CHLORIDE; POTASSIUM CHLORIDE 0.037% IN DEXTROSE 10% AND SODIUM CHLORIDE 0.2% IN PLASTIC CONTAINER; POTASSIUM CHLORIDE 0.037% IN DEXTROSE 10% AND SODIUM CHLORIDE 0.45% IN PLASTIC CONTAINER; POTASSIUM CHLORIDE 0.037% IN DEXTROSE 10% AND SODIUM CHLORIDE 0.9% IN PLASTIC CONTAINER; POTASSIUM CHLORIDE 0.037% IN DEXTROSE 5% AND SODIUM CHLORIDE 0.11% IN PLASTIC CONTAINER; POTASSIUM CHLORIDE 0.037% IN DEXTROSE 5% AND SODIUM CHLORIDE 0.2% IN PLASTIC CONTAINER; POTASSIUM CHLORIDE 0.037% IN DEXTROSE 5% AND SODIUM CHLORIDE 0.33% IN PLASTIC CONTAINER; POTASSIUM CHLORIDE 0.037% IN DEXTROSE 5% AND SODIUM CHLORIDE 0.45% IN PLASTIC CONTAINER; POTASSIUM CHLORIDE 0.037% IN DEXTROSE 5% AND SODIUM CHLORIDE 0.9% IN PLASTIC CONTAINER; POTASSIUM CHLORIDE 0.075% IN DEXTROSE 10% AND SODIUM CHLORIDE 0.2% IN PLASTIC CONTAINER; POTASSIUM CHLORIDE 0.075% IN DEXTROSE 10% AND SODIUM CHLORIDE 0.45% IN PLASTIC CONTAINER; POTASSIUM CHLORIDE 0.075% IN DEXTROSE 10% AND SODIUM CHLORIDE 0.9% IN PLASTIC CONTAINER; POTASSIUM CHLORIDE 0.075% IN DEXTROSE 3.3% AND SODIUM CHLORIDE 0.3% IN PLASTIC CONTAINER; POTASSIUM CHLORIDE 0.075% IN DEXTROSE 5% AND SODIUM CHLORIDE 0.11% IN PLASTIC CONTAINER; POTASSIUM CHLORIDE 0.075% IN DEXTROSE 5% AND SODIUM CHLORIDE 0.2% IN PLASTIC CONTAINER; POTASSIUM CHLORIDE 0.075% IN DEXTROSE 5% AND SODIUM CHLORIDE 0.33% IN PLASTIC CONTAINER; POTASSIUM CHLORIDE 0.075% IN DEXTROSE 5% AND SODIUM CHLORIDE 0.45% IN PLASTIC CONTAINER; POTASSIUM CHLORIDE 0.075% IN DEXTROSE 5% AND SODIUM CHLORIDE 0.9% IN PLASTIC CONTAINER; POTASSIUM CHLORIDE 0.11% IN DEXTROSE 10% AND SODIUM CHLORIDE 0.2% IN PLASTIC CONTAINER; POTASSIUM CHLORIDE 0.11% IN DEXTROSE 10% AND SODIUM CHLORIDE 0.45% IN PLASTIC CONTAINER; POTASSIUM CHLORIDE 0.11% IN DEXTROSE 10% AND SODIUM CHLORIDE 0.9% IN PLASTIC CONTAINER; POTASSIUM CHLORIDE 0.11% IN DEXTROSE 3.3% AND SODIUM CHLORIDE 0.3% IN PLASTIC CONTAINER; POTASSIUM CHLORIDE 0.11% IN DEXTROSE 5% AND SODIUM CHLORIDE 0.11% IN PLASTIC CONTAINER; POTASSIUM CHLORIDE 0.11% IN DEXTROSE 5% AND SODIUM CHLORIDE 0.2% IN PLASTIC CONTAINER; POTASSIUM CHLORIDE 0.11% IN DEXTROSE 5% AND SODIUM CHLORIDE 0.33% IN PLASTIC CONTAINER; POTASSIUM CHLORIDE 0.11% IN DEXTROSE 5% AND SODIUM CHLORIDE 0.45% IN PLASTIC CONTAINER; POTASSIUM CHLORIDE 0.11% IN DEXTROSE 5% AND SODIUM CHLORIDE 0.9% IN PLASTIC CONTAINER; POTASSIUM CHLORIDE 0.149% IN SODIUM CHLORIDE 0.45% IN PLASTIC CONTAINER, POTASSIUM; POTASSIUM CHLORIDE 0.15% IN DEXTROSE 10% AND SODIUM CHLORIDE 0.2% IN PLASTIC CONTAINER; POTASSIUM CHLORIDE 0.15% IN DEXTROSE 10% AND SODIUM CHLORIDE 0.45% IN PLASTIC CONTAINER; POTASSIUM CHLORIDE 0.15% IN DEXTROSE 10% AND SODIUM CHLORIDE 0.9% IN PLASTIC CONTAINER; POTASSIUM CHLORIDE 0.15% IN DEXTROSE 3.3% AND SODIUM CHLORIDE 0.3% IN PLASTIC CONTAINER; POTASSIUM CHLORIDE 0.15% IN DEXTROSE 5% AND SODIUM CHLORIDE 0.11% IN PLASTIC CONTAINER; POTASSIUM CHLORIDE 0.15% IN DEXTROSE 5% AND SODIUM CHLORIDE 0.2% IN PLASTIC CONTAINER; POTASSIUM CHLORIDE 0.15% IN DEXTROSE 5% AND SODIUM CHLORIDE 0.33% IN PLASTIC CONTAINER; POTASSIUM CHLORIDE 0.15% IN DEXTROSE 5% AND SODIUM CHLORIDE 0.45% IN PLASTIC CONTAINER; POTASSIUM CHLORIDE 0.15% IN DEXTROSE 5% AND SODIUM CHLORIDE 0.9% IN PLASTIC CONTAINER; POTASSIUM CHLORIDE 0.15% IN DEXTROSE 5% IN PLASTIC CONTAINER, DEXTROSE; POTASSIUM CHLORIDE 0.15% IN SODIUM CHLORIDE 0.45%, POTASSIUM CHLORIDE; POTASSIUM CHLORIDE 0.15% IN SODIUM CHLORIDE 0.9% IN PLASTIC CONTAINER, POTASSIUM CHLORIDE; POTASSIUM CHLORIDE 0.15% IN SODIUM CHLORIDE 0.9%, POTASSIUM CHLORIDE; POTASSIUM CHLORIDE 0.22% IN DEXTROSE 10% AND SODIUM CHLORIDE 0.2% IN PLASTIC CONTAINER; POTASSIUM CHLORIDE 0.22% IN DEXTROSE 10% AND SODIUM CHLORIDE 0.45% IN PLASTIC CONTAINER; POTASSIUM CHLORIDE 0.22% IN DEXTROSE 10% AND SODIUM CHLORIDE 0.9% IN PLASTIC CONTAINER; POTASSIUM CHLORIDE 0.22% IN DEXTROSE 3.3% AND SODIUM CHLORIDE 0.3% IN PLASTIC CONTAINER; POTASSIUM CHLORIDE 0.22% IN DEXTROSE 5% AND SODIUM CHLORIDE 0.11% IN PLASTIC CONTAINER; POTASSIUM CHLORIDE 0.22% IN DEXTROSE 5% AND SODIUM CHLORIDE 0.2% IN PLASTIC CONTAINER; POTASSIUM CHLORIDE 0.22% IN DEXTROSE 5% AND SODIUM CHLORIDE 0.33% IN PLASTIC CONTAINER; POTASSIUM CHLORIDE 0.22% IN DEXTROSE 5% AND SODIUM CHLORIDE 0.45% IN PLASTIC CONTAINER; POTASSIUM CHLORIDE 0.22% IN DEXTROSE 5% AND SODIUM CHLORIDE 0.9% IN PLASTIC CONTAINER; POTASSIUM CHLORIDE 0.3% AND SODIUM CHLORIDE 0.9%, POTASSIUM CHLORIDE; POTASSIUM CHLORIDE 0.3% IN DEXTROSE 10% AND SODIUM CHLORIDE 0.2% IN PLASTIC CONTAINER; POTASSIUM CHLORIDE 0.3% IN DEXTROSE 10% AND SODIUM CHLORIDE 0.45% IN PLASTIC CONTAINER; POTASSIUM CHLORIDE 0.3% IN DEXTROSE 10% AND SODIUM CHLORIDE 0.9% IN PLASTIC CONTAINER; POTASSIUM CHLORIDE 0.3% IN DEXTROSE 3.3% AND SODIUM CHLORIDE 0.3% IN PLASTIC CONTAINER; POTASSIUM CHLORIDE 0.3% IN DEXTROSE 5% AND SODIUM CHLORIDE 0.11% IN PLASTIC CONTAINER; POTASSIUM CHLORIDE 0.3% IN DEXTROSE 5% AND SODIUM CHLORIDE 0.2% IN PLASTIC CONTAINER; POTASSIUM CHLORIDE 0.3% IN DEXTROSE 5% AND SODIUM CHLORIDE 0.33% IN PLASTIC CONTAINER; POTASSIUM CHLORIDE 0.3% IN DEXTROSE 5% AND SODIUM CHLORIDE 0.45% IN PLASTIC CONTAINER; POTASSIUM CHLORIDE 0.3% IN DEXTROSE 5% AND SODIUM CHLORIDE 0.9% IN PLASTIC CONTAINER; POTASSIUM CHLORIDE 0.3% IN DEXTROSE 5% IN PLASTIC CONTAINER, DEXTROSE; POTASSIUM CHLORIDE 10 MEQ IN DEXTROSE 5% AND LACTATED RINGER'S IN PLASTIC CONTAINER; POTASSIUM CHLORIDE 10 MEQ IN DEXTROSE 5% AND SODIUM CHLORIDE 0.225% IN PLASTIC CONTAINER; POTASSIUM CHLORIDE 10 MEQ IN DEXTROSE 5% AND SODIUM CHLORIDE 0.45% DEXTROSE; POTASSIUM CHLORIDE 10 MEQ IN DEXTROSE 5% AND SODIUM CHLORIDE 0.45% IN PLASTIC CONTAINER; POTASSIUM CHLORIDE 10 MEQ IN DEXTROSE 5% AND SODIUM CHLORIDE 0.9% IN PLASTIC CONTAINER; POTASSIUM CHLORIDE 10 MEQ IN PLASTIC CONTAINER, POTASSIUM CHLORIDE; POTASSIUM CHLORIDE 10 MEQ, POTASSIUM CHLORIDE; POTASSIUM CHLORIDE 15 MEQ IN DEXTROSE 5% AND LACTATED RINGER'S IN PLASTIC CONTAINER; POTASSIUM CHLORIDE 20 MEQ IN DEXTROSE 5% AND LACTATED RINGER'S IN PLASTIC CONTAINER; POTASSIUM CHLORIDE 20 MEQ IN DEXTROSE 5% AND SODIUM CHLORIDE 0.225% IN PLASTIC CONTAINER; POTASSIUM CHLORIDE 20 MEQ IN DEXTROSE 5% AND SODIUM CHLORIDE 0.45% DEXTROSE; POTASSIUM CHLORIDE 20 MEQ IN DEXTROSE 5% AND SODIUM CHLORIDE 0.45% IN PLASTIC CONTAINER; POTASSIUM CHLORIDE 20 MEQ IN DEXTROSE 5% AND SODIUM CHLORIDE 0.9% IN PLASTIC CONTAINER; POTASSIUM CHLORIDE 20 MEQ IN DEXTROSE 5% AND SODIUM CHLORIDE 0.9%, DEXTROSE; POTASSIUM CHLORIDE 20 MEQ IN DEXTROSE 5% IN PLASTIC CONTAINER, DEXTROSE; POTASSIUM CHLORIDE 20 MEQ IN PLASTIC CONTAINER, POTASSIUM CHLORIDE; POTASSIUM CHLORIDE 20 MEQ IN SODIUM CHLORIDE 0.9% IN PLASTIC CONTAINER, POTASSIUM CHLORIDE; POTASSIUM CHLORIDE 20 MEQ, POTASSIUM CHLORIDE; POTASSIUM CHLORIDE 30 MEQ IN DEXTROSE 5% AND LACTATED RINGER'S IN PLASTIC CONTAINER; POTASSIUM CHLORIDE 30 MEQ IN DEXTROSE 5% AND SODIUM CHLORIDE 0.45% DEXTROSE; POTASSIUM CHLORIDE 30 MEQ IN DEXTROSE 5% AND SODIUM CHLORIDE 0.45% IN PLASTIC CONTAINER; POTASSIUM CHLORIDE 30 MEQ IN DEXTROSE 5% AND SODIUM CHLORIDE 0.9% IN PLASTIC CONTAINER; POTASSIUM CHLORIDE 30 MEQ IN PLASTIC CONTAINER, POTASSIUM CHLORIDE; POTASSIUM CHLORIDE 40 MEQ IN DEXTROSE 5% AND LACTATED RINGER'S IN PLASTIC CONTAINER; POTASSIUM CHLORIDE 40 MEQ IN DEXTROSE 5% AND SODIUM CHLORIDE 0.45% IN PLASTIC CONTAINER; POTASSIUM CHLORIDE 40 MEQ IN DEXTROSE 5% AND SODIUM CHLORIDE 0.45%, DEXTROSE; POTASSIUM CHLORIDE 40 MEQ IN DEXTROSE 5% AND SODIUM CHLORIDE 0.9% DEXTROSE; POTASSIUM CHLORIDE 40 MEQ IN DEXTROSE 5% AND SODIUM CHLORIDE 0.9% IN PLASTIC CONTAINER; POTASSIUM CHLORIDE 40 MEQ IN PLASTIC CONTAINER, POTASSIUM CHLORIDE; POTASSIUM CHLORIDE 40 MEQ IN SODIUM CHLORIDE 0.9% IN PLASTIC CONTAINER, POTASSIUM CHLORIDE; POTASSIUM CHLORIDE 40 MEQ, POTASSIUM CHLORIDE; POTASSIUM CHLORIDE 5 MEQ IN DEXTROSE 5% AND LACTATED RINGER'S IN PLASTIC CONTAINER, CALCIUM; POTASSIUM CHLORIDE 5 MEQ IN DEXTROSE 5% AND SODIUM CHLORIDE 0.45% IN PLASTIC CONTAINER; POTASSIUM CHLORIDE 5 MEQ IN DEXTROSE 5% AND SODIUM CHLORIDE 0.9% IN PLASTIC CONTAINER; POTASSIUM CHLORIDE IN PLASTIC CONTAINER, POTASSIUM CHLORIDE; POTASSIUM CHLORIDE, POTASSIUM CHLORIDE; POTASSIUM CITRATE, POTASSIUM CITRATE; POTASSIUM IODIDE, POTASSIUM IODIDE (OTC); POTASSIUM PHOSPHATES, POTASSIUM PHOSPHATE; POVIDONE IODINE, POVIDONE-IODINE (OTC); PRALSETINIB; PRAMIPEXOLE DIHYDROCHLORIDE, PRAMIPEXOLE DIHYDROCHLORIDE; PRAMLINTIDE ACETATE; PRASUGREL HYDROCHLORIDE, PRASUGREL HYDROCHLORIDE; PRASUGREL, PRASUGREL HYDROCHLORIDE; PRAVASTATIN SODIUM, PRAVASTATIN SODIUM; PRAZIQUANTEL, PRAZIQUANTEL; PRAZOSIN HYDROCHLORIDE, PRAZOSIN HYDROCHLORIDE; PREDNICARBATE, PREDNICARBATE; PREDNISOLONE ACETATE; PREDNISOLONE SODIUM PHOSPHATE, PREDNISOLONE SODIUM PHOSPHATE; PREDNISOLONE, PREDNISOLONE; PREDNISONE, PREDNISONE; PREGABALIN, PREGABALIN; PRETOMANID, PRETOMANID; PRILOCAINE HYDROCHLORIDE AND EPINEPHRINE BITARTRATE, EPINEPHRINE BITARTRATE; PRILOCAINE HYDROCHLORIDE, PRILOCAINE HYDROCHLORIDE; PRIMAQUINE PHOSPHATE, PRIMAQUINE PHOSPHATE; PRIMAQUINE, PRIMAQUINE PHOSPHATE; PRIMIDONE, PRIMIDONE; PROBENECID AND COLCHICINE, COLCHICINE; PROBENECID, PROBENECID; PROCAINAMIDE HYDROCHLORIDE, PROCAINAMIDE HYDROCHLORIDE; PROCARBAZINE HYDROCHLORIDE; PROCHLORPERAZINE EDISYLATE, PROCHLORPERAZINE EDISYLATE; PROCHLORPERAZINE MALEATE, PROCHLORPERAZINE MALEATE; PROCHLORPERAZINE, PROCHLORPERAZINE; PROGESTERONE, PROGESTERONE; PROMETH HYDROCHLORIDE, PHENYLEPHRINE HYDROCHLORIDE W/CODEINE PHOSPHATE; PROMETHAZINE DM, DEXTROMETHORPHAN HYDROBROMIDE; PROMETHAZINE HYDROCHLORIDE AND CODEINE PHOSPHATE, CODEINE PHOSPHATE; PROMETHAZINE HYDROCHLORIDE AND DEXTROMETHORPHAN HYDROBROMIDE, DEXTROMETHORPHAN; PROMETHAZINE HYDROCHLORIDE AND PHENYLEPHRINE HYDROCHLORIDE, PHENYLEPHRINE HYDROCHLORIDE; PROMETHAZINE HYDROCHLORIDE, PHENYLEPHRINE HYDROCHLORIDE W/CODEINE PHOSPHATE; PROMETHAZINE HYDROCHLORIDE, PROMETHAZINE HYDROCHLORIDE; PROMETHAZINE PLAIN, PROMETHAZINE HYDROCHLORIDE; PROMETHAZINE W/DEXTROMETHORPHAN, DEXTROMETHORPHAN HYDROBROMIDE; PROMETHAZINE WITH CODEINE, CODEINE PHOSPHATE; PROPAFENONE HYDROCHLORIDE, PROPAFENONE HYDROCHLORIDE; PROPARACAINE HYDROCHLORIDE, PROPARACAINE HYDROCHLORIDE; PROPOFOL, PROPOFOL; PROPRANOLOL HYDROCHLORIDE, PROPRANOLOL HYDROCHLORIDE; PROPYLTHIOURACIL, PROPYLTHIOURACIL; PROTAMINE SULFATE, PROTAMINE SULFATE; PROTRIPTYLINE HYDROCHLORIDE, PROTRIPTYLINE HYDROCHLORIDE; PSEUDOEPHEDRINE HYDROCHLORIDE, PSEUDOEPHEDRINE HYDROCHLORIDE (OTC); PURIFIED CORTROPHIN GEL, CORTICOTROPIN; PYRAZINAMIDE, PYRAZINAMIDE; PYRIDOSTIGMINE BROMIDE, PYRIDOSTIGMINE BROMIDE; PYRIDOXINE HYDROCHLORIDE, PYRIDOXINE HYDROCHLORIDE; PYRIMETHAMINE, PYRIMETHAMINE; QUETIAPINE FUMARATE, QUETIAPINE FUMARATE; QUINAPRIL HYDROCHLORIDE AND HYDROCHLOROTHIAZIDE, HYDROCHLOROTHIAZIDE; QUINAPRIL HYDROCHLORIDE, QUINAPRIL HYDROCHLORIDE; QUINIDINE GLUCONATE, QUINIDINE GLUCONATE; QUINIDINE SULFATE, QUINIDINE SULFATE; QUININE SULFATE, QUININE SULFATE; QUIZARTINIB DIHYDROCHLORIDE; RABEPRAZOLE SODIUM, RABEPRAZOLE SODIUM; RALOXIFENE HYDROCHLORIDE, RALOXIFENE HYDROCHLORIDE; RALTEGRAVIR POTASSIUM; RAMELTEON, RAMELTEON; RAMIPRIL, RAMIPRIL; RANITIDINE HYDROCHLORIDE, RANITIDINE HYDROCHLORIDE; RANITIDINE HYDROCHLORIDE, RANITIDINE HYDROCHLORIDE (OTC); RANOLAZINE, RANOLAZINE; RASAGILINE MESYLATE, RASAGILINE MESYLATE; REGADENOSON, REGADENOSON; REGORAFENIB; RELUGOLIX; REMIFENTANIL HYDROCHLORIDE, REMIFENTANIL HYDROCHLORIDE; REPAGLINIDE, REPAGLINIDE; REVEFENACIN; RIBAVIRIN, RIBAVIRIN; RIBOCICLIB SUCCINATE; RIFABUTIN, RIFABUTIN; RIFAMPIN, RIFAMPIN; RIFAPENTINE; RIFAXIMIN; RILUZOLE, RILUZOLE; RIMANTADINE HYDROCHLORIDE, RIMANTADINE HYDROCHLORIDE; RINGER'S IN PLASTIC CONTAINER, CALCIUM CHLORIDE; RIOCIGUAT, RIOCIGUAT; RIPRETINIB; RISDIPLAM; RISEDRONATE SODIUM, RISEDRONATE SODIUM; RISPERIDONE, RISPERIDONE; RITONAVIR, RITONAVIR; RIVAROXABAN; RIVASTIGMINE TARTRATE, RIVASTIGMINE TARTRATE; RIVASTIGMINE, RIVASTIGMINE; RIZATRIPTAN BENZOATE, RIZATRIPTAN BENZOATE; ROCURONIUM BROMIDE, ROCURONIUM BROMIDE; ROFLUMILAST, ROFLUMILAST; ROLAPITANT HYDROCHLORIDE; ROMIDEPSIN, ROMIDEPSIN; ROPINIROLE HYDROCHLORIDE, ROPINIROLE HYDROCHLORIDE; ROPIVACAINE HYDROCHLORIDE, ROPIVACAINE HYDROCHLORIDE; ROSUVASTATIN CALCIUM, ROSUVASTATIN CALCIUM; ROTIGOTINE; RUCAPARIB CAMSYLATE; RUFINAMIDE, RUFINAMIDE; RUXOLITINIB PHOSPHATE; SACUBITRIL; SAFINAMIDE MESYLATE; SAPROPTERIN DIHYDROCHLORIDE, SAPROPTERIN DIHYDROCHLORIDE; SCOPOLAMINE, SCOPOLAMINE; SECNIDAZOLE; SELEGILINE; SELEGILINE HYDROCHLORIDE, SELEGILINE HYDROCHLORIDE; SELENIOUS ACID, SELENIOUS ACID; SELENIUM SULFIDE, SELENIUM SULFIDE; SELEXIPAG, SELEXIPAG; SELINEXOR; SELPERCATINIB; SELUMETINIB SULFATE; SEMAGLUTIDE; SERTRALINE HYDROCHLORIDE, SERTRALINE HYDROCHLORIDE; SEVELAMER CARBONATE, SEVELAMER CARBONATE; SEVELAMER HYDROCHLORIDE, SEVELAMER HYDROCHLORIDE; SEVOFLURANE, SEVOFLURANE; SILDENAFIL CITRATE, SILDENAFIL CITRATE; SILODOSIN, SILODOSIN; SILVER SULFADIAZINE; SIMVASTATIN, SIMVASTATIN; SINCALIDE, SINCALIDE; SIROLIMUS, SIROLIMUS; SITAGLIPTIN PHOSPHATE; SODIUM ACETATE, SODIUM ACETATE; SODIUM BICARBONATE, SODIUM BICARBONATE; SODIUM CHLORIDE 0.45% IN PLASTIC CONTAINER, SODIUM CHLORIDE; SODIUM CHLORIDE 0.9% IN PLASTIC CONTAINER, SODIUM CHLORIDE; SODIUM CHLORIDE 0.9% IN STERILE PLASTIC CONTAINER, SODIUM CHLORIDE; SODIUM CHLORIDE 0.9%, SODIUM CHLORIDE; SODIUM CHLORIDE 14.6%, SODIUM CHLORIDE; SODIUM CHLORIDE 23.4%, SODIUM CHLORIDE; SODIUM CHLORIDE 3% IN PLASTIC CONTAINER, SODIUM CHLORIDE; SODIUM CHLORIDE 5% IN PLASTIC CONTAINER, SODIUM CHLORIDE; SODIUM FERRIC GLUCONATE COMPLEX IN SUCROSE, FERRIC OXYHYDROXIDE; SODIUM FLUORIDE F-18, SODIUM FLUORIDE F-18; SODIUM IODIDE I 123, SODIUM IODIDE I-123; SODIUM IODIDE I 131, SODIUM IODIDE I-131; SODIUM NITRITE, SODIUM NITRITE; SODIUM NITROPRUSSIDE, SODIUM NITROPRUSSIDE; SODIUM OXYBATE; SODIUM PHENYLACETATE AND SODIUM BENZOATE, SODIUM BENZOATE; SODIUM PHENYLBUTYRATE, SODIUM PHENYLBUTYRATE; SODIUM PHOSPHATES IN PLASTIC CONTAINER, SODIUM PHOSPHATE; SODIUM PHOSPHATES, SODIUM PHOSPHATE; SODIUM PICOSULFATE, MAGNESIUM OXIDE AND ANHYDROUS CITRIC ACID; SODIUM POLYSTYRENE SULFONATE, SODIUM POLYSTYRENE SULFONATE; SODIUM SULFATE, POTASSIUM SULFATE AND MAGNESIUM SULFATE; SODIUM TETRADECYL SULFATE, SODIUM TETRADECYL SULFATE; SODIUM THIOSULFATE, SODIUM THIOSULFATE; SODIUM ZIRCONIUM CYCLOSILICATE; SOLIFENACIN SUCCINATE, SOLIFENACIN SUCCINATE; SOLRIAMFETOL HYDROCHLORIDE; SONIDEGIB PHOSPHATE; SORAFENIB TOSYLATE, SORAFENIB TOSYLATE; SORBITOL 3.3% IN PLASTIC CONTAINER, SORBITOL; SORBITOL 3% IN PLASTIC CONTAINER, SORBITOL; SORBITOL-MANNITOL IN PLASTIC CONTAINER, MANNITOL; SOTALOL HYDROCHLORIDE, SOTALOL HYDROCHLORIDE; SOTORASIB; SPARSENTAN; SPIRONOLACTONE AND HYDROCHLOROTHIAZIDE, HYDROCHLOROTHIAZIDE; SPIRONOLACTONE, SPIRONOLACTONE; STERILE WATER FOR INJECTION IN PLASTIC CONTAINER, STERILE WATER FOR INJECTION; STERILE WATER FOR INJECTION, STERILE WATER FOR INJECTION; STERILE WATER IN PLASTIC CONTAINER, STERILE WATER FOR IRRIGATION; STERILE WATER, STERILE WATER FOR IRRIGATION; STIRIPENTOL; STREPTOMYCIN SULFATE, STREPTOMYCIN SULFATE; STRONTIUM CHLORIDE SR-89, STRONTIUM CHLORIDE SR-89; SUCCIMER; SUCCINYLCHOLINE CHLORIDE, SUCCINYLCHOLINE CHLORIDE; SUCRALFATE, SUCRALFATE; SUFENTANIL CITRATE, SUFENTANIL CITRATE; SUGAMMADEX SODIUM, SUGAMMADEX SODIUM; SULFACETAMIDE SODIUM AND PREDNISOLONE SODIUM PHOSPHATE, PREDNISOLONE SODIUM PHOSPHATE; SULFACETAMIDE SODIUM, SULFACETAMIDE SODIUM; SULFADIAZINE, SULFADIAZINE; SULFAMETHOXAZOLE AND TRIMETHOPRIM, SULFAMETHOXAZOLE; SULFASALAZINE, SULFASALAZINE; SULINDAC, SULINDAC; SUMATRIPTAN AND NAPROXEN SODIUM, NAPROXEN SODIUM; SUMATRIPTAN SUCCINATE, SUMATRIPTAN SUCCINATE; SUMATRIPTAN, SUMATRIPTAN; SUNITINIB MALATE, SUNITINIB MALATE; SUVOREXANT; TACROLIMUS, TACROLIMUS; TADALAFIL, TADALAFIL; TAFAMIDIS; TAFAMIDIS MEGLUMINE; TAFENOQUINE SUCCINATE; TAFLUPROST, TAFLUPROST; TALAZOPARIB TOSYLATE; TALC, TALC; TAMOXIFEN CITRATE, TAMOXIFEN CITRATE; TAMSULOSIN HYDROCHLORIDE, TAMSULOSIN HYDROCHLORIDE; TASIMELTEON, TASIMELTEON; TAVABOROLE, TAVABOROLE; TAZAROTENE, TAZAROTENE; TAZEMETOSTAT HYDROBROMIDE; TECHNETIUM TC 99M MERTIATIDE KIT, TECHNETIUM TC-99M MERTIATIDE KIT; TECHNETIUM TC 99M SESTAMIBI, TECHNETIUM TC-99M SESTAMIBI KIT; TECHNETIUM TC-99M MEBROFENIN, TECHNETIUM TC-99M MEBROFENIN KIT; TECHNETIUM TC-99M MEDRONATE KIT, TECHNETIUM TC-99M MEDRONATE KIT; TECHNETIUM TC-99M SULFUR COLLOID KIT, TECHNETIUM TC-99M SULFUR COLLOID KIT; TECHNETIUM TC99M MERTIATIDE KIT, TECHNETIUM TC-99M MERTIATIDE KIT; TEDIZOLID PHOSPHATE; TEDUGLUTIDE RECOMBINANT; TELMISARTAN AND AMLODIPINE, AMLODIPINE BESYLATE; TELMISARTAN AND HYDROCHLOROTHIAZIDE, HYDROCHLOROTHIAZIDE; TELMISARTAN, TELMISARTAN; TELOTRISTAT ETIPRATE; TEMAZEPAM, TEMAZEPAM; TEMOZOLOMIDE, TEMOZOLOMIDE; TEMSIROLIMUS, TEMSIROLIMUS; TENOFOVIR ALAFENAMIDE FUMARATE; TENOFOVIR ALAFENAMIDE, TENOFOVIR ALAFENAMIDE FUMARATE; TENOFOVIR DISOPROXIL FUMARATE, TENOFOVIR DISOPROXIL FUMARATE; TEPOTINIB HYDROCHLORIDE; TERAZOSIN HYDROCHLORIDE, TERAZOSIN HYDROCHLORIDE; TERBINAFINE HYDROCHLORIDE, TERBINAFINE HYDROCHLORIDE; TERBINAFINE HYDROCHLORIDE, TERBINAFINE HYDROCHLORIDE (OTC); TERBUTALINE SULFATE, TERBUTALINE SULFATE; TERCONAZOLE, TERCONAZOLE; TERIFLUNOMIDE, TERIFLUNOMIDE; TERIPARATIDE, TERIPARATIDE; TESTOSTERONE CYPIONATE, TESTOSTERONE CYPIONATE; TESTOSTERONE ENANTHATE, TESTOSTERONE ENANTHATE; TESTOSTERONE, TESTOSTERONE; TETRABENAZINE, TETRABENAZINE; TETRACAINE HYDROCHLORIDE, TETRACAINE HYDROCHLORIDE; TETRACYCLINE HYDROCHLORIDE, TETRACYCLINE HYDROCHLORIDE; THALIDOMIDE; THALLOUS CHLORIDE TL 201, THALLOUS CHLORIDE TL-201; THEOPHYLLINE, THEOPHYLLINE; THIAMINE HYDROCHLORIDE, THIAMINE HYDROCHLORIDE; THIOGUANINE, THIOGUANINE; THIORIDAZINE HYDROCHLORIDE, THIORIDAZINE HYDROCHLORIDE; THIOTEPA, THIOTEPA; THIOTHIXENE, THIOTHIXENE; TIAGABINE HYDROCHLORIDE, TIAGABINE HYDROCHLORIDE; TICAGRELOR, TICAGRELOR; TICLOPIDINE HYDROCHLORIDE, TICLOPIDINE HYDROCHLORIDE; TIGECYCLINE, TIGECYCLINE; TIMOLOL MALEATE, TIMOLOL MALEATE; TINIDAZOLE, TINIDAZOLE; TIOCONAZOLE, TIOCONAZOLE (OTC); TIOPRONIN, TIOPRONIN; TIOTROPIUM BROMIDE, TIOTROPIUM BROMIDE; TIPIRACIL HYDROCHLORIDE; TIPIRACIL HYDROCHLORIDE AND TRIFLURIDINE, TIPIRACIL HYDROCHLORIDE; TIPRANAVIR; TIRBANIBULIN; TIROFIBAN HYDROCHLORIDE, TIROFIBAN HYDROCHLORIDE; TIVOZANIB HYDROCHLORIDE; TIZANIDINE HYDROCHLORIDE, TIZANIDINE HYDROCHLORIDE; TOBRAMYCIN AND DEXAMETHASONE, DEXAMETHASONE; TOBRAMYCIN SULFATE (PHARMACY BULK), TOBRAMYCIN SULFATE; TOBRAMYCIN SULFATE, TOBRAMYCIN SULFATE; TOBRAMYCIN, TOBRAMYCIN; TOFACITINIB CITRATE, TOFACITINIB CITRATE; TOLCAPONE, TOLCAPONE; TOLMETIN SODIUM, TOLMETIN SODIUM; TOLTERODINE TARTRATE, TOLTERODINE TARTRATE; TOLVAPTAN, TOLVAPTAN; TOPIRAMATE, TOPIRAMATE; TOPOTECAN HYDROCHLORIDE, TOPOTECAN HYDROCHLORIDE; TOREMIFENE CITRATE, TOREMIFENE CITRATE; TORSEMIDE, TORSEMIDE; TPN ELECTROLYTES IN PLASTIC CONTAINER, CALCIUM CHLORIDE; TRAMADOL HYDROCHLORIDE AND ACETAMINOPHEN, ACETAMINOPHEN; TRAMADOL HYDROCHLORIDE, TRAMADOL HYDROCHLORIDE; TRAMETINIB DIMETHYL SULFOXIDE; TRANDOLAPRIL AND VERAPAMIL HYDROCHLORIDE, TRANDOLAPRIL; TRANDOLAPRIL, TRANDOLAPRIL; TRANEXAMIC ACID, TRANEXAMIC ACID; TRANYLCYPROMINE SULFATE, TRANYLCYPROMINE SULFATE; TRAVOPROST, TRAVOPROST; TRAZODONE HYDROCHLORIDE, TRAZODONE HYDROCHLORIDE; TREPROSTINIL DIOLAMINE; TREPROSTINIL, TREPROSTINIL; TRETINOIN MICROSPHERE, TRETINOIN; TRETINOIN, TRETINOIN; TRIAMCINOLONE ACETONIDE, TRIAMCINOLONE ACETONIDE; TRIAMCINOLONE ACETONIDE, TRIAMCINOLONE ACETONIDE (OTC); TRIAMTERENE AND HYDROCHLOROTHIAZIDE, HYDROCHLOROTHIAZIDE; TRIAMTERENE, TRIAMTERENE; TRIAZOLAM, TRIAZOLAM; TRIENTINE HYDROCHLORIDE, TRIENTINE HYDROCHLORIDE; TRIENTINE TETRAHYDROCHLORIDE; TRIFLUOPERAZINE HYDROCHLORIDE, TRIFLUOPERAZINE HYDROCHLORIDE; TRIFLURIDINE, TRIFLURIDINE; TRIHEPTANOIN; TRIHEXYPHENIDYL HYDROCHLORIDE, TRIHEXYPHENIDYL HYDROCHLORIDE; TRIMETHOBENZAMIDE HYDROCHLORIDE, TRIMETHOBENZAMIDE HYDROCHLORIDE; TRIMETHOPRIM SULFATE AND POLYMYXIN B SULFATE, POLYMYXIN B SULFATE; TRIMETHOPRIM, TRIMETHOPRIM; TRIMIPRAMINE MALEATE, TRIMIPRAMINE MALEATE; TRIPTORELIN PAMOATE; TROFINETIDE; TROPICAMIDE, TROPICAMIDE; TROSPIUM CHLORIDE, TROSPIUM CHLORIDE; TUCATINIB; UBROGEPANT; ULIPRISTAL ACETATE; UMECLIDINIUM BROMIDE; UPADACITINIB; URSODIOL, URSODIOL; VALACYCLOVIR HYDROCHLORIDE, VALACYCLOVIR HYDROCHLORIDE; VALBENAZINE TOSYLATE; VALGANCICLOVIR HYDROCHLORIDE, VALGANCICLOVIR HYDROCHLORIDE; VALPROATE SODIUM, VALPROATE SODIUM; VALPROIC ACID, VALPROIC ACID; VALRUBICIN, VALRUBICIN; VALSARTAN AND HYDROCHLOROTHIAZIDE, HYDROCHLOROTHIAZIDE; VALSARTAN, VALSARTAN; VANCOMYCIN HYDROCHLORIDE, VANCOMYCIN HYDROCHLORIDE; VANDETANIB; VARDENAFIL HYDROCHLORIDE, VARDENAFIL HYDROCHLORIDE; VARENICLINE TARTRATE, VARENICLINE TARTRATE; VASOPRESSIN IN SODIUM CHLORIDE 0.9%, VASOPRESSIN; VASOPRESSIN, VASOPRESSIN; VECURONIUM BROMIDE, VECURONIUM BROMIDE; VEMURAFENIB; VENETOCLAX; VENLAFAXINE BESYLATE, VENLAFAXINE BESYLATE; VENLAFAXINE HYDROCHLORIDE, VENLAFAXINE HYDROCHLORIDE; VERAPAMIL HYDROCHLORIDE, VERAPAMIL HYDROCHLORIDE; VERICIGUAT; VIBEGRON; VIGABATRIN, VIGABATRIN; VILAZODONE HYDROCHLORIDE, VILAZODONE HYDROCHLORIDE; VINBLASTINE SULFATE, VINBLASTINE SULFATE; VINCRISTINE SULFATE PFS, VINCRISTINE SULFATE; VINORELBINE TARTRATE, VINORELBINE TARTRATE; VISMODEGIB; VITAMIN D, ERGOCALCIFEROL; VITAMIN KI, PHYTONADIONE; VOCLOSPORIN; VORAPAXAR SULFATE; VORICONAZOLE, VORICONAZOLE; VORINOSTAT; VORTIOXETINE HYDROBROMIDE; VOXELOTOR; WARFARIN SODIUM, WARFARIN SODIUM; XENON XE 133, XENON XE-133; ZAFIRLUKAST, ZAFIRLUKAST; ZALEPLON, ZALEPLON; ZANAMIVIR; ZANUBRUTINIB; ZIDOVUDINE, ZIDOVUDINE; ZILEUTON, ZILEUTON; ZINC CHLORIDE IN PLASTIC CONTAINER, ZINC CHLORIDE; ZINC CHLORIDE, ZINC CHLORIDE; ZINC SULFATE, ZINC SULFATE; ZIPRASIDONE HYDROCHLORIDE, ZIPRASIDONE HYDROCHLORIDE; ZIPRASIDONE MESYLATE, ZIPRASIDONE MESYLATE; ZOLEDRONIC ACID, ZOLEDRONIC ACID; ZOLEDRONIC, ZOLEDRONIC ACID; ZOLMITRIPTAN, ZOLMITRIPTAN; ZOLPIDEM TARTRATE, ZOLPIDEM TARTRATE; or ZONISAMIDE, ZONISAMIDE; or
for indications—DYSMENORRHEA; CHOLANGIOPANCREATOGRAPHY; INTRAVENOUS DIGITAL SUBTRACTION ANGIOGRAPHY; PERIPHERAL VENOGRAPHY (PHLEBOGRAPHY); HYSTEROSALPINGOGRAPHY; TREATMENT OF JUVENILE ARTHRITIS; BIOPSY PROVEN MINIMAL CHANGE NEPHROTIC SYNDROME IN CHILDREN; ADULT INTRAVENOUS CONTRAST-ENHANCED COMPUTED TOMOGRAPHY OF THE HEAD AND BODY; PREVENTION OF POSTOPERATIVE NAUSEA AND VOMITING; PREVENTION OF POSTOPERATIVE DEEP VENOUS THROMBOSIS AND PULMONARY EMBOLISM IN TOTAL HIP REPLACEMENT SURGERY; RELIEF OF MILD TO MODERATE PAIN; TREATMENT OF CUTANEOUS CANDIDIASIS; URINARY TRACT INFECTION (UTI) PREVENTION FOR PERIODS UP TO FIVE MONTHS IN WOMEN WITH A HISTORY OF RECURRENT UTI; SEBORRHEIC DERMATITIS; PHOTOPHERESIS IN THE PALLIATIVE TREATMENT OF SKIN MANIFESTATIONS OF CUTANEOUS T-CELL LYMPHOMA IN PERSONS NOT RESPONSIVE TO OTHER TREATMENT; STIMULATE THE DEVELOPMENT OF MULTIPLE FOLLICLES/OOCYTES IN OVULATORY PATIENTS PARTICIPATING IN AN IN VITRO FERTILIZATION PROGRAM; MANAGEMENT OF CONGESTIVE HEART FAILURE; ENDOSCOPIC RETROGRADE PANCREATOGRAPHY; HERNIOGRAPHY; KNEE ARTHROGRAPHY; HIGH DOSE METHOTREXATE WITH LEUCOVORIN RESCUE IN COMBINATION WITH OTHER CHEMOTHERAPEUTIC AGENTS TO DELAY RECURRENCE IN PATIENTS WITH NONMETASTATIC OSTEOSARCOMA WHO HAVE UNDERGONE SURGICAL RESECTION OR AMPUTATION FOR THE PRIMARY TUMOR; RESCUE AFTER HIGH-DOSE METHOTREXATE THERAPY IN OSTEOSARCOMA; SHORT-TERM TREATMENT OF ACTIVE BENIGN GASTRIC ULCER; TREATMENT OF RHEUMATOID ARTHRITIS; ADULT INTRA-ARTERIAL DIGITAL SUBTRACTION ANGIOGRAPHY OF THE HEAD, NECK, ABDOMINAL, RENAL AND PERIPHERAL VESSELS; TREATMENT OF LIVER FLUKES; ADJUNCTIVE THERAPY TO DIET TO REDUCE THE RISK OF CORONARY ARTERY DISEASE; SELECTIVE ADULT VISCERAL ARTERIOGRAPHY; METASTATIC BREAST CANCER IN PREMENOPAUSAL WOMEN AS AN ALTERNATIVE TO OOPHORECTOMY OR OVARIAN IRRADIATION; TREATMENT OF TINEA PEDIS; CONTRAST ENHANCEMENT AGENT TO FACILITATE VISUALIZATION OF LESIONS IN THE SPINE AND ASSOCIATED TISSUES; PEDIATRIC MYELOGRAPHY; ORAL USE OF DILUTED OMNIPAQUE INJECTION IN ADULTS FOR CONTRAST ENHANCED COMPUTED TOMOGRAPHY OF THE ABDOMEN; ORAL USE IN ADULTS FOR PASS-THROUGH EXAMINATION OF THE GASTROINTESTINAL TRACT; PEDIATRIC CONTRAST ENHANCEMENT OF COMPUTED TOMOGRAPHIC HEAD IMAGING; ARTHROGRAPHY OF THE SHOULDER JOINTS IN ADULTS; RADIOGRAPHY OF THE TEMPOROMANDIBULAR JOINT IN ADULTS; CONTRAST ENHANCEMENT AGENT TO FACILITATE VISUALIZATION OF LESIONS OF THE CENTRAL NERVOUS SYSTEM IN CHILDREN (2 YEARS OF AGE AND OLDER); TREATMENT OF ACUTE MYOCARDIAL INFARCTION; PRIMARY NOCTURNAL ENURESIS; MIGRAINE HEADACHE PROPHYLAXIS; HERPES ZOSTER; HERPES SIMPLEX ENCEPHALITIS; MAINTENANCE THERAPY IN HEALED DUODENAL ULCER PATIENTS AT DOSE OF 1 GRAM TWICE DAILY; ACUTE TREATMENT OF VARICELLA ZOSTER VIRUS; USE IN PEDIATRIC COMPUTED TOMOGRAPHIC HEAD AND BODY IMAGING; TREATMENT OF PEDIATRIC PATIENTS WITH SYMPTOMATIC HUMAN IMMUNODEFICIENCY VIRUS (HIV) DISEASE; PEDIATRIC ANGIOCARDIOGRAPHY; TREATMENT OF TRAVELERS' DIARRHEA DUE TO SUSCEPTIBLE STRAINS OF ENTEROTOXIGENIC ESCHERICHIA COLI; FOR USE IN WOMEN WITH AXILLARY NODE-NEGATIVE BREAST CANCER; TREATMENT OF PRIMARY DYSMENORRHEA AND FOR THE TREATMENT OF IDIOPATHIC HEAVY MENSTRUAL BLOOD LOSS; PEDIATRIC EXCRETORY UROGRAPHY; TREATMENT OF PANIC DISORDER, WITH OR WITHOUT AGORAPHOBIA; RENAL CONCENTRATION CAPACITY TEST; HYPERTENSION; EROSIVE GASTROESOPHAGEAL REFLUX DISEASE; SHORT-TERM TREATMENT OF ACTIVE DUODENAL ULCER; INITIAL TREATMENT OF ADVANCED OVARIAN CARCINOMA IN COMBINATION WITH OTHER APPROVED CHEMOTHERAPEUTIC AGENTS; ENDOSCOPICALLY DIAGNOSED ESOPHAGITIS, INCLUDING EROSIVE AND ULCERATIVE ESOPHAGITIS, AND ASSOCIATED HEARTBURN DUE TO GASTROESOPHAGEAL REFLUX DISEASE; SINGLE APPLICATION TREATMENT OF HEAD LICE IN CHILDREN TWO MONTHS TO TWO YEARS IN AGE; FEMALE ANDROGENETIC ALOPECIA; PREVENTION AND TREATMENT OF POSTMENOPAUSAL OSTEOPOROSIS; ONCE DAILY TREATMENT AS INITIAL THERAPY IN THE TREATMENT OF HYPERTENSION; PREVENTION OF SUPRAVENTRICULAR TACHYCARDIAS; PREVENTION OF UPPER GASTROINTESTINAL BLEEDING IN CRITICALLY ILL PATIENTS; UNCOMPLICATED GONORRHEA; TREATMENT OF ACUTE ASTHMATIC ATTACKS IN CHILDREN SIX YEARS OF AGE AND OLDER; CENTRAL PRECOCIOUS PUBERTY; SHORT TERM TREATMENT OF PATIENTS WITH SYMPTOMS OF GASTROESOPHAGEAL REFLUX DISEASE (GERD), AND FOR THE SHORT TERM TREATMENT OF ESOPHAGITIS DUE TO GERD INCLUDING ULCERATIVE DISEASE DIAGNOSED BY ENDOSCOPY; USE IN COMBINATION WITH 5-FLUOROURACIL TO PROLONG SURVIVAL IN THE PALLIATIVE TREATMENT OF PATIENTS WITH ADVANCED COLORECTAL CANCER; VARICELLA INFECTIONS (CHICKENPOX); PREVENTION OF CMV DISEASE IN TRANSPLANT PATIENTS AT RISK FOR CMV DISEASE; INITIATE AND MAINTAIN MONITORED ANESTHESIA CARE (MAC) SEDATION DURING DIAGNOSTIC PROCEDURES; INTRAVENOUS DIGITAL SUBTRACTION ANGIOGRAPHY; TREATMENT OF ENDOSCOPICALLY DIAGNOSED EROSIVE ESOPHAGITIS; PREVENTION OF OSTEOPOROSIS; DERMAL INFECTIONS-TINEA PEDIS, TINEA CORPORIS, TINEA CRURIS DUE TO EPIDERMOPHYTON FLOCCOSUM; CONTRAST ENHANCED COMPUTED TOMOGRAPHIC IMAGING OF THE HEAD AND BODY AND INTRAVENOUS EXCRETORY UROGRAPHY; MANAGEMENT OF CHRONIC STABLE ANGINA AND ANGINA DUE TO CORONARY ARTERY SPASM; DIAGNOSIS AND LOCALIZATION OF ISCHEMIA AND CORONARY HEART DISEASE; PROPHYLAXIS IN DESIGNATED IMMUNOCOMPROMISED CONDITIONS TO REDUCE THE INCIDENCE OF OROPHARYNGEAL CANDIDIASIS; TREATMENT OF TRAVELERS' DIARRHEA; ANGIOCARDIOGRAPHY, CONTRAST ENHANCED COMPUTED TOMOGRAPHIC IMAGING OF THE HEAD AND BODY, AND INTRAVENOUS EXCRETORY UROGRAPHY IN CHILDREN; INTRAOPERATIVE AND POSTOPERATIVE TACHYCARDIA AND/OR HYPERTENSION; TREATMENT OF ANOREXIA ASSOCIATED WITH WEIGHT LOSS IN PATIENTS WITH AIDS; TREATMENT OF SECONDARY CARNITINE DEFICIENCY; RENAL IMAGING AGENT FOR USE IN CHILDREN; MANAGEMENT OF ENDOMETRIOSIS; EPIDURAL USE IN LABOR AND DELIVERY AS AN ANALGESIC ADJUNCT TO BUPIVACAINE; INTENSIVE CARE UNIT SEDATION; MONOTHERAPY USE FOR HYPERTENSION; ADJUNCTIVE THERAPY IN THE MANAGEMENT OF HEART FAILURE; PREVENTION OF EXERCISE-INDUCED BRONCHOSPASM IN CHILDREN AGES 4-11 YEARS; USE WITH MRI IN ADULTS TO PROVIDE CONTRAST ENHANCEMENT AND FACILITATE VISUALIZATION OF LESIONS IN THE BODY [EXCLUDING THE HEART; TREATMENT OF LEFT VENTRICULAR DYSFUNCTION FOLLOWING MYOCARDIAL INFARCTION; TREATMENT OF SYMPTOMATIC BENIGN PROSTATIC HYPERPLASIA; ORAL OR RECTAL USE IN CHILDREN FOR THE EXAMINATION OF THE GASTROINTESTINAL TRACT; TREATMENT OF CHILDREN WHO HAVE GROWTH FAILURE ASSOCIATED WITH CHRONIC RENAL INSUFFICIENCY; PEDIATRIC ANESTHESIA IN CHILDREN 3 YEARS AND OLDER; TO DECREASE THE INCIDENCE OF CANDIDIASIS IN PATIENTS UNDERGOING BONE MARROW TRANSPLANTATION WHO RECEIVE CYTOTOXIC CHEMOTHERAPY AND/OR RADIATION THERAPY; TREATMENT OF DIABETIC NEPHROPATHY IN PATIENTS WITH TYPE I INSULIN-DEPENDENT DIABETES MELLITUS AND RETINOPATHY; TREATMENT OF OBSESSIVE-COMPULSIVE DISORDER; PROPHYLAXIS AGAINST PNEUMOCYSTIS CARINII PNEUMONIA IN INDIVIDUALS WHO ARE IMMUNOCOMPROMISED AND CONSIDERED TO BE AT RISK OF DEVELOPING PNEUMOCYSTIS CARINII PNEUMONIA; TREATMENT OF PULMONARY AND EXTRAPULMONARY ASPERGILLOSIS IN PATIENTS WHO ARE INTOLERANT OF OR WHO ARE REFRACTORY TO AMPHOTERICIN B THERAPY; TREATMENT OF METASTATIC CARCINOMA OF THE BREAST AFTER FAILURE OF FIRST-LINE OR SUBSEQUENT CHEMOTHERAPY; TREATMENT OF ACROMEGALY VAGINAL CANDIDIASIS; EXPANDED USE-FOR ICU PATIENTS UNDERGOING LONG-TERM INFUSION DURING MECHANICAL VENTILATION; TYPHOID FEVER; PREVENTION OF NAUSEA AND VOMITING ASSOCIATED WITH RADIOTHERAPY; TREATMENT OF PAGET'S DISEASE OF BONE; MANAGEMENT OF MODERATE TO SEVERE PAIN; TREATMENT OF PROSTATITIS; USE IN CHILDREN TO VISUALIZE LESIONS WITH ABNORMAL VASCULARITY IN THE BRAIN (INTRACRANIAL LESIONS), SPINE, AND ASSOCIATED TISSUE; USE IN MRI IN ADULTS TO VISUALIZE LESIONS IN THE HEAD AND NECK; MAINTENANCE OF HEALING OF EROSIVE ESOPHAGITIS; TO SLOW THE PROGRESSION FO CORONARY ATHEROSCLEROSIS IN PATIENTS WITH CORONARY HEART DISEASE; PREVENTION OF DEEP VEIN THROMBOSIS, WHICH MAY LEAD TO PULMONARY EMBOLISM FOLLOWING KNEE REPLACEMENT SURGERY; TREATMENT OF ANEMIA CAUSED BY UTERINE LEIOMYOMATA IN WOMEN WHO FAIL IRON THERAPY; MAINTENANCE THERAPY FOR GASTRIC ULCER PATIENTS AT REDUCED DOSAGE AFTER HEALING ACUTE ULCERS; EXPANDED PATIENT POPULATION—USE IN ICU PATIENTS; PSORIASIS OF THE SCALP; RELIEF OF MILD TO MODERATE PAIN IN PATIENTS AGED 6 MONTHS AND OLDER; LEUKOCYTE LABELED SCINTIGRAPHY AS AN ADJUNCT IN THE LOCALIZATION OF INTRA-ABDOMINAL INFECTION AND INFLAMMATORY BOWEL DISEASE; EXPANSION OF CONSCIOUS SEDATION INDICATION TO INCLUDE SHORT THERAPEUTIC PROCEDURES; ADJUNCT TO THALLIUM-201 MYOCARDIAL PERFUSION IN PATIENTS UNABLE TO EXERCISE ADEQUATELY; TREATMENT OF ACYCLOVIR-RESISTANT HERPES IN IMMUNOCOMPROMISED PATIENTS; IN PT W/CH DISEASE AND HYPERCHOLESTEROLEMIA, REDUCE RISK TOTAL MORTALITY BY REDUCING CORONARY DEATH; REDUCE RISK NON-FATAL MI; REDUCE RISK UNDERGOING MYOCARDIAL REVASCULARIZATION PROCEDURES; REDUCTION ELEVATED TOTAL AND LDL CHOL LEVELS . . . ; TREATMENT OF ALCOHOL DEPENDENCE; MAINTENANCE OF HEALING OF EROSIVE ESOPHAGITIS; PERIPHERAL ARTERIOGRAPHY; TREATMENT OF MANIC PHASE OF BIPOLAR DISORDER; MANAGEMENT OF CHRONIC STABLE ANGINA; HEART FAILURE POST MYOCARDIAL INFARCTION; BONE METASTASES ASSOCIATED WITH MULTIPLE MYELOMA; IDIOPATHIC CHRONIC URTICARIA; PREVENTION OF METAL-INDUCED HEART BURN, ACID INDIGESTION, AND SOUR STOMACH WHEN TAKEN 30 MINUTES PRIOR TO CONSUMING FOOD OR BEVERAGES; TREATMENT OF ACUTE RECURRENT GENITAL HERPES; PALLIATIVE TREATMENT OF ADVANCED BREAST CANCER IN PRE- AND PERIMENOPAUSAL WOMEN; PREVENTION OF CYTOMEGALOVIRUS (CMV) DISEASE IN INDIVIDUALS WITH HIV INFECTION AT RISK FOR DEVELOPING CMV DISEASE; TREATMENT OF HEMODYNAMICALLY STABLE PATIENTS WITHIN 24 HOURS OF ACUTE MYOCARDIAL INFARCTION TO IMPROVE SURVIVAL; LOCALIZE MYOCARDIAL ISCHEMIA(REVERSIBLE DEFECT) AND INFARCTION (NON-REVERSIBLE DEFECTS) IN EVALUATING MYOCARDIAL FUNCTION; EPISODIC TREATMENT OF RECURRENT GENITAL HERPES IN IMMUNOCOMPETENT ADULTS; ENHANCEMENT OF MRI OF THE ADULT BODY INTERNAL ORGANS; 0.1 MMOL/KG AS A SINGLE INTRAVENOUS BOLUS FOR MRI OF THE CNS IN CHILDREN; CONTRAST ENHANCEMENT AND FACILITATION OF VISUALIZATION OF EXTRACRANIAL HEAD AND NECK LESIONS; PREVENTION OF GALLSTONE FORMATION IN OBESE PATIENTS EXPERIENCING RAPID WEIGHT LOSS; TREATMENT OF ACUTE PNEUMOCYSTIS CARINI PNEUMONIA (PCP) IN HIV-INFECTED PATIENTS WHOSE ALVEOLAR-ARTERIAL OXYGEN DIFFERENCE (AaDO2) IS LESS THAN OR EQUAL TO 55 TORR; TREATMENT OF PATIENTS WITH NON-SMALL CELL LUNG CANCER; TREATMENT OF OBSESSIVE COMPULSIVE DISORDER AND PANIC DISORDER; PREVENTION OF AND PREVENTION OF FURTHER POSTOPERATIVE NAUSEA AND VOMITING IN PEDIATRIC PATIENTS RECEIVING GENERAL ANESTHESIA; SLOWING THE PROGRESSION OF CORONARY ATHEROSCLEROSIS AND REDUCING THE RISK OF ACUTE CORONARY EVENTS; MANAGEMENT OF SEVERE SPASTICITY [ENCOMPASSES SPINAL AND CEREBRAL ORIGIN; PATIENT POPULATION ALTERED TO INCLUDE PEDIATRIC USE; TREATMENT OF ONYCHOMYCOSIS DUE TO DERMATOPHYTES (TINEA UNGUIUM) OF THE TOENAIL WITH OR WITHOUT FINGERNAIL INVOLVEMENT; ADDITIONAL DATA REGARDING THE SAFE USE OF NORVASC IN PATIENTS WITH HEART FAILURE; TREATMENT OF ACUTE UNCOMPLICATED CYSTITIS IN FEMALES; TREATMENT OF OSTEOLYTIC BONE METASTASES OF BREAST CANCER; FOR HYPERCHOLESTEROLEMIC PATIENTS WITHOUT CLINICALLY EVIDENT HEART DISEASE REDUCE THE RISK OF MYOCARDIAL INFARCTION, REVASCULARIZATION, AND DEATH DUE TO CARDIOVASCULAR CAUSES WITH NO INCREASE IN DEATH FROM NON-CARDIOVASCULAR CAUSES; TREATMENT OF BACTERIAL CORNEAL ULCERS; TREATMENT OF ADULT-ONSET OR CHILDHOOD-ONSET ADULT GROWTH HORMONE DEFICIENCY; FOR USE IN PATIENTS 6-11 YEARS OF AGE; TREATMENT OF PHOTOPHOBIA; CHRONIC BACTERIAL PROSTATITIS; MANAGEMENT OF ADULTS WITH ACTIVE, CLASSIC AND DEFINITIVE RHEUMATOID ARTHRITIS WHO HAVE HAD INSUFFICIENT THERAPEUTIC RESPONSE TO OR ARE INTOLERANT OF AN ADEQUATE TRIAL OF FULL DOSES OF ONE OR MORE NON-STEROIDAL ANTI-INFLAMMATORY DRUGS; TREATMENT OF BULIMIA; COMPLICATED INTRA-ABDOMINAL INFECTIONS (USED IN COMBINATION WITH METRONIDAZOLE) CAUSED BY MIXED AEROBIC/ANAEROBIC PATHOGENS; MANAGEMENT OF LOCALLY CONFINED STAGE B2-C METASTATIC CARCINOMA OF THE PROSTATE (IN COMBINATION WITH LHRH AGONISTS); USE IN COMBINATION WITH CORTICOSTEROIDS AS INITIAL CHEMOTHERAPY FOR THE TREATMENT OF PATIENTS WITH PAIN RELATED TO ADVANCED HORMONE-REFRACTORY PROSTATE CANCER; PROPHYLACTIC USE DURING HEAD LICE EPIDEMICS; RELIEF OF SYMPTOMS OF THE COMMON COLD; TREATMENT OF INITIAL EPISODE OF GENITAL HERPES; PREOPERATIVELY FOR THE PREVENTION OF INFECTION IN TRANSRECTAL PROSTATE BIOPSY; PELVIC INFLAMMATORY DISEASE; TREATMENT OF TINEA CORPORIS AND TINEA CRURIS; TREATMENT OF POSTOPERATIVE INFLAMMATION IN PATIENTS WHO HAVE UNDERGONE CATARACT EXTRACTION; TX OF MODERATE ACNE VULGARIS IN FEMALES, GREATER OR EQUAL TO 15YRS OF AGE, WHO HAVE NO KNOWN CONTRAINDICATIONS TO ORAL CONTRACEPTIVE THERAPY, DESIRE CONTRACEPTION, HAVE ACHIEVED MENARCHE AND ARE UNRESPONSIVE TO TOPICAL ANTI-ACNE MEDICATIONS; TREATMENT OF ONYCHOMYCOSIS OF THE FINGERNAIL WITHOUT CONCOMITANT ONCHOMYCOSIS OF THE TOENAIL WITH A PULSE DOSING REGIMEN; NOSOCOMIAL PNEUMONIA-MILD TO MODERATE AND SEVERE CAUSED BY HAEMOPHILUS INFLUENZAE OR KLEBSIELLA PNEUMONIAE; TREATMENT OF PLANTAR TINEA PEDIS (MOCCASIN TYPE); TREATMENT OF PATIENTS WITH COMPLEX PARTIAL SEIZURES WITH AND WITHOUT SECONDARY GENERALIZATION; TREATMENT OF GROWTH FAILURE ASSOCIATED WITH TURNER SYNDROME; MAINTENANCE THERAPY IN THE MANAGEMENT OF MILD TO MODERATE ASTHMA IN PEDIATRIC PATIENTS AGES 6-11; TREATMENT OF PANIC DISORDER AT A RECOMMENDED DOSE RANGE OF 1 TO 2 MG/DAY (MAXIMUM OF 4 MG); PREVENTION OF OSTEOPOROSIS IN POSTMENOPAUSAL WOMEN; TREATMENT OF TINEA (PITYRIASIS) VERSICOLOR CAUSED BY OR PRESUMED TO BE CAUSED BY PITYROSPORUM ORBICULARE (ALSO KNOWN AS MALASSEZIA FURFUR OR M. ORBICULARE); PREVENTION OF FRACTURES IN THE TREATMENT OF POSTMENOPAUSAL OSTEOPOROSIS; TREATMENT OF ACUTE SINUSITIS AND ACUTE EXACERBATION OF CHRONIC SINUSITIS; TREATMENT OF ACUTE OTITIS MEDIA IN PEDIATRIC PATIENTS; PLANAR IMAGING AS A SECOND LINE DIAGNOSTIC DRUG AFTER MAMMOGRAPHY TO ASSIST IN THE EVALUATION OF BREAST LESIONS IN PATIENTS WITH AN ABNORMAL MAMMOGRAM OR A PALPABLE BREAST MASS; ENDOMETRIAL THINNING AGENT PRIOR TO ENDOMETRIAL ABLATION FOR DYSFUNCTIONAL UTERINE BLEEDING; THE PREVENTION OF DEEP VEIN THROMBOSIS, WHICH MAY LEAD TO PULMONARY EMBOLISM, IN PATIENTS UNDERGOING ABDOMINAL SURGERY WHO ARE AT RISK FOR THROMBOEMBOLIC COMPLICATIONS AND A NEW DOSAGE REGIMEN, 40 MG ONCE DAILY, FOR THIS INDICATION; TREATMENT OF PANIC DISORDER IN A RECOMMENDED DOSE RANGE OF 50 TO 200 MG/DAY; CONGESTIVE HEART FAILURE; FOR USE OF LANSOPRAZOLE IN COMBINATION WITH CLARITHROMYCIN AND AMOXICILLIN FOR THE ERADICATION OF HELICOBACTER PYLORI IN PATIENTS WITH ACTIVE DUODENAL ULCER DISEASE OR A ONE-YEAR HISTORY OF DUODENAL ULCER; ACUTE TREATMENT OF ACTIVE BENIGN GASTRIC ULCER; MAINTENANCE OF HEALING OF DUODENAL ULCER; FOR THE USE OF LANSOPRAZOLE IN COMBINATION WITH AMOXICILLIN FOR THE ERADICATION OF HELICOBACTER PYLORI IN PATIENTS WITH ACTIVE DUODENAL ULCER DISEASE OR A ONE-YEAR HISTORY OF A DUODENAL ULCER; MONOTHERAPY AND COMBINATION THERAPY WITH SULFONYLUREA IN THE TREATMENT OF TYPE II DIABETES; TREATMENT OF TINEA (PITYRIASIS) VERSICOLOR; EMPIRICAL THERAPY FOR FEBRILE NEUTROPENIC PATIENTS; SECOND-LINE TREATMENT OF AIDS-RELATED KAPOSI'S SARCOMA; MAINTENANCE OF REMISSION OF ULCERATIVE COLITIS; USE IN PEDIATRIC PATIENTS BETWEEN THE AGES OF 6 AND 11 FOR THE TREATMENT OF THE NASAL SYMPTOMS OF SEASONAL AND PERENNIAL ALLERGIC RHINITIS; INITIAL ANTICONVULSANT TREATMENT OF STATUS EPILEPTICUS; TREATMENT OF EDEMA ASSOCIATED WITH CHRONIC RENAL FAILURE; FOR THE SUPPRESSION OF RECURRENT EPISODES OF GENITAL HERPES IN IMMUNOCOMPETENT ADULTS; TREATMENT OF OBSESSIVE COMPULSIVE DISORDER IN THE PEDIATRIC POPULATION; PAROXYSMAL SUPRAVENTRICULAR TACHYCARDIA (PSVT); TO SLOW THE PROGRESSION OF CORONARY ATHEROSCLEROSIS IN PATIENTS WITH CORONARY HEART DISEASE AS PART OF A TREATMENT STRATEGY TO LOWER TOTAL AND LDL CHOLESTEROL TO TARGET LEVELS; FOR USE IN PEDIATRIC POPULATION; TREATMENT OF SYMPTOMS OF DRY MOUTH IN PATIENTS WITH SJOGREN'S SYNDROME; TEMPORARY RELIEF OF PAIN AND PHOTOPHOBIA IN PATIENTS UNDERGOING CORNEAL REFRACTIVE SURGERY; TREATMENT OF OSTEOPOROSIS; PRE-PROCEDURAL APPLICATION TO ADULT MALE GENITAL SKIN PRIOR TO SITE-SPECIFIC SUBCUTANEOUS INFILTRATION WITH LIDOCAINE FOR THE REMOVAL OF GENITAL WARTS; FOR THE LONG-TERM TWICE-DAILY (MORNING AND EVENING) ADMINISTRATION IN THE MAINTENANCE TREATMENT OF BRONCHOSPASM ASSOCIATED WITH COPD, INCLUDING CHRONIC BRONCHITIS AND EMPHYSEMA; PREVENTION (DURING AND FOLLOWING HOSPITALIZATION) OF DEEP VEIN THROMBOSIS, WHICH MAY LEAD TO PULMONARY EMBOLISM, IN PATIENTS UNDERGOING HIP REPLACEMENT SURGERY; USE OF LIPITOR AS AN ADJUNCTIVE THERAPY TO DIET FOR THE TREATMENT OF PATIENTS WITH ELEVATED SERUM TRIGLYCERIDE LEVELS (FREDERICKSON TYPE IV); USE OF LIPITOR BY PATIENTS WITH PRIMARY DYSBETALIPOPROTEINEMIA (FREDERICKSON TYPE III) WHO DO NOT RESPOND ADEQUATELY TO DIET; TREATMENT OF EPISODIC-HEARTBURN, ACID INDIGESTION AND SOUR STOMACH; TREATMENT OF BENIGN PROSTATIC HYPERPLASIA (BPH) IN MEN WITH AN ENLARGED PROSTATE TO IMPROVE SYMPTOMS, REDUCE THE RISK OF ACUTE URINARY RETENTION AND REDUCE THE RISK OF THE NEED OF SURGERY; PREVENTION OF ISCHEMIC COMPLICATIONS OF UNSTABLE ANGINA AND NON-Q-WAVE MYOCARDIAL INFARCTION, WHEN CONCURRENTLY ADMINISTERED WITH ASPIRIN; USE IN THE SYMPTOMATIC RELIEF OF RHINORRHEA ASSOCIATED WITH ALLERGIC AND NONALLERGIC-PERENNIAL RHINITIS IN CHILDREN AGE 6-11 YEARS; FOR THE USE IN PEDIATRIC PATIENTS 4 TO 11 YEARS OF AGE FOR THE MANAGEMENT OF THE NASAL SYMPTOMS OF SEASONAL AND PERENNIAL ALLERGIC RHINITIS; USE IN PATIENTS WITH PREVIOUS MI AND NORMAL CHOLESTEROL LEVELS, TO REDUCE RISK OF RECURRENT MI, MYOCARDIAL REVASCULARIZATION, AND CEREBROVASCULAR DISEASE EVENTS; FIRST-LINE THERAPY FOR THE TREATMENT OF ADVANCED CARCINOMA OF THE OVARY IN COMBINATION WITH CISPLATIN; SHORT-TERM TREATMENT OF SYMPTOMATIC GASTROESPHAGEAL REFLUX DISEASE (GERD); PREVENTION OF MEAL INDUCED HEARTBURN AT A DOSE OF 75 MG TAKEN 30-60 MIN PRIOR TO A MEAL; PRILOSEC (OMEPRAZOLE), AMOXICILLIN, AND CLARITHROMYCIN FOR THE ERADICATION OF H. PYLORI IN PATIENTS WITH DUODENAL ULCER DISEASE; IN COMBINATION WITH CIS-PLATIN, FOR THE FIRST LINE TREATMENT OF NON-SMALL CELL LUNG CANCER IN PATIENTS WHO ARE NOT CANDIDATES FOR POTENTIALLY CURATIVE SURGERY AND/OR RADIATION; TREATMENT OF PATIENTS WITH LOCALLY ADVANCED OR METASTATIC BREAST CANCER AFTER FAILURE OF PRIOR CHEMOTHERAPY; TREATMENT OF RECURRENT MUCOCUTANEOUS HERPES SIMPLEX INFECTIONS IN HIV-AFFECTED PATIENTS AT A DOSE OF 500 MG TWICE DAILY; PROPHYLACTIC USE TO REDUCE PERIOPERATIVE BLOOD LOSS AND THE NEED FOR BLOOD TRANSFUSION IN PATIENTS UNDERGOING CARDIOPULMONARY BYPASS IN THE COURSE OF CORONARY ARTERY BYPASS GRAFT SURGERY; FOR USE IN COMBINATION WITH CISPLATIN FOR THE FIRST-LINE TREATMENT OF PATIENTS WITH INOPERABLE LOCALLY ADVANCED (STAGE IIIA OR IIIB) OR METASTATIC (STAGE IV) NON-SMALL CELL LUNG CANCER; PREVENTION OF EXERCISE-INDUCED BRONCHOSPASM IN PATIENTS 12 YEARS OF AGE AND OLDER; PREVENTION OF EXERCISE-INDUCED BRONCHOSPASM IN PATIENTS 4 YEARS OF AGE AND OLDER; MAINTENANCE TREATMENT OF ASTHMA AND PREVENTION OF BRONCHOSPASM IN PATIENTS 4 YEARS OF AGE AND OLDER; ADJUNCTIVE TREATMENT OF LENNOX-GASTAUT SYNDROME IN PEDIATRIC AND ADULT PATIENTS; TREATMENT OF PATIENTS WITH HOMOZYGOUS FAMILIAL HYPERCHOLESTEROLEMIA; MANAGEMENT OF SECONDARY HYPERPARATHYROIDISM AND RESULTANT METABOLIC BONE DISEASE IN PATIENTS WITH MODERATE TO SEVERE CHRONIC RENAL FAILURE (CCR 15 TO 55ML/MIN) NOT YET ON DIALYSIS; USE IN PHOTODYNAMIC THERAPY (PDT) FOR REDUCTION OF OBSTRUCTION AND PALLIATION OF SYMPTOMS IN PATIENTS WITH COMPLETELY OR PARTIALLY OBSTRUCTING ENDOBRONCHIAL NONSMALL CELL LUNG CANCER; TREATMENT OF MODERATE TO SEVERE VASOMOTOR SYMPTOMS ASSOCIATED WITH THE MENOPAUSE AND IN THE TREATMENT OF VULVAR AND VAGINAL ATROPHY IN WOMEN WITH AN INTACT UTERUS; USE IN THE SYMPTOMATIC RELIEF OF RHINORRHEA ASSOCIATED WITH THE COMMON COLD IN CHILDREN AGE 5 TO 11 YEARS; REDUCE THE INCIDENCE OF BREAST CANCER IN WOMEN AT HIGH RISK FOR BREAST CANCER; TREATMENT OF ACUTE SINUSITIS; TREATMENT OF UNCOMPLICATED URINARY TRACT INFECTIONS; USE IN CONVERSION TO MONOTHERAPY IN ADULTS WITH PARTIAL SEIZURES WHO ARE RECEIVING TREATMENT WITH A SINGLE ENZYME-INDUCING ANTIEPILEPTIC DRUG; INPATIENT TREATMENT OF ACUTE DEEP VEIN THROMBOSIS WITH/WITHOUT PULMONARY EMBOLISM WHEN ADMIN WITH WARFARIN SODIUM AND OUTPATIENT TREATMENT OF ACUTE DEEP VEIN THROMBOSIS WITHOUT PULMONARY EMBOLISM WHEN ADMIN WITH WARFARIN SODIUM; TREATMENT OF CHRONIC HEPATITIS C IN PATIENTS WITH COMPENSATED LIVER DISEASE PREVIOUSLY UNTREATED WITH ALPHA INTERFERON THERAPY; PRIMARY PREVENTION OF CORONARY HEART DISEASE IN PATIENTS WITHOUT SYMPATOMATIC CARDIOVASCULAR DISEASE WHO HAVE AVERAGE TO MODERATELY ELEVATED TOTAL-C AND LDL-C AND BELOW AVERAGE HDL-C; TREATMENT OF GENERALIZED ANXIETY DISORDER; NEW COMBINATION USE OF PRECOSE FOR PATIENTS WITH TYPE 2 DIABETES TREATED WITH DIET PLUS METFORMIN; COMBINATION USE OF PRECOSE FOR PATIENTS WITH TYPE 2 DIABETES TREATED WITH DIET PLUS INSULIN; PREVENTION OF POSTMENOPAUSAL OSTEOPOROSIS (LOSS OF BONE MASS); PREVENTION OF PNEUMOCYSTIS CARINII PNEUMONIA (PCP); USE IN TREATMENT OF SMALL CELL LUNG CANCER SENSITIVE DISEASE AFTER FAILURE OF FIRST-LINE CHEMOTHERAPY; TREATMENT OF CHRONIC HEPATITIS B ASSOCIATED WITH EVIDENCE OF HEPATITIS B VIRAL REPLICATION AND ACTIVE LIVER INFLAMMATION; FOR PERENNIAL NONALLERGIC RHINITIS FOR AGES 4 AND ABOVE; PROPHYLAXIS OF DEEP VEIN THROMBOSIS (DVT), WHICH MAY LEAD TO PULMONARY EMBOLISM, IN PATIENTS UNDERGOING HIP REPLACEMENT SURGERY; EXPANDED PEDIATRIC USE IN CHILDREN YOUNGER THAN ONE MONTH OF AGE TO BIRTH (WITH A GESTATIONAL AGE OF 37 WEEKS OR GREATER); TREATMENT OF SOCIAL ANXIETY DISORDER; TREATMENT OR PREVENTION OF BRONCHOSPASM WITH REVERSIBLE OBSTRUCTIVE AIRWAY DISEASE AND FOR THE PREVENTION OF EXERCISE INDUCED BRONCHOSPASM IN CHILDREN AGES 4-12; TREATMENT OF UNSTABLE ANGINA AND NON-Q-WAVE MYOCARDIAL INFARCTION FOR THE PREVENTION OF ISCHEMIC COMPLICATIONS IN PATIENTS ON CONCURRENT ASPIRIN THERAPY; PREVENTION OF NAUSEA AND VOMITING ASSOCIATED WITH RADIATION, INCLUDING TOTAL BODY IRRADIATION (TBI) AND FRACTIONATED ABDOMINAL RADIATION; TREATMENT OF ATOPIC DERMATITIS IN PEDIATRIC PATIENTS 6 YEARS AND OLDER; USE OF TOPAMAX AS ADJUNCTIVE THERAPY IN PEDIATRIC PATIENTS AGES 2-16 YEARS WITH PARTIAL ONSET SEIZURES; USE IN PEDIATRIC PATIENTS 3 MONTHS OLD AND OLDER—FOR CORTICOSTEROID-RESPONSIVE DERMATOSES; PROPHYLAXIS AND CHRONIC TREATMENT OF ASTHMA IN PATIENTS 7-11 YEARS OF AGE; PREVENTION OF NAUSEA AND VOMITING ASSOCIATED WITH HIGHLY EMETOGENIC CANCER CHEMOTHERAPY, INCLUDING CISPLATIN; ADJUVANT TREATMENT OF NODE-POSITIVE BREAST CANCER ADMINISTERED SEQUENTIALLY TO STANDARD DOXORUBICIN-CONTAINING COMBINATION CHEMOTHERAPY; TREATMENT OF OSTEOPOROSIS IN POSTMENOPAUSAL WOMEN; TREATMENT OF GLUCOCORTICOID-INDUCED OSTEOPOROSIS IN MEN AND WOMEN RECEIVING GLUCOCORTICOIDS IN A DAILY DOSE EQUIVALENT TO 7.5 MG OR GREATER OF PREDNISONE AND WHO HAVE LOW BONE MINERAL DENSITY; ADJUNCT TO DIET TO INCREASE HDL-C IN PATIENTS WITH PRIMARY HYPERCHOLESTEROLEMIA (HETEROZYGOUS FAMILIAL AND NON FAMILIAL) AND MIXED DYSLIPIDEMIA (FREDERICKSON TYPES IIA AND IIB); USE OF TOPAMAX AS ADJUNCTIVE THERAPY IN THE TREATMENT OF PRIMARY GENERALIZED TONIC-CLONIC SEIZURES; USE IN COMBINATION WITH METFORMIN AND SULFONYLUREA IN PATIENTS WITH TYPE 2 DIABETES; USE OF REZULIN IN COMBINATION WITH METFORMIN AND SULFONYLUREAS IN PATIENTS WITH TYPE 2 DIABETES; TREATMENT OF TYPE III HYPERLIPOPROTEINEMIA; TREATMENT OF PATIENTS WITH ISOLATED HYPERTRIGLYCERIDEMIA (FREDERICKSON TYPE IV); TREATMENT OF POST-TRAUMATIC STRESS DISORDER; USE OF CARNITOR INJECTION FOR THE PREVENTION AND TREATMENT OF CARNITINE DEFICIENCY IN PATIENTS WITH END STAGE RENAL DISEASE WHO ARE UNDERGOING DIALYSIS; INCREASING HDL-C IN PATIENTS WITH PRIMARY HYPERCHOLESTEROLEMIA (HETEROZYGOUS FAMILIAL AND NONFAMILIAL) AND MIXED DYSLIPIDEMIA (FREDERICKSON TYPES IIA AND IIB); TREATMENT OF PATIENTS WITH LOCALLY ADVANCED OR METASTATIC NON-SMALL CELL LUNG CANCER AFTER FAILURE OF PRIOR PLATINUM-BASED CHEMOTHERAPY; TO REDUCE THE INCIDENCE OF MODERATE TO SEVERE XEROSTOMIA IN PATIENTS UNDERGOING POST-OPERATIVE RADIATION TREATMENT FOR HEAD AND NECK CANCER, WHERE THE RADIATION PORT INCLUDES A SUBSTANTIAL PORTION OF THE PAROTID GLANDS; TO REDUCE THE NUMBER OF ADENOMATOUS COLORECTAL POLYPS IN FAMILIAL ADENOMATOUS POLYPOSIS PATIENTS AS AN ADJUNCT TO USUAL CARE; TREATMENT OF NASAL SYMPTOMS OF SEASONAL AND PERENNIAL RHINITIS IN ADULTS AND CHILDREN 3 YEARS OF AGE AND OLDER; TREATMENT OF PATIENTS WITH FREDERICKSON TYPE III; USE OF PRAVASTATIN IN PATIENTS WITH EVIDENT CORONARY HEART DISEASE TO REDUCE THE RISK OF TOTAL MORTALITY BY REDUCING CORONARY DEATH; CHANGES IN SEVERAL SECTIONS OF THE INSERT TO INCORPORATE STATEMENTS CONCERNING THE USE OF HIGH DOSES OF LISINOPRIL TO REDUCE THE RISK OF THE COMBINED OUTCOMES OF MORTALITY AND HOSPITALIZATION IN PATIENTS WITH CONGESTIVE HEART FAILURE; USE OF AVANDIA IN COMBINATION WITH A SULFONYLUREA IN PATIENTS WITH TYPE 2 DIABETES MELLITUS WHEN DIET AND EXERCISE WITH EITHER SINGLE AGENT DOES NOT ACHIEVE ADEQUATE GLYCEMIC CONTROL; PREVENTION OF CORTICOSTEROID-INDUCED OSTEOPOROSIS; PREVENTION OF POSTMENOPAUSAL OSTEOPOROSIS; TREATMENT OF POSTMENOPAUSAL OSTEOPOROSIS; TREATMENT OF CORTICOSTEROID-INDUCED OSTEOPOROSIS; TREATMENT OF UNCOMPLICATED ACUTE ILLNESS DUE TO INFLUENZA A AND B IN PEDIATRIC PATIENTS 7 YEARS AND OLDER WHO HAVE BEEN SYMPTOMATIC FOR NO MORE THAN 2 DAYS; PREVENTION OF POSTMENOPAUSAL OSTEOPOROSIS FOR WOMEN WITH AN INTACT UTERUS; LONG-TERM INTRAVENOUS TREATMENT OF PULMONARY HYPERTENSION ASSOCIATED WITH THE SCLERODERMA SPECTRUM OF DISEASE IN NYHA CLASS III AND CLASS IV PATIENTS WHO DO NOT RESPOND TO CONVENTIONAL THERAPY; SHORT-TERM TREATMENT OF ACUTE MANIC EPISODES ASSOCIATED WITH BIPOLAR I DISORDER; TREATMENT OF PATIENTS WITH FREDERICKSON TYPE IIA AND IIB HYPERLIPOPROTEINEMIA; USE OF CAMPTOSAR AS A COMPONENT OF FIRST-LINE THERAPY IN COMBINATION WITH 5-FLUOROURACIL AND LEUCOVORIN FOR PATIENTS WITH METASTATIC CARCINOMA OF THE COLON OR RECTUM; PROPHYLAXIS FOR ASTHMA IN CHILDREN 2-5 YEARS OF AGE; TREATMENT OF SIGNS AND SYMPTOMS OF ALLERGIC CONJUNCTIVITIS; TREATMENT OF PEDIATRIC PATIENTS WITH PRADER-WILLI SYNDROME; INCREASING HDL-CHOLESTEROL IN PATIENTS WITH PRIMARY HYPERCHOLESTEROLEMIA AND MIXED DYSLIPIDEMIAS; TREATMENT OF PATIENTS WITH FREDERICKSON TYPE IV; TREATMENT OF LEVOFLOXACIN SUSCEPTIBLE STRAINS OF PENICILLIN-RESISTANT STREPTOCOCCUS PNEUMONIAE IN PATIENTS WITH COMMUNITY ACQUIRED PNEUMONIA; INDUCTION OF SPERMATOGENESIS IN MEN WITH PRIMARY AND SECONDARY HYPOGONADOTROPIC HYPOGONADISM IN WHOM THE CAUSE OF INFERTILITY IS NOT DUE TO PRIMARY TESTICULAR FAILURE; NEW COMBINATION USE OF METFORMIN AND INSULIN IN TYPE 2 DIABETES; TREATMENT OF PEDIATRIC PATIENTS WITH POLYARTICULAR COURSE JUVENILE RHEUMATOID ARTHRITIS WHO RESPONDED INADEQUATELY TO SALICYLATES OR OTHER NSAIDS; USE OF ACTONEL 35 MG ONCE A WEEK TO INCREASE BONE MASS IN MEN WITH OSTEOPOROSIS; REDUCTION IN RISK OF MYOCARDIAL INFARCTION, STROKE, AND DEATH FROM CARDIOVASCULAR CAUSES; ADJUNCTIVE THERAPY IN THE TREATMENT OF PARTIAL SEIZURES IN PEDIATRIC PATIENTS AGE 3 TO 12 YEARS; FIRST LINE TREATMENT OF POSTMENOPAUSAL WOMEN WITH HORMONE RECEPTOR POSITIVE OR HORMONE RECEPTOR UNKNOWN LOCALLY ADVANCED OR METASTATIC BREAST CANCER; EXTENSION OF INDICATION TO PROVIDE FOR MAINTENANCE OF RESPONSE; TOPICAL ANESTHETIC FOR SUPERFICIAL MINOR SURGERY OF GENITAL MUCOUS MEMBRANES AND AS AN ADJUNCT FOR LOCAL INFILTRATION ANESTHESIA IN GENITAL MUCOUS MEMBRANES; THROMBOPROPHYLAXIS OF DEEP VEIN THROMBOSIS, WHICH MAY LEAD TO PULMONARY EMBOLISM, IN MEDICAL PATIENTS WHO ARE AT RISK FOR THROMBOEMBOLIC COMPLICATIONS DUE TO SEVERELY RESTRICTED MOBILITY DURING ACUTE ILLNESS; TREATMENT OF NSAID-ASSOCIATED GASTRIC ULCER PATIENTS WHO CONTINUE NSAID USE AND REDUCING RISK OF NSAID-ASSOCIATED GASTRIC ULCERS IN PATIENTS WITH HISTORY OF DOCUMENTED GASTRIC ULCER WHO REQUIRE USE OF AN NSAID; PROPHYLAXIS OF INFLUENZA IN ADULTS AND ADOLESCENTS 13 YEARS AND OLDER; FIRSTLINE TREATMENT OF POSTMENOPAUSAL WOMEN WITH HORMONE RECEPTOR POSITIVE OR HORMONE RECEPTOR UNKNOWN LOCALLY ADVANCED OR METASTATIC BREAST CANCER; USE FOR SUSPECTED OR CONFIRMED METHANOL POISONING, EITHER ALONE OR IN COMBINATION WITH HEMODIALYSIS; TREATMENT OF TYPE 2 DIABETES IN PEDIATRIC PATIENTS (AGES 10-16 YEARS); JUVENILE RHEUMATOID ARTHRITIS; USE OF DIPRIVAN IN PATIENTS 3 MONTHS TO 16 YEARS; COLORECTAL CANCER; REDUCING NEUROLOGIC DISABILITY AND/OR FREQUENCY OF CLINICAL RELAPSES IN PATIENTS WITH SECONDARY (CHRONIC) PROGRESSIVE, PROGRESSIVE RELAPSING, OR WORSENING RELAPSING-REMITTING MULTIPLE SCLEROSIS; PREVENTION OF RELAPSE AND RECURRENCE OF DEPRESSION; GENERALIZED ANXIETY DISORDER; SYMPTOMATIC RELIEF OF RHINORRHEA ASSOCIATED WITH SEASONAL ALLERGIC RHINITIS IN PATIENTS 5 YEARS AND OLDER; PROPHYLAXIS AND CHRONIC TREATMENT OF ASTHMA IN PATIENTS 5-6 YEARS OF AGE; UNCOMPLICATED SKIN AND SKIN STRUCTURE INFECTIONS; MAINTENANCE OF HEALING OF EROSIVE ESOPHAGITIS AND CONTROL OF DAYTIME AND NIGHTTIME HEARTBURN SYMPTOMS IN PATIENTS WITH GERD; TREATMENT OF MODERATE ACNE VULGARIS; EMPIRIC THERAPY IN FEBRILE NEUTROPENIC PATIENTS WITH SUSPECTED FUNGAL INFECTIONS (EFTN); TOPICAL TREATMENT OF TINEA (PITYRIASIS) VERSICOLOR DUE TO MALASSEZIA FURFUR (FORMERLY PITYROSPORUM ORBICULARE); LONG-TERM TREATMENT OF GROWTH FAILURE IN CHILDREN BORN SMALL FOR GESTATIONAL AGE WHO FAIL TO MANIFEST CATCH-UP GROWTH BY TWO YEARS OF AGE; ADJUNCTIVE THERAPY IN PATIENTS TWO YEARS AND OLDER WITH SEIZURES ASSOCIATED WITH LENNOX-GASTAUT SYNDROME; EXPANSION OF INDICATION TO INCLUDE THE TREATMENT OF PATIENTS WITH PREDOMINATELY CLASSIC SUBFOVEAL CHOROIDAL NEOVASCULARIZATION DUE TO PATHOLOGIC MYOPIA OR PRESUMED OCULAR HISTOPLASMOSIS; PATHOLOGICAL HYPERSECRETION ASSOCIATED WITH ZOLLINGER-ELLISON SNYDROME; MANAGEMENT OF ACUTE PAIN IN ADULTS AND TREATMENT OF PRIMARY DYSMENORRHEA; TREATMENT OF HEPATITIS B IN PEDIATRIC PATIENTS AGES 2-17 YEARS; ATOPIC DERMATITIS IN PEDIATRIC PATIENTS AGES 2-5; BREAST CANCER COMBINATION THERAPY; USE OF FORADIL FOR LONG-TERM, TWICE DAILY (MORNING AND EVENING) ADMINISTRATION IN THE MAINTENANCE TREATMENT OF BRONCHO-CONSTRICTION IN PATIENTS WITH COPD INCLUDING CHRONIC BRONCHITIS AND EMPHYSEMA; USE OF COREG FOR SEVERE HEART FAILURE; ACNE VULGARIS; TREATMENT OF POSTTRAUMATIC STRESS DISORDER; TREATMENT OF SYMPTOMATIC GASTRO ESOPHAGEAL REFLUX DISEASE (GERD); TREATMENT OR PREVENTION OF BRONCHOSPASM IN CHILDREN 6 YEARS OF AGE AND OLDER WITH OBSTRUCTIVE AIRWAY DISEASE; LONG-TERM, TWICE-DAILY (MORNING AND EVENING) ADMINISTRATION IN THE MAINTENANCE TREATMENT OF BRONCHOSPASM ASSOCIATED WITH COPD (INCLUDING EMPHYSEMA AND CHRONIC BRONCHITIS); ACUTE CORONARY SYNDROME; TREATMENT OF HETEROZYGOUS FAMILIAL HYPERCHOLESTEROLEMIA IN ADOLESCENT BOYS AND GIRLS AT LEAST ONE YEAR POSTMENARCHAL, AGES 10 TO 17 YEARS, WITH A RECOMMENDED DOSING RANGE OF 10 TO 40 MG ONCE DAILY; PREVENTION OF POSTMENOPAUSAL OSTEOPOROSIS FOR ALL STRENGTHS; ANTICOAGULANT IN PATIENTS WITH OR AT RISK FOR HEPARIN-INDUCED THROMBOCYTOPENIA UNDERGOING PERCUTANEOUS CORONARY INTERVENTIONS (PCI); TREATMENT OF SIGNS AND SYMPTOMS OF RHEUMATOID ARTHRITIS; MANAGEMENT OF POST HERPETIC NEURALGIA; PREMENSTRUAL DYSPHORIC DISORDER; TREATMENT OF PATHOLOGICAL HYPERSECRETORY CONDITIONS, INCLUDING ZOLLINGER-ELLISON SYNDROME; TREATMENT OF COMPLICATED SKIN AND SKIN STRUCTURE INFECTIONS; TREATMENT OF PANIC DISORDER; TREATMENT OF VULVAR AND VAGINAL ATROPHY ASSOCIATED WITH MENOPAUSE; TREATMENT OF NASAL SYMPTOMS OF SEASONAL AND PERENNIAL RHINITIS IN CHILDREN AGES TWO UP TO AGE THREE; TREATMENT OF MULTIPLE MYELOMA AND DOCUMENTED BONE METASTASES FROM SOLID TUMORS, IN CONJUNCTION WITH STANDARD ANTINEOPLASTIC THERAPY, PROSTATE CANCER SHOULD HAVE PROGRESSED AFTER TREATMENT WITH AT LEAST ONE HORMONAL THERAPY; TREATMENT OF PANIC DISORDER, WITH OR WITHOUT AGORAPHOBIA; ADJUVANT TREATMENT OF POST MENOPAUSAL WOMEN WITH HORMONE RECEPTOR POSITIVE EARLY BREAST CANCER; TREATMENT OF COMMUNITY-ACQUIRED PNEUMONIA IN ADULTS; TREATMENT OF HEART FAILURE (NYHA CLASS II-IV) IN PATIENTS WHO ARE INTOLERANT TO AN ACE INHIBITOR; PREVENTION OF RELAPSE FOLLOWING LONG-TERM TREATMENT OF MAJOR DEPRESSIVE DISORDER; COMBINATION THERAPY WITH THIAZOLIDINEDIONE TO LOWER BLOOD GLUCOSE IN PTS WHOSE HYPERGLYCEMIA CANNOT BE CONTROLLED BY DIET/EXERCISE PLUS MONOTHERAPY WITH ANY OF THE FOLLOWING AGENTS, METFORMIN, SULFONYLUREAS, REPAGLINIDE, OR THIAZOLIDINEDIONES; USE OF GLUCOVANCE WITH A THIAZOLIDINEDIONE WHEN GLYCEMIC CONTROL IS NOT OBTAINED WITH GLUCOVANCE ALONE; PREVENTION AND TREATMENT OF POSTOPERATIVE NAUSEA AND VOMITING; TREATMENT OF HETEROZYGOUS FAMILIAL HYPERCHOLESTEROLEMIA IN CHILDREN, AGES 8-13 YEARS, WITH RECOMMENDED DOSE OF 20 MG ONCE DAILY AND IN ADOLESCENTS, AGES 14-18 WITH A RECOMMENDED DOSE OF 40 MG ONCE DAILY; HELICOBACTER PYLORI ERADICATION TO REDUCE THE RISK OF DUODENAL ULCER RECURRENCE; NOSOCOMIAL PNEUMONIA; TREATMENT OF TYPE 2 DIABETIC NEPHROPATHY; SHORT TERM TOPICAL TREATMENT OF MILD TO MODERATE PLAQUE-TYPE PSORIASIS OF NON SCALP REGIONS; FIRST LINE THERAPY FOR THE REDUCTION OF INTRAOCULAR PRESSURE IN PATIENTS WITH OPEN-ANGLE GLAUCOMA OR OCULAR HYPERTENSION; TREATMENT OF NEWLY DIAGNOSED ADULT PATIENTS WITH PHILADELPHIA CHROMOSOME POSITIVE CHRONIC MYELOID LEUKEMIA (CML); USE OF BRAVELLE FOR MULTIPLE FOLLICULAR DEVELOPMENT (CONTROLLED OVARIAN STIMULATION) DURING ASSISTED REPRODUCTIVE TECHNOLOGY CYCLES IN PATIENTS WHO HAVE PREVIOUSLY RECEIVED PITUITARY SUPPRESSION; RELIEF OF SYMPTOMS OF SEASONAL ALLERGIC RHINITIS IN ADULTS AND PEDIATRIC PATIENTS 2 YEARS OF AGE AND OLDER; USE TAXOTERE IN COMBINATION WITH CISPLATIN FOR THE TREATMENT OF PATIENTS WITH UNRESECTABLE, LOCALLY ADVANCED OR METASTATIC NON-SMALL CELL LUNG CANCER WHO HAVE NOT PREVIOUSLY RECEIVED CHEMOTHERAPY FOR THIS CONDITION; TO TREAT PATIENTS WITH SCHIZOPHRENIA OR SCHIZOAFFECTIVE DISORDER AT RISK FOR EMERGENT SUICIDAL BEHAVIOR; TREATMENT OF COLD SORES (HERPES LABIALIS) IN ADULT AND ADOLESCENT PATIENTS 12 YEARS OF AGE AND OLDER; FOR NEWLY-DIAGNOSED HIGH GRADE MALIGNANT GLIOMA PATIENTS AS AN ADJUNCT TO SURGERY AND RADIATION; TREATMENT OF TYPE 2 DIABETIC NEPHROPATHY; USE IN COMBINATION WITH INSULIN FOR THE TREATMENT OF PATIENTS WITH TYPE 2 DIABETES MELLITUS; MODIFICATION OF THE INDICATION FOR COMMUNITY ACQUIRED PNEUMONIA TO ADD ‘INCLUDING PENICILLIN-RESISTANT STRAINS, MIC PENICILLIN>=2 MCG/ML’ TO STREPTOCOCCUS PNEUMONIAE; RAPAMUNE (SIROLIMUS) WITHIN AN IMMUNOSUPPRESSIVE REGIMEN THAT WOULD ALLOW FOR THE WITHDRAWAL OF CYCLOSPORINE 2 TO 4 MONTHS AFTER RENAL TRANSPLANTATION IN PATIENTS CONSIDERED AT LOW TO MODERATE IMMUNOLOGIC RISK FOR RENAL TRANSPLANT REJECTION; ADJUNCTIVE THERAPY OF PARTIAL SEIZURES IN PEDIATRIC PATIENTS GREATER THAN OR EQUAL TO 2 YEARS OF AGE; TREATMENT OF PATIENTS WITH LEFT VENTRICULAR DYSFUNCTION FOLLOWING MYOCARDIAL INFARCTION; SUPPRESSION OF RECURRENT GENITAL HERPES IN HIV-INFECTED INDIVIDUALS; USE IN PTS AT HIGH RISK CORONARY EVENTS DUE TO EXISTING CORONARY HEART DISEASE, DIABETES, PERIPHERAL VESSEL DISEASE, STROKE HISTORY, OTHER CV DISEASE TO REDUCE RISK TOTAL MORTALITY BY REDUCING CORONARY DEATH, REDUCE NONFATAL MI & STROKE . . . ; ABLATION OF HIGH-GRADE DYSPLASIA IN BARRETT'S ESOPHAGUS PATIENTS WHO DO NOT UNDERGO ESOPHAGECTOMY; TX OF PED PATIENTS W/PH+ CHRONIC PHASE CML DISEASE RECURRENCE AFTER STEM CELL TRANSPLANT OR RESISTANCE TO INTERFERON ALPHA THERAPY, NO CONTROLLED TRIALS DEMONSTRATING A CLINICAL BENEFIT SUCH AS IMPROVEMENT IN DISEASE RELATED SX OR INCREASED SURVIVAL; CHRONIC BACTERIAL PROSTATITIS; USE IN PATIENTS WITH CORONARY HEART DISEASE TO REDUCE THE RISK OF UNDERGOING CORONARY REVASCULARIZATION PROCEDURES; TO IMPROVE PHYSICAL FUNCTION; EXPANDED INDICATION TO INCLUDE THE ASSESSMENT OF VENTRICULAR FUNCTION IN SUBJECTS BEING EVALUATED FOR HEART DISEASE AND/OR VENTRICULAR FUNCTION; EXTENDED PROPHYLAXIS IN PATIENTS UNDERGOING HIP FRACTURE SURGERY; IDIOPATHIC SHORT STATURE; TREATMENT OF CANDIDEMIA AND THE FOLLOWING CANDIDA INFECTIONS, INTRA-ABDOMINAL ABSCESSES, PERITONITIS AND PLEURAL SPACE INFECTIONS; USE OF OLANZAPINE IN COMBINATION WITH LITHIUM OR VALPROATE FOR THE TREATMENT OF ACUTE MANIC EPISODES ASSOCIATED WITH BIPOLAR DISORDER; LONGER-TERM EFFICACY OF ARIPIPRAZOLE IN THE TREATMENT OF SCHIZOPHRENIA; DIABETIC FOOT INFECTIONS WITHOUT CONCOMITANT OSTEOMYELITIS; USE OF VALTREX IN COMBINATION WITH SAFER SEX PRACTICES FOR THE REDUCTION OF THE RISK OF TRANSMISSION OF GENITAL HERPES DURING SUPPRESSIVE THERAPY OF THE SOURCE PARTNER IN A HETEROSEXUAL COUPLE; MAINTENANCE TREATMENT OF BIPOLAR I DISORDER TO DELAY THE TIME TO OCCURRENCE OF MOOD EPISODES (DEPRESSION, MANIA, HYPOMANIA, MIXED EPISODES) IN PATIENTS TREATED FOR ACUTE MOOD EPISODES WITH STANDARD THERAPY; TREATMENT OF PREMENSTRUAL DYSPHORIC DISORDER (PMDD) USING AN INTERMITTENT DOSING REGIMEN; PREVENTION OF CYTOMEGALOVIRUS DISEASE IN KIDNEY, HEART, AND KIDNEY-PANCREAS TRANSPLANT PATIENTS AT HIGH RISK (DONOR CMV SEROPOSITIVE/RECIPIENT CMV SERONEGATIVE); IMPROVE SURVIVAL OF STABLE PATIENTS WITH LEFT VENTRICULAR SYSTOLIC DYSFUNCTION (EJECTION FRACTION<=40%) AND CLINICAL EVIDENCE OF CONGESTIVE HEART FAILURE AFTER AN ACUTE MYOCARDIAL INFARCTION; STIMULATION OF PANCREATIC SECRETIONS TO FACILITATE THE IDENTIFICATION OF THE AMPULLA OF VATER AND ACCESSORY PAPILLA DURING ENDOSCOPIC RETROGRADE CHOLANGIO-PANCREATOGRAPHY (ERCP); ESOPHAGEAL CANDIDIASIS; USE OF ADVAIR DISKUS 250/50 FOR CHRONIC OBSTRUCTIVE PULMONARY DISEASE (COPD) ASSOCIATED WITH CHRONIC BRONCHITIS; EXPANDED INDICATION FOR USE IN COMBINATION WITH ANTIDIABETIC DRUGS IN THE THIAZOLIDINEDIONE CLASS; MONOTHERAPY FOR THE SHORT TERM TREATMENT OF ACUTE MANIC OR MIXED EPISODES ASSOCIATED WITH BIPOLAR I DISORDER; ADJUNCTIVE THERAPY FOR THE SHORT TERM TREATMENT OF ACUTE MANIC OR MIXED EPISODES ASSOCIATED WITH BIPOLAR I DISORDER; PROPHYLAXIS OF DEEP VEIN THROMBOSIS (DVT), WHICH MAY LEAD TO PULMONARY EMBOLISM (PE) IN MEDICAL PATIENTS WHO ARE AT RISK FOR THROMBOEMBOLIC COMPLICATIONS DUE TO SEVERELY RESTRICTED MOBILITY DURING ACUTE ILLNESS; SEVERE HYPERTENSION WHEN THE VALUE OF ACHIEVING PROMPT BLOOD PRESSURE CONTROL EXCEEDS THE RISK OF INITIATING COMBINATION THERAPY; THE USE OF CIPRO XR FOR COMPLICATED URINARY TRACT INFECTIONS AND ACUTE UNCOMPLICATED PYELONEPHRITIS; USE IN THE LONG TERM TREATMENT OF BIPOLAR I DISORDER; ADJUNCTIVE THERAPY W/MOOD STABILIZERS (LITHIUM OR DIVALPROEX) IN THE TREATMENT OF ACUTE MANIC EPISODES ASSOCIATED WITH BIPOLAR I DISORDERS; MONOTHERAPY IN THE TREATMENT OF ACUTE MANIC EPISODES ASSOCIATED WITH BIPOLAR I DISORDER; TOPICAL TREATMENT OF CLINICALLY TYPICAL, NONHYPERKERATOTIC, NONHYPERTROPHIC ACTINIC KERATOSES ON THE FACE OR SCALP IN IMMUNOCOMPETENT ADULTS; TREATMENT OF COMPLICATED URINARY TRACT INFECTIONS AND PYELONEPHRITIS DUE TO E. COLI FOR PED PATIENTS (1-17) NOT AS FIRST CHOICE; INDICATED FOR THE IN-HOSPITAL SHORT-TERM (UP TO 4 HOURS) REDUCTION IN BLOOD PRESSURE IN PEDIATRIC PATIENTS; ACUTE TREATMENT OF MIGRAINE ATTACKS WITH OR WITHOUT AURA IN ADULTS; MANAGEMENT OF SECONDARY HYPERPARATHYROIDISM IN PATIENTS WITH MODERATE TO SEVERE CHRONIC RENAL INSUFFICIENCY NOT YET ON DIALYSIS; ELOXATIN IN COMBINATION WITH INFUSIONAL 5-FLUOROURACIL (5-FU) AND LEUCOVORIN (LV) FOR THE TREATMENT OF PATIENTS PREVIOUSLY UNTREATED FOR ADVANCED COLORECTAL CANCER; TREATMENT OF ACUTE PULMONARY EMBOLISM WHEN ADMINISTERED IN CONJUNCTION WITH WARFARIN SODIUM; TREATMENT OF ACUTE DEEP VEIN THROMBOSIS WITHOUT PULMONARY EMBOLISM WHEN ADMINISTERED IN CONJUNCTION WITH WARFARIN SODIUM; FOR USE IN COMBINATION WITH PACLITAXEL FOR THE FIRST-LINE TREATMENT OF PATIENTS WITH METASTATIC BREAST CANCER AFTER FAILURE OF PRIOR ANTHRACYCLINE CONTAINING ADJUVANT CHEMOTHERAPY UNLESS ANTHRACYCLINES WERE CLINICALLY CONTRAINDICATED; FOR USE IN COMBINATION WITH PREDNISONE FOR THE TREATMENT OF PATIENTS WITH ANDROGEN INDEPENDENT (HORMONE REFRACTORY) METASTATIC PROSTATE CANCER; FOR USE IN THE RELIEF OF THE SIGNS AND SYMPTOMS OF RHEUMATOID ARTHRITIS IN ADULTS; NOSOCOMIAL PNEUMONIA AND COMMUNITY-ACQUIRED PNEUMONIA CAUSED BY STREPTOCOCCUS PNEUMONIAE INDICATION EXPANDED TO INCLUDE MULTI-DRUG RESISTANT STRAINS; TREATMENT OF COMMUNITY ACQUIRED PNEUMONIA CAUSED BY MULTI-DRUG RESISTANT STREPTOCOCCUS PNEUMONIAE; TREATMENT OF BIOPSY-CONFIRMED, PRIMARY SUPERFICIAL BASAL CELL CARCINOMA IN IMMUNOCOMPETENT ADULTS, WITH A MAXIMUM TUMOR DIAMETER OF 2.0CM, LOCATED ON THE TRUNK (EXCLUDING ANOGENITAL SKIN), NECK, OR EXTREMITIES (EXCLUDING HANDS AND FEET); PREVENTION OF CARDIOVASCULAR DISEASE IN ADULT PATIENTS WITHOUT CLINICALLY EVIDENT HEART DISEASE, BUT WITH MULTIPLE RISK FACTORS FOR CORONARY HEART DISEASE TO REDUCE RISK OF MI AND RISK FOR REVASCULARIZATION PROCEDURES AND ANGINA; CHRONIC IDIOPATHIC CONSTIPATION; FOR USE IN COMBINATION WITH DOXORUBICIN AND CYCLOPHOSPHAMIDE FOR THE ADJUVANT TREATMENT OF PATIENTS WITH OPERABLE NODE-POSITIVE BREAST CANCER; TREATMENT OF ACUTE MANIC AND MIXED EPISODES ASSOCIATED WITH BIPOLAR DISORDER; EMPIRICAL THERAPY FOR PRESUMED FUNGAL INFECTIONS IN FEBRILE, NEUTROPENIC PATIENTS; USED TO TREAT ADULTS WITH GROWTH HORMONE DEFICIENCY; FOR THE REPLACEMENT OF ENDOGENOUS GROWTH HORMONE IN ADULTS WITH GROWTH HORMONE DEFICIENCY; USE COMBINATION WITH INFUSIONAL 5-FU/LV FOR ADJUVANT TREATMENT STAGE III COLON CANCER PTS WHO HAVE UNDERGONE COMPLETE RESECTION PRIMARY TUMOR-BASED ON IMPROVEMENT IN DISEASE FREE SURVIVAL, NO DEMONSTRATED BENEFIT OVERALL SURVIVAL AFTER 4YRS; USED FOR CANDIDEMIA IN NONNEUTROPENIC PATIENTS AND THE FOLLOWING CANDIDA INFECTIONS, DISSEMINATED INFECTIONS IN SKIN & INFECTIONS IN ABDOMEN, KIDNEY, BLADDER WALL, AND WOUNDS; TREATMENT OF NASAL POLYPS IN PATIENTS 18 YEARS OF AGE AND OLDER; USE OF PROTONIX IV FOR INJECTION AS STAND ALONE THERAPY FOR THE SHORT-TERM TREATMENT OF PATIENTS HAVING GASTROESOPHAGEAL REFLUX (GERD) WITH A HISTORY OF EROSIVE ESOPHAGITIS; TO IMPROVE (COMPARED TO 4.25% DEXTROSE) LONG-DWELL ULTRAFILTRATION AND CLEARANCE OF CREATININE AND UREA NITROGEN IN PATIENTS WITH HIGH AVERAGE OR GREATER TRANSPORT CHARACTERISTICS, AS DEFINED USING THE PERITONEAL EQUILIBRATION TEST (PET); EXTENDED ADJUVANT TREATMENT OF EARLY BREAST CANCER IN POSTMENOPAUSAL WOMEN WHO HAVE RECEIVED 5 YRS ADJUVANT TAMOXIFEN THERAPY-EFFECTIVENESS BASED ON AN ANALYSIS OF DISEASE FREE SURVIVAL IN PATIENTS TREATED FOR A MEDIAN 24 MONTHS; USE OF COPEGUS (RIBAVIRIN) FOR TREATMENT OF CHRONIC HEPATITIS C IN ADULT PATIENTS COINFECTED WITH HIV IN COMBINATION WITH PEGASYS (PEGINTERFERON ALFA-2A); TREATMENT OF HEART FAILURE (NYHA CLASS II-IV AND EJECTION FRACTION <=40%) TO REDUCE THE RISK OF DEATH FROM CARDIOVASCULAR CAUSES AND TO REDUCE HOSPITALIZATIONS FOR HEART FAILURE; TO IMPROVE WAKEFULNESS IN TWO NEW PATIENT POPULATIONS WITH EXCESSIVE SLEEPINESS, THOSE WITH OBSTRUCTIVE SLEEP APNEA/HYPOPNEA SYNDROME AND THOSE WITH SHIFT WORK SLEEP DISORDER; TREATMENT OF PATIENTS WITH NEWLY DIAGNOSED HIGH GRADE GLIOMAS CONCOMITANTLY WITH RADIOTHERAPY AND THEN AS ADJUVANT TREATMENT; MANAGEMENT OF ENDOMETRIOSIS ASSOCIATED PAIN; EXPANDED INDICATION TO INCLUDE TREATMENT OF MULTIPLE MYELOMA PATIENTS WHO HAVE RECEIVED AT LEAST 1 PRIOR THERAPY; USE IN COMBINATION WITH A SULFONYLUREA PLUS METFORMIN WHEN DIET, EXERCISE AND BOTH AGENTS DO NOT RESULT IN ADEQUATE GLYCEMIC CONTROL (TRIPLE THERAPY); MAINTENANCE OF CLINICAL REMISSION OF MILD TO MODERATE CROHN'S DISEASE INVOLVING THE ILEUM AND/OR THE ASCENDING COLON FOR UP TO 3 MONTHS; MODIFIED HEART FAILURE INDICATION TO INCLUDE TREATMENT OF HEART FAILURE IN PATIENTS WITH LEFT VENTRICULAR SYSTOLIC DYSFUNCTION (NYHA CLASS II-IV; EJECTION FRACTION LESS THAN OR EQUAL TO 40%); TO REDUCE CARDIOVASCULAR DEATH AND TO REDUCE HEART FAILURE HOSPITALIZATIONS, INCLUDES ADDITIONAL INFORMATION ON THE ADDED EFFECT ON THESE OUTCOMES WHEN USED WITH AN ACE INHIBITOR; TREATMENT OF PATIENTS UNDERGOING ABDOMINAL SURGERY WHO ARE AT RISK FOR THROMBOEMBOLIC COMPLICATIONS; USE OF BIVALIRUDIN FOR INJECTION WITH PROVISIONAL USE OF GLYCOPROTEIN IIB/IIIA INHIBITOR (GPI) AS LISTED IN THE CLINICAL TRIALS REPLACE-2 SECTION FOR USE AS AN ANTICOAGULANT IN PATIENTS UNDERGOING PERCUTANEOUS CORONARY INTERVENTION (PCI); NON-DIALYSIS DEPENDENT CHRONIC KIDNEY DISEASE (NDD-CKD) PATIENTS RECEIVING OR NOT RECEIVING AN ERYTHROPOIETIN; TREATMENT OF DIARRHEA CAUSED BY CRYPTOSPORIDIUM PARVUM IN NON-HIV INFECTED PATIENTS 12 YEARS OF AGE AND OLDER; USE AS A SINGLE AGENT FOR ADJUVANT TREATMENT IN PATIENTS WITH DUKES' C COLON CANCER WHO HAVE UNDERGONE COMPLETE RESECTION OF THE PRIMARY TUMOR WHEN TREATMENT WITH FLUOROPYRIMIDINE THERAPY ALONE IS PREFERRED; LONG TERM TREATMENT OF IDIOPATHIC SHORT STATURE; TREATMENT OF PATIENTS POST MYOCARDIAL INFARCTION; TREATMENT OF MODERATE TO SEVERE PRIMARY RESTLESS LEGS SYNDROME; PERENNIAL ALLERGIC RHINITIS IN ADULTS AND PEDIATRIC PATIENTS 6 MONTHS OF AGE AND OLDER; FOR RELIEF OF THE SIGNS AND SYMPTOMS OF ANKYLOSING SPONDYLITIS; USE OF TOPIRAMATE AS INITIAL MONOTHERAPY IN PATIENTS 10 YEARS OF AGE AND OLDER WITH PARTIAL ONSET OR PRIMARY GENERALIZED TONIC CLONIC SEIZURES; USE IN PATIENTS WITH STABLE CORONARY ARTERY DISEASE TO REDUCE THE RISK OF CARDIOVASCULAR MORTALITY OR NON-FATAL MYOCARDIAL INFARCTION; RELIEF OF THE SIGNS AND SYMPTOMS OF PAUCIARTICULAR OR POLYARTICULAR COURSE JUVENILE RHEUMATOID ARTHRITIS IN PATIENTS 2 YEARS OF AGE AND OLDER; DIABETIC PERIPHERAL NEUROPATHIC PAIN; INDICATED TO REDUCE THE RISK OF MYOCARDIAL INFARCTION AND STROKE IN PATIENTS WITH TYPE 2 DIABETES AND WITHOUT CLINICALLY EVIDENT CORONARY HEART DISEASE BUT WITH MULTIPLE RISK FACTORS FOR CORONARY HEART DISEASE; USE IN PATIENTS WITH ANGIOGRAPHICALLY DOCUMENTED CORONARY ARTERY DISEASE; USE IN COMBINATION WITH GEMCITABINE FOR THE FIRST LINE TREATMENT OF PATIENTS WITH LOCALLY ADVANCED UNRESECTABLE OR METASTATIC PANCREATIC CANCER; TREATMENT OF IRON DEFICIENCY ANEMIA IN PERITONEAL DIALYSIS DEPENDANT CHRONIC KIDNEY DISEASE IN PATIENTS RECEIVING AN ERYTHROPOIETIN; PREVENTION OF NAUSEA AND VOMITING ASSOCIATED WITH INITIAL AND REPEAT COURSES OF MODERATELY EMETOGENIC CANCER CHEMOTHERAPY; TREATMENT OF DIABETIC FOOT INFECTIONS WITHOUT OSTEOMYELITIS; TREATMENT OF COMPLICATED SKIN AND SKIN STRUCTURE INFECTIONS CAUSED BY METHICILLIN SUSCEPTIBLE STAPHYLOCOCCUS AUREUS, ESCHERICHIA COLI, KLEBSIELLA PNEUMONIAE, OR ENTEROBACTER CLOACAE; FOR USE AS ADJUNCTIVE THERAPY IN THE TREATMENT OF PARTIAL SEIZURES IN CHILDREN WITH EPILEPSY AGED 2-4 YEARS; TREATMENT OF COMPLICATED INTRA-ABDOMINAL INFECTIONS CAUSED BY E. COLI, B. FRAGILIS, S. ANGINOSUS, S. CONSTELLATUS, E. FAECALIS, P. MIRABILIS, C. PERFRINGENS, B. THETAIOTAOMICRON OR PEPTOSTREPTOCOCCUS SPECIES; PROPHYLAXIS OF INFLUENZA FOR PATIENTS BETWEEN 1-12 YEARS OF AGE; INDICATED FOR THE ADJUVANT TREATMENT OF POSTMENOPAUSAL WOMEN WITH HORMONE RECEPTOR POSITIVE EARLY BREAST CANCER; TREATMENT OF ACUTE MANIC OR MIXED EPISODES ASSOCIATED WITH BIPOLAR I DISORDER WITH OR WITHOUT PSYCHOTIC FEATURES; PREVENTION OF POSTMENOPAUSAL OSTEOPOROSIS; FOR THE RISK REDUCTION OF NSAID-ASSOCIATED GASTRIC ULCERS; TREATMENT OF POSTOPERATIVE INFLAMMATION AND REDUCTION OF OCULAR PAIN IN PATIENTS WHO HAVE UNDERGONE CATARACT EXTRACTION; ANGIOMAX IS INDICATED FOR PATIENTS WITH, OR AT RISK OF, HIT/HITTS UNDERGOING PCI; INDICATED FOR THE RELIEF OF THE INFLAMMATORY AND PRURITIC MANIFESTATIONS OF CORTICOSTEROID RESPONSIVE DERMATOSES IN PATIENTS 12 YRS OF AGE OR OLDER; MAINTENANCE THERAPY IN BIPOLAR I DISORDER; FOR USE IN PEDIATRIC PATIENTS WITH TYPE I DIABETES; FOR USE IN COMBINATION WITH CISPLATIN AND FLUOROURACIL FOR THE TREATMENT OF PATIENTS WITH ADVANCED GASTRIC ADENOCARCINOMA, INCLUDING ADENOCARCINOMA OF GASTROESOPHAGEAL JUNCTION, WHO HAVE NOT RECEIVED PRIOR CHEMOTHERAPY FOR ADVANCED DISEASE; INFLUENZA PROPHYLAXIS; MONOTHERAPY IN THE TREATMENT OF ACUTE MANIC OR MIXED EPISODES IN BIPOLAR I DISORDER, WITH OR WITHOUT PSYCHOTIC FEATURES; ADMINISTERED IN COMBINATION WITH FENOFIBRATE, AS ADJUNCTIVE THERAPY TO DIET FOR THE REDUCTION OF ELEVATED TOTAL-C, LDL-C, APO B, AND NON-HDL-C IN PATIENTS WITH MIXED HYPERLIPIDEMIA; CLINICAL DATA IN SUPPORT OF AVANDAMET AS AN ADJUNCT TO DIET AND EXERCISE TO IMPROVE GLYCEMIC CONTROL IN PATIENTS WITH TYPE 2 DIABETES MELLITUS WHEN TREATMENT WITH DUAL ROSIGLITAZONE AND METFORMIN THERAPY IS APPROPRIATE; ADJUVANT TX OF POSTMENOPAUSAL WOMEN WITH ESTROGEN-RECEPTOR POSITIVE EARLY BREAST CANCER WHO HAVE RECEIVED 2 TO 3 YRS OF TAMOXIFEN AND ARE SWITCHED TO AROMASIN FOR COMPLETION OF A TOTAL OF 5 CONSECUTIVE YRS OF ADJUVANT HORMONAL THERAPY; LONG TERM TREATMENT OF GROWTH FAILURE ASSOCIATED WITH TURNER SYNDROME IN PATIENTS WHO HAVE OPEN EPIPHYSES; PREVENTION OF SEASONAL MAJOR DEPRESSIVE EPISODES IN PATIENTS WITH SEASONAL AFFECTIVE DISORDER; PREVENTION OF POSTOPERATIVE NAUSEA AND VOMITING; USE OF GEMZAR IN COMBINATION WITH CARBOPLATIN FOR THE TREATMENT OF PATIENTS WITH ADVANCED OVARIAN CANCER THAT HAS RELAPSED AT LEAST 6 MONTHS AFTER COMPLETION OF PLATINUM-BASED THERAPY; FOR USE IN COMBINATION WITH DEXAMETHASONE FOR THE TREATMENT OF MULTIPLE MYELOMA PATIENTS WHO HAVE RECEIVED AT LEAST ONE PRIOR THERAPY; TREATMENT OF RECURRENT HERPES LABIALIS (COLD SORES) IN IMMUNOCOMPETENT PATIENTS WITH A SINGLE DOSE OF FAMCICLOVIR 1500 MG; FOR PTS WITH ST-SEGMENT ELEVATION ACUTE MYOCARDIAL INFARCTION, PLAVIX TO REDUCE RATE OF DEATH FROM ANY CAUSE AND THE RATE OF A COMBINED ENDPOINT OF DEATH, REINFARCTION OR STROKE, NOT KNOWN TO PERTAIN TO PTS WHO RECEIVE PRIMARY ANGIOPLASTY; TREATMENT OF MAJOR DEPRESSIVE EPISODES ASSOCIATED WITH BIPOLAR DISORDER; TREATMENT OF PATHOLOGICAL HYPERSECRETORY CONDITIONS INCLUDING ZOLLINGER-ELLISON SYNDROME; TREATMENT OF STAPHYLOCOCCUS AUREUS BLOODSTREAM INFECTIONS (BACTEREMIA), INCLUDING THOSE WITH RIGHT SIDED INFECTIVE ENDOCARDITIS, CAUSED BY METHICILLIN-SUSCEPTIBLE AND METHICILLIN-RESISTANT ISOLATES; ADJUNCTIVE THERAPY OF MYOCLONIC SEIZURES IN ADULTS AND ADOLESCENTS AGE 12 AND OVER WITH JUVENILE MYOCLONIC EPILEPSY; ADJUNCT TO DIET TO REDUCE TOTAL-C, LDL-C AND APO B LEVELS IN ADOLESCENT BOYS AND GIRLS WHO ARE AT LEAST ONE YEAR POST-MENARCHE, 10-16 YEARS OF AGE, WITH HETEROZYGOUS FAMILIAL HYPERCHOLESTEROLEMIA; PREMENSTRUAL DYSPHONIC DISORDER; TREATMENT OF IRRITABILITY ASSOCIATED WITH AUTISTIC DISORDER; ADULT DERMATOFIBROSARCOMA PROTUBERANS (DFSP); ADULT MYELODYSPLASTIC SYNDROME/MYELOPROLIFERATIVE DISEASES (MDS/MDP); ADULT PH+ ACUTE LYMPHOBLASTIC LEUKEMIA (ALL) MONOTHERAPY; ADULT AGGRESSIVE SYSTEMIC MASTOCYTOSIS (ASM); ADULT HYPEREOSINOPHILIC SYNDROME/CHRONIC EOSINOPHILIC LEUKEMIA (HES/CEL); PROPHYLAXIS OF SURGICAL SITE INFECTION FOLLOWING ELECTIVE COLORECTAL SURGERY; PRIMARY GENERALIZED TONIC CLONIC SEIZURES IN ADULTS AND PEDIATRIC PATIENTS 2 YEARS OF AGE AND OLDER; TREATMENT OF MODERATE TO SEVERE PRIMARY RESTLESS LEG SYNDROME (RLS); TREATMENT OF SHORT STATURE OR GROWTH FAILURE IN CHILDREN WITH SHOX (SHORT STATURE HOMEOBOX CONTAINING GENE) DEFICIENCY WHOSE EPIPHYSES ARE NOT CLOSED; USE OF TAXOTERE (DOCETAXEL) INJECTION CONCENTRATE IN COMBINATION WITH CISPLATIN AND FLUOROURACIL FOR THE INDUCTION OF PATIENTS WITH INOPERABLE LOCALLY ADVANCED SQUAMOUS CELL CARCINOMA OF THE HEAD AND NECK (SCCHN); USE OF EXENATIDE IN PATIENTS WITH TYPE 2 DIABETES MELLITUS WHO ARE USING A THIAZOLIDINEDIONE ALONE OR IN COMBINATION WITH METFORMIN BUT HAVE NOT ACHIEVED ADEQUATE GLYCEMIC CONTROL; TREATMENT OF PATIENTS WITH MANTLE CELL LYMPHOMA WHO HAVE RECEIVED AT LEAST 1 YEAR PRIOR THERAPY; TREATMENT OF MODERATE ACNE VULGARIS IN WOMEN AT LEAST 14 YRS OF AGE, WHO HAVE NO KNOWN CONTRAINDICATIONS TO ORAL CONTRACEPTIVE THERAPY, AND HAVE ACHIEVED MENARCHE, IF THE PATIENT DESIRES AN ORAL CONTRACEPTIVE FOR BIRTH CONTROL; USE IN ADULT PATIENTS WITH CLINICALLY EVIDENT CORONARY HEART DISEASE TO REDUCE THE RISK OF NONFATAL MYOCARDIAL INFARCTION, FATAL AND NONFATAL STROKE, ANGINA, REVASCULARIZATION PROCEDURES AND HOSPITALIZATION FOR CONGESTIVE HEART FAILURE; GENERALIZED ANXIETY DISORDER (GAD); USE OF 0.5 MG/0.1 MG FOR PREVENTION OF POST-MENOPAUSAL OSTEOPOROSIS; TREATMENT OF HYPONATREMIA IN HOSPITALIZED PATIENTS; ADJUNCTIVE THERAPY IN THE TREATMENT OF PRIMARY GENERALIZED TONIC-CLONIC SEIZURES IN ADULTS AND CHILDREN 6 YEARS OF AGE AND OLDER WITH IDIOPATHIC GENERALIZED EPILEPSY; TREATMENT OF MODERATE TO SEVERE VAGINAL DRYNESS AND PAIN WITH INTERCOURSE, SYMPTOMS OF VULVAR AND VAGINAL ATROPHY ASSOCIATED WITH MENOPAUSE; TREATMENT OF DEMENTIA OF THE ALZHEIMER'S TYPE IN PATIENTS WITH SEVERE ALZHEIMER'S DISEASE; PREVENTION OF EXERCISE-INDUCED BRONCHOCONSTRICTION IN PATIENTS 15 YEARS OF AGE AND OLDER; MAINTENANCE TREATMENT OF SCHIZOPHRENIA; TREATMENT OF BACTERIAL VAGINOSIS IN NON-PREGNANT FEMALES; ACUTE ST-SEGMENT ELEVATION MYOCARDIAL INFARCTION (STEMI); EXTENDED TREATMENT OF SYMPTOMATIC VENOUS THROMBOEMBOLISM (VTE) AND/OR PULMONARY EMBOLISM TO REDUCE THE RECURRENCE OF VTE IN PATIENTS WITH CANCER; MANAGEMENT OF FIBROMYALGIA; FOR THE TREATMENT OF SHORT STATURE IN CHILDREN WITH NOONAN SYNDROME; LONG TERM TREATMENT OF PANIC DISORDER; SHORT TERM TREATMENT OF PANIC DISORDER; REDUCTION IN RISK OF INVASIVE BREAST CANCER IN POSTMENOPAUSAL WOMEN WITH OSTEOPOROSIS OR AT HIGH RISK FOR INVASIVE BREAST CANCER; TREATMENT OF SCHIZOPHRENIA IN ADOLESCENTS AGES 13-17; TREATMENT OF BIPOLAR I DISORDER IN CHILDREN AGES 10-12 AND ADOLESCENTS AGES 13-17; EXPANSION OF PATIENT POPULATION FOR HEAD AND NECK CANCER FROM ‘INOPERABLE’ PATIENTS TO ALL PATIENTS; USE IN COMBINATION WITH CISPLATIN AND FLUOROURACIL FOR THE INDUCTION TREATMENT OF PATIENTS WITH LOCALLY ADVANCED SQUAMOUS CELL CARCINOMA OF THE HEAD AND NECK (SCCHN); ADJUNCTIVE THERAPY OF MYOCLONIC SEIZURES IN ADULTS AND ADOLESCENTS AGE 16 AND OVER WITH JUVENILE MYOCLONIC EPILEPSY; ADJUNCTIVE TREATMENT TO TREAT PATIENTS WITH MAJOR DEPRESSIVE DISORDER; TREATMENT OF UNRESECTABLE HEPATOCELLULAR CARCINOMA; ADJUNCTIVE THERAPY TO DIET TO SLOW THE PROGRESSION OF ARTHEROSCLEROSIS IN ADULT PATIENTS AS PART OF A TREATMENT STRATEGY TO LOWER TOTAL-C AND LDL-C TO TARGET LEVELS; SEASONAL ALLERGIC RHINITIS IN PATIENTS 6 THROUGH LESS THAN 12 YEARS OF AGE; USE OF AVALIDE TABLETS AS INITIAL THERAPY IN PATIENTS WHO ARE LIKELY TO NEED MULTIPLE DRUGS TO ACHIEVE THEIR BLOOD PRESSURE GOALS; TREATMENT OF HYPERTENSION IN PEDIATRIC PATIENTS 6-16 YEARS OF AGE; TREATMENT OF SHORT STATURE IN CHILDREN WITH TURNER'S SYNDROME; ADJUNCTIVE TREATMENT FOR RADIOIODINE ABLATION OF THYROID TISSUE REMNANTS IN PATIENTS WHO HAVE UNDERGONE THYROIDECTOMY FOR WELL-DIFFERENTIATED THYROID CANCER AND WHO DO NOT HAVE EVIDENCE OF METASTATIC THYROID CANCER; FOR USE AS AN ADJUNCT TO DIET AND EXERCISE TO IMPROVE GLYCEMIC CONTROL IN ADULTS WITH TYPE 2 DIABETES MELLITUS; TREATMENT OF PATIENTS WITH CANDIDEMIA, ACUTE DISSEMINATED CANDIDIASIS, CANDIDA PERITONITIS AND ABSCESSES; TREATMENT OF ACUTE MANIC OR MIXED EPISODES ASSOCIATED WITH BIPOLAR I DISORDER IN PEDIATRIC PATIENTS AGED 10-17 YEARS; PREVENTION OF POST OPERATIVE NAUSEA AND VOMITING FOR UP TO 24 HOURS FOLLOWING SURGERY; USE OF AMITIZA (LUBIPROSTONE) 8 MCG TWICE DAILY FOR TREATMENT OF IRRITABLE BOWEL SYNDROME WITH CONSTIPATION IN WOMEN GREATER THAN OR EQUAL TO 18 YEARS OLD; MAINTENANCE TREATMENT OF AIRFLOW OBSTRUCTION AND REDUCING EXACERBATIONS IN PATIENTS WITH CHRONIC OBSTRUCTIVE PULMONARY DISEASE (COPD) INCLUDING CHRONIC BRONCHITIS AND EMPHYSEMA; ADJUNCTIVE THERAPY ADDED TO LITHIUM OR VALPROATE IN SHORT TERM TREATMENT OF BIPOLAR DISORDER, MANIC OR MIXED; MAINTENANCE TREATMENT FOR BIPOLAR I DISORDER, AS ADJUNCTIVE THERAPY TO LITHIUM OR DIVALPROEX; LONG-TERM TREATMENT OF SOCIAL ANXIETY DISORDER; MAINTENANCE TREATMENT OF ATTENTION-DEFICIT HYPERACTIVITY DISORDER (ADHD) IN CHILDREN AND ADOLESCENTS; ADJUNCTIVE THERAPY IN THE TREATMENT OF PRIMARY GENERALIZED TONIC-CLONIC SIEZURES IN ADULTS AND CHILDREN 16 YEARS OF AGE AND OLDER WITH IDIOPATHIC GENERALIZED EPILEPSY; TREATMENT OF PATIENTS WITH MULTIPLE MYELOMA; USE OF DUTASTERIDE IN COMBINATION WITH TAMSULOSIN FOR THE TREATMENT OF SYMPTOMATIC BENIGN PROSTATIC HYPERPLASIA (BPH); MANAGEMENT OF FIBROMYALGIA; INITIAL THERAPY IN PATIENTS LIKELY TO NEED MULTIPLE DRUGS TO ACHIEVE THEIR BLOOD PRESSURE GOALS; USE OF APTIVUS, CO-ADMINISTERED W/RITONAVIR, FOR COMBINATION ANTIRETROVIRAL TREATMENT OF HIV-1 INFECTED PED (AGE 2-18 YRS) PATIENTS WHO ARE TREATMENT-EXPERIENCED AND INFECTED W/HIV-1 STRAINS RESISTANT TO MORE THAN ONE PROTEASE INHIBITOR; TREATMENT OF CHRONIC HEPATITIS B; TREATMENT OF CHICKEN POX IN IMMUNOCOMPETENT PEDIATRIC PATIENTS 2 TO <18 YEARS OF AGE; NON-SMALL CELL LUNG CANCER IN COMBINATION WITH CISPLATIN AND AS SINGLE AGENT FOR NONSQAUMOUS NON-SMALL CELL LUNG CANCER; TREATMENT OF GROWTH FAILURE IN CHILDREN BORN SMALL FOR GESTATIONAL AGE (SGA) WITH NO CATCH-UP BY AGE 2-4 YRS; TO TREAT PATIENTS WITH PRIMARY DYSBETALIPOPROTEINEMIA (FREDRICKSON TYPE III HYPERLIPOPROTEINEMIA) AS AN ADJUNCT TO DIET; MONOTHERAPY IN THE TREATMENT OF BIPOLAR DEPRESSION; MONOTHERAPY IN THE TREATMENT OF BIPOLAR MANIA; ADJUNCTIVE THERAPY IN THE TREATMENT OF BIPOLAR MANIA; SEDATION OF NON-INTUBATED PATIENTS PRIOR TO AND/OR DURING SURGICAL AND OTHER PROCEDURES; EXPANSION OF INDICATION TO INCLUDE TREATMENT OF HIV IN TREATMENT NAIVE ADULTS; TREATMENT OF MODERATE TO SEVERE DYSPAREUNIA, A SYMPTOM OF VULVAR AND VAGINAL ATROPHY, DUE TO MENOPAUSE AND NEW TWICE WEEKLY DOSING REGIMEN FOR THIS INDICATION; INDOLENT B-CELL NON-HODGKINS LYMPHOMA (NHL) THAT HAS PROGRESSED DURING OR WITHIN SIX MONTHS OF TREATMENT WITH RITUXIMAB OR A RITUXIMAB CONTAINING REGIMEN; TREATMENT TO INCREASE BONE MASS IN MEN WITH OSTEOPOROSIS; TREATMENT OF CHRONIC OBSTRUCTIVE PULMONARY DISEASE; ADJUVANT TREATMENT OF ADULT PATIENTS FOLLOWING COMPLETE GROSS RESECTION OF KIT (CD117) POSITIVE GASTOINTESTINAL STROMAL TUMORS (GIST); TREATMENT AND PREVENTION OF GLUCOCORTICOID-INDUCED OSTEOPOROSIS IN PATIENTS EXPECTED TO BE ON GLUCOCORTICOIDS FOR AT LEAST 12 MONTHS; TREATMENT OF SHORT STATURE IN PEDIATRIC PATIENTS SMALL FOR GESTATIONAL AGE WHO DO NOT MANIFEST CATCH UP GROWTH BY AGE 2 TO 4 YEARS; COMMUNITY ACQUIRED BACTERIAL PNEUMONIA; ADDITIONAL PATHOGENS TO COMPLICATED SKIN AND SKIN STRUCTURE INFECTIONS INDICATION; ADDITIONAL PATHOGENS TO COMPLICATED INTRA-ABDOMINAL INFECTIONS INDICATION; TREATMENT OF TREATMENT RESISTANT DEPRESSION (TRD) IN COMBINATION WITH OLANZAPINE; ACUTE TREATMENT OF DEPRESSIVE EPISODES ASSOCIATED WITH BIPOLAR DISORDER (IN COMBINATION WITH OLANZAPINE); TREATMENT OF TREATMENT RESISTANT DEPRESSION (TRD) IN COMBINATION WITH FLUOXETINE; ACUTE TREATMENT OF DEPRESSIVE EPISODES ASSOCIATED WITH BIPOLAR DISORDER (IN COMBINATION WITH FLUOXETINE); TREATMENT OF TREATMENT RESISTANT DEPRESSION (TRD); INDICATION EXPANDED TO INCLUDE PATIENTS WHO HAVE EXPERIENCED A FIRST CLINICAL EPISODE AND HAVE MRI FEATURES CONSISTENT WITH MULTIPLE SCLEROSIS; PREVENTION OF OSTEOPOROSIS IN POSTMENOPAUSAL WOMEN; USE AS ADJUNCTIVE THERAPY WITH LITHIUM OR VALPROATE FOR THE MAINTENANCE TREATMENT OF BIPOLAR I DISORDER; MONOTHERAPY FOR THE MAINTENANCE TREATMENT OF BIPOLAR I DISORDER; TREATMENT OF PULMONARY ARTERIAL HYPERTENSION INDICATION EXPANDED TO INCLUDE DELAY IN CLINICAL WORSENING; PREVENTION AND TREATMENT OF SECONDARY HYPERPARATHYROIDISM ASSOCIATED WITH CHRONIC KIDNEY DISEASE (CKD) STAGE 5 IN PATIENTS ON HEMODIALYSIS OR PERITONEAL DIALYSIS; FOR USE AS INITIAL THERAPY IN PATIENTS WHO ARE LIKELY TO NEED MULTIPLE DRUGS TO ACHIEVE THEIR BLOOD PRESSURE GOALS; MAINTENANCE TREATMENT IN PATIENTS WITH ADVANCED OR METASTATIC NONSQUAMOUS NON-SMALL CELL LUNG CANCER WHOSE DISEASE HAS NOT PROGRESSED AFTER FOUR CYCLES OF PLATINUM-BASED FIRST LINE CHEMOTHERAPY; TREATMENT OF MEN AND WOMEN WITH OSTEOPOROSIS ASSOCIATED WITH SUSTAINED SYSTEMIC GLUCOCORTICOID THERAPY AT HIGH RISK FOR FRACTURE; GOUT FLARES; PREVENTION OF CMV DISEASE IN KIDNEY AND HEART TRANSPLANT PATIENTS 4 MONTHS TO 16 YEARS AT HIGH RISK; ADJUNCT TO MOOD STABILIZERS AND/OR ANTIDEPRESSANTS FOR SCHIZOAFFECTIVE DISORDER; TREATMENT OF SCHIZOAFFECTIVE DISORDER AS MONOTHERAPY; INDICATION EXPANDED TO INCLUDE TREATMENT OF PULMONARY ARTERIAL HYPERTENSION (WHO GROUP I) IN PATIENTS WITH CLASS II SYMPTOMS; REDUCE LDL-C LEVELS IN BOYS AND POSTMENARCHAL GIRLS, 10 TO 17 YEARS OF AGE, WITH HETEROZYGOUS FAMILIAL HYPERCHOLESTEROLEMIA AS MONOTHERAPY OR IN COMBINATION WITH A STATIN AFTER FAILING AN ADEQUATE TRIAL OF DIET THERAPY; TREATMENT OF HEAVY MENSTRUAL BLEEDING FOR WOMEN WHO CHOOSE TO USE INTRAUTERINE CONTRACEPTION AS THEIR METHOD OF CONTRACEPTION; TREATMENT OF HETEROZYGOUS FAMILIAL HYPERCHOLESTEROLEMIA IN ADOLESCENT BOYS AND POSTMENARCHAL GIRLS, AGES 10 TO 17 YEARS, WITH A RECOMMENDATION DOSING RANGE OF 5 TO 20 MG ONCE DAILY; MICARDIS 80 MG FOR REDUCTION OF THE RISK OF MYOCARDIAL INFARCTION, STROKE, OR DEATH FROM CARDIOVASCULAR CAUSES IN PATIENTS 55 YEARS OF AGE OR OLDER AT HIGH RISK OF DEVELOPING MAJOR CARDIOVASCULAR EVENTS WHO ARE UNABLE TO TAKE ACE INHIBITORS; MILD TO MODERATE ATOPIC DERMATITIS IN PATIENTS 3 MONTHS OF AGE TO LESS THAN 18 YEARS OF AGE; SHORT TERM TREATMENT OF EROSIVE ESOPHAGITIS ASSOCIATED WITH GERD IN PEDIATRIC PATIENTS AGES FIVE YEARS AND OLDER; MAINTENANCE TREATMENT OF BIPOLAR DISORDER AS AN ADJUNCT TO LITHIUM OR VALPROATE; TREATMENT OF IRRITABILITY ASSOCIATED WITH AUTISTIC DISORDER IN PEDIATRIC PATIENTS AGES 6-17 YEARS OF AGE; MAINTENANCE OF GENERALIZED ANXIETY DISORDER (GAD); ADJUNCTIVE THERAPY IN THE TREATMENT OF MAJOR DEPRESSIVE DISORDER (MDD); INTRAVENOUS CONTRAST ENHANCED COMPUTER TOMOGRAPHY OF THE HEAD AND BODY; FOR USE IN COMBINATION WITH LETROZOLE FOR THE TREATMENT OF POSTMENOPAUSAL WOMEN WITH HORMONE RECEPTOR POSITIVE METASTATIC BREAST CANCER THAT OVEREXPRESSES THE HER2 RECEPTOR FOR WHOM HORMONAL THERAPY IS INDICATED; PRIMARY PREVENTION OF CARDIOVASCULAR DISEASE, BASED ON THE RESULTS OF JUSTIFICATION FOR THE USE OF STATINS IN PRIMARY PREVENTION; AN INTERVENTION TRIAL EVALUATING ROSUVASTATIN (JUPITER); ADJUNCTIVE THERAPY FOR PRIMARY GENERALIZED TONIC-CLONIC SEIZURES IN PATIENTS THIRTEEN YEARS OF AGE AND OLDER; TREATMENT OF SIGNS AND SYMPTOMS OF ADVANCED IDIOPATHIC PARKINSON'S DISEASE; MAINTENANCE TREATMENT OF PATIENTS WITH LOCALLY ADVANCED OR METASTATIC NON-SMALL CELL LUNG CANCER WHOSE DISEASE HAS NOT PROGRESSED AFTER FOUR CYCLES OF PLATINUM-BASED FIRST-LINE CHEMOTHERAPY; PANCREATIC INSUFFICIENCY DUE TO CHRONIC PANCREATITIS AND PANCREATECTOMY; RELIEF OF NASAL CONGESTION ASSOCIATED WITH SEASONAL ALLERGIC RHINITIS IN ADULTS AND PEDIATRIC PATIENTS 2 YEARS OF AGE AND OLDER; TREATMENT OF NEWLY DIAGNOSED ADULT PATIENTS WITH PHILADELPHIA CHROMOSOME POSITIVE CHRONIC MYELOID LEUKEMIA (PH & CML) IN CHRONIC PHASE; MAINTENANCE TREATMENT OF SCHIZOPHRENIA IN ADULTS; ADJUNCTIVE THERAPY WITH EITHER LITHIUM OR VALPROATE FOR THE ACUTE TREATMENT OF MANIC OR MIXED EPISODES ASSOCIATED WITH BIPOLAR I DISORDER; TREATMENT OF PATIENTS WITH SUBEPENDYMAL GIANT CELL ASTROCYTOMA (SEGA) ASSOCIATED WITH TUBEROUS SCLEROSIS (TS) WHO REQUIRE THERAPEUTIC INTERVENTION BUT ARE NOT CANDIDATES FOR CURATIVE SURGICAL RESECTION; PREVENTION OF RELAPSE TO OPIOID DEPENDENCE FOLLOWING OPIOID DETOXIFICATION; MANAGEMENT OF CHRONIC MUSCULOSKELETAL PAIN; MAINTENANCE TREATMENT OF BIPOLAR I DISORDER AS AN ADJUNCT TO LITHIUM OR VALPROATE; TREATMENT OF SEVERE HYPERCALCEMIA IN PATIENTS WITH PRIMARY HYPERPARATHYROIDISM WHO ARE UNABLE TO UNDERGO PARATHYROIDECTOMY; ADJUNCTIVE TREATMENT WITH LONG-ACTING ORAL PSYCHOSTIMULANTS FOR THE TREATMENT OF ATTENTION DEFICIT HYPERACTIVITY DISORDER (ADHD); TREATMENT OF EXTERNAL GENITAL AND PERIANAL WARTS/CONDYLOMA ACUMINATA IN PATIENTS 12 YEARS OR OLDER; USE IN COMBINATION CHEMOTHERAPY WITH 5-FLUOROURACIL IN THE PALLIATIVE TREATMENT OF PATIENTS WITH ADVANCED METASTATIC COLORECTAL CANCER; FOR PATIENTS WITH PROGRESSIVE NEUROENDOCRINE TUMORS OF PANCREATIC ORIGIN (PNET) THAT ARE UNRESECTABLE, LOCALLY ADVANCED, OR METASTATIC; TREATMENT OF PROGRESSIVE, WELL-DIFFERENTIATED PANCREATIC NEUROENDOCRINE TUMORS IN PATIENTS WITH UNRESECTABLE, LOCALLY ADVANCED, OR METASTATIC DISEASE; MAINTENANCE OF REMISSION OF ULCERATIVE COLITIS; TREATMENT OF THE SIGNS AND SYMPTOMS OF BENIGN PROSTATIC HYPERPLASIA (BPH); TREATMENT OF ERECTILE DYSFUNCTION (ED) AND THE SIGNS AND SYMPTOMS OF BENIGN PROSTATIC HYPERPLASIA (BPH); REDUCE THE RISK OF STROKE AND SYSTEMIC EMBOLISM IN PATIENTS WITH NONVALVULAR ATRIAL FIBRILLATION; MONOTHERAPY IN PATIENTS 13 YEARS OF AGE AND OLDER WITH PARTIAL SEIZURES WHO ARE RECEIVING THERAPY WITH A SINGLE ANTIEPILEPTIC DRUG (AED); MAINTENANCE TREATMENT OF ATTENTION DEFICIT HYPERACTIVITY DISORDER (ADHD) IN ADULTS; SIGNS AND SYMPTOMS OF ADVANCED PARKINSON'S DISEASE (APD); SIGNS AND SYMPTOMS OF MODERATE TO SEVERE PRIMARY RESTLESS LEGS SYNDROME (RLS); TREATMENT OF HEAVY MENSTRUAL BLEEDING IN WOMEN WITHOUT ORGANIC PATHOLOGY WHO CHOOSE TO USE AN ORAL CONTRACEPTIVE AS THEIR METHOD OF CONTRACEPTION; TREATMENT OF PATIENTS WITH ADVANCED SOFT TISSUE SARCOMA (STS) WHO HAVE RECEIVED PRIOR CHEMOTHERAPY; TREATMENT OF ADULTS WITH RENAL ANGIOMYOLIPOMA AND TUBEROUS SCLEROSIS COMPLEX (TSC), NOT REQUIRING IMMEDIATE SURGERY; MANAGEMENT OF NEUROPATHIC PAIN ASSOCIATED WITH SPINAL CORD INJURY; MANAGEMENT OF POSTHERPETIC NEURALGIA; TREATMENT OF ENDOGENOUS ANTERIOR UVEITIS; MAGNETIC RESONANCE ANGIOGRAPHY (MRA) TO EVALUATE ADULTS WITH KNOWN OR SUSPECTED RENAL OR AORTO-ILIO-FEMORAL OCCLUSIVE VASCULAR DISEASE; TREATMENT OF POSTMENOPAUSAL WOMEN WITH ADVANCED HORMONE RECEPTOR-POSITIVE, HER2-NEGATIVE BREAST CANCER (ADVANCED HR+BC) IN COMBINATION WITH EXEMESTANE, AFTER FAILURE OF TREATMENT WITH LETROZOLE OR ANASTROZOLE; MANAGEMENT OF NEUROPATHIC PAIN ASSOCIATED WITH DIABETIC PERIPHERAL NEUROPATHY (DPN) IN ADULTS WHEN A CONTINUOUS, AROUND-THE-CLOCK OPIOID ANALGESIC IS NEEDED FOR AN EXTENDED PERIOD OF TIME; PLAQUE PSORIASIS OF THE SCALP; FIRST-LINE TREATMENT OF LOCALLY ADVANCED OR METASTATIC NON-SMALL CELL LUNG CANCER, IN COMBINATION WITH CARBOPLATIN, IN PATIENTS WHO ARE NOT CANDIDATES FOR CURATIVE SURGERY OR RADIATION THERAPY; PLAQUE PSORIASIS OF THE BODY; TREATMENT OF DEEP VEIN THROMBOSIS; TREATMENT OF PULMONARY EMBOLISM; REDUCTION IN RISK FOR DEEP VEIN THROMBOSIS AND THE REDUCTION IN RISK FOR PULMONARY EMBOLISM; IN COMBINATION WITH PREDNISONE FOR THE TREATMENT OF PATIENTS WITH METASTATIC CASTRATION-RESISTANT PROSTATE CANCER; TREATMENT OF THROMBOCYTOPENIA IN PATIENTS WITH CHRONIC HEPATITIS C TO ALLOW THE INITIATION AND MAINTENANCE OF INTERFERON-BASED THERAPY; TREATMENT OF CHRONIC IRON OVERLOAD IN PATIENTS 10 YRS OF AGE AND OLDER WITH (NTDT)SYNDROMES AND WITH A (LIC) OF AT LEAST 5 MG OF IRON PER GRAM OF LIVER DRY WEIGHT (MG FE/G DW) AND SERUM FERRITIN GREATER THAN 300 MCG/L; TREATMENT OF PEDIATRIC PATIENTS WITH NEWLY DIAGNOSED PHILADELPHIA CHROMOSOME-POSITIVE ACUTE LYMPHOBLASTIC LEUKEMIA (PH+ALL) IN COMBINATION WITH CHEMOTHERAPY; TREATMENT OF PATIENTS WITH LOCALLY ADVANCED, UNRESECTABLE OR METASTATIC GASTROINTESTINAL STROMAL TUMOR (GIST) WHO HAVE BEEN PREVIOUSLY TREATED WITH IMATINIB MESYLATE AND SUNITINIB MALATE; PROPHYLAXIS OF ALLOGRAFT REJECTION IN ADULT PATIENTS RECEIVING A LIVER TRANSPLANT; SCINTIGRAPHIC ASSESSMENT OF SYMPATHETIC INNERVATION OF THE MYOCARDIUM BY MEASUREMENT OF THE HEART TO MEDIASTINUM (H/M) RATIO OF RADIOACTIVITY UPTAKE IN PATIENTS WITH NYHA CLASS II OR CLASS III HEART FAILURE AND LVEF LESS THAN 35%; TREATMENT OF OPIOID-INDUCED CONSTIPATION (OIC) IN ADULTS WITH CHRONIC, NON-CANCER PAIN; FIRSTLINE TREATMENT OF PATIENTS WITH METASTATIC NON-SMALL CELL LUNG CANCER (NSCLC) WHOSE TUMORS HAVE EPIDERMAL GROWTH FACTOR RECEPTOR (EGFR) EXON 19 DELETIONS OR EXON 21(L858R) SUBSTITUTION MUTATIONS AS DETECTED BY AN FDA-APPROVED TEST; USE IN PATIENTS WITH MANTLE CELL LYMPHOMA WHOSE DISEASE HAS RELAPSED OR PROGRESSED AFTER TWO PRIOR THERAPIES, ONE OF WHICH INCLUDED BORTEZOMIB; TREATMENT OF HOSPITAL-ACQUIRED BACTERIAL PNEUMONIA/VENTILATOR-ASSOCIATED BACTERIAL PNEUMONIA (HABP/VABP) CAUSED BY SUSCEPTIBLE ISOLATES OF S. AUREUS (INCLUDING METHICILLIN-SUSCEPTIBLE AND RESISTANT ISOLATES) WHEN ALTERNATIVE TREATMENTS ARE NOT SUITABLE; TREATMENT OF PATIENTS WITH DEPRESSIVE EPISODES ASSOCIATED WITH BIPOLAR I DISORDER (BIPOLAR DEPRESSION) AS MONOTHERAPY AND AS ADJUNCTIVE THERAPY WITH LITHIUM OR VALPROATE; MAINTENANCE TREATMENT OF MAJOR DEPRESSIVE DISORDER; FIRST-LINE TREATMENT OF PATIENTS WITH METASTATIC ADENOCARCINOMA OF THE PANCREAS, IN COMBINATION WITH GEMCITABINE; TREATMENT OF PATIENTS WITH LOCALLY RECURRENT OR METASTATIC, PROGRESSIVE, DIFFERENTIATED THYROID CARCINOMA (DTC) THAT IS REFRACTORY TO RADIOACTIVE IODINE TREATMENT; TRAMETINIB, IN COMBINATION WITH DABRAFENIB, FOR THE TREATMENT OF PATIENTS WITH UNRESECTABLE OR METASTATIC MELANOMA WITH BRAF V600E OR V600K MUTATIONS AS DETECTED BY AN FDA-APPROVED TEST; RISK REDUCTION OF REBLEEDING OF GASTRIC OR DUODENAL ULCERS FOLLOWING THERAPEUTIC ENDOSCOPY IN ADULTS; TREATMENT OF PATIENTS WITH CHRONIC LYMPHOCYTIC LEUKEMIA (CLL) WHO HAVE RECEIVED AT LEAST ONE PRIOR THERAPY; PROPHYLAXIS OF DEEP VEIN THROMBOSIS (DVT) WHICH MAY LEAD TO PULMONARY EMBOLISM (PE), IN ADULT PATIENTS WHO HAVE UNDERGONE HIP OR KNEE REPLACEMENT; TREATMENT OF DEEP VEIN THROMBOSIS (DVT) AND PULMONARY EMBOLISM (PE) IN PATIENTS WHO HAVE BEEN TREATED WITH A PARENTERAL ANTICOAGULANT FOR 5-10 DAYS; TO REDUCE THE RISK OF RECURRENCE OF DVT AND PE IN PATIENTS WHO HAVE BEEN PREVIOUSLY TREATED; PREVENTION OF ACUTE NAUSEA AND VOMITING ASSOCIATED WITH INITIAL AND REPEAT COURSES OF EMETOGENIC CANCER CHEMOTHERAPY, INCLUDING HIGHLY EMETOGENIC CANCER CHEMOTHERAPY IN PEDIATRIC PATIENTS AGED 1 MONTH TO LESS THAN 17 YEARS; EXPANDED INDICATION OF RASAGILINE AS AN ADD-ON THERAPY TO STABLE DOSES OF DOPAMINE AGONISTS IN THE TREATMENT OF EARLY PARKINSON'S DISEASE; INDICATED FOR THE TREATMENT OF DIABETIC MACULAR EDEMA IN PATIENTS WHO ARE PSEUDOPHAKIC OR ARE PHAKIC AND SCHEDULED FOR CATARACT SURGERY; GUIDING SENTINEL LYMPH NODE BIOPSY, USING A HAND-HELD GAMMA COUNTER IN PATIENTS WITH CLINICALLY NODE NEGATIVE SQUAMOUS CELL CARCINOMA OF THE ORAL CAVITY; GADAVIST IS INDICATED WITH MRI TO DETECT THE PRESENCE AND EXTENT OF MALIGNANT BREAST DISEASE; TREATMENT OF PATIENTS WITH CHRONIC LYMPHOCYTIC LEUKEMIA (CLL) WITH 17P DELETION; INDICATED FOR THE TREATMENT OF DEEP VEIN THROMBOSIS (DVT); INDICATED TO REDUCE THE RISK OF RECURRENT DEEP VEIN THROMBOSIS (DVT) AND PULMONARY EMBOLISM (PE) FOLLOWING INITIAL THERAPY; INDICATED FOR MANAGEMENT OF OSTEOARTHRITIS PAIN; TREATMENT OF PATIENTS WITH METASTATIC CASTRATION-RESISTANT PROSTATE CANCER (CRPC); TREATMENT OF PATIENTS WITH MODERATE TO SEVERE PLAQUE PSORIASIS WHO ARE CANDIDATES FOR PHOTOTHERAPY OR SYSTEMIC THERAPY; REVISED INDICATION FOR BORTEZOMIB IN THE TREATMENT OF PATIENTS WITH MANTLE CELL LYMPHOMA; USE AS MONOTHERAPY IN THE TREATMENT OF PARTIAL-ONSET SEIZURES IN PATIENTS WITH EPILEPSY AGE 17 YEARS AND OLDER; FOR USE IN COMBINATION WITH SOFOSBUVIR FOR THE TREATMENT OF PATIENTS WITH CHRONIC HEPATITIS C VIRUS GENOTYPE 1 INFECTION; SCHIZOAFFECTIVE DISORDER AS MONOTHERAPY AND AS AN ADJUNCT TO MOOD STABILIZERS OR ANTIDEPRESSANTS; FOR TREATMENT OF PATIENTS WITH POLYCYTHEMIA VERA WHO HAVE HAD AN INADEQUATE RESPONSE TO OR ARE INTOLERANT OF HYDROXYUREA; TREATMENT OF PEDIATRIC PATIENTS WITH TOURETTE'S DISORDER (6-18 YEARS); FOR THE TREATMENT OF PATIENTS WITH UNRESECTABLE, WELL-OR MODERATELY-DIFFERENTIATED, LOCALLY ADVANCED OR METASTATIC GASTROENTEROPANCREATIC NEUROENDOCRINE TUMORS (GEP-NETS) TO IMPROVE PROGRESSION FREE SURVIVAL; FOR THE TREATMENT OF PATIENTS WITH WALDENSTROM MACROGLOBULINEMIA; MODERATE TO SEVERE BINGE EATING DISORDER (BED); EXPANDED INDICATION TO INCLUDE PATIENTS WHO ARE VIROLOGICALLY-SUPPRESSED (HIV-1 RNA<50 COPIES/ML) ON A STABLE ANTIRETROVIRAL REGIMEN FOR AT LEAST 6 MONTHS WITH NO HISTORY OF TREATMENT FAILURE IN ORDER TO REPLACE THEIR CURRENT REGIMEN; TREATMENT OF CYSTIC FIBROSIS IN PATIENTS 6 YEARS AND OLDER WHO HAVE AN R117H MUTATION IN THE CFTR GENE; EXPANDED INDICATION FOR THE TREATMENT OF PATIENTS WITH MULTIPLE MYELOMA; POMALYST, IN COMBINATION WITH DEXAMETHASONE, IS INDICATED FOR PATIENTS WITH MULTIPLE MYELOMA WHO HAVE RECEIVED AT LEAST 2 PRIOR THERAPIES AND HAVE DEMONSTRATED DISEASE PROGRESSION ON OR WITHIN 60 DAYS OF COMPLETION OF THE LAST THERAPY; DAILY TREATMENT OF ASTHMA IN PATIENTS AGED 18 YEARS AND OLDER; TREATMENT OF IRRITABLE BOWEL SYNDROME WITH DIARRHEA (IBS-D) IN ADULTS; ADJUNCTIVE THERAPY FOR THE TREATMENT OF PRIMARY GENERALIZED TONIC-CLONIC (PG TC) SEIZURES IN PATIENTS WITH EPILEPSY 12 YEARS OF AGE OR OLDER; INCLUSION OF PEDIATRIC PATIENTS AGES 6 YRS AND OLDER FOR THE TREATMENT OF THROMBOCYTOPENIA IN PATIENTS WITH CHRONIC ITP WHO HAVE HAD AN INSUFFICIENT RESPONSE TO CORTICOSTEROIDS, IMMUNOGLOBULINS, OR SPLENECTOMY; EXPANDED INDICATION FOR USE IN COMBINATION WITH LENALIDOMIDE AND DEXAMETHASONE FOR THE TREATMENT OF PATIENTS WITH RELAPSED MULTIPLE MYELOMA WHO HAVE RECEIVED ONE TO THREE PRIOR LINES OF THERAPY; REVISIONS TO THE LABELING TO PERMIT THE USE OF ZUBSOLV AS INITIAL (‘INDUCTION’) TREATMENT OF OPIOID DEPENDENCE; EXTENDS THE 2011 APPROVAL OF BRILINTA FOR USE BEGINNING WITH ACS TO USE BEGINNING MORE REMOTE FROM MYOCARDIAL INFARCTION; FOR THE ADDITION OF THE INDICATION FOR MONOTHERAPY TREATMENT IN PARTIAL-ONSET SEIZURES IN ADULTS; REVISED INDICATION TO INCLUDE LANGUAGE ABOUT THE BENEFITS OF USING LETAIRIS IN COMBINATION WITH TADALAFIL TO REDUCE THE RISK OF DISEASE PROGRESSION AND HOSPITALIZATION FOR WORSENING PAH AND TO IMPROVE EXERCISE ABILITY, BASED ON THE AMBITION STUDY; EXPANDED INDICATION TO INCLUDE THE TREATMENT OF CHRONIC HEPATITIS C GENOTYPE 4; EXPANDED INDICATION TO INCLUDE SUBJECTS INFECTED WITH CHRONIC HEPATITIS C, GENOTYPE 6 VIRUS INFECTION BASED UPON THE RESULTS OF THE ELECTRON-2 STUDY; EXPANDED INDICATION TO INCLUDE THE TREATMENT OF SUBJECTS WITH GENOTYPE 5 CHRONIC HEPATITIS C VIRUS INFECTION BASED ON THE RESULTS FROM STUDY GS-US-337-119; EXPANDED INDICATION TO INCLUDE TREATMENT OF GENOTYPE 4, CHRONIC HEPATITIS C VIRUS INFECTION BASED UPON THE RESULTS FROM STUDIES ION-4 AND GS-US-337-119; TREATMENT OF PATIENTS WITH UNRESECTABLE OR METASTATIC LIPOSARCOMA WHO HAVE RECEIVED A PRIOR ANTHRACYCLINE-CONTAINING REGIMEN; REVISED INDICATION FOR USE IN COMBINATION WITH DEXAMETHASONE OR WITH LENALIDOMIDE PLUS DEXAMETHASONE FOR THE TREATMENT OF PATIENTS WITH RELAPSED OR REFRACTORY MULTIPLE MYELOMA WHO HAVE RECEIVED ONE TO THREE LINES OF THERAPY; AS A SINGLE AGENT FOR THE TREATMENT OF PATIENTS WITH RELAPSED OR REFRACTORY MULTIPLE MYELOMA WHO HAVE RECEIVED ONE OR MORE LINES OF THERAPY; TREATMENT OF ADULT PATIENTS WITH PROGRESSIVE, WELL DIFFERENTIATED, NON-FUNCTIONAL NEUROENDOCRINE TUMORS (NET) OF GI OR LUNG ORIGIN WITH UNRESECTABLE, LOCALLY ADVANCED OR METASTATIC DISEASE; TREATMENT OF HORMONE RECEPTOR (HR)-POSITIVE, HER2-NEGATIVE ADVANCED OR METASTATIC BREAST CANCER IN COMBINATION THERAPY WITH PALBOCICLIB AND FULVESTRANT IN WOMEN WITH DISEASE PROGRESSION FOLLOWING ENDOCRINE THERAPY; EXPANSION OF THE PATIENT POPULATION TO INCLUDE PATIENTS WITH RECURRENCE OF HEPATITIS C VIRUS (HCV) GENOTYPE 1 OR 3 AFTER LIVER TRANSPLANTATION; EXPANSION OF THE INDICATION TO INCLUDE TREATMENT OF SUBJECTS WITH GENOTYPE-1 CHRONIC HEPATITIS C VIRUS INFECTION, INCLUDING SUBJECTS WHO ARE CO-INFECTED WITH THE HUMAN IMMUNODEFICIENCY VIRUS (HIV-1) BASED ON THE RESULTS FROM THE ALLY-2 CLINICAL TRIAL; EXPANDED INDICATION FOR USE IN ULTRASONOGRAPHY OF THE LIVER FOR CHARACTERIZATION OF FOCAL LIVER LESIONS IN ADULT AND PEDIATRIC PATIENTS; PROVIDES FOR THE FRONTLINE INDICATION FOR THE TREATMENT OF CHRONIC LYMPHOCYTIC LEUKEMIA; NEW INDICATION FOR THE TREATMENT OF PATIENTS WITH METASTATIC, SQUAMOUS, NON-SMALL CELL LUNG CANCER PROGRESSING AFTER PLATINUM-BASED CHEMOTHERAPY; FOR USE IN MAGNETIC RESONANCE ANGIOGRAPHY IN ADULT AND PEDIATRIC PATIENTS (INCLUDING TERM NEONATES) TO EVALUATE KNOWN OR SUSPECTED SUPRA-AORTIC OR RENAL ARTERY DISEASE; TREATMENT OF PEDIATRIC PATIENTS 7 TO 17 YEARS OF AGE WITH HOMOZYGOUS FAMILIAL HYPERCHOLESTEROLEMIA TO REDUCE LDL-C, TOTAL C, NONHDL-C AND APOB AS AN ADJUNCT TO DIET, EITHER ALONE OR WITH OTHER LIPID-LOWERING TREATMENTS; USE OF CANAGLIFLOZIN FOR INITIAL THERAPY IN COMBINATION WITH METFORMIN; EXPANDED INDICATION FOR THE USE OF LENVIMA IN COMBINATION WITH EVEROLIMUS FOR THE TREATMENT OF PATIENTS WITH ADVANCED RCC FOLLOWING ONE PRIOR ANTI-ANGIOGENIC THERAPY; AS AN ADJUNCT TO DIET AND EXERCISE TO IMPROVE GLYCEMIC CONTROL IN ADULTS WITH TYPE 2 DIABETES MELLITUS WHEN TREATMENT WITH BOTH CANAGLIFLOZIN AND METFORMIN IS APPROPRIATE; REVISED INDICATION TO INCLUDE THE TREATMENT OF PATIENTS WITH SMALL LYMPHOCYTIC LEUKEMIA (SLL); REVISED INDICATION TO INCLUDE THE TREATMENT OF PATIENTS WITH SMALL LYMPHOCYTIC LEUKEMIA (SLL) WITH 17P DELETION; REVISIONS TO THE INDICATIONS AND USAGE SECTION WITH RESPECT TO COMPLICATED INTRA-ABDOMINAL INFECTIONS; TO REDUCE THE RISK OF CARDIOVASCULAR DEATH IN ADULT PATIENTS WITH TYPE 2 DIABETES MELLITUS AND ESTABLISHED CARDIOVASCULAR DISEASE; EXPANDED INDICATION FOR THE TREATMENT OF CYSTIC FIBROSIS IN PATIENTS AGE 6 YEARS AND OLDER TO INCLUDE THE G1244E, G1349D, G178R, G551S, 51251N, 51255P, S549N, OR S549R MUTATION IN THE CFTR GENE; TREATMENT OF PATIENTS WITH MARGINAL ZONE LYMPHOMA (MZL) WHO REQUIRE SYSTEMIC THERAPY AND HAVE RECEIVED AT LEAST ONE PRIOR ANTI-CD20-BASED THERAPY; TREATMENT OF NODAL MARGINAL ZONE LYMPHOMA; INFORMATION ADDED TO THE LABELING FOR THE ADDITION OF THE TREATMENT OF CHRONIC HEPATITIS C VIRUS (HCV) GENOTYPE 4 (GT4) INFECTED PATIENTS WITH COMPENSATED CIRRHOSIS BASED ON RESULTS FROM STUDY M11-665; TREATMENT OF PATIENTS WITH HEPATOCELLULAR CARCINOMA (HCC) WHO HAVE BEEN PREVIOUSLY TREATED WITH SORAFENIB; MEKINIST, IN COMBINATION WITH DABRAFENIB, FOR THE TREATMENT OF PATIENTS WITH METASTATIC NON-SMALL CELL LUNG CANCER (NSCLC) WITH BRAF V600E MUTATION AS DETECTED BY AN FDA-APPROVED TEST; NEW INDICATION OF MAINTENANCE MONOTHERAPY TREATMENT OF BIPOLAR I DISORDER IN ADULTS; FOR REDUCING THE RISK OF GRAFT REJECTION WHEN USED WITH HIGH-DOSE BUSULFAN AND CYCLOPHOSPHAMIDE AS A PREPARATIVE REGIMEN FOR ALLOGENEIC HEMATOPOIETIC PROGENITOR (STEM) CELL TRANSPLANTATION FOR PEDIATRIC PATIENTS WITH CLASS 3 BETA-THALASSEMIA; TO REDUCE THE ACUTE COMPLICATIONS OF SICKLE CELL DISEASE IN ADULT AND PEDIATRIC PATIENTS FIVE YEARS OF AGE AND OLDER; MONOTHERAPY FOR THE TREATMENT OF HORMONE RECEPTOR (HR) POSITIVE, HUMAN EPIDERMAL GROWTH FACTOR RECEPTOR 2 (HER2)-NEGATIVE, ADVANCED BREAST CANCER IN POSTMENOPAUSAL WOMEN NOT PREVIOUSLY TREATED WITH ENDOCRINE THERAPY; REDUCE THE RISK OF MAJOR ADVERSE CARDIOVASCULAR EVENTS IN ADULTS WITH TYPE 2 DIABETES MELLITUS AND ESTABLISHED CARDIOVASCULAR DISEASE; TREATMENT OF TARDIVE DYSKINESIA; CORONARY COMPUTED TOMOGRAPHY ANGIOGRAPHY (CCTA) TO ASSIST DIAGNOSTIC EVALUATION OF PATIENTS WITH SUSPECTED CORONARY ARTERY DISEASE; TREATMENT OF ADULT PATIENTS WITH CHRONIC GRAFT VERSUS HOST DISEASE (CGVHD) AFTER FAILURE OF ONE OR MORE LINES OF SYSTEMIC THERAPY; TO REDUCE THE FREQUENCY OF SHORT-ACTING SOMATOSTATIN ANALOG RESCUE THERAPY WHEN USED FOR THE TREATMENT OF ADULTS WITH CARCINOID SYNDROME; ADJUVANT TREATMENT OF ADULT PATIENTS AT HIGH RISK OF RECURRENT RENAL CELL CARCINOMA (RCC) FOLLOWING NEPHRECTOMY; EXPANDED THE APPROVED INDICATION BY REMOVING THE RESTRICTION FOR USE ONLY IN PATIENTS WHO HAVE PROGRESSED ON OR ARE INTOLERANT TO CRIZOTINIB; TREATMENT OF PATIENTS WITH ERDHEIM-CHESTER DISEASE WITH BRAF V600 MUTATION; FOR USE WITH RILPIVIRINE AS A COMPLETE REGIMEN TO REPLACE THE CURRENT ARV REGIMEN IN VIROLOGICALLY SUPPRESSED PATIENTS ON A STABLE ARV REGIMEN FOR AT LEAST 6 MONTHS WITH NO HISTORY OF TX FAILURE OR KNOWN SUBSTITUTIONS ASSOC. WITH RESISTANCE TO EITHER ARV; TREATMENT OF ADULT PATIENTS WITH NEWLY-DIAGNOSED CHRONIC PHASE (CP) PHILADELPHIA CHROMOSOME-POSITIVE CHRONIC MYELOGENOUS LEUKEMIA (PH+CML); FOR THE TREATMENT OF PATIENTS WITH ADVANCED RENAL CELL CARCINOMA; TREATMENT OF ADULT PATIENTS WITH ACTIVE PSORIATIC ARTHRITIS WHO HAVE HAD AN INADEQUATE RESPONSE OR INTOLERANCE TO METHOTREXATE OR OTHER NON-BIOLOGIC DISEASE-MODIFYING ANTIRHEUMATIC DRUGS; TREATMENT OF DELETERIOUS OR SUSPECTED DELETERIOUS GERMLINE BRCA-MUTATED, HUMAN EPIDERMAL GROWTH FACTOR RECEPTOR 2-NEGATIVE METASTATIC BREAST CANCER WHO HAVE BEEN TREATED WITH CHEMOTHERAPY IN THE NEOADJUVANT, ADJUVANT OR METASTATIC SETTING; TREATMENT OF PATIENTS WITH METASTATIC NON-SMALL CELL LUNG CANCER WHOSE TUMORS HAVE NON-RESISTANT EPIDERMAL GROWTH FACTOR RECEPTOR MUTATIONS AS DETECTED BY AN FDA-APPROVED TEST; TREATMENT IN ADULT PATIENTS FOR IRRITABLE BOWEL SYNDROME WITH CONSTIPATION (IBS-C); ABIRATERONE ACETATE IN COMBINATION WITH PREDNISONE FOR THE TREATMENT OF PATIENTS WITH METASTATIC HIGH-RISK CASTRATION-SENSITIVE PROSTATE CANCER; TREATMENT OF MINIMALLY TO MODERATELY THICK ACTINIC KERATOSIS OF THE UPPER EXTREMITIES IN CONJUNCTION WITH A BLUE LIGHT PHOTODYNAMIC THERAPY ILLUMINATOR; TREATMENT OF IRON DEFICIENCY ANEMIA IN ADULT PATIENTS WHO HAVE INTOLERANCE TO ORAL IRON OR HAVE HAD UNSATISFACTORY RESPONSE TO ORAL IRON; IN COMBINATION WITH AN AROMATASE INHIBITOR AS INITIAL ENDOCRINE-BASED THERAPY FOR THE TREATMENT OF POSTMENOPAUSAL WOMEN WITH HORMONE RECEPTOR-POSITIVE, HUMAN EPIDERMAL GROWTH FACTOR RECEPTOR 2-NEGATIVE ADVANCED OR METASTATIC BREAST CANCER; TREATMENT OF DYSKINESIA IN PATIENTS WITH PARKINSON'S DISEASE RECEIVING LEVODOPA-BASED THERAPY, WITH OR WITHOUT CONCOMITANT DOPAMINERGIC MEDICATIONS; TREATMENT OF ACUTE OTITIS EXTERNA IN PATIENTS 6 MONTHS OF AGE AND OLDER DUE TO PSEUDOMONAS AERUGINOSA AND STAPHYLOCOCCUS AUREUS; REVISION OF THE INDICATION SECTION OF THE PACKAGE INSERT REGARDING AN INTERSCALENE BRACHIAL PLEXUS NERVE BLOCK TO PRODUCE POSTSURGICAL REGIONAL ANALGESIA; FOR THE MAINTENANCE TREATMENT OF ADULT PATIENTS WITH RECURRENT EPITHELIAL OVARIAN, FALLOPIAN TUBE, OR PRIMARY PERITONEAL CANCER WHO ARE IN A COMPLETE OR PARTIAL RESPONSE TO PLATINUM-BASED CHEMOTHERAPY; FOR THE ADJUNCTIVE TREATMENT OF ADULT AND PEDIATRIC PATIENTS AGE 2 YEARS AND OLDER WITH TUBEROUS SCLEROSIS COMPLEX (TSC)-ASSOCIATED PARTIAL-ONSET SEIZURES; TO ALLOW FOR FIRST-LINE TREATMENT OF PATIENTS WITH METASTATIC NON-SMALL CELL LUNG CANCER (NSCLC) WHOSE TUMORS HAVE EPIDERMAL GROWTH FACTOR RECEPTOR (EGFR) EXON 19 DELETIONS OR EXON 21 (L858R) SUBSTITUTION MUTATIONS, AS DETECTED BY AN FDA APPROVED TEST; REVISED INDICATION FOR FIXED-DOSE COMBINATION OF FLUTICASONE FUROATE, UMECLIDINIUM, AND VILANTEROL TO TREAT AIRFLOW OBSTRUCTION IN CHRONIC OBSTRUCTIVE PULMONARY DISEASE (COPD) AND TO REDUCE COPD EXACERBATIONS IN PTS WITH HISTORY OF EXACERBATIONS; FIRSTLINE MAINTENANCE TX IN PTS W/DELETERIOUS OR SUSPECTED DELETERIOUS GERMLINE, SOMATIC BRCA-MUTATED ADVANCED EPITHELIAL OVARIAN, FALLOPIAN TUBE OR PRIMARY PERITONEAL CA WHO ARE IN COMPLETE OR PARTIAL RESPONSE TO FIRSTLINE PLATINUM-BASED CHEMOTHERAPY; CO-ADMINISTRATION THERAPY OF MIRABEGRON WITH SOLIFENACIN SUCCINATE FOR TREATMENT OF OVERACTIVE BLADDER WITH SYMPTOMS OF URGE URINARY INCONTINENCE, URGENCY, AND URINARY FREQUENCY; DABRAFENIB IN COMBINATION WITH TRAMETINIB FOR THE ADJUVANT TREATMENT OF PATIENTS WITH MELANOMA WITH BRAF V600E OR V600K MUTATIONS, AS DETECTED BY AN FDA-APPROVED TEST, AND INVOLVEMENT OF LYMPH NODE(S), FOLLOWING COMPLETE RESECTION; USE OF TOLVAPTAN TO SLOW KIDNEY FUNCTION DECLINE IN ADULTS AT RISK OF RAPIDLY PROGRESSING AUTOSOMAL DOMINANT POLYCYSTIC KIDNEY DISEASE (ADPKD); TREATMENT OF ADULT PATIENTS WITH MODERATELY TO SEVERELY ACTIVE ULCERATIVE COLITIS (UC); DABRAFENIB IN COMBINATION WITH TRAMETINIB FOR THE TREATMENT OF PATIENTS WITH LOCALLY ADVANCED OR METASTATIC ANAPLASTIC THYROID CANCER (ATC) WITH BRAF V600E MUTATION AND WITH NO SATISFACTORY LOCOREGIONAL TREATMENT OPTIONS; REVISIONS TO INDICATION FOR THE TREATMENT OF PATIENTS WITH CHRONIC LYMPHOCYTIC LEUKEMIA (CLL) OR SMALL LYMPHOCYTIC LYMPHOMA (SLL), WITH OR WITHOUT 17P DELETION, WHO HAVE RECEIVED AT LEAST ONE PRIOR THERAPY; EXPANDED INDICATION TO INCLUDE RIBOCICLIB WITH AN AROMATASE INHIBITOR IN PRE/PERIMENOPAUSAL WOMEN WITH HORMONE RECEPTOR-POSITIVE, HUMAN EPIDERMAL GROWTH FACTOR RECEPTOR 2-NEGATIVE ADVANCED OR METASTATIC BREAST CANCER, AS INITIAL ENDOCRINE-BASED THERAPY; RIBOCICLIB WITH FULVESTRANT FOR THE TREATMENT OF POSTMENOPAUSAL WOMEN WITH HR-POSITIVE, HER2-NEGATIVE ADVANCED OR METASTATIC BREAST CANCER, AS INITIAL ENDOCRINE BASED THERAPY OR FOLLOWING DISEASE PROGRESSION ON ENDOCRINE THERAPY; TREATMENT OF PATIENTS WITH CUSHING'S DISEASE FOR WHOM PITUITARY SURGERY IS NOT AN OPTION OR HAS NOT BEEN CURATIVE; TREATMENT OF PATIENTS WITH NON-METASTATIC CASTRATION-RESISTANT PROSTATE CANCER; FIRST-LINE TREATMENT OF PATIENTS WITH UNRESECTABLE HEPATOCELLULAR CARCINOMA (HCC); NEW INDICATION FOR CANAGLIFLOZIN TO REDUCE THE RISK OF MAJOR ADVERSE CARDIOVASCULAR EVENTS (CARDIOVASCULAR DEATH, NONFATAL MYOCARDIAL INFARCTION AND NONFATAL STROKE) IN ADULTS WITH TYPE 2 DIABETES MELLITUS AND ESTABLISHED CARDIOVASCULAR DISEASE (CVD); VENETOCLAX IN COMBO WITH AZACITIDINE OR DECITABINE OR LOW-DOSE CYTARABINE FOR THE TX OF NEWLY-DIAGNOSED ACUTE MYELOID LEUKEMIA IN ADULTS WHO ARE AGE 75 YEARS OR OLDER, OR WHO HAVE COMORBIDITIES THAT PRECLUDE USE OF INTENSIVE INDUCTION CHEMOTHERAPY; USE OF FERRIC CITRATE FOR THE TREATMENT OF IRON DEFICIENCY ANEMIA IN ADULT PATIENTS WITH CKD NOT ON DIALYSIS; TREATMENT OF PEDIATRIC PATIENTS ONE YEAR OF AGE AND OLDER WITH NEWLY DIAGNOSED PHILADELPHIA CHROMOSOME-POSITIVE (PH+) ACUTE LYMPHOBLASTIC LEUKEMIA (ALL) IN COMBINATION WITH CHEMOTHERAPY; TREATMENT OF PATIENTS WITH HEPATOCELLULAR CARCINOMA WHO HAVE BEEN PREVIOUSLY TREATED WITH SORAFENIB; TREATMENT OF MODERATE TO SEVERE VAGINAL DRYNESS, A SYMPTOM OF VULVAR AND VAGINAL ATROPHY, DUE TO MENOPAUSE; TREATMENT OF ADULT PATIENTS WITH METASTATIC GASTRIC OR GASTROESOPHAGEAL JUNCTION ADENOCARCINOMA PREVIOUSLY TREATED WITH AT LEAST TWO PRIOR LINES OF CHEMOTHERAPY, AND IF APPROPRIATE, HER2/NEU-TARGETED THERAPY; VENETOCLAX IN COMBINATION WITH OBINUTUZUMAB IN PREVIOUSLY UNTREATED PATIENTS WITH CHRONIC LYMPHOCYTIC LEUKEMIA OR SMALL LYMPHOCYTIC LYMPHOMA; USED IN COMBINATION WITH A RITUXIMAB PRODUCT, ARE INDICATED FOR THE TREATMENT OF ADULT PATIENTS WITH PREVIOUSLY TREATED FOLLICULAR LYMPHOMA (FL); USED IN COMBINATION WITH A RITUXIMAB PRODUCT, ARE INDICATED FOR THE TREATMENT OF ADULT PATIENTS WITH PREVIOUSLY TREATED MARGINAL ZONE LYMPHOMA (MZL); TREATMENT OF DEPRESSIVE EPISODES ASSOCIATED WITH BIPOLAR I DISORDER (BIPOLAR DEPRESSION); TREATMENT OF STEROID-REFRACTORY ACUTE GRAFT-VERSUS-HOST DISEASE (GVHD) IN ADULT AND PEDIATRIC PATIENTS 12 YEARS AND OLDER; TREATMENT OF OCULAR INFLAMMATION FOLLOWING OPHTHALMIC SURGERY; USE IN CARDIAC MRI TO ASSESS MYOCARDIAL PERFUSION (STRESS, REST) AND LATE GADOLINIUM ENHANCEMENT IN ADULT PATIENTS WITH KNOWN OR SUSPECTED CORONARY ARTERY DISEASE (CAD); TREATMENT OF THROMBOCYTOPENIA IN ADULT PATIENTS WITH CHRONIC IMMUNE THROMBOCYTOPENIA WHO HAVE HAD AN INSUFFICIENT RESPONSE TO A PREVIOUS TREATMENT; TREATMENT OF ADULT PATIENTS WITH ORAL ULCERS ASSOCIATED WITH BEHCETS DISEASE; EXPANDED INDICATION FOR USE AS AN ADJUNCT TO DIET AND EXERCISE TO IMPROVE GLYCEMIC CONTROL IN ADULTS WITH TYPE 2 DIABETES MELLITUS; SLOW THE RATE OF DECLINE IN PULMONARY FUNCTION IN PATIENTS WITH SYSTEMIC SCLEROSIS-ASSOCIATED INTERSTITIAL LUNG DISEASE; EXPANDED INDICATION FOR PTS WHO ARE VIROLOGICALLY SUPPRESSED (HIV-1 RNA LESS THAN 50 COPIES/ML) ON A STABLE ARV REGIMEN WITH NO HX OF TX FAILURE AND NO KNOWN SUBSTITUTIONS ASSOCIATED W RESISTANCE TO DORAVIRINE, LAMIVUDINE OR TENOFOVIR DISOPROXIL FUMARATE; TREATMENT OF ADVANCED ENDOMETRIAL CARCINOMA THAT IS NOT MICROSATELLITE INSTABILITY-HIGH OR MISMATCH REPAIR DEFICIENT, WHO HAVE DISEASE PROGRESSION FOLLOWING PRIOR SYSTEMIC THERAPY AND ARE NOT CANDIDATES FOR CURATIVE SURGERY OR RADIATION; TREATMENT OF PATIENTS WITH METASTATIC CASTRATION-SENSITIVE PROSTATE CANCER (MCSPC); TO REDUCE THE RISK OF END-STAGE KIDNEY DISEASE, DOUBLING OF SERUM CREATININE, CARDIOVASCULAR DEATH, AND HOSPITALIZATION FOR HEART FAILURE IN ADULTS WITH TYPE 2 DIABETES MELLITUS AND DIABETIC NEPHROPATHY WITH ALBUMINURIA>300 MG/DAY; PROPHYLAXIS OF VENOUS THROMBOEMBOLISM IN ACUTELY ILL MEDICAL PATIENTS AT RISK FOR THROMBOEMBOLIC COMPLICATIONS NOT AT HIGH RISK OF BLEEDING; TREATMENT OF ACUTE UNCOMPLICATED INFLUENZA IN PATIENTS 12 YEARS OF AGE OR OLDER, WHO HAVE BEEN SYMPTOMATIC FOR NO MORE THAN 48 HOURS AND ARE AT HIGH RISK OF DEVELOPING INFLUENZA-RELATED COMPLICATIONS; FOR USE IN AT RISK ADULTS AND ADOLESCENTS WEIGHING AT LEAST 35 KG FOR PRE-EXPOSURE PROPHYLAXIS TO REDUCE THE RISK OF HIV-1 INFECTION FROM SEXUAL ACQUISITION, EXCLUDING INDIVIDUALS AT RISK FROM RECEPTIVE VAGINAL SEX; TX OF ADULT PTS W/ADV OVARIAN FALLOPIAN TUBE OR PRIMARY PERITONEAL CANCER TREATED W/>=3 PRIOR CHEMO REGIMENS & ASSOCIATED W/HRD DEFICIENCY POSITIVE STATUS DEFINED BY A DELETERIOUS OR SUSPECTED DELETERIOUS BRCA MUTATION; TX OF ADV OVARIAN FALLOPIAN TUBE OR PRIMARY PERITONEAL CANCER TREATED W/ >=3 PRIOR CHEMO REGIMENS & ASSOCIATED W/HRD DEFICIENCY DEFINED BY POSITIVE STATUS GENOMIC INSTABILITY & WHO HAVE PROGRESSED >6MO AFTER RESPONSE TO LAST PLATINUM-BASED CHEMO; TREATMENT OF COMMUNITY ACQUIRED BACTERIAL PNEUMONIA (CABP) CAUSED BY DESIGNATED SUSCEPTIBLE BACTERIA IN ADULTS; TREATMENT OF ADULT PATIENTS WITH NEWLY-DIAGNOSED ACUTE MYELOID LEUKEMIA (AML) WHO ARE >=75 YEARS OLD OR WHO HAVE COMORBIDITIES THAT PRECLUDE USE OF INTENSIVE INDUCTION CHEMOTHERAPY; TREATMENT OF ADULT PATIENTS WITH CHRONIC LYMPHOCYTIC LEUKEMIA (CLL) OR SMALL LYMPHOCYTIC LYMPHOMA (SLL); MAINTENANCE TREATMENT OF ADULT PATIENTS WITH DELETERIOUS OR SUSPECTED DELETERIOUS GBRCAM METASTATIC PANCREATIC ADENOCARCINOMA WHOSE DISEASE HAS NOT PROGRESSED ON AT LEAST 16 WEEKS OF A FIRST-LINE PLATINUM-BASED CHEMOTHERAPY REGIMEN; ADJUNCT TO MAX TOLERATED STATIN TX TO REDUCE RISK OF MI, STROKE, CORONARY REVASCULARIZATION, & UNSTABLE ANGINA REQUIRING HOSPITALIZATION IN ADULTS W/ELEVATED TG LEVELS & ESTABLISHED CV DISEASE OR DIABETES MELLITUS & 2+ RISK FACTORS FOR CV DISEASE; INDICATED FOR THE TREATMENT OF PULMONARY ARTERIAL HYPERTENSION (PAH) (WHO GROUP 1) TO DELAY DISEASE PROGRESSION; TREATMENT OF CANDIDEMIA, ACUTE DISSEMINATED CANDIDIASIS, CANDIDA PERITONITIS AND ABSCESSES WITHOUT MENINGOENCEPHALITIS AND/OR OCULAR DISSEMINATION IN PEDIATRIC PATIENTS YOUNGER THAN 4 MONTHS OF AGE; REDUCE THE RISK OF MAJOR ADVERSE CARDIOVASCULAR EVENTS (CARDIOVASCULAR DEATH, NON-FATAL MYOCARDIAL INFARCTION OR NON-FATAL STROKE) IN ADULTS WITH TYPE 2 DIABETES MELLITUS AND ESTABLISHED CARDIOVASCULAR DISEASE; USE IN COMBINATION WITH CAPECITABINE, FOR THE TREATMENT OF ADULT PATIENTS WITH ADVANCED OR METASTATIC HER2-POSITIVE BREAST CANCER WHO HAVE RECEIVED TWO OR MORE PRIOR ANTI-HER2 BASED REGIMENS IN THE METASTATIC SETTING; RIVAROXABAN IN COMBINATION WITH ASPIRIN, IS INDICATED TO REDUCE THE RISK OF MAJOR CV EVENTS (CV DEATH, MI, AND STROKE) IN PATIENTS WITH CHRONIC CORONARY ARTERY DISEASE (CAD) OR PERIPHERAL ARTERY DISEASE (PAD); TREATMENT FOR CHRONIC FIBROSING INTERSTITIAL LUNG DISEASES WITH A PROGRESSIVE PHENOTYPE; ENCORAFENIB, IN COMBINATION WITH CETUXIMAB, FOR THE TREATMENT OF ADULT PATIENTS WITH METASTATIC COLORECTAL CANCER WITH A BRAF V600E MUTATION, AS DETECTED BY AN FDA-APPROVED TEST, AFTER PRIOR THERAPY; EXPANDED INDICATION FOR PATIENTS WHO ARE VIROLOGICALLY SUPPRESSED (HIV-1 RNA LESS THAN 50 COPIES/ML) ON A STABLE ARV REGIMEN WITH NO HISTORY OF TREATMENT FAILURE AND NO KNOWN SUBSTITUTIONS ASSOCIATED WITH RESISTANCE TO DORAVIRINE; INDICATED FOR THE TREATMENT OF ADULT PATIENTS WITH CUSHING'S DISEASE FOR WHOM PITUITARY SURGERY IS NOT AN OPTION OR HAS NOT BEEN CURATIVE; TREATMENT OF PEDIATRIC PATIENTS 2 YEARS OF AGE AND OLDER WITH CENTRAL PRECOCIOUS PUBERTY (CPP); TREATMENT OF ADULT PATIENTS WITH A DELETERIOUS BRCA MUTATION (GERMLINE AND/OR SOMATIC)-ASSOCIATED METASTATIC CASTRATION-RESISTANT PROSTATE CANCER WHO HAVE BEEN TREATED WITH ANDROGEN RECEPTOR-DIRECTED THERAPY AND A TAXANE-BASED CHEMOTHERAPY; W/BEVACIZUMAB FOR MAINTENANCE TX OF ADULTS W/ADV. EPITHELIAL OVARIAN, FALLOPIAN TUBE OR PRIMARY PERITONEAL CA IN COMPLETE OR PARTIAL RESPONSE TO FIRST-LINE PLATINUM-BASED CHEMO & CA ASSOCIATED W/HOMOLOGOUS RECOMBINATION DEFICIENCY POSITIVE STATUS; TX OF ADULT PTS WITH DELETERIOUS OR SUSPECTED DELETERIOUS GERMLINE OR SOMATIC HOMOLOGOUS RECOMBINATION REPAIR GENE-MUTATED METASTATIC CASTRATION-RESISTANT PROSTATE CANCER WHO HAVE PROGRESSED FOLLOWING PRIOR TREATMENT WITH ENZALUTAMIDE OR ABIRATERONE; MAINTENANCE TREATMENT OF ADULT PATIENTS WITH ADVANCED EPITHELIAL OVARIAN, FALLOPIAN TUBE, OR PRIMARY PERITONEAL CANCER WHO ARE IN A COMPLETE OR PARTIAL RESPONSE TO FIRST-LINE PLATINUM-BASED CHEMOTHERAPY; TO REDUCE THE RISK OF CARDIOVASCULAR DEATH AND HOSPITALIZATION FOR HEART FAILURE IN ADULTS WITH HEART FAILURE (NYHA CLASS II-IV) WITH REDUCED EJECTION FRACTION; TREATMENT OF ADULT PATIENTS WITH RELAPSED OR REFRACTORY (R/R) FOLLICULAR LYMPHOMA (FL) WHOSE TUMORS ARE POSITIVE FOR AN EZH2 MUTATION AS DETECTED BY AN FDA-APPROVED TEST AND WHO HAVE RECEIVED AT LEAST 2 PRIOR SYSTEMIC THERAPIES; TREATMENT OF ADULT PATIENTS WITH RELAPSED OR REFRACTORY (R/R) FOLLICULAR LYMPHOMA (FL) WHO HAVE NO SATISFACTORY ALTERNATIVE TREATMENT OPTIONS; TREATMENT OF ADULT PATIENTS WITH RELAPSED OR REFRACTORY DIFFUSE LARGE B-CELL LYMPHOMA (DLBCL), NOT OTHERWISE SPECIFIED, INCLUDING DLBCL ARISING FROM FOLLICULAR LYMPHOMA, AFTER AT LEAST 2 LINES OF SYSTEMIC THERAPY; TREATMENT OF NEUROPATHIC PAIN ASSOCIATED WITH DIABETIC PERIPHERAL NEUROPATHY (DPN) OF THE FEET; TO REPLACE THE CURRENT ANTIRETROVIRAL REGIMEN IN THOSE WHO ARE VIROLOGICALLY SUPRESSED (HIV-1 RNA LESS THAN 50 COPIES PER ML) ON A STABLE ANTIRETROVIRAL REGIMEN WITH NO HISTORY OF TREATMENT FAILURE; TREATMENT OF SYMPTOMS IN ADULTS WITH MAJOR DEPRESSIVE DISORDER (MDD) WITH ACUTE SUICIDAL IDEATION OR BEHAVIOR; TO REDUCE THE RISK OF HOSPITALIZATION FOR HEART FAILURE IN ADULTS WITH TYPE 2 DIABETES MELLITUS AND ESTABLISHED CARDIOVASCULAR DISEASE OR MULTIPLE CARDIOVASCULAR RISK FACTORS; TREATMENT OF ADULT PATIENTS WITH RELAPSED ORREFRACTORY MULTIPLE MYELOMA WHO HAVE RECEIVED ONE TO THREE LINES OF THERAPY IN COMBINATION WITH DARATUMUMAB AND DEXAMETHASONE; MAINTENANCE TREATMENT OF ASTHMA IN PATIENTS AGED 18 YEARS AND OLDER; INDICATED IN PATIENTS 18 YEARS OF AGE AND OLDER FOR THE TREATMENT OF HOSPITAL ACQUIRED BACTERIAL PNEUMONIA AND VENTILATOR-ASSOCIATED BACTERIAL PNEUMONIA (HABP/VABP) CAUSED BY THE FOLLOWING SUSCEPTIBLE GRAM-NEGATIVE MICROORGANISMS, ACINETOBACTER BAUMANNII COMPLEX, ESCHERICHIA COLI, ENTEROBACTER CLOACAE COMPLEX, KLEBSIELLA PNEUMONIAE, PSEUDOMONAS AERUGINOSA, AND SERRATIA MARCESCENS; TREATMENT OF ADULT PATIENTS WITH ANAPLASTIC LYMPHOMA KINASE (ALK)-POSITIVE METASTATIC NON-SMALL CELL LUNG CANCER (NSCLC) AS DETECTED BY AN FDA-APPROVED TEST; TREATMENT OF CATAPLEXY IN ADULT PATIENTS WITH NARCOLEPSY; EXPANDED INDICATION OF THE TREATMENT OF ADULT PATIENTS WITH ANAPLASTIC LYMPHOMA KINASE (ALK)-POSITIVE METASTATIC NON-SMALL CELL LUNG CANCER (NSCLC) AS DETECTED BY AN FDA-APPROVED TEST; REDUCE THE RISK OF STROKE IN PATIENTS WITH ACUTE ISCHEMIC STROKE (NIH STROKE SCALE SCORE<=5) OR HIGH-RISK TRANSIENT ISCHEMIC ATTACK (TIA); CHRONIC PHASE (CP) CHRONIC MYELOID LEUKEMIA (CML) WITH RESISTANCE OR INTOLERANCE TO AT LEAST TWO PRIOR KINASE INHIBITORS; TREATMENT OF NIGHTTIME SLEEP DISTURBANCES IN SMITH-MAGENIS SYNDROME (SMS) IN PATIENTS 16 YEARS OF AGE AND OLDER; TO REDUCE THE RISK OF A FIRST MYOCARDIAL INFARCTION (MI) OR STROKE IN PATIENTS WITH CORONARY ARTERY DISEASE (CAD) AT HIGH RISK FOR SUCH EVENTS; TREATMENT OF PEDIATRIC PATIENTS 1 YEAR OF AGE AND OLDER AND YOUNG ADULTS WITH RELAPSED OR REFRACTORY, SYSTEMIC ANAPLASTIC LARGE CELL LYMPHOMA (ALCL) THAT IS ALK-POSITIVE; INDICATION OF OSIMERTINIB AS ADJUVANT THERAPY AFTER TUMOR RESECTION IN ADULT PATIENTS WITH NON-SMALL CELL LUNG CANCER (NSCLC) WHOSE TUMORS HAVE EPIDERMAL GROWTH FACTOR RECEPTOR (EGFR) EXON 19 DELETIONS OR EXON 21 L858R MUTATIONS, AS DETECTED BY AN FDA-APPROVED TEST; FOR THE TREATMENT OF PATIENTS WITH ADVANCED RENAL CELL CARCINOMA, AS A FIRST-LINE TREATMENT IN COMBINATION WITH NIVOLUMAB; TREATMENT OF NEUROGENIC DETRUSOR OVERACTIVITY (NDO) IN PEDIATRIC PATIENTS 3 YEARS AND OLDER AND WEIGHING 35 KILOGRAMS OR MORE; INDICATION FOR THE TREATMENT OF PULMONARY HYPERTENSION ASSOCIATED WITH INTERSTITIAL LUNG DISEASE TO IMPROVE EXERCISE ABILITY; TO REDUCE THE RISK OF SUSTAINED EGFR DECLINE, END-STAGE KIDNEY DISEASE, CARDIOVASCULAR DEATH, AND HOSPITALIZATION FOR HEART FAILURE IN ADULTS WITH CHRONIC KIDNEY DISEASE AT RISK OF PROGRESSION; FOR THE TOPICAL TREATMENT OF SCABIES INFESTATIONS IN ADULT AND PEDIATRIC PATIENTS 4 YEARS OF AGE AND OLDER; TREATMENT OF PATIENTS WITH TRANSFUSIONAL IRON OVERLOAD DUE TO SICKLE CELL DISEASE OR OTHER ANEMIAS; FOR THE TREATMENT OF MODERATELY TO SEVERELY ACTIVE ULCERATIVE COLITIS IN ADULT PATIENTS; TREATMENT OF NEUROGENIC DETRUSOR OVERACTIVITY (NDO) IN PEDIATRIC PATIENTS 6 YEARS OF AGE AND OLDER AND WEIGHING GREATER THAN 25 KG; TREATMENT OF VENOUS THROMBOEMBOLIC EVENTS (VTE) IN PEDIATRIC PATIENTS 8 TO LESS THAN 18 YEARS OF AGE WHO HAVE BEEN TREATED WITH A PARENTERAL ANTICOAGULANT FOR AT LEAST 5 DAYS AND TO REDUCE THE RISK OF RECURRENCE OF VTE IN PEDIATRIC PATIENTS 8 TO LESS THAN 18 YEARS OF AGE WHO HAVE BEEN PREVIOUSLY TREATED; TREATMENT OF ADULT PATIENTS WITH ADVANCED SYSTEMIC MASTOCYTOSIS (ADVSM), INCLUDING PATIENTS WITH AGGRESSIVE SYSTEMIC MASTOCYTOSIS (ASM) AND SYSTEMIC MASTOCYTOSIS WITH AN ASSOCIATED HEMATOLOGICAL NEOPLASM (SM-AHN); TREATMENT OF ADULT PATIENTS WITH MAST CELL LEUKEMIA (MCL); FOR THE PREVENTIVE TREATMENT OF EPISODIC MIGRAINE IN ADULTS; FOR THE TREATMENT OF TRICHOMONIASIS CAUSED BY TRICHOMONAS VAGINALIS IN ADULTS; INDICATED TO REDUCE THE RISK OF MAJOR THROMBOTIC VASCULAR EVENTS (MYOCARDIAL INFARCTION, ISCHEMIC STROKE, ACUTE LIMB ISCHEMIA, AND MAJOR AMPUTATION OF VASCULAR ETIOLOGY) IN PATIENTS WITH PAD, INCLUDING PATIENTS WHO HAVE RECENTLY UNDERGONE A LOWER EXTREMITY REVASCULARIZATION PROCEDURE DUE TO SYMPTOMATIC PAD; LENVATINIB IN COMBINATION WITH PEMBROLIZUMAB, IS INDICATED FOR THE FIRST-LINE TREATMENT OF ADULT PATIENTS WITH ADVANCED RENAL CELL CARCINOMA (RCC); REDUCE THE RISK OF CARDIOVASCULAR DEATH AND HOSPITALIZATION FOR HEART FAILURE IN ADULTS WITH HEART FAILURE AND REDUCED EJECTION FRACTION; INDICATED FOR THE TREATMENT OF IDIOPATHIC HYPERSOMNIA (IH) IN ADULTS; TREATMENT OF ADULT PATIENTS WITH WALDENSTROM'S MACROGLOBULINEMIA (WM); ADDITION OF THE INDICATION OF TREATMENT OF CHRONIC GRAFT-VERSUS-HOST DISEASE (CGVHD) AFTER FAILURE OF ONE OR TWO LINES OF SYSTEMIC THERAPY IN ADULT AND PEDIATRIC PATIENTS 12 YEARS AND OLDER; TREATMENT OF ADULT AND PEDIATRIC PATIENTS 12 YEARS OF AGE AND OLDER WITH LOCALLY ADVANCED OR METASTATIC DIFFERENTIATED THYROID CANCER (DTC) THAT HAS PROGRESSED FOLLOWING PRIOR VEGFR-TARGETED THERAPY AND WHO ARE RADIOACTIVE IODINE-REFRACTORY OR INELIGIBLE; TREATMENT OF ADULT PATIENTS WITH RELAPSED OR REFRACTORY MARGINAL ZONE LYMPHOMA (MZL) WHO HAVE RECEIVED AT LEAST ONE ANTI-CD20-BASED REGIMEN; FOR THE TREATMENT OF ADULT PATIENTS WITH PREVIOUSLY TREATED, LOCALLY ADVANCED OR METASTATIC CHOLANGIOCARCINOMA WITH AN IDH1 MUTATION AS DETECTED BY AN FDA-APPROVED TEST; TREATMENT OF OCULAR ITCHING ASSOCIATED WITH ALLERGIC CONJUNCTIVITIS; INDICATION FOR THE USE OF ABEMACICLIB IN COMBINATION WITH ENDOCRINE THERAPY (TAMOXIFEN OR AN AROMATASE INHIBITOR) FOR THE ADJUVANT TREATMENT OF ADULT PATIENTS WITH HORMONE RECEPTOR (HR)-POSITIVE, HUMAN EPIDERMAL GROWTH FACTOR RECEPTOR 2 (HER2)-NEGATIVE, NODE-POSITIVE, EARLY CANCER (EBC) AT HIGH RISK OF RECURRENCE AND A KI-67 SCORE>20% AS DETERMINED BY AN FDA APPROVED TEST; ADDITION OF A NEW INDICATION FOR ADJUNCTIVE THERAPY IN THE TREATMENT OF PRIMARY GENERALIZED TONIC-CLONIC SEIZURES IN PATIENTS 4 YEARS OF AGE AND OLDER; TREATMENT OF ADULT PATIENTS WITH ACTIVE ANKYLOSING SPONDYLITIS WHO HAVE HAD AN INADEQUATE RESPONSE OR INTOLERANCE TO ONE OR MORE TNF BLOCKERS, TO THE PRESCRIBING INFORMATION; TREATMENT OF ADULTS WITH ACTIVE PSORIATIC ARTHRITIS WHO HAVE HAD AN INADEQUATE RESPONSE OR INTOLERANCE TO ONE OR MORE TNF BLOCKERS; FOR THE TREATMENT OF INVASIVE ASPERGILLOSIS IN PATIENTS 13 YEARS OF AGE AND OLDER; INDICATED FOR DEPRESSIVE EPISODES ASSOCIATED WITH BIPOLAR I OR II DISORDER (BIPOLAR DEPRESSION) IN ADULTS, AS MONOTHERAPY AND AS ADJUNCTIVE THERAPY WITH LITHIUM OR VALPROATE; TREATMENT OF ADULTS AND PEDIATRIC PATIENTS 12 YEARS OF AGE AND OLDER WITH REFRACTORY, MODERATE-TO-SEVERE ATOPIC DERMATITIS WHOSE DISEASE IS NOT ADEQUATELY CONTROLLED WITH OTHER SYSTEMIC DRUG PRODUCTS, INCLUDING BIOLOGICS, OR WHEN USE OF THOSE THERAPIES ARE INADVISABLE; REVISIONS TO THE LABELING TO INCLUDE DATA FOR SUBJECTS WITH MILD TO MODERATE PLAQUE PSORIASIS, AND TO ALLOW FOR AN EXPANSION OF THE INDICATION; FOR THE ADJUVANT TREATMENT OF ADULT PATIENTS WITH DELETERIOUS OR SUSPECTED DELETERIOUS GBRCA MUTATED HER2-NEGATIVE HIGH-RISK EARLY BREAST CANCER WHO HAVE PREVIOUSLY BEEN TREATED WITH NEOADJUVANT OR ADJUVANT CHEMOTHERAPY; TREATMENT OF ADULT PATIENTS WITH MODERATELY TO SEVERELY ACTIVE ULCERATIVE COLITIS WHO HAVE HAD AN INADEQUATE RESPONSE OR INTOLERANCE TO ONE OR MORE TNF BLOCKERS; INDICATION FOR THE TREATMENT OF SEIZURES ASSOCIATED WITH LENNOX-GASTAUT SYNDROME (LGS) IN PATIENTS WHO ARE 2 YEARS OF AGE AND OLDER; TREATMENT OF ADULTS WITH ACTIVE ANKYLOSING SPONDYLITIS WHO HAVE HAD AN INADEQUATE RESPONSE OR INTOLERANCE TO ONE OR MORE TNF BLOCKERS; TREATMENT OF PEDIATRIC PATIENTS AGED ONE MONTH AND OLDER WITH NEWLY DIAGNOSED JUVENILE MYELOMONOCYTIC LEUKEMIA (JMML); TREATMENT OF ADULT PATIENTS WITH SEVERE ALOPECIA AREATA; TREATMENT OF COVID-19 IN HOSPITALIZED ADULTS REQUIRING SUPPLEMENTAL OXYGEN, NONINVASIVE OR INVASIVE MECHANICAL VENTILATION, OR EXTRACORPOREAL MEMBRANE OXYGENATION (ECMO); CHRONIC WEIGHT MANAGEMENT IN ADULT AND PEDIATRIC PATIENTS 6 YEARS OF AGE AND OLDER WITH BARDET-BIEDL SYNDROME (BBS); IN COMBINATION WITH AZACITIDINE OR AS MONOTHERAPY FOR THE TREATMENT OF NEWLY DIAGNOSED ACUTE MYELOID LEUKEMIA (AML) WITH A SUSCEPTIBLE ISOCITRATE DEHYDROGENASE-1 (IDH1) MUTATION AS DETECTED BY AN FDA-APPROVED TEST IN ADULTS 75 YEARS OR OLDER, OR WHO HAVE COMORBIDITIES THAT PRECLUDE USE OF INTENSIVE INDUCTION CHEMOTHERAPY; DABRAFENIB IS INDICATED IN COMBINATION WITH TRAMETINIB, FOR THE TREATMENT OF ADULT AND PEDIATRIC PATIENTS 6 YEARS OF AGE AND OLDER WITH UNRESECTABLE OR METASTATIC SOLID TUMORS WITH BRAF V600E MUTATION WHO HAVE PROGRESSED FOLLOWING PRIOR TREATMENT AND HAVE NO SATISFACTORY ALTERNATIVE TREATMENT OPTIONS; TRAMETINIB IS INDICATED IN COMBINATION WITH DABRAFENIB, FOR THE TREATMENT OF ADULT AND PEDIATRIC PATIENTS 6 YEARS OF AGE AND OLDER WITH UNRESECTABLE OR METASTATIC SOLID TUMORS WITH BRAF V600E MUTATION WHO HAVE PROGRESSED FOLLOWING PRIOR TREATMENT AND HAVE NO SATISFACTORY ALTERNATIVE TREATMENT OPTIONS; INDICATED FOR THE TOPICAL TREATMENT OF NONSEGMENTAL VITILIGO IN ADULT AND PEDIATRIC PATIENTS 12 YEARS OF AGE AND OLDER; TREATMENT OF ADULT AND PEDIATRIC PATIENTS 1 YEAR OF AGE AND OLDER WITH UNRESECTABLE, RECURRENT OR REFRACTORY INFLAMMATORY MYOFIBROBLASTIC TUMOR (IMT) THAT IS ANAPLASTIC LYMPHOMA KINASE (ALK) POSITIVE; FOR THE MANAGEMENT OF MODERATE TO SEVERE PAIN ASSOCIATED WITH ENDOMETRIOSIS; TREATMENT OF ADULTS WITH RELAPSED OR REFRACTORY MYELOID/LYMPHOID NEOPLASMS (MLNS) WITH FIBROBLAST GROWTH FACTOR RECEPTOR 1 (FGFR1) REARRANGEMENT; TREATMENT OF ADULT PATIENTS WITH METASTATIC HORMONE-SENSITIVE PROSTATE CANCER (MHSPC) IN COMBINATION WITH DOCETAXEL; EXPANDED INDICATION TO INCLUDE LOWERING OF PLASMA OXALATE LEVELS IN ADULT AND PEDIATRIC PATIENTS WITH PRIMARY HYPEROXALURIA TYPE 1 (PHI); TREATMENT OF ADULT PATIENTS WITH HISTIOCYTIC NEOPLASMS; REDUCTION IN THE INCIDENCE OF RECURRENT VULVOVAGINAL CANDIDIASIS (RVVC) IN ADULT AND POST-MENARCHAL PEDIATRIC FEMALES; ADJUNCTIVE THERAPY TO ANTIDEPRESSANTS FOR THE TREATMENT OF MAJOR DEPRESSIVE DISORDER IN ADULTS; ADJUNCT FOR INTRAOPERATIVE IDENTIFICATION OF MALIGNANT AND NONMALIGNANT PULMONARY LESIONS IN ADULT PATIENTS WITH KNOWN OR SUSPECTED CANCER IN THE LUNG; TUCATINIB IN COMBINATION WITH TRASTUZUMAB FOR THE TREATMENT OF ADULT PATIENTS WITH RAS WILD-TYPE, HER2-POSITIVE, UNRESECTABLE OR METASTATIC COLORECTAL CANCER THAT HAS PROGRESSED FOLLOWING TREATMENT WITH FLUOROPYRIMIDINE-, OXALIPLATIN-, AND IRINOTECAN-BASED CHEMOTHERAPY; TO INCREASE BONE DENSITY IN MEN WITH OSTEOPOROSIS AT HIGH RISK FOR FRACTURE (DEFINED AS A HISTORY OF OSTEOPOROTIC FRACTURE OR MULTIPLE RISK FACTORS FOR FRACTURE), OR PATIENTS WHO HAVE FAILED OR ARE INTOLERANT TO OTHER AVAILABLE OSTEOPOROSIS THERAPY; TREATMENT OF PEDIATRIC PATIENTS 1 YEAR OF AGE AND OLDER WITH LOW-GRADE GLIOMA WITH A BRAF V600 MUTATION WHO REQUIRE SYSTEMIC THERAPY; PREVENTIVE TREATMENT OF MIGRAINE IN ADULTS; TREATMENT OF PEDIATRIC PATIENTS 1 YEAR OF AGE AND OLDER WITH LOW-GRADE GLIOMA WITH A BRAF V600E MUTATION WHO REQUIRE SYSTEMIC THERAPY; FOR FLUORESCENCE IMAGING OF LYMPH NODES AND DELINEATION OF LYMPHATIC VESSELS DURINGLYMPHATIC MAPPING IN ADULTS WITH BREAST CANCER FOR WHICH THIS PROCEDURE IS A COMPONENT OF INTRAOPERATIVEMANAGEMENT; TREATMENT OF ADULT PATIENTS WITH INDOLENT SYSTEMIC MASTOCYTOSIS (ISM); TREATMENT OF AGITATION ASSOCIATED WITH DEMENTIA DUE TO ALZHEIMER'S DISEASE; IN COMBINATION WITH ABIRATERONE AND PREDNISONE OR PREDNISOLONE FOR THETREATMENT OF ADULT PATIENTS WITH DELETERIOUS OR SUSPECTED DELETERIOUS BRCA-MUTATED (BRCAM) METASTATIC CASTRATION-RESISTANT PROSTATE CANCER (MCRPC); TREATMENT OF IRON DEFICIENCY IN ADULT PATIENTS WITH HEART FAILURE AND NEW YORK HEARTASSOCIATION CLASS II/III TO IMPROVE EXERCISE CAPACITY; PROPHYLAXIS OF CYTOMEGALOVIRUS (CMV) DISEASE IN ADULT KIDNEY TRANSPLANT RECIPIENTS AT HIGH RISK (DONOR CMV SEROPOSITIVE/RECIPIENT CMV SERONEGATIVE [D+/R-); TREATMENT OF HEAVY MENSTRUAL BLEEDING FOR UP TO 5 YEARS IN PATIENTS WHO CHOOSE INTRAUTERINE CONTRACEPTION AS THEIR METHOD OF CONTRACEPTION; TREATMENT OF CHOLESTATIC PRURITUS IN PATIENTS 12 MONTHS OF AGE AND OLDER WITH ALAGILLE SYNDROME (ALGS); TREATMENT OF ADULT PATIENTS WITH MODERATELY TO SEVERELY ACTIVE CROHN'S DISEASE WHO HAVE HAD AN INADEQUATE RESPONSE OR INTOLERANCE TO ONE OR MORE TNF BLOCKERS; USE OF TALAZOPARIB IN COMBINATION WITH ENZALUTAMIDE FOR THE TREATMENT OF ADULT PATIENTS WITHHOMOLOGOUS RECOMBINATION REPAIR (HRR) GENE-MUTATED METASTATIC CASTRATION-RESISTANT PROSTATE CANCER (MCRPC); TREATMENT OF FUNCTIONAL CONSTIPATION IN PEDIATRIC PATIENTS 6 TO 17 YEARS OF AGE; USE OF EMPAGLIFLOZIN TO REDUCE THE RISK OF SUSTAINED DECLINE IN EGFR, END-STAGE KIDNEY DISEASE, CARDIOVASCULAR DEATH, AND HOSPITALIZATION IN ADULTS WITH CHRONIC KIDNEY DISEASE AT RISK OF PROGRESSION; FOR THE TREATMENT OF PEDIATRIC PATIENTS 1 YEAR OF AGE AND OLDER WITH CHRONIC PHASE (CP) PHILADELPHIA CHROMOSOME-POSITIVE CHRONIC MYELOGENOUS LEUKEMIA (PH+CML), NEWLY-DIAGNOSED OR RESISTANT OR INTOLERANT TO PRIOR THERAPY; FOR THE TREATMENT OF ADULT PATIENTS WITH RELAPSED OR REFRACTORY MYELODYSPLASTIC SYNDROMES (MDS) WITH A SUSCEPTIBLE ISOCITRATE DEHYDROGENASE-1 (IDH1) MUTATION AS DETECTED BY AN FDA-APPROVED TEST; TREATMENT OF ADULTS WITH CHOREA ASSOCIATED WITH HUNTINGTON'S DISEASE; TREATMENT OF PATIENTS WITH NON-METASTATIC CASTRATION-SENSITIVE PROSTATE CANCER (NMCSPC) WITH BIOCHEMICAL RECURRENCE AT HIGH RISK FOR METASTASIS; or ENCORAFENIB IN COMBINATION WITH BINIMETINIB, IS INDICATED FOR THE TREATMENT OF ADULT PATIENTS WITH METASTATIC NON-SMALL CELL LUNG CANCER (NSCLC) WITH BRAF V600E MUTATION, AS DETECTED BY AN FDA-APPROVED TEST.
